SEQUENCE LISTING (76 sequences)

SEQ ID NO: 1 - DNA, 3888 bp, synthetic construct
  misc_feature 1..3888: note = Nucleotide Sequence of rBoNT/A(0)
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatcaactgatctacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, synthetic construct
  REGION 1..1296: note = Polypeptide Sequence of rBoNT/A(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 2634 bp, synthetic construct
  misc_feature 1..2634: note = Nucleotide Sequence of rLHN/A
  source 1..2634: mol_type = other DNA
atggagttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctgacgatgacgataaaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaagcacaccatcatcaccaccatcaccatcaccat

SEQ ID NO: 4 - protein, 878 aa, synthetic construct
  REGION 1..878: note = Polypeptide Sequence of rLHN/A
MEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSDDDDKNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEAHHHHHHHHHH

SEQ ID NO: 5 - DNA, 1329 bp, synthetic construct
  misc_feature 1..1329: note = Nucleotide Sequence of rL/A
  source 1..1329: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgggtctagaagcacaccatcatcaccaccatcaccatcaccat

SEQ ID NO: 6 - protein, 443 aa, synthetic construct
  REGION 1..443: note = Polypeptide Sequence of rL/A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLGLEAHHHHHHHHHH

SEQ ID NO: 7 - DNA, 1299 bp, synthetic construct
  misc_feature 1..1299: note = Nucleotide Sequence of rHC/A
  source 1..1299: mol_type = other DNA
atgcatcatcaccatcaccacaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 8 - protein, 433 aa, synthetic construct
  REGION 1..433: note = Polypeptide Sequence of rHC/A
MHHHHHHKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 9 - DNA, 3873 bp, synthetic construct
  misc_feature 1..3873: note = Nucleotide Sequence of rBoNT/B(0)
  source 1..3873: mol_type = other DNA
atgccggtgacgattaacaacttcaactacaacgacccgattgacaacaacaacattatcatgatggaaccgccgtttgcacgcggcacgggccgttattacaaagcgtttaaaatcaccgatcgtatttggattatcccggaacgctacacgtttggttataaaccggaagacttcaacaaaagctctggcatcttcaaccgtgatgtttgcgaatactacgatccggactacctgaacaccaacgataagaaaaacatttttctgcaaacgatgatcaaactgttcaatcgcattaaaagcaaaccgctgggtgaaaaactgctggaaatgattatcaatggcattccgtatctgggtgatcgtcgcgtgccgctggaagaatttaacaccaatatcgcgagtgttacggtcaacaaactgatttccaatccgggtgaagtcgaacgtaaaaaaggcatcttcgccaacctgatcatcttcggcccgggtccggtgctgaacgaaaatgaaaccattgatatcggtattcagaaccattttgcctcacgcgaaggcttcggcggtattatgcaaatgaaattttgcccggaatatgtgtcggttttcaacaatgttcaggaaaacaaaggtgcaagcatctttaatcgtcgcggctatttctctgatccggctctgatcctgatgcaccaactgatttatgtgctgcacggcctgtatggtatcaaagtggatgacctgccgatcgttccgaacgagaaaaaatttttcatgcagagcaccgacgcaattcaagctgaagaactgtatacgtttggcggtcaggacccgtctattatcaccccgagcaccgacaaaagcatctacgataaagtgctgcaaaactttcgtggcattgttgaccgcctgaataaagtcctggtgtgtatctctgatccgaacatcaacatcaacatctacaaaaacaaattcaaagacaaatacaaattcgttgaagattctgaaggcaaatatagtattgacgtcgaatcctttgataaactgtacaaaagtctgatgttcggtttcaccgaaacgaacatcgcggaaaactacaaaatcaaaacccgcgcctcctatttcagcgactctctgccgccggttaaaatcaaaaatctgctggataacgaaatttatacgatcgaagaaggtttcaacatcagcgataaagacatggaaaaagaataccgtggccagaataaagcaatcaacaaacaggcgtatgaagaaattagtaaagaacatctggcggtctacaaaattcagatgtgcaaatccgtgaaagccccgggtatttgtatcgatgttgacaatgaagacctgtttttcatcgccgataaaaacagtttttccgatgacctgtcaaaaaatgaacgcatcgaatacaacacccaatcgaactacatcgaaaacgatttcccgatcaacgaactgattctggatacggacctgattagtaaaatcgaactgccgtcagaaaacaccgaatcgctgacggactttaatgttgatgtcccggtgtatgaaaaacagccggcaattaagaaaatttttaccgatgaaaacacgatcttccagtacctgtacagccaaacctttccgctggacattcgcgatatctctctgacgagttcctttgatgacgcactgctgttcagcaacaaagtgtactcctttttctcaatggattacatcaaaaccgctaacaaagtggttgaagcgggcctgtttgccggttgggtgaaacagatcgttaacgatttcgtcatcgaagccaacaaaagtaacacgatggataaaattgctgatatctccctgattgtcccgtatattggcctggcactgaatgtgggtaacgaaacggcgaaaggcaattttgaaaacgccttcgaaattgcaggcgcttcaatcctgctggaatttattccggaactgctgatcccggtcgtgggtgcgttcctgctggaatcttacatcgacaacaaaaacaaaatcatcaaaaccattgataacgcgctgacgaaacgtaacgaaaaatggtcagatatgtacggcctgattgttgcccagtggctgagcaccgtcaacacgcaattttacaccatcaaagaaggtatgtacaaagcgctgaattatcaggcgcaagccctggaagaaatcatcaaataccgctacaacatctacagcgaaaaagaaaaatctaacatcaacatcgactttaatgatatcaacagcaaactgaacgaaggtatcaaccaggcaatcgataacatcaacaacttcatcaacggctgctcagtgtcgtatctgatgaagaaaatgatcccgctggctgttgaaaaactgctggattttgacaacaccctgaagaaaaacctgctgaactacatcgatgaaaacaaactgtacctgatcggctcagccgaatacgaaaaatcgaaagtgaacaaatacctgaaaaccatcatgccgtttgacctgagtatttacaccaacgatacgatcctgatcgaaatgttcaacaaatacaactccgaaattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaagagaaactgttcctggccccgattagcgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaa

SEQ ID NO: 10 - protein, 1291 aa, synthetic construct
  REGION 1..1291: note = Polypeptide Sequence of rBoNT/B(0)
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 11 - DNA, 3873 bp, synthetic construct
  misc_feature 1..3873: note = Nucleotide Sequence of rBoNT/C(0)
  source 1..3873: mol_type = other DNA
atgccgatcacgattaataatttcaactatagcgatccggtggacaataagaatattctgtatctggatactcatctgaatacgctggctaacgaaccggagaaagcgttccgcatcacaggcaacatctgggttattcccgatcgcttttcacgcaacagcaaccctaatctgaacaaacctcctcgtgtcaccagtcctaaatccggttattacgacccaaactatctgagtacggatagcgataaagatccctttctgaaagagatcattaagctgttcaaacgcattaactctcgcgaaattggggaagagctgatctatcggctttcgacagatatcccgttcccaggtaacaataataccccgattaatactttcgactttgatgttgatttcaattctgtggatgtgaaaacgcgtcaaggcaataattgggtgaaaactggtagcattaacccgagtgtaattatcacaggtccccgtgagaacatcatcgacccggaaacctctaccttcaagctgacgaacaacacgtttgctgcacaggaagggtttggtgccctgtcaatcatttccatctcaccgcgtttcatgttaacctactccaatgccacaaatgatgttggcgaaggacgttttagcaaatcagaattttgcatggacccaattctcattctgatgggcacgctgaacaatgcgatgcacaacttgtatggcattgctattccaaacgatcaaaccattagctccgttaccagtaatatcttctatagccagtataatgtcaaattggagtatgccgaaatttacgcctttggaggcccgaccattgacctgattccgaaatctgcacgcaaatacttcgaagaaaaggcgttagattactatcgcagcatcgcgaaacgcctgaactcgattaccacggccaatccgtcgtcgttcaacaaatacattggtgaatataaacagaaactgattcgcaaatatcggtttgtcgtagaaagctctggtgaagtgactgtaaaccgcaacaaatttgtcgaactctacaacgagttgacccaaatctttaccgagtttaactacgcaaagatctataacgtacagaaccgcaagatttatcttagcaatgtatacacaccggttactgcgaacatcttagacgacaatgtgtatgatattcagaatggctttaacatcccgaaatcaaatctgaacgttctgtttatgggccagaacctgagtcgtaatccagcactgcgtaaagtgaacccggaaaatatgctctacttgtttaccaaattttgccacaaagcgattgatggccgctctctctataacaaaacgctggattgtcgtgagttacttgtgaagaacactgatttaccgttcattggggatatctccgacgtgaaaaccgatatcttcctgcgcaaagacattaatgaagaaacggaagtcatctattaccccgacaatgtgagcgttgatcaggtcattttatcgaagaacacctccgaacatggtcagttggatttgctgtaccctagcattgactcggagagtgaaatccttccgggcgaaaatcaagtgttttacgacaaccgtacccaaaatgttgattatttgaattcttattactacctggaatctcagaaattgagcgacaatgtggaagatttcacgttcacacgctccattgaggaagcgctggataatagcgcgaaagtgtatacgtatttccctaccttggcgaataaagtaaatgctggtgtccagggaggcttatttctgatgtgggcgaatgatgtggtagaagattttacgaccaatattttgcgtaaggacaccttagataaaattagcgatgttagcgccatcatcccctatattggcccagcactgaatatctcgaactctgtgcgtcgcggaaacttcaccgaagcatttgcggtgaccggggttactattctgttggaagcctttccggagtttactattccggcgctgggtgcgtttgtgatttattcgaaagtacaagaacgcaatgaaattatcaaaaccatcgataattgcctggaacaacgcattaaacgctggaaggattcttatgaatggatgatgggcacctggttatcccgtattatcacacagtttaacaacatctcgtatcagatgtacgattcactgaactaccaagcaggggcgatcaaagccaagatcgacttagaatacaagaaatattcaggtagcgataaagagaatattaaaagccaggttgaaaacctgaagaactctctggatgtcaaaatttcagaggctatgaacaacattaacaaatttatccgcgaatgtagcgtcacgtatctgtttaaaaacatgctcccgaaagtgattgatgagctcaacgagtttgatcgcaacacaaaggccaaactgattaacctgattgatagtcacaatattattttagtcggtgaagttgacaagctgaaggctaaggtcaataacagctttcagaacactattccgtttaatattttctcctatacgaacaatagtctgctgaaagacattatcaacgaatacttcaacaatattaatgacagcaaaattctgagcctgcagaatcgtaagaatacgctggtagataccagtggatataatgcggaagtctcagaagagggtgatgtacagctgaacccgatctttccgttcgactttaaactggggtctagtggtgaagatcgcggtaaagtgatcgttacccaaaacgagaacattgtgtataacagcatgtacgagagtttctcaatttctttctggattcgcatcaataaatgggtttctaatttgcctggctataccatcattgatagcgtcaaaaacaactcgggctggtcgattggcattattagcaactttctggtgtttaccctgaaacagaatgaggattcggaacagagcattaacttctcctacgacatcagcaacaatgcaccagggtataacaaatggttcttcgtaacggtgacgaacaatatgatgggcaatatgaaaatctacattaacgggaaacttatcgacaccattaaagtgaaagagcttactgggatcaattttagtaaaaccattacctttgagatcaacaaaattccggacacgggtctgattacctccgattcggataatatcaatatgtggattcgcgacttttatatcttcgccaaagaacttgatggcaaagatatcaacattttgtttaattccctgcagtataccaatgtcgttaaggactattggggcaatgatctccgctacaataaagaatactacatggttaacatcgactatctcaatcgctacatgtatgctaactcgcgtcaaattgtgtttaacacacgtcgtaacaacaacgattttaacgaaggttataaaatcattatcaaacggatccgcggcaatacgaacgatactcgtgttcgtggcggtgacattctgtatttcgacatgacgattaataataaagcgtacaatctgttcatgaagaacgaaaccatgtacgccgataaccattccactgaagatatctacgcaatcggacttcgcgaacagaccaaagacattaacgacaacatcatctttcagattcaaccgatgaataatacctactactatgcctcccagatcttcaaaagtaatttcaacggcgaaaacatttcaggcatttgctcaatcggcacttatcggttccggttaggtggtgattggtatcgtcacaactaccttgttcccacagtgaaacaaggcaactatgcatcgctcttagaaagcacatctacgcattggggttttgtgccagtcagtgaa

SEQ ID NO: 12 - protein, 1291 aa, synthetic construct
  REGION 1..1291: note = Polypeptide Sequence of rBoNT/C(0)
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMGTLNNAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 13 - DNA, 3756 bp, synthetic construct
  misc_feature 1..3756: note = Nucleotide Sequence of rBoNT/E(0)
  source 1..3756: mol_type = other DNA
atgccgaaaatcaactctttcaactacaacgacccggttaacgaccgtaccatcctgtatatcaaaccgggtggttgccaggagttctacaaatctttcaacatcatgaaaaacatctggatcatcccggaacgtaacgttatcggtaccaccccgcaggacttccacccgccgacctctctgaaaaacggtgactcttcttactacgacccgaactacctccagtctgacgaagaaaaagaccgtttcctgaaaatcgttaccaaaatcttcaaccgtatcaacaacaacctgtctggtggtatcctgctggaagaactgtctaaagctaacccgtacctgggtaacgacaacaccccggacaaccagttccacatcggtgacgcttctgctgttgaaatcaaattctctaacggttctcaggacatcctgctgccgaacgttatcatcatgggtgctgaaccggacctgttcgaaaccaactcttctaacatctctctgcgtaacaactacatgccgtctaaccacggtttcggttctatcgctatcgttaccttctctccggaatactctttccgtttcaacgacaacagcatgaacgagttcatccaggacccggctctgaccctgatgcaccaactgatctactctctgcacggtctgtacggtgctaaaggtatcaccaccaaatacaccatcacccagaaacagaacccgctgatcaccaacatccgtggtaccaacatcgaagagttcctgaccttcggtggtaccgacctgaacatcatcacctctgctcagtctaacgacatctacaccaacctgctggctgactacaaaaaaatcgcttctaaactgtctaaagttcaggtttctaacccgctgctgaacccgtacaaagacgttttcgaagctaaatacggtctggacaaagacgcttctggtatctactctgttaacatcaacaaattcaacgacatcttcaaaaaactgtactctttcaccgagttcgacctggcgaccaaattccaggttaaatgccgtcagacctacatcggtcagtacaaatacttcaaactgtctaacctgctgaacgactctatctacaacatctctgaaggttacaacatcaacaacctgaaagttaacttccgtggtcagaacgctaacctgaacccgcgtatcatcaccccgatcaccggtcgtggtctggttaaaaaaatcatccgtttctgcaagaatattgtaagcgttaaaggaataagaaaaagtatctgcatcgaaatcaacaacggtgaactgttcttcgttgcttctgaaaactcttacaacgacgacaacatcaacaccccgaaagaaatcgacgacaccgttacctctaacaacaactacgaaaacgacctggaccaggttatcctgaacttcaactctgaatctgctccgggtctgtctgacgaaaaactgaacctgaccatccagaacgacgcttacatcccgaaatacgactctaacggtacctctgacatcgaacagcacgacgttaacgaactgaacgttttcttctacctggacgctcagaaagttccggaaggtgaaaacaacgttaacctgacctcttctatcgacaccgctctgctggaacagccgaaaatctacaccttcttctcttctgagttcatcaacaacgttaacaaaccggttcaggctgctctgttcgtttcttggattcagcaggttctggttgacttcaccaccgaagctaaccagaaatctaccgttgacaaaatcgctgacatctctatcgttgttccgtacatcggtctggctctgaacatcggtaacgaagctcagaaaggtaacttcaaagacgctctggaactgctgggtgctggtatcctgctggagttcgaaccggaactgctgatcccgaccatcctggttttcaccatcaaatctttcctgggttcttctgacaacaaaaacaaagttatcaaagctatcaacaacgctctgaaagaacgtgacgaaaaatggaaagaagtttactctttcatcgtttctaactggatgaccaaaatcaacacccagttcaacaaacgtaaagaacagatgtaccaggctctccagaaccaggttaacgctatcaaaaccatcatcgaatctaaatacaactcttacaccctggaagaaaaaaacgaactgaccaacaaatacgacatcaaacagatcgaaaacgaactgaaccagaaagtttctatcgctatgaacaacatcgaccgtttcctgaccgaatcttctatctcttacctgatgaaactcatcaacgaagttaaaatcaacaaactgcgtgaatacgacgaaaacgttaaaacctacctgctgaactacatcatccagcacggttctatcctgggtgaatctcagcaggaactgaactctatggttaccgacaccctgaacaactctatcccgttcaaactgtcttcttacaccgacgacaaaatcctgatctcttacttcaacaaattctttaaacgcattaagagttcatcggttctgaatatgcggtacaaaaatgataaatatgtcgatacttctggatatgatagcaatatcaacattaacggcgacgtgtataaatatccgacaaataaaaaccagtttgggatatataacgacaagctgtcggaggtcaatatttctcaaaacgactatatcatttacgataataaatataaaaactttagcattagtttttgggttcgtatacctaattatgacaataaaattgtaaatgtgaataacgagtataccattataaactgtatgcgcgacaataacagtggttggaaggtatcgctgaaccataatgagattatctggaccctgcaggataatgcaggtataaaccagaaactggcttttaactatggaaacgcaaatgggatctcagattacattaataaatggatttttgttaccattacgaacgatcgcttaggcgactcaaaactttatattaatggcaatctgatagatcagaaatcaatcttaaatttgggcaatattcatgtctctgataacatcttgttcaagatcgttaattgcagttacactcgttatattggcattcgttactttaatatcttcgataaagaactggacgagacggaaatccagactctgtattcaaacgagcccaatactaatatattgaaagatttttggggtaactatcttttatatgataaagaatactatctcctgaatgtattgaagccaaacaatttcatagatagacgcaaggatagcacattaagtatcaacaatatcagatctactatactgttagcaaatcgcctctactccggtattaaagtgaagattcagcgggttaataactccagtaccaatgataatctggtccgtaagaacgatcaggtatacatcaatttcgtcgcgagcaaaactcatctcttcccgctttacgccgatacagctacgacaaacaaggaaaaaaccataaaaatttccagctccggaaacagattcaatcaagtagttgtaatgaactctgtgggtaataattgtacgatgaactttaagaataacaatgggaacaatattggacttttgggcttcaaagccgacacagtggtggcgtccacctggtattacacgcacatgcgggaccatacgaattcgaacggttgcttctggaactttatctcggaagaacacgggtggcaagaaaaa

SEQ ID NO: 14 - protein, 1252 aa, synthetic construct
  REGION 1..1252: note = Polypeptide Sequence of rBoNT/E(0)
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHQLIYSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 15 - DNA, 3834 bp, synthetic construct
  misc_feature 1..3834: note = Nucleotide Sequence of rBoNT/F(0)
  source 1..3834: mol_type = other DNA
atgccggtggtcatcaacagcttcaactacaacgacccagtaaacgacgacacgatcctgtatatgcaaatcccgtatgaagagaagagcaagaagtactataaggcctttgaaatcatgcgcaatgtgtggattattccggagcgtaatacgattggtactgacccaagcgacttcgatccacctgcgtctttggaaaacggctcgtccgcatattacgacccgaattacctgaccaccgatgcggagaaagatcgttatttgaaaaccaccatcaagctgttcaaacgcattaacagcaatccggcaggtgaggtcctgctgcaagagattagctacgcaaagccttatctgggtaatgagcatacgcctattaacgagtttcacccggttacccgcactaccagcgttaacatcaagtcctcgaccaacgtgaagtctagcattatcctgaacctgctggttctgggtgccggtccggacatcttcgaaaactctagctacccggtgcgtaaactgatggatagcggcggtgtttatgacccgagcaatgacggttttggcagcatcaatatcgtgacgtttagcccggagtacgagtacaccttcaatgatatcagcggtggttacaattcttctaccgagagcttcatcgccgacccggcgatcagcctggcacaccaactgatctatgcattgcatggcttgtacggtgcccgtggtgtgacgtataaagagactatcaaggttaagcaggcacctctgatgattgcggaaaagccgattcgcctggaagagttcctgaccttcggcggtcaagatttgaacatcattacctcggccatgaaagagaaaatctataacaatttgctggccaactatgaaaagattgcaacgcgcttgtctcgtgttaactccgctccgccggaatacgacattaatgagtacaaagactactttcaatggaaatatggcctggacaaaaatgcggatggttcttataccgtgaatgaaaacaaattcaatgaaatctacaagaaactgtacagcttcaccgaaatcgatctggcgaacaagttcaaagtcaaatgtcgtaatacctacttcatcaaatatggcttcctgaaagtcccgaacctgctggacgatgacatctataccgtcagcgaaggcttcaacatcggcaatctggccgtgaataatcgtggtcagaacatcaaactgaatccgaaaatcattgactccatcccagacaagggcctggttgagaaaatcgtgaagttctgcaaaagcgttattccgcgtaaaggtacgaaagcaccgcctcgcctgtgcattcgcgttaacaaccgtgagttgttctttgtggcatctgaaagcagctacaacgagaacgacatcaacacccctaaagaaattgatgataccacgaacctgaataacaattatcgcaacaatctggacgaggtgatcctggattacaattcggaaaccattccgcaaattagcaatcagacgctgaacaccctggttcaggacgatagctacgttccgcgttacgactccaatggtactagcgagattgaagaacacaacgtagtggacttgaacgttttcttttatctgcacgcccagaaggttccggagggcgaaaccaatattagcctgaccagctcgatcgacaccgcgctgtctgaggagagccaagtctacacctttttcagcagcgagtttatcaacactattaacaagccagttcatgctgcattgtttatctcttggattaaccaggtgattcgcgactttacgacggaggcgacccagaagtctaccttcgacaaaattgcagacatctccctggtcgtcccatacgtcggcctggcgttgaatattggcaatgaagttcaaaaagagaacttcaaagaagcgttcgagctgctgggtgcaggcatcctgctggagttcgtgccggaactgttgatcccgaccatcctggtgttcaccattaagagcttcattggatcctccgagaataagaacaagatcatcaaggcgatcaataacagcctgatggagcgtgaaacgaagtggaaagaaatctatagctggattgttagcaattggctgactcgtattaacacgcaattcaacaagcgtaaagagcaaatgtaccaagccctgcaaaaccaagttgacgccatcaaaacggtaattgaatacaagtacaacaattacacgagcgatgagcgcaaccgcctggaaagcgaatacaacatcaacaacattcgcgaagaattgaacaagaaagtgagcctggcgatggagaacattgagcgttttatcaccgaaagcagcatcttttacctgatgaaattgattaatgaggcgaaagtctcgaaactgcgtgagtacgacgaaggtgtgaaagagtatctgctggattacattagcgagcaccgtagcatcttgggtaactcggttcaggagctgaacgatctggtgacctctaccctgaacaatagcatcccgttcgaactgagcagctataccaatgacaagattctgattctgtatttcaataaactgtataagaagatcaaggataacagcattctggatatgcgttacgaaaacaataagtttatcgacatttctggttacggcagcaacatttccatcaatggcgatgtctacatctacagcaccaatcgcaaccagttcggcatctactctagcaaaccgagcgaagttaacatcgcacagaacaatgatattatttataacggtcgttatcaaaacttctctatcagcttttgggtccgtatcccgaagtacttcaataaagtcaatctgaataatgaatacacgatcatcgactgcattcgcaataacaacagcggttggaaaatcagcctgaattacaacaaaattatttggaccctgcaagatacggcgggtaacaatcagaaactggtgtttaactacacgcaaatgatcagcatttctgactatatcaacaagtggatctttgttaccatcaccaataatcgtctgggcaatagccgtatttacatcaacggtaacctgattgatgagaaaagcatcagcaacctgggcgatattcacgtcagcgacaacattctgttcaaaattgttggttgtaacgatacccgttacgtcggcatccgttatttcaaggttttcgatacggagctgggtaaaacggaaatcgaaacgttgtactccgatgaaccagatccgagcattctgaaggacttttggggtaactacttgctgtacaataaacgttactatctgctgaatctgttgcgcaccgacaagagcattacccaaaacagcaatttcctgaacattaatcagcaacgcggcgtataccaaaaaccgaacatcttcagcaatacgcgcctgtatactggtgttgaagtgatcattcgtaagaacggtagcaccgacattagcaacacggacaatttcgtccgtaagaatgacctggcgtacattaacgtcgtggaccgtgatgtcgagtatcgtctgtacgcagacatcagcattgcgaaaccggaaaagattatcaagctgatccgtaccagcaacagcaacaacagcctgggtcagatcattgtgatggacagcattggtaataactgcacgatgaacttccagaacaacaatggtggtaatatcggtctgctgggttttcacagcaataatctggttgcttccagctggtactacaataacattcgtaaaaacacgtctagcaatggttgtttttggagctttatcagcaaagagcacggctggcaagaaaat

SEQ ID NO: 16 - protein, 1278 aa, synthetic construct
  REGION 1..1278: note = Polypeptide Sequence of rBoNT/F(0)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 17 - DNA, 3942 bp, synthetic construct
  misc_feature 1..3942: note = Nucleotide Sequence of rBoNT/A(0) (His-tagged)
  source 1..3942: mol_type = other DNA
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaaagctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacaccagctgatttatgccggtcatcgtctgtatggtattgccattaatccgaatcgtgtgttcaaagtgaataccaacgcctattatgaaatgagcggtctggaagtgagttttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaacctttaccgaatacattaagaacatcatcaacaccagcattctgaacctgcgttatgaaagcaatcatctgattgatctgagccgttatgccagcaaaatcaatataggcagcaaggttaacttcgacccgattgacaaaaatcagatacagctgtttaatctggaaagcagcaaaattgaggtgatcctgaaaaacgccattgtgtataatagcatgtacgagaatttctcgaccagcttttggattcgtatcccgaaatactttaatagcatcagcctgaacaacgagtacaccattattaactgcatggaaaacaatagcggctggaaagttagcctgaattatggcgaaattatctggaccctgcaggatacccaagaaatcaaacagcgtgtggttttcaaatacagccagatgattaatatcagcgactatatcaaccgctggatttttgtgaccattaccaataatcgcctgaataacagcaagatctatattaacggtcgtctgattgaccagaaaccgattagtaatctgggtaatattcatgcgagcaacaacatcatgtttaaactggatggttgtcgtgatacccatcgttatatttggatcaagtacttcaacctgttcgataaagagttgaacgaaaaagaaattaaagacctgtatgataaccagagcaacagcggtattctgaaggatttttggggagattatctgcagtatgacaaaccgtattatatgctgaatctgtacgacccgaataaatacgtggatgtgaataatgttggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaatgatcagggcatcaccaacaagtgcaaaatgaatctgcaggacaataacggcaacgatattggttttattggcttccaccagttcaacaatattgcgaaactggttgcaagcaattggtataatcgtcagattgaacgtagcagtcgtaccctgggttgtagctgggaatttatccctgtggatgatggttggggtgaacgtccgctggaaaacctgtattttcaaggtgcaagtcatcatcaccatcaccaccatcattaa

SEQ ID NO: 18 - protein, 1313 aa, synthetic construct
  REGION 1..1313: note = Polypeptide Sequence of rBoNT/A(0) (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLENLYFQGASHHHHHHHH

SEQ ID NO: 19 - DNA, 2649 bp, synthetic construct
  misc_feature 1..2649: note = Nucleotide Sequence of rLHN/A (His-tagged)
  source 1..2649: mol_type = other DNA
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaaccgaaaatctgtatttccagggtgcaagtcatcatcaccatcaccaccatcattaa

SEQ ID NO: 20 - protein, 882 aa, synthetic construct
  REGION 1..882: note = Polypeptide Sequence of rLHN/A (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTENLYFQGASHHHHHHHH

SEQ ID NO: 21 - DNA, 1323 bp, synthetic construct
  misc_feature 1..1323: note = Nucleotide Sequence of rHC/A (His-tagged)
  source 1..1323: mol_type = other DNA
atgcatcatcaccatcaccacgaaaatctatacttccaaggaaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctgtaa

SEQ ID NO: 22 - protein, 440 aa, synthetic construct
  REGION 1..440: note = Polypeptide Sequence of rHC/A (His-tagged)
MHHHHHHENLYFQGKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 23 - DNA, 1326 bp, synthetic construct
  misc_feature 1..1326: note = Nucleotide Sequence of rLC/A (His-tagged)
  source 1..1326: mol_type = other DNA
atgccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgtttgaagagaatctgtatttccagggtgcaagtcatcatcaccatcaccaccatcattaa

SEQ ID NO: 24 - protein, 441 aa, synthetic construct
  REGION 1..441: note = Polypeptide Sequence of rLC/A (His-tagged)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEENLYFQGASHHHHHHHH

SEQ ID NO: 25 - DNA, 3918 bp, synthetic construct
  misc_feature 1..3918: note = Nucleotide Sequence of rBoNT/FA(0) (His-tagged)
  source 1..3918: mol_type = other DNA
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcaccagctgatttatgttctgcatggtctgtatggcacctatatcaccgaatacaaaattaccccgaatgtggttcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcaataccaaaaacagcctgtgcattaccgttaataatcgcgacctgttttttatcgcaagccaagaaagctatggcgagaataccattaacacctataaagagattgacgataccaccacactggatccgagctttgaagatattctggataaagtgatcctgaacttcaacgaacaggttattccgcagatgccgaatcgtaatgttagcaccgatattcagaaagacaactacatcccgaaatacgattataaccgcaccgacattatcgatagctatgaagttggtcgcaactacaacacctttttctatctgaatgcccagaaatttagcccgaacgaaagcaatattaccctgaccagcagctttgatacaggtctgttagaaggtagcaaagtgtatacctttttcagcagcgatttcattaacaacatcaacaaaccggttcaggccctgctgtttattgaatgggttaaacaggtgattcgcgattttaccaccgaagcaaccaaaacctcaaccgttgataaactgaaagatattagcctggtggtgccgtatattggtctggcactgaatattggtgatgagatctacaaacagcattttgcagaagcagttgaactggttggtgcaggtctgctgctggaattttcaccggaatttcttattccgacgctgctgatttttaccatcaaaggttatctgaccggtagcattcgcgataaagacaaaatcattaaaaccctggataacgccctgaatgttcgtgatcagaaatggaaagaactgtatcgttgggttgttagcaaatggctgaccaccattaatacgcagttcaacaaacgcaaagaacaaatgtacaaagccctgaaaaatcaggccaccgccattaaaaagatcatcgagaacaaatataacaactataccaccgatgaaaaaagcaagatcgatagcagctataacatcaacgaaattgaacgcaccctgaacgaaaaaatcaatctggccatgaaaaacatcgagcagtttattaccgaaagcagcattgcctatctgatcaatatcatcaacaacgaaacgatccagaaactgaaaagctatgatgacctggttcgtcgttatctgctgggttatattcgtaatcatagcagcattctgggcaatagcgttgaagaactgaattccaaagtgaacaaccatctggataatggcattccgtttgaactgagcagttataccaatgatagcctgctgatccgctacttcaataaaaactatggcgaactgaagtacaactgcattctgaacatcaaatatgagatggatcgtgacaaactggttgatagcagcggttatcgtagccgtatcaatattggtacaggcgtcaaatttagcgagatcgataaaaatcaagtgcagctgagcaatctggaatccagcaaaattgaagtcattctgaataacggcgtcatctataacagcatgtatgaaaacttttcgaccagcttttggattcgcattccgaaatactttcgcaacatcaataacgagtacaagatcatcagctgtatgcagaataatagcggttgggaagtgagcctgaattttagcaatatgaactcgaaaatcatctggaccctgcaggataccgaaggtatcaaaaaaaccgttgtgtttcagtacacccagaacattaacattagcgactatatcaaccgctggatctttgtgaccattacaaataatcgtctgagcaacagcaaaatctacattaatggtcgcctgatcaacgaagaaagcattagcgatctgggtaatatccatgccagcaacaacattatgtttaaactggatggttgccgtgatccgcatcgttatatctggattaaatactttaacctgtttgacaaagagctgaacaagaaagaaattaaagatctgtacgacaaccagagcaatagcggtattctgaaagatttctggggtgattatctgcagtatgacaaaccgtattatatgctgaatctgtatgacccgaataagtatctggatgtgaataatgttggcatccgtggctatatgtatctgaaaggtccgcgtggtcgtattgtgaccaccaacatttatctgaatagcaccctgtatatgggcaccaaattcatcattaagaaatatgccagcggcaacaaagataacattgtgcgtaataatgatcgcgtgtatattaacgtggtggtgaagaataaagaatatcgcctggcaaccaatgcaagccaggcaggcgttgaaaaaattctgagcgcagttgaaatcccggatgttggtaatctgagccaggttgttgtgatgaaaagcgaaaatgatcagggcattcgcaacaagtgtaaaatgaatctgcaagacaataacggcaacgatattggctttatcggctttcaccagtttaataacattgcaaaactggtggccagcaactggtataaccgtcagattggtaaagcaagccgtacctttggttgtagctgggaatttatcccggttgatgatggttggggtgaaagcagcctggaaaatctgtatttccagggtgccagtcatcatcaccaccatcaccatcactga

SEQ ID NO: 26 - protein, 1305 aa, synthetic construct
  REGION 1..1305: note = Polypeptide Sequence of rBoNT/FA(0) (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHQLIYVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSLENLYFQGASHHHHHHHH

SEQ ID NO: 27 - DNA, 2634 bp, synthetic construct
  misc_feature 1..2634: note = Nucleotide Sequence of rLHN/FA (His-tagged)
  source 1..2634: mol_type = other DNA
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcatgaactgattcatgttctgcatggtctgtatggcacctatattaccgaatacaaaattaccccgaatgtggtgcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcaataccaaaaacagcctgtgcattaccgttaataatcgcgacctgttttttatcgcaagccaagaaagctatggcgagaataccattaacacctataaagagattgacgataccaccacactggatccgagctttgaagatattctggataaagtgatcctgaacttcaacgaacaggttattccgcagatgccgaatcgtaatgttagcaccgatattcagaaagacaactacatcccgaaatacgattataaccgcaccgacattatcgatagctatgaagttggtcgcaactacaacacctttttctatctgaatgcccagaaatttagcccgaacgaaagcaatattaccctgaccagcagctttgatacaggtctgttagaaggtagcaaagtgtatacctttttcagcagcgatttcattaacaacatcaacaaaccggttcaggccctgctgtttattgaatgggttaaacaggtgattcgcgattttaccaccgaagcaaccaaaacctcaaccgttgataaactgaaagatattagcctggtggtgccgtatattggtctggcactgaatattggtgatgagatctacaaacagcattttgcagaagcagttgaactggttggtgcaggtctgctgctggaattttcaccggaatttcttattccgacgctgctgatttttaccatcaaaggttatctgaccggtagcattcgcgataaagacaaaatcattaaaaccctggataacgccctgaatgttcgtgatcagaaatggaaagaactgtatcgttgggttgttagcaaatggctgaccaccattaatacgcagttcaacaaacgcaaagaacaaatgtacaaagccctgaaaaatcaggccaccgccattaaaaagatcatcgagaacaaatataacaactataccaccgatgaaaaaagcaagatcgatagcagctataacatcaacgaaattgaacgcaccctgaacgaaaaaatcaatctggccatgaaaaacatcgagcagtttattacagaaagcagcattgcctacctgatcaatatcatcaacaacgaaaccattcagaaactgaaaagctatgatgacctggttcgtcgttatctgctgggttatattcgtaatcatagcagcattctgggcaatagcgttgaagaactgaattccaaagtgaacaaccatctggataatggcattccgtttgaactgagcagttataccaatgatagcctgctgatccgctacttcaataaaaactatggcgaagagaacctgtatttccagggtgccagtcatcatcaccaccatcaccatcactga

SEQ ID NO: 28 - protein, 877 aa, synthetic construct
  REGION 1..877: note = Polypeptide Sequence of rLHN/FA (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGEENLYFQGASHHHHHHHH

SEQ ID NO: 29 - DNA, 1341 bp, synthetic construct
  misc_feature 1..1341: note = Nucleotide Sequence of rHC/FA (His-tagged)
  source 1..1341: mol_type = other DNA
atgctgaagtataactgcatcctgaacatcaaatatgagatggatcgtgataaactggttgatagcagcggttatcgtagccgtatcaatattggcaccggtgtgaaatttagcgagatcgataaaaatcaggtgcagctgagcaatctggaaagcagcaaaattgaagtgattctgaataacggcgtgatctacaatagcatgtatgaaaacttttcgaccagcttctggattcgcattccgaaatactttcgcaacatcaacaacgagtacaagattatcagctgtatgcagaataatagcggttgggaagttagcctgaatttcagcaatatgaacagcaaaatcatttggaccctgcaggataccgaaggtatcaaaaaaaccgttgtgtttcagtacacccagaacattaacatcagcgattacattaaccgctggatctttgtgaccattaccaataatcgtctgagcaacagcaagatctatattaacggtcgcctgattaacgaagagagcattagcgatctgggtaatattcatgccagcaacaacatcatgtttaaactggatggttgtcgtgatccgcatcgttatatttggatcaaatacttcaacctgtttgataaagaactgaacaaaaaagaaatcaaagacctgtatgataaccagagcaatagcggcattctgaaagatttttggggtgattatctgcagtatgacaaaccgtattacatgctgaatctgtacgatccgaacaaatatctggatgtgaataatgtgggtatccgtggctatatgtatctgaaaggtccgcgtggtcgtattgttaccaccaacatttatctgaatagcaccctgtatatgggcaccaaattcatcattaaaaagtatgccagcggcaacaaagataacattgtgcgtaataatgatcgcgtgtatatcaatgtggtggtgaagaataaagaatatcgtctggccaccaatgcaagccaggcaggcgttgaaaaaattctgagcgcagttgaaattccggatgttggtaatctgagccaggttgttgttatgaaaagcgaaaatgatcagggcattcgcaacaaatgcaaaatgaatctgcaggacaataacggcaacgatattggttttattggcttccaccagttcaacaacattgcaaaactggtggcgagcaattggtataatcgtcagattggtaaagcaagccgtacctttggttgtagctgggaatttattccggttgatgatggttggggtgaaagcagcctggaaaatctgtattttcagggtgcaagtcatcatcaccaccatcaccatcattaa

SEQ ID NO: 30 - protein, 446 aa, synthetic construct
  REGION 1..446: note = Polypeptide Sequence of rHC/FA (His-tagged)
MLKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSLENLYFQGASHHHHHHHH

SEQ ID NO: 31 - DNA, 1347 bp, synthetic construct
  misc_feature 1..1347: note = Nucleotide Sequence of rLC/FA (His-tagged)
  source 1..1347: mol_type = other DNA
atgccggttgtgattaacagcttcaattatgatgatccggtgaacgataacaccatcatttatatccgtccgccttattatgaaaccagcaacacctatttcaaagccttccagattatggataacgtgtggattattccggaacgttatcgtctgggtattgatccgagcctgtttaatccgcctgttagcctgaaagcaggtagtgatggttattttgatccgaattatctgagcaccaacaccgagaaaaacaaatacctgcagattatgatcaagctgttcaaacgcattaatagcaaaccggcaggtcagattctgctggaagaaatcaaaaatgcaattccgtatctgggcaacagctatacccaagaagaacagtttaccaccaataatcgtaccgtgagctttaatgttaaactggccaatggtaatatcgttcagcagatggcaaatctgattatttggggtccgggtcctgatctgaccacaaataaaaccggtggtatcatctatagcccgtatcagagcatggaagcaaccccgtataaagatggttttggtagcattatgaccgtggaatttagtccggaatatgcaaccgcctttaacgatatttcaattgcaagccatagtccgtcgctgtttatcaaagatccggcactgattctgatgcatgaactgattcatgttctgcatggtctgtatggcacctatattaccgaatacaaaattaccccgaatgtggtgcagagctatatgaaagttaccaaaccgattaccagcgcagaatttctgacctttggtggtcgtgatcgcaatattgttccgcagagcattcagagccagctgtataacaaagttctgagcgattataaacgtattgccagccgtctgaataaagttaataccgcaaccgcactgatcaacatcgatgaattcaaaaacctgtacgagtggaaataccagtttgccaaagatagcaatggtgtgtatagcgtggatctgaacaaatttgagcagctgtacaaaaaaatctatagcttcaccgaattcaacctggcctatgagtttaaaatcaaaacccgtctgggttatctggccgaaaattttggtccgttttatctgccgaatctgctggatgatagcatttataccgaagtggatggttttaacattggtgcactgagcattaactatcagggtcagaatattggcagcgatatcaacagcatcaaaaaactgcaaggtcagggtgttgttagccgtgttgttcgtctgtgtagcaatagcgaaaatctgtattttcagggtgccagtcatcatcaccaccatcaccatcactga

SEQ ID NO: 32 - protein, 448 aa, synthetic construct
  REGION 1..448: note = Polypeptide Sequence of rLC/FA (His-tagged)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSENLYFQGASHHHHHHHH

SEQ ID NO: 33 - DNA, 3888 bp, synthetic construct
  misc_feature 1..3888: note = Nucleotide Sequence of rBoNT/F(0) (His-tagged)
  source 1..3888: mol_type = other DNA
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacaccagctgatttatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgtgattccgcgtaaaggcaccaaagcaccgcctcgtctgtgtattcgtgtgaataatcgtgaactgttttttgttgcaagcgagagcagctataacgagaatgatattaacaccccgaaagagattgacgataccaccaatctgaataacaactatcgcaacaatctggatgaagtgatcctggattataacagcgaaaccattccgcagattagcaatcagaccctgaataccctggttcaggatgatagctatgttccgcgttatgatagcaatggcaccagcgaaattgaagaacataatgtggttgatctgaacgtgttcttttatctgcatgcacagaaagtgccggaaggtgaaaccaatattagcctgaccagcagcattgataccgcactgagcgaagaaagccaggtttataccttttttagcagcgaattcatcaacaccattaacaaaccggttcatgcagcactgtttattagctggattaatcaggtgattcgcgattttaccaccgaagcaacccagaaaagcacctttgataaaattgccgatattagtctggtggtgccgtatgttggtctggcactgaatattggtaatgaagtgcagaaagagaactttaaagaagccttcgaactgttaggtgccggtattctgctggaatttgtgccggaactgctgattccgaccattctggtttttaccattaagagctttattggcagcagcgagaacaagaacaaaatcattaaagccatcaacaacagcctgatggaacgcgaaaccaaatggaaagaaatttacagctggattgtgagcaattggctgacccgtatcaatacccagtttaacaaacgcaaagaacaaatgtatcaggccctgcagaatcaggttgatgcaattaaaaccgtgatcgaatacaaatacaacaactataccagcgacgaacgtaatcgcctggaaagcgaatacaacattaataacattcgcgaagaactgaacaaaaaagtgagcctggcaatggaaaacatcgaacgttttattaccgaaagcagcatcttctacctgatgaaactgattaacgaagccaaagttagcaaactgcgcgaatatgatgaaggcgttaaagaatatctgctggactatattagcgaacatcgtagcattctgggtaatagcgttcaagagctgaatgatctggttaccagcacactgaataatagcattccgtttgaactgagcagctacaccaacgataaaatcctgatcctgtacttcaacaaactgtacaagaagatcaaggacaacagcatactggatatgcgctatgaaaacaacaagttcattgatatcagcggctatggtagcaacattagcattaatggtgatgtgtatatctacagcaccaaccgcaatcagtttggtatttatagcagcaaaccgagcgaagttaatattgcgcagaataacgatatcatctacaacggtcgctatcagaactttagcattagcttttgggttcgcattccgaaatactttaacaaggtgaacctgaacaacgagtacaccattattgattgcattcgcaataataacagcggctggaaaatcagcctgaactataacaaaattatctggaccctgcaggataccgcaggtaataatcagaaactggtgtttaactacacccagatgattagcatcagcgactatatcaacaaatggatctttgtgaccattaccaacaatcgtctgggtaacagccgcatttatatcaatggcaatctgatcgacgaaaaaagcatttcaaatctgggcgatattcacgtgagcgataacattctgttcaaaattgttggctgcaacgatacccgttatgttggtattcgttacttcaaagtgtttgatacggaactgggcaaaacggaaattgaaaccctgtatagtgatgaaccggatccgagcattctgaaagatttttggggtaattatctgctgtacaacaaacgctactatctgctgaacctgctgcgtaccgataaaagcattacacagaatagcaactttctgaacatcaatcagcagcgtggtgtttatcagaaaccgaacatttttagcaacacccgtctgtataccggtgtggaagttattattcgtaaaaacggtagcaccgatatcagcaacaccgataactttgtgcgtaaaaatgacctggcctatattaacgttgttgatcgtgatgttgagtatcgtctgtatgcggatattagcattgccaaaccggaaaagattatcaaactgatccgtaccagcaacagcaataattcactgggtcagattatcgtgatggacagcattggtaacaattgcaccatgaatttccagaacaataacggtggtaatattggcctgctgggctttcatagcaataatctggttgcaagcagctggtattacaacaacatccgtaaaaataccagcagtaatggttgcttttggagctttatcagtaaagaacatggctggcaagaaaacgagaacctgtattttcagggtgcaagtcatcatcaccatcaccaccatcattaa

SEQ ID NO: 34 - protein, 1295 aa, synthetic construct
  REGION 1..1295: note = Polypeptide Sequence of rBoNT/F(0) (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQENENLYFQGASHHHHHHHH

SEQ ID NO: 35 - DNA, 2649 bp, synthetic construct
  misc_feature 1..2649: note = Nucleotide Sequence of rLHN/F (His-tagged)
  source 1..2649: mol_type = other DNA
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacatgaactgattcatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgtgattccgcgtaaaggcaccaaagcaccgcctcgtctgtgtattcgtgtgaataatcgtgaactgttttttgttgcaagcgagagcagctataacgagaatgatattaacaccccgaaagagattgacgataccaccaatctgaataacaactatcgcaacaatctggatgaagtgatcctggattataacagcgaaaccattccgcagattagcaatcagaccctgaataccctggttcaggatgatagctatgttccgcgttatgatagcaatggcaccagcgaaattgaagaacataatgtggttgatctgaacgtgttcttttatctgcatgcacagaaagtgccggaaggtgaaaccaatattagcctgaccagcagcattgataccgcactgagcgaagaaagccaggtttataccttttttagcagcgaattcatcaacaccattaacaaaccggttcatgcagcactgtttattagctggattaatcaggtgattcgcgattttaccaccgaagcaacccagaaaagcacctttgataaaattgccgatattagtctggtggtgccgtatgttggtctggcactgaatattggtaatgaagtgcagaaagagaactttaaagaagccttcgaactgttaggtgccggtattctgctggaatttgtgccggaactgctgattccgaccattctggtttttaccattaagagctttattggcagcagcgagaacaagaacaaaatcattaaagccatcaacaacagcctgatggaacgcgaaaccaaatggaaagaaatttacagctggattgtgagcaattggctgacccgtatcaatacccagtttaacaaacgcaaagaacaaatgtatcaggccctgcagaatcaggttgatgcaattaaaaccgtgatcgaatacaaatacaacaactataccagcgacgaacgtaatcgcctggaaagcgaatacaacattaataacattcgcgaagaactgaacaaaaaagtgagcctggcaatggaaaacatcgaacgttttattaccgaaagcagcatcttctacctgatgaaactgattaacgaagccaaagttagcaaactgcgcgaatatgatgaaggcgttaaagaatatctgctggactatattagcgaacatcgtagcattctgggtaatagcgttcaagagctgaatgatctggttaccagcacactgaataatagcattccgtttgaactgagcagctacaccaacgataaaatcctgatcctgtacttcaacaaactgtacaagaaagaaaacctgtattttcagggtgcaagccatcatcaccaccatcaccatcattaa

SEQ ID NO: 36 - protein, 882 aa, synthetic construct
  REGION 1..882: note = Polypeptide Sequence of rLHN/F (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKENLYFQGASHHHHHHHH

SEQ ID NO: 37 - DNA, 1296 bp, synthetic construct
  misc_feature 1..1296: note = Nucleotide Sequence of rHC/F (His-tagged)
  source 1..1296: mol_type = other DNA
atgatcaaggataacagcattctggatatgcgctatgagaacaacaaattcattgatattagcggctatggcagcaacattagcattaatggtgatgtgtatatctacagcaccaaccgtaatcagtttggcatttatagcagcaaaccgagcgaagttaatattgcccagaacaacgatatcatctataacggtcgctatcagaacttcagcattagcttttgggttcgcattccgaaatacttcaataaggtgaacctgaacaacgagtataccatcattgattgcattcgcaataataacagcggctggaaaattagcctgaactacaacaaaattatctggaccctgcaggataccgcaggtaataatcagaaactggtgtttaactacacccagatgattagcatcagcgactatatcaacaaatggatctttgtgaccattaccaataatcgcctgggtaatagccgcatttatatcaatggtaacctgatcgatgagaaaagcattagcaatctgggtgatattcatgtgagcgataacatcctgtttaaaatcgtgggttgtaacgatacccgttatgttggtattcgctacttcaaagtgtttgataccgaactgggtaaaaccgaaattgaaaccctgtatagtgatgaaccggatccgagcattctgaaagatttttggggtaattatctgctgtacaacaaacgctactatctgctgaatctgctgcgtaccgataaatcaattacccagaatagcaacttcctgaacattaatcagcagcgtggtgtttatcagaaaccgaacatttttagcaacacccgtctgtataccggtgtggaagttattattcgtaaaaatggcagcaccgatatcagcaacaccgataactttgttcgcaaaaatgatctggcgtatatcaacgttgttgatcgtgatgttgaatatcgtctgtatgccgatattagcattgccaaaccggaaaaaatcatcaaactgatccgtaccagcaacagcaataattcactgggtcagattattgtgatggatagcattggtaataactgcaccatgaactttcagaacaataacggtggtaatattggtctgctgggctttcatagtaataatctggttgcaagcagctggtattataacaacatccgtaaaaataccagcagcaatggttgcttttggagctttattagcaaagaacatggctggcaagaaaacgagaatctgtattttcagggtgcaagtcatcatcaccaccatcaccatcattaa

SEQ ID NO: 38 - protein, 431 aa, synthetic construct
  REGION 1..431: note = Polypeptide Sequence of rHC/F (His-tagged)
MIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQENENLYFQGASHHHHHHHH

SEQ ID NO: 39 - DNA, 1347 bp, synthetic construct
  misc_feature 1..1347: note = Nucleotide Sequence of rLC/F (His-tagged)
  source 1..1347: mol_type = other DNA
atgccggttgtgattaacagcttcaattataacgatccggtgaacgatgataccatcctgtatatgcagattccgtatgaagagaaaagcaaaaagtactacaaagcctttgagatcatgcgcaacgtttggattattccggaacgtaataccattggcaccgatccgagcgattttgatccgcctgcaagcctggaaaatggtagcagcgcatattatgatccgaattatctgaccaccgatgccgaaaaagatcgttatctgaaaaccaccatcaaactgttcaaacgcattaatagcaatccggcaggcgaagttctgctgcaagaaattagctatgcaaaaccgtatctgggcaatgaacataccccgattaatgaatttcatccggttacacgtaccacgagcgttaacattaaaagcagcaccaatgtgaagtccagcattattctgaatctgctggttttaggtgcaggtccggatatttttgaaaattcaagctatccggtgcgcaaactgatggatagcggtggtgtgtatgatccgtcaaatgatggttttggcagcattaacattgtgacctttagtccggaatatgaatacaccttcaacgatattagcggtggctataatagcagcaccgaaagttttattgcagatccggcaattagcctggcacatgaactgattcatgcactgcatggtctgtatggtgcacgtggtgttacctataaagaaaccattaaagttaaacaggcaccgctgatgattgcggaaaaaccgattcgtctggaagaatttctgacctttggtggtcaggatctgaacattattaccagcgcaatgaaagagaaaatctataataacctgctggccaactatgagaaaattgcaacccgtctgagccgtgttaatagcgcacctcctgaatatgatatcaacgagtataaagactattttcagtggaaatacggcctggataaaaatgcagatggtagctataccgtgaacgagaacaaatttaacgagatctacaaaaaactgtatagcttcaccgaaatcgatctggccaacaaattcaaagtgaaatgccgcaacacctacttcatcaaatatggctttctgaaagttccgaacctgcttgatgatgatatctataccgttagcgaaggctttaacattggtaatctggccgttaataatcgcggtcagaacattaaactgaacccgaaaattatcgatagcatcccggataaaggcctggttgaaaaaattgtgaaattctgcaaaagcgagaacctgtattttcagggtgcaagtcatcatcaccatcaccaccatcattaa

SEQ ID NO: 40 - protein, 448 aa, synthetic construct
  REGION 1..448: note = Polypeptide Sequence of rLC/F (His-tagged)
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSENLYFQGASHHHHHHHH

SEQ ID NO: 41 - DNA, 1317 bp, synthetic construct
  misc_feature 1..1317: note = Nucleotide Sequence of Cationic rHC/A (His-tagged)
  source 1..1317: mol_type = other DNA
atgatcatcaacaccagcattctgaacctgcgttatgaaagcaaacatctgattgatctgagccgttatgccagcaaaatcaatataggcagcaaggttaacttcgacccgattgacaaaaatcagatacagctgtttaatctggaaagcagcaaaattgaggtgatcctgaaaaaagcgatcgtgtataatagcatgtacgagaatttttcgaccagcttttggattcgcatcccgaaatactttaacaagattagcctgaacaacgagtataccatcattaactgcatggaaaacaatagcggttggaaagtcagcctgaattatggcgaaattatctggaccctgcaggataccaaagaaatcaaacagcgtgtggtgttcaaatacagccagatgattaatatcagcgactatatcaaccgctggatttttgtgaccattaccaataatcggctgaacaagagcaagatctatattaacggtcgtctgattgaccagaaaccgattagtaatctgggtaatattcatgcgagcaacaaaatcatgtttaaactggatggttgccgtgatacccatcgttatatttggatcaaatacttcaacctgttcgataaagagttgaacgaaaaagaaattaaagacctgtacgataaccagagcaatagcggcatactgaaagatttttggggagattatctgcagtatgacaaaccgtattatatgctgaatctgtacgacccgaataaatacgtggatgttaataatgtgggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaacgataaaggcatcaccaacaaatgcaagatgaatctgcaggacaataacggcaatgatattggcttcattggctttcaccagtttaacaacattgcaaaactggttgcgagcaattggtataatcgtcagattgaacgtagcagtcgtaccctgggttgtagctgggaatttatccctgtggatgatggttggggtgaacgtccgctgaagcttgcggccgcactcgagcaccaccaccaccaccactga

SEQ ID NO: 42 - protein, 438 aa, synthetic construct
  REGION 1..438: note = Polypeptide Sequence of Cationic rHC/A (His-tagged)
MIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPLKLAAALEHHHHHH

SEQ ID NO: 43 - DNA, 1341 bp, synthetic construct
  misc_feature 1..1341: note = Nucleotide Sequence of rHC/AB (His-tagged)
  source 1..1341: mol_type = other DNA
atgattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaaatgaaactgttcctggccccgatttacgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaaaagcttgcggccgcactcgagcaccaccaccaccaccactga

SEQ ID NO: 44 - protein, 446 aa, synthetic construct
  REGION 1..446: note = Polypeptide Sequence of rHC/AB (His-tagged)
MILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEKLAAALEHHHHHH

SEQ ID NO: 45 - DNA, 1306 bp, synthetic construct
  misc_feature 1..1306: note = Nucleotide Sequence of rHC/A Variant Y1117V H1253K (His-tagged)
  source 1..1306: mol_type = other DNA
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttcaaacagttcaacaatattgctaaactggtagcgagcaattggtacaatcgtcagattgagcgcagcagccgtactttgggctgtagctgggagtttatcccggtcgatgatggttggggcgaacgtccgctgcaccatcaccatcaccatcaccatcaccatt

SEQ ID NO: 46 - protein, 435 aa, synthetic construct
  REGION 1..435: note = Polypeptide Sequence of rHC/A Variant Y1117V H1253K (His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFKQFNNIAKLVASNWYNRQIERSSRTLGCSWEFIPVDDGWGERPLHHHHHHHHHH

SEQ ID NO: 47 - DNA, 1308 bp, synthetic construct
  misc_feature 1..1308: note = Nucleotide Sequence of rHC/A Variant Y1117V F1252Y H1253K L1278F(His-tagged)
  source 1..1308: mol_type = other DNA
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggttacaaacagttcaacaatattgctaaactggtagcgagcaattggtacaatcgtcagattgagcgcagcagccgtacttttggctgtagctgggagtttatcccggtcgatgatggttggggcgaacgtccgctgcaccatcaccatcaccatcaccatcaccattaa

SEQ ID NO: 48 - protein, 435 aa, synthetic construct
  REGION 1..435: note = Polypeptide Sequence of rHC/A Variant Y1117V F1252Y H1253K L1278F(His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGYKQFNNIAKLVASNWYNRQIERSSRTFGCSWEFIPVDDGWGERPLHHHHHHHHHH

SEQ ID NO: 49 - DNA, 1293 bp, synthetic construct
  misc_feature 1..1293: note = Nucleotide Sequence of rHC/A Variant Y1117V F1252Y H1253K L1278H(His-tagged)
  source 1..1293: mol_type = other DNA
atgatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctggttgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggttacaaacagttcaacaatattgctaaactggtagcgagcaattggtacaatcgtcagattgagcgcagcagccgtactcatggctgtagctgggagtttatcccggtcgatgatggttggggcgaacgtccgctgcaccatcaccatcaccat

SEQ ID NO: 50 - protein, 431 aa, synthetic construct
  REGION 1..431: note = Polypeptide Sequence of rHC/A Variant Y1117V F1252Y H1253K L1278H(His-tagged)
MIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLVDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGYKQFNNIAKLVASNWYNRQIERSSRTHGCSWEFIPVDDGWGERPLHHHHHH

SEQ ID NO: 51 - protein, 1296 aa, Clostridium botulinum [sequence omitted]

SEQ ID NO: 52 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 53 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 54 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 55 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 56 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 57 - protein, 1297 aa, Clostridium botulinum
  VARIANT 7: note = Amino acid at position 7 can be any naturally-occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 58 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 59 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 60 - DNA, 3888 bp, synthetic construct
  misc_feature 1..3888: note = Nucleotide Sequence of mrBoNT/A
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 61 - protein, 1296 aa, synthetic construct
  REGION 1..1296: note = Polypeptide Sequence of mrBoNT/A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 62 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 63 - protein, 1304 aa, synthetic construct
  REGION 1..1304: note = Polypeptide Sequence of mrBoNT/AB
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 64 - protein, 1304 aa, synthetic construct
  REGION 1..1304: note = Polypeptide Sequence of mrBoNT/AB(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 65 - protein, 1296 aa, synthetic construct
  REGION 1..1296: note = Polypeptide Sequence of mrBoNT/A(0)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 66-70 - skipped (5 entries)

SEQ ID NO: 71 - protein, 6 aa, Clostridium botulinum
NIINTS

SEQ ID NO: 72 - protein, 6 aa, Clostridium botulinum
NIVNTS

SEQ ID NO: 73 - protein, 6 aa, Clostridium botulinum
NITNAS

SEQ ID NO: 74 - protein, 6 aa, Clostridium botulinum
NITNTS

SEQ ID NO: 75 - protein, 6 aa, Clostridium botulinum
EILNNI

SEQ ID NO: 76 - protein, 6 aa, Clostridium botulinum
DILNNI

(19) United States
(12) Patent Application Publication
(10) Pub. No.: US 2025/0082738 A1
(43) Pub. Date: Mar. 13, 2025

(54) TREATMENT OF VISCERAL PAIN

(71) Applicant: IPSEN BIOPHARM LIMITED, Wrexham (GB)

(72) Inventors: Jaqueline Caroline MAIGNEL, Wrexham (GB); Stephane LEZMI, Wrexham (GB); Vincent MARTIN, Wrexham (GB)

(21) Appl. No.: 18/711,047

(22) PCT Filed: Nov. 21, 2022
(86) PCT No.: PCT/GB2022/052947

§ 371 Date: May 16, 2024

(30) Foreign Application Priority Data
Nov. 22, 2021 (GB) 2116795.2

(51) Int. Cl. A61K 38/48; A61P 29/00

(52) U.S. Cl. CPC A61K 38/4893; A61P 29/00

(57) ABSTRACT

The present invention provides a clostridial neurotoxin for use in a method of suppressing visceral pain in a patient. The method comprises: (a) identifying an organ in the patient that is causing said visceral pain; (b) mapping said organ to a specific dermatome nerve according to the following instructions, thereby matching said organ to one or more specifically designated dermatome nerve(s): urinary bladder maps to the T11, T12, L1, S1, S2, S3 and/or S4 dermatome nerve(s); fallopian tube(s) maps to the T11 dermatome nerve(s); uterus maps to the T12 and/or L1 dermatome nerve(s); cervix maps to the S2, S3 10 and/or S4 dermatome nerve(s); stomach maps to the T8, T9 and/or T10 dermatome nerve(s); liver maps to the T8, T9, T10 and/or T11 dermatome nerve(s); gall bladder maps to the T8, T9, T10 and/or T11 dermatome nerve(s); pancreas maps to the T7, T8, T9, T10, T11 and/or T12 dermatome nerve(s); small intestine maps to the T10, T11 and/or T12 dermatome nerve(s); colon maps to the T11, S1, S2, S3 and/or S4 dermatome nerve(s); kidney maps to the T10, T11, T12 and/or L1 dermatome nerve(s); ureter maps to the T11, T12 and/or L1 dermatome nerve(s); ovary maps to the T11 dermatome nerve(s); testes maps to the T10, T11, L1, L2, S1, S2, S3 and/or S4 dermatome nerve(s); epididymis maps to the T10, T11, L1, L2, S1, S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

### FIELD OF THE INVENTION

The present invention relates to methods of treating visceral pain in a patient, more particularly by administration of a clostridial neurotoxin.

### BACKGROUND

Visceral pain is pain that emerges from one's internal organs, such as the stomach, bladder, uterus, or rectum, usually because of the presence of an injury or insult to the organ caused by an underlying disease. For example, visceral pain (a type of nociceptive pain) can be caused by medical conditions that produce inflammation, pressure, or other types of injury to organs. Pelvic pain caused by a bladder infection and abdominal pain caused by irritable bowel syndrome are types of visceral pain. It represents a widespread medical issue, and indeed it has been reported that more than 20% of the world's population suffer from visceral pain at some point in their lives.

One reason that visceral pain is common is because of the wide variety of different conditions/diseases that can cause such pain, which include Crohn's disease, ulcerative colitis, irritable bowel syndrome, coeliac disease, endometriosis, gastric ulcers, kidney and bladder stones, gallbladder stones, pancreatitis and peritonitis. Very few treatments are tailored specifically for treating pain that emanates/emerges from the viscera, and as an alternative a remedy for the underlying cause of the pain is often sought. By way of example, where peritonitis (an inflammation of the peritoneum due to infection) causes pain of the abdominal organs, the patient may be treated with an aim to remove the cause of inflammation e.g. via antibiotic administration.

Naturally, a significant amount of time may be required to remove such underlying insult, such that there remains a need to suppress visceral pain, which may otherwise lead to a significant decrease to a patient's quality of life. This is made difficult because the viscera are by their very nature ‘internal’ to the subject, such that targeting the viscera (organs) typically requires invasive, and significantly unpleasant, administration routes. The need to suppress the actual pain (visceral pain) is of particular importance where the underlying condition exists ‘long-term’ (the visceral pain being suffered also ‘long-term’), and even more so where the condition is not curable. An example of such condition (not curable) is endometriosis, which instead relies on treatments to help ease the associated symptoms. In this regard, clostridial neurotoxin treatment (BoNT/A) has previously been tested for alleviating pain associated with acute dysmenorrhoea (menstrual cramps) and pelvic pain syndrome of uterine origin, and allowed for improvement of quality of life scores in the majority of patients to which BoNT/A was administered. However, such treatment required injection of botulinum toxin directly into the myometrial wall under hysteroscopic control. This is highly invasive and inconvenient, particularly since repeated administration may be required over the long term in such incurable disease scenarios.

Thus, there is an unmet need for pain relief strategies for alleviating visceral pain, with a particularly unmet need being represented by the need for therapy that is minimally invasive.

The present invention solves one or more of the above-mentioned problems.

### SUMMARY OF THE INVENTION

In more detail, the invention is predicated on the surprising finding that administration (e.g. intradermal administration) of a clostridial neurotoxin to a dermatome nerve, that maps to a thoracic, pelvic, or abdominal organ/viscera contributing to visceral pain, provides for visceral pain suppression.

The inventors have provided data to evidence that, upon administration of clostridial neurotoxin to a dermatome/dermatome nerve of a patient, retrograde axonal transport of the neurotoxin (more particularly, via a spinal nerve also referred to as a dermatome nerve herein) allows for delivery of the neurotoxin to the spinal cord to act at (e.g. cause SNARE protein cleavage) or target the same area in the spinal cord dorsal horns in which visceral pain is integrated. Experiments on pain suppression were performed using a rodent model of visceral (bladder) pain. To complement this rodent model data, the inventors also analysed SNAP-25 cleavage in the spinal cord of pigs following administration of clostridial neurotoxin into a dermatome. The pig advantageously shares similarities with human skin in terms of structure, thickness, and nerve innervation (as well as other properties such as pigmentation, collagen and lipid composition, wound-healing and immune responses).

Notably, SNAP-25 cleavage (used as a readout of clostridial neurotoxin transport and activity) was detected in the spinal cords of both animal models tested (rodent and pig) following intradermal administration of clostridial neurotoxin to a dermatome, with the spinal cord area having cleaved SNAP-25 being consistent with the dermatome to which the clostridial neurotoxin was injected, particularly in the ipsilateral dorsal horn. Once the clostridial neurotoxin has thus gained access to the appropriate area of the spinal cord, mechanisms involved in visceral pain integration are inhibited by the activity of the neurotoxin. In this way, the strength of the pain perceived by the patient is reduced (in other words, visceral pain is suppressed or reduced).

By choosing an appropriate dermatome in which to inject the clostridial neurotoxin, visceral pain that emerges from any given viscera/organ can be suppressed or reduced. This is because sensory nerve fibers (e.g. spinal nerves) from particular dermatomes come together at the same spinal cord level as do the general visceral afferent fibers from particular viscera/organs. By way of example, spinal nerves T8, T9 and T10, which connect the spinal cord to respectively named dermatomes (i.e. the T8, T9 and T10 dermatomes, respectively), converge with visceral afferent fibers from the stomach at the same level of the spinal cord. Thus, by administering a clostridial neurotoxin to a T8, T9 and/or T10 dermatome, the neurotoxin can be delivered (retrograde transport via dermatome/spinal nerve(s)) to the same level of the spinal cord that visceral afferent fibers from the stomach can be found. Once there, the clostridial neurotoxin can inhibit the activity of this visceral afferent fiber in the spinal cord, and thus suppress the integration of stomach pain. A suppression of perceived pain thus follows.

Various particular dermatome-viscera interactions have previously been mapped out (e.g. in addition to the above described T8/T9/T10-stomach interaction), for example from studies into the phenomenon of referred pain. Indeed, dermatome maps are used clinically to demonstrate the site(s) of pathology of a patient with a suspected spinal-nerve lesion. A common dermatome map to be used clinically is the American Spinal Injury Association's (ASIA) worksheet produced as the International Standards for Neurological Classification of Spinal Cord Injury (ISNCSCI). At the time of writing, the 2019 edition of said worksheet is obtainable here: https://asia-spinalinjury.org/wp-content/uploads/2019/10/ASIA-ISCOS-Worksheet_10.2019_PRINT-Page-1-2.pdf. An illustration of various dermatome-viscera interactions is provided in the bottom left of FIG. 1.

A brief summary of clostridial neurotoxins is now provided (detailed information is outlined below). Bacteria in the genus Clostridia produce highly potent and specific protein toxins, which can (temporarily) impair function of neurons and other cells to which they are delivered. Examples of such clostridial toxins include the neurotoxins produced by C. tetani (TeNT) and by C. botulinum (BoNT) serotypes A-G, and X (see WO 2018/009903 A2), as well as those produced by C. baratii and C. butyricum. Both tetanus and botulinum toxins act by inhibiting the function of affected neurons, specifically the release of neurotransmitters.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the He domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Clostridial neurotoxins provide non-cytotoxic protease activity. Non-cytotoxic proteases act by proteolytically cleaving intracellular transport proteins known as SNARE proteins (e.g. SNAP-25, VAMP, or Syntaxin). The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum toxin have been successfully employed in a wide range of therapies. The inventors have demonstrated a surprising utility of clostridial neurotoxins in suppressing visceral pain, for example by suppressing visceral pain integration at the spinal cord.

### DETAILED DESCRIPTION

Broad aspects of the invention provide any of the following:
a clostridial neurotoxin for use in a method of suppressing visceral pain;
a method of suppressing visceral pain in a patient, the method comprising administering a clostridial neurotoxin to the patient;
use of a clostridial neurotoxin in the manufacture of a medicament to suppress visceral pain in a patient.

Visceral pain is pain that results from the activation of nociceptors of the thoracic, pelvic, or abdominal viscera (organs) and glands. Thus, the term “visceral pain” as used throughout the present disclosure may mean pain that is contributed by a thoracic, pelvic, or abdominal viscera and/or gland. Examples of such viscera (organ) include: urinary bladder, uterus, stomach, liver, gall bladder, pancreas, small intestine, colon, kidney, ureter, ovary, fallopian tube(s), cervix, testes, and/or epididymis.

A method of the invention may comprise identifying an organ in the patient that is causing said visceral pain, preferably wherein said organ is selected from the urinary bladder, uterus, stomach, liver, gall bladder, pancreas, small intestine, colon, kidney, ureter, ovary, fallopian tube(s), cervix, testes, and epididymis. A method of the invention may comprise mapping said organ to a specific dermatome nerve; for example, according to the following instructions, thereby matching said organ to one or more specifically designated dermatome nerve(s):
i. urinary bladder maps to the T11, T12, L1, S1, S2, S3 and/or S4 dermatome nerve(s);
ii. uterus maps to the T12 and/or L1 dermatome nerve(s);
iii. stomach maps to the T8, T9 and/or T10 dermatome nerve(s);
iv. liver maps to the T8, T9, T10 and/or T11 dermatome nerve(s);
v. gall bladder maps to the T8, T9, T10 and/or T11 dermatome nerve(s);
vi. pancreas maps to the T7, T8, T9, T10, T11 and/or T12 dermatome nerve(s);
vii. small intestine maps to the T10, T11 and/or T12 dermatome nerve(s);
viii. colon maps to the T11, S1, S2, S3 and/or S4 dermatome nerve(s);
ix. kidney maps to the T10, T11, T12 and/or L1 dermatome nerve(s);
x. ureter maps to the T11, T12 and/or L1 dermatome nerve(s);
xi. ovary maps to the T11 dermatome nerve(s);
xii. fallopian tube(s) maps to the T11 dermatome nerve(s);
xiii. cervix maps to the S2, S3 and/or S4 dermatome nerve(s);
xiv. testes maps to the T10, T11, L1, L2, S1, S2, S3 and/or S4 dermatome nerve(s);
xv. epididymis maps to the T10, T11, L1, L2, S1, S2, S3 and/or S4 dermatome nerve(s).

The clostridial neurotoxin may be administered to (or proximal to) a “dermatome” toward administering the neurotoxin to a “dermatome nerve”; in which case, the method may comprise mapping said organ to a specific dermatome according to instructions outlined in the paragraph above, thereby matching said organ to one or more specifically designated dermatome(s).

A method of the invention may comprise administering a therapeutically effective amount of a clostridial neurotoxin at a site proximal to said one or more specifically designated dermatome nerve(s). A method of the invention may comprise administering a therapeutically effective amount of a clostridial neurotoxin to said one or more specifically designated dermatome nerve(s). A method of the invention may comprise administering a therapeutically effective amount of a clostridial neurotoxin at a site proximal to said one or more specifically designated dermatome(s). A method of the invention may comprise administering a therapeutically effective amount of a clostridial neurotoxin to said one or more specifically designated dermatome(s).

The clostridial neurotoxin may suppress visceral pain following administration. Suitably, following administration of said clostridial neurotoxin to the patient, the clostridial neurotoxin is transported via retrograde axonal transport to the spinal cord (e.g. the dorsal horn(s)) where it binds sensory afferent nerve cells, enters said cells (e.g. by receptor-mediated endocytosis) and suppresses neurotransmitter release therefrom, thereby suppressing said visceral pain.

A more particular aspect provides a clostridial neurotoxin for use in a method of suppressing visceral pain in a patient, the method comprising:
a. identifying an organ in the patient that is causing said visceral pain;
b. mapping said organ to a specific dermatome nerve according to the following instructions, thereby matching said organ to one or more specifically designated dermatome nerve(s):
   i. urinary bladder maps to the T11, T12, L1, S1, S2, S3 and/or S4 dermatome nerve(s);
   ii. uterus maps to the T12 and/or L1 dermatome nerve(s);
   iii. stomach maps to the T8, T9 and/or T10 dermatome nerve(s);
   iv. liver maps to the T8, T9, T10 and/or T11 dermatome nerve(s);
   v. gall bladder maps to the T8, T9, T10 and/or T11 dermatome nerve(s);
   vi. pancreas maps to the T7, T8, T9, T10, T11 and/or T12 dermatome nerve(s);
   vii. small intestine maps to the T10, T11 and/or T12 dermatome nerve(s);
   viii. colon maps to the T11, S1, S2, S3 and/or S4 dermatome nerve(s);
   ix. kidney maps to the T10, T11, T12 and/or L1 dermatome nerve(s);
   x. ureter maps to the T11, T12 and/or L1 dermatome nerve(s);
   xi. ovary maps to the T11 dermatome nerve(s);
   xii. fallopian tube(s) maps to the T11 dermatome nerve(s);
   xiii. cervix maps to the S2, S3 and/or S4 dermatome nerve(s);
   xiv. testes maps to the T10, T11, L1, L2, S1, S2, S3 and/or S4 dermatome nerve(s);
   xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds sensory afferent nerve cells, enters said cells (e.g. by receptor-mediated endocytosis) and suppresses neurotransmitter release therefrom, thereby suppressing said visceral pain.

One aspect provides a method of suppressing visceral pain in a patient, the method comprising:
a. identifying an organ in the patient that is causing said visceral pain;
b. mapping said organ to a specific dermatome nerve according to the following instructions, thereby matching said organ to one or more specifically designated dermatome nerve(s):
   i. urinary bladder maps to the T11, T12, L1, S1, S2, S3 and/or S4 dermatome nerve(s);
   ii. uterus maps to the T12 and/or L1 dermatome nerve(s);
   iii. stomach maps to the T8, T9 and/or T10 dermatome nerve(s);
   iv. liver maps to the T8, T9, T10 and/or T11 dermatome nerve(s);
   v. gall bladder maps to the T8, T9, T10 and/or T11 dermatome nerve(s);
   vi. pancreas maps to the T7, T8, T9, T10, T11 and/or T12 dermatome nerve(s);
   vii. small intestine maps to the T10, T11 and/or T12 dermatome nerve(s);
   viii. colon maps to the T11, S1, S2, S3 and/or S4 dermatome nerve(s);
   ix. kidney maps to the T10, T11, T12 and/or L1 dermatome nerve(s);
   x. ureter maps to the T11, T12 and/or L1 dermatome nerve(s);
   xi. ovary maps to the T11 dermatome nerve(s);
   xii. fallopian tube(s) maps to the T11 dermatome nerve(s);
   xiii. cervix maps to the S2, S3 and/or S4 dermatome nerve(s);
   xiv. testes maps to the T10, T11, L1, L2, S1, S2, S3 and/or S4 dermatome nerve(s);
   xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds sensory afferent nerve cells, enters said cells (e.g. by receptor-mediated endocytosis) and suppresses neurotransmitter release therefrom, thereby suppressing said visceral pain.

Also provided is use of a clostridial neurotoxin in the manufacture of a medicament for suppressing visceral pain in a patient, wherein suppressing visceral pain comprises:
a. identifying an organ in the patient that is causing said visceral pain;
b. mapping said organ to a specific dermatome nerve according to the following instructions, thereby matching said organ to one or more specifically designated dermatome nerve(s):
   i. urinary bladder maps to the T11, T12, L1, S1, S2, S3 and/or S4 dermatome nerve(s);
   ii. fallopian tube(s) maps to the T11 dermatome nerve(s);
   iii. uterus maps to the T12 and/or L1 dermatome nerve(s);
   iv. cervix maps to the S2, S3 and/or S4 dermatome nerve(s);
   v. stomach maps to the T8, T9 and/or T10 dermatome nerve(s);
   vi. liver maps to the T8, T9, T10 and/or T11 dermatome nerve(s);
   vii. gall bladder maps to the T8, T9, T10 and/or T11 dermatome nerve(s);
   viii. pancreas maps to the T7, T8, T9, T10, T11 and/or T12 dermatome nerve(s);
   ix. small intestine maps to the T10, T11 and/or T12 dermatome nerve(s);
   x. colon maps to the T11, S1, S2, S3 and/or S4 dermatome nerve(s);
   xi. kidney maps to the T10, T11, T12 and/or L1 dermatome nerve(s);
   xii. ureter maps to the T11, T12 and/or L1 dermatome nerve(s);
   xiii. ovary maps to the T11 dermatome nerve(s);
   xiv. testes maps to the T10, T11, L1, L2, S1, S2, S3 and/or S4 dermatome nerve(s);
   xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

Throughout this specification, the term “sensory afferent nerve cells” may be used synonymously with the term “visceral afferent nerve cells”. The visceral afferent nerve cells (also known as visceral afferent fibres, or general visceral afferent (GVA) fibers) conduct sensory impulses (usually pain or reflex sensations) from the viscera (e.g. internal organs, glands) to the central nervous system.

One aspect provides a clostridial neurotoxin for use in a method of suppressing visceral pain in a patient, the method comprising:
a. identifying an organ in the patient that is causing said visceral pain;
b. mapping said organ to a specific dermatome nerve according to the following instructions, thereby matching said organ to one or more specifically designated dermatome nerve(s):
   i. urinary bladder maps to the T11, T12, L1, S1, S2, S3 and/or S4 dermatome nerve(s);
   ii. uterus maps to the T12 and/or L1 dermatome nerve(s);
   iii. stomach maps to the T8, T9 and/or T10 dermatome nerve(s);
   iv. liver maps to the T8, T9, T10 and/or T11 dermatome nerve(s);
   v. gall bladder maps to the T8, T9, T10 and/or T11 dermatome nerve(s);
   vi. pancreas maps to the T7, T8, T9, T10, T11 and/or T12 dermatome nerve(s);
   vii. small intestine maps to the T10, T11 and/or T12 dermatome nerve(s);
   viii. colon maps to the T11, S1, S2, S3 and/or S4 dermatome nerve(s);
   ix. kidney maps to the T10, T11, T12 and/or L1 dermatome nerve(s);
   x. ureter maps to the T11, T12 and/or L1 dermatome nerve(s);
   xi. ovary maps to the T11 dermatome nerve(s);
   xii. fallopian tube(s) maps to the T11 dermatome nerve(s);
   xiii. cervix maps to the S2, S3 and/or S4 dermatome nerve(s);
   xiv. testes maps to the T10, T11, L1, L2, S1, S2, S3 and/or S4 dermatome nerve(s);
   xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds visceral afferent nerve cells, enters said cells (e.g. by receptor-mediated endocytosis) and suppresses neurotransmitter release therefrom, thereby suppressing said visceral pain.

One aspect provides a method of suppressing visceral pain in a patient, the method comprising:
a. identifying an organ in the patient that is causing said visceral pain;
b. mapping said organ to a specific dermatome nerve according to the following instructions, thereby matching said organ to one or more specifically designated dermatome nerve(s):
   i. urinary bladder maps to the T11, T12, L1, S1, S2, S3 and/or S4 dermatome nerve(s);
   ii. uterus maps to the T12 and/or L1 dermatome nerve(s);
   iii. stomach maps to the T8, T9 and/or T10 dermatome nerve(s);
   iv. liver maps to the T8, T9, T10 and/or T11 dermatome nerve(s);
   v. gall bladder maps to the T8, T9, T10 and/or T11 dermatome nerve(s);
   vi. pancreas maps to the T7, T8, T9, T10, T11 and/or T12 dermatome nerve(s);
   vii. small intestine maps to the T10, T11 and/or T12 dermatome nerve(s);
   viii. colon maps to the T11, S1, S2, S3 and/or S4 dermatome nerve(s);
   ix. kidney maps to the T10, T11, T12 and/or L1 dermatome nerve(s);
   x. ureter maps to the T11, T12 and/or L1 dermatome nerve(s);
   xi. ovary maps to the T11 dermatome nerve(s);
   xii. fallopian tube(s) maps to the T11 dermatome nerve(s);
   xiii. cervix maps to the S2, S3 and/or S4 dermatome nerve(s);
   xiv. testes maps to the T10, T11, L1, L2, S1, S2, S3 and/or S4 dermatome nerve(s);
   xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds visceral afferent nerve cells, enters said cells (e.g. by receptor-mediated endocytosis) and suppresses neurotransmitter release therefrom, thereby suppressing said visceral pain.

One aspect provides use of a clostridial neurotoxin in the manufacture of a medicament for suppressing visceral pain in a patient, wherein suppressing visceral pain comprises:
a. identifying an organ in the patient that is causing said visceral pain;
b. mapping said organ to a specific dermatome nerve according to the following instructions, thereby matching said organ to one or more specifically designated dermatome nerve(s):
   i. urinary bladder maps to the T11, T12, L1, S1, S2, S3 and/or S4 dermatome nerve(s);
   ii. fallopian tube(s) maps to the T11 dermatome nerve(s);
   iii. uterus maps to the T12 and/or L1 dermatome nerve(s);
   iv. cervix maps to the S2, S3 and/or S4 dermatome nerve(s);
   v. stomach maps to the T8, T9 and/or T10 dermatome nerve(s);
   vi. liver maps to the T8, T9, T10 and/or T11 dermatome nerve(s);
   vii. gall bladder maps to the T8, T9, T10 and/or T11 dermatome nerve(s);
   viii. pancreas maps to the T7, T8, T9, T10, T11 and/or T12 dermatome nerve(s);
   ix. small intestine maps to the T10, T11 and/or T12 dermatome nerve(s);
   x. colon maps to the T11, S1, S2, S3 and/or S4 dermatome nerve(s);
   xi. kidney maps to the T10, T11, T12 and/or L1 dermatome nerve(s);
   xii. ureter maps to the T11, T12 and/or L1 dermatome nerve(s);
   xiii. ovary maps to the T11 dermatome nerve(s);
   xiv. testes maps to the T10, T11, L1, L2, S1, S2, S3 and/or S4 dermatome nerve(s);
   xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds visceral afferent nerve cells, enters said cells by receptor-mediated endocytosis and suppresses neurotransmitter release therefrom, thereby suppressing said visceral pain.

One aspect provides a clostridial neurotoxin for use in a method of suppressing visceral pain in a patient, the method comprising:
a. identifying an organ in the patient that is causing said visceral pain;
b. mapping said organ to a specific dermatome according to the following instructions, thereby matching said organ to one or more specifically designated dermatome(s):
   i. urinary bladder maps to the T11, T12, L1, S1, S2, S3 and/or S4 dermatome(s);
   ii. uterus maps to the T12 and/or L1 dermatome(s);
   iii. stomach maps to the T8, T9 and/or T10 dermatome(s);
   iv. liver maps to the T8, T9, T10 and/or T11 dermatome(s);
   v. gall bladder maps to the T8, T9, T10 and/or T11 dermatome(s);
   vi. pancreas maps to the T7, T8, T9, T10, T11 and/or T12 dermatome(s);
   vii. small intestine maps to the T10, T11 and/or T12 dermatome(s);
   viii. colon maps to the T11, S1, S2, S3 and/or S4 dermatome(s);
   ix. kidney maps to the T10, T11, T12 and/or L1 dermatome(s);
   x. ureter maps to the T11, T12 and/or L1 dermatome(s);
   xi. ovary maps to the T11 dermatome(s);
   xii. fallopian tube(s) maps to the T11 dermatome(s);
   xiii. cervix maps to the S2, S3 and/or S4 dermatome(s);
   xiv. testes maps to the T10, T11, L1, L2, S1, S2, S3 and/or S4 dermatome(s);
   xv. epididymis maps to the T10, T11, L1, L2, S1, S2, S3 and/or S4 dermatome(s); and
c. administering a therapeutically effective amount of a clostridial neurotoxin at a site proximal to said one or more specifically designated dermatome(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds sensory afferent nerve cells, enters said cells (e.g. by receptor-mediated endocytosis) and suppresses neurotransmitter release therefrom, thereby suppressing said visceral pain.

One aspect provides a method of suppressing visceral pain in a patient, the method comprising:
a. identifying an organ in the patient that is causing said visceral pain;
b. mapping said organ to a specific dermatome according to the following instructions, thereby matching said organ to one or more specifically designated dermatome(s):
   i. urinary bladder maps to the T11, T12, L1, S1, S2, S3 and/or S4 dermatome(s);
   ii. uterus maps to the T12 and/or L1 dermatome(s);
   iii. stomach maps to the T8, T9 and/or T10 dermatome(s);
   iv. liver maps to the T8, T9, T10 and/or T11 dermatome(s);
   v. gall bladder maps to the T8, T9, T10 and/or T11 dermatome(s);
   vi. pancreas maps to the T7, T8, T9, T10, T11 and/or T12 dermatome(s);
   vii. small intestine maps to the T10, T11 and/or T12 dermatome(s);
   viii. colon maps to the T11, S1, S2, S3 and/or S4 dermatome(s);
   ix. kidney maps to the T10, T11, T12 and/or L1 dermatome(s);
   x. ureter maps to the T11, T12 and/or L1 dermatome(s);
   xi. ovary maps to the T11 dermatome(s);
   xii. fallopian tube(s) maps to the T11 dermatome(s);
   xiii. cervix maps to the S2, S3 and/or S4 dermatome(s);
   xiv. testes maps to the T10, T11, L1, L2, S1, S2, S3 and/or S4 dermatome(s);
   xv. epididymis maps to the T10, T11, L1, L2, S1, S2, S3 and/or S4 dermatome(s); and
c. administering a therapeutically effective amount of a clostridial neurotoxin at a site proximal to said one or more specifically designated dermatome(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds sensory afferent nerve cells, enters said cells (e.g. by receptor-mediated endocytosis) and suppresses neurotransmitter release therefrom, thereby suppressing said visceral pain.

One aspect provides use of a clostridial neurotoxin in the manufacture of a medicament for suppressing visceral pain in a patient, wherein suppressing visceral pain comprises:
a. identifying an organ in the patient that is causing said visceral pain;
b. mapping said organ to a specific dermatome according to the following instructions, thereby matching said organ to one or more specifically designated dermatome(s):
   i. urinary bladder maps to the T11, T12, L1, S1, S2, S3 and/or S4 dermatome(s);
   ii. uterus maps to the T12 and/or L1 dermatome(s);
   iii. stomach maps to the T8, T9 and/or T10 dermatome(s);
   iv. liver maps to the T8, T9, T10 and/or T11 dermatome(s);
   v. gall bladder maps to the T8, T9, T10 and/or T11 dermatome(s);
   vi. pancreas maps to the T7, T8, T9, T10, T11 and/or T12 dermatome(s);
   vii. small intestine maps to the T10, T11 and/or T12 dermatome(s);
   viii. colon maps to the T11, S1, S2, S3 and/or S4 dermatome(s);
   ix. kidney maps to the T10, T11, T12 and/or L1 dermatome(s);
   x. ureter maps to the T11, T12 and/or L1 dermatome(s);
   xi. ovary maps to the T11 dermatome(s);
   xii. fallopian tube(s) maps to the T11 dermatome(s);
   xiii. cervix maps to the S2, S3 and/or S4 dermatome(s);
   xiv. testes maps to the T10, T11, L1, L2, S1, S2, S3 and/or S4 dermatome(s);
   xv. epididymis maps to the T10, T11, L1, L2, S1, S2, S3 and/or S4 dermatome(s); and
c. administering a therapeutically effective amount of a clostridial neurotoxin at a site proximal to said one or more specifically designated dermatome(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds sensory afferent nerve cells, enters said cells (e.g. by receptor-mediated endocytosis) and suppresses neurotransmitter release therefrom, thereby suppressing said visceral pain.

One aspect provides a clostridial neurotoxin for use in a method of suppressing visceral pain in a patient, the method comprising:
a. identifying an organ in the patient that is causing said visceral pain;
b. mapping said organ to a specific dermatome according to the following instructions, thereby matching said organ to one or more specifically designated dermatome(s):
   i. urinary bladder maps to the T11, T12, L1, S1, S2, S3 and/or S4 dermatome(s);
   ii. uterus maps to the T12 and/or L1 dermatome(s);
   iii. stomach maps to the T8, T9 and/or T10 dermatome(s);
   iv. liver maps to the T8, T9, T10 and/or T11 dermatome(s);
   v. gall bladder maps to the T8, T9, T10 and/or T11 dermatome(s);
   vi. pancreas maps to the T7, T8, T9, T10, T11 and/or T12 dermatome(s);
   vii. small intestine maps to the T10, T11 and/or T12 dermatome(s);
   viii. colon maps to the T11, S1, S2, S3 and/or S4 dermatome(s);
   ix. kidney maps to the T10, T11, T12 and/or L1 dermatome(s);
   x. ureter maps to the T11, T12 and/or L1 dermatome(s);
   xi. ovary maps to the T11 dermatome(s);
   xii. fallopian tube(s) maps to the T11 dermatome(s);
   xiii. cervix maps to the S2, S3 and/or S4 dermatome(s);
   xiv. testes maps to the T10, T11, L1, L2, S1, S2, S3 and/or S4 dermatome(s);
   xv. epididymis maps to the T10, T11, L1, L2, S1, S2, S3 and/or S4 dermatome(s); and
c. administering a therapeutically effective amount of a clostridial neurotoxin at a site proximal to said one or more specifically designated dermatome(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds visceral afferent nerve cells, enters said cells (e.g. by receptor-mediated endocytosis) and suppresses neurotransmitter release therefrom, thereby suppressing said visceral pain.

One aspect provides a method of suppressing visceral pain in a patient, the method comprising:
a. identifying an organ in the patient that is causing said visceral pain;
b. mapping said organ to a specific dermatome according to the following instructions, thereby matching said organ to one or more specifically designated dermatome(s):
   i. urinary bladder maps to the T11, T12, L1, S1, S2, S3 and/or S4 dermatome(s);
   ii. uterus maps to the T12 and/or L1 dermatome(s);
   iii. stomach maps to the T8, T9 and/or T10 dermatome(s);
   iv. liver maps to the T8, T9, T10 and/or T11 dermatome(s);
   v. gall bladder maps to the T8, T9, T10 and/or T11 dermatome(s);
   vi. pancreas maps to the T7, T8, T9, T10, T11 and/or T12 dermatome(s);
   vii. small intestine maps to the T10, T11 and/or T12 dermatome(s);
   viii. colon maps to the T11, S1, S2, S3 and/or S4 dermatome(s);
   ix. kidney maps to the T10, T11, T12 and/or L1 dermatome(s);
   x. ureter maps to the T11, T12 and/or L1 dermatome(s);
   xi. ovary maps to the T11 dermatome(s);
   xii. fallopian tube(s) maps to the T11 dermatome(s);
   xiii. cervix maps to the S2, S3 and/or S4 dermatome(s);
   xiv. testes maps to the T10, T11, L1, L2, S1, S2, S3 and/or S4 dermatome(s);
   xv. epididymis maps to the T10, T11, L1, L2, S1, S2, S3 and/or S4 dermatome(s); and
c. administering a therapeutically effective amount of a clostridial neurotoxin at a site proximal to said one or more specifically designated dermatome(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds visceral afferent nerve cells, enters said cells (e.g. by receptor-mediated endocytosis) and suppresses neurotransmitter release therefrom, thereby suppressing said visceral pain.

One aspect provides use of a clostridial neurotoxin in the manufacture of a medicament for suppressing visceral pain in a patient, wherein suppressing visceral pain comprises:
a. identifying an organ in the patient that is causing said visceral pain;
b. mapping said organ to a specific dermatome according to the following instructions, thereby matching said organ to one or more specifically designated dermatome(s):
   i. urinary bladder maps to the T11, T12, L1, S1, S2, S3 and/or S4 dermatome(s);
   ii. uterus maps to the T12 and/or L1 dermatome(s);
   iii. stomach maps to the T8, T9 and/or T10 dermatome(s);
   iv. liver maps to the T8, T9, T10 and/or T11 dermatome(s);
   v. gall bladder maps to the T8, T9, T10 and/or T11 dermatome(s);
   vi. pancreas maps to the T7, T8, T9, T10, T11 and/or T12 dermatome(s);
   vii. small intestine maps to the T10, T11 and/or T12 dermatome(s);
   viii. colon maps to the T11, S1, S2, S3 and/or S4 dermatome(s);
   ix. kidney maps to the T10, T11, T12 and/or L1 dermatome(s);
   x. ureter maps to the T11, T12 and/or L1 dermatome(s);
   xi. ovary maps to the T11 dermatome(s);
   xii. fallopian tube(s) maps to the T11 dermatome(s);
   xiii. cervix maps to the S2, S3 and/or S4 dermatome(s);
   xiv. testes maps to the T10, T11, L1, L2, S1, S2, S3 and/or S4 dermatome(s);
   xv. epididymis maps to the T10, T11, L1, L2, S1, S2, S3 and/or S4 dermatome(s); and
c. administering a therapeutically effective amount of a clostridial neurotoxin at a site proximal to said one or more specifically designated dermatome(s), wherein following administration of said clostridial neurotoxin to the patient, the clostridial neurotoxin is transported via retrograde axonal transport to the spinal cord (e.g. the dorsal horn(s)) where it (the clostridial neurotoxin) binds visceral afferent nerve cells, enters said cells (e.g. by receptor-mediated endocytosis) and suppresses neurotransmitter release therefrom, thereby suppressing said visceral pain.

As mentioned above, broad aspects of the invention provide any of the following:
a clostridial neurotoxin for use in a method of suppressing visceral pain;
a method of suppressing visceral pain in a patient, the method comprising administering a clostridial neurotoxin to the patient;
use of a clostridial neurotoxin in the manufacture of a medicament to suppress visceral pain in a patient.

For example, a method of the invention may comprise identifying an organ in the patient that is “contributing to” (aka “causing”) said visceral pain. A method of the invention may comprise mapping said organ to a specific dermatome nerve; for example, selected from the following combinations:
i. stomach maps to the T8, T9 and/or T10 dermatome(s);
ii. liver maps to the T8, T9, T10 and/or T11 dermatome(s) (preferably the T8, T9 and/or T10 dermatome(s));
iii. gall bladder maps to the T8, T9, T10 and/or T11 dermatome(s) (preferably the T8, T9 and/or T10 dermatome(s));
iv. pancreas maps to the T7, T8, T9, T10, T11 and/or T12 dermatome(s);
v. small intestine maps to the T10, T11 and/or T12 dermatome(s);
vi. colon maps to the T11, S1, S2, S3 and/or S4 dermatome(s) (preferably the S1, S2, S3 and/or S4 dermatome(s));
vii. kidney maps to the T10, T11, T12 and/or L1 dermatome(s) (preferably the T11, T12 and/or L1 dermatome(s));
viii. ureter maps to the T11, T12 and/or L1 dermatome(s);
ix. urinary bladder maps to the T12, L1, S2, S3 and/or S4 dermatome(s);
x. ovary maps to the T11 dermatome(s);
xi. fallopian tube(s) maps to the T11 dermatome(s);
xii. uterus maps to the T12 and/or L1 dermatome(s);
xiii. cervix maps to the S2, S3 and/or S4 dermatome(s);
xiv. testes maps to the T10, T11, L1, L2, S1, S2, S3 and/or S4 dermatome(s) (preferably the T11, L1, L2, S1, S2, S3 and/or S4 dermatome(s));
xv. epididymis maps to the T10, T11, L1, L2, S1, S2, S3 and/or S4 dermatome(s) (preferably the T11, L1, L2, S1, S2, S3 and/or S4 dermatome(s));
xvi. heart maps to the T3 and/or T4 dermatome(s);
xvii. esophagus maps to the T4 and/or T5 dermatome(s).

A method of the invention may comprise administering a clostridial neurotoxin at a site proximal to the same one or more dermatome nerve that map(s) to the organ identified as contributing to the visceral pain. The clostridial neurotoxin may suppress visceral pain following administration. Suitably, following administration of said clostridial neurotoxin to the patient, the neurotoxin is transported via retrograde axonal transport to the spinal cord (e.g. the dorsal horn(s)) and suppresses the underlying visceral pain integration.

A more particular aspect provides a clostridial neurotoxin for use in a method of suppressing visceral pain in a patient, the method comprising:
a. identifying an organ in the patient that is contributing to said visceral pain;
b. mapping said organ to a specific dermatome nerve selected from the following combinations:
   i. stomach maps to the T8, T9 and/or T10 dermatome(s);
   ii. liver maps to the T8, T9, T10 and/or T11 dermatome(s) (preferably the T8, T9 and/or T10 dermatome(s));
   iii. gall bladder maps to the T8, T9, T10 and/or T11 dermatome(s) (preferably the T8, T9 and/or T10 dermatome(s));
   iv. pancreas maps to the T7, T8, T9, T10, T11 and/or T12 dermatome(s);
   v. small intestine maps to the T10, T11 and/or T12 dermatome(s);
   vi. colon maps to the T11, S1, S2, S3 and/or S4 dermatome(s) (preferably the S1, S2, S3 and/or S4 dermatome(s));
   vii. kidney maps to the T10, T11, T12 and/or L1 dermatome(s) (preferably the T11, T12 and/or L1 dermatome(s));
   viii. ureter maps to the T11, T12 and/or L1 dermatome(s);
   ix. urinary bladder maps to the T12, L1, S2, S3 and/or S4 dermatome(s);
   x. ovary maps to the T11 dermatome(s);
   xi. fallopian tube(s) maps to the T11 dermatome(s);
   xii. uterus maps to the T12 and/or L1 dermatome(s);
   xiii. cervix maps to the S2, S3 and/or S4 dermatome(s);
   xiv. testes maps to the T10, T11, L1, L2, S1, S2, S3 and/or S4 dermatome(s) (preferably the T11, L1, L2, S1, S2, S3 and/or S4 dermatome(s));
   xv. epididymis maps to the T10, T11, L1, L2, S1, S2, S3 and/or S4 dermatome(s) (preferably the T11, L1, L2, S1, S2, S3 and/or S4 dermatome(s)); and
c. administering a clostridial neurotoxin at a site proximal to the same one or more dermatome nerve that map(s) to the organ identified as contributing to the visceral pain, and wherein following administration of said clostridial neurotoxin to the patient, the clostridial neurotoxin is transported via retrograde axonal transport to the spinal cord (e.g. the dorsal horn(s)) and suppresses the underlying visceral pain integration.

In other words, one aspect provides a method of suppressing visceral pain in a patient in need thereof, the method comprising:
a. identifying an organ in the patient that is contributing to said visceral pain;
b. mapping said organ to a specific dermatome nerve selected from the following combinations:
   i. stomach maps to the T8, T9 and/or T10 dermatome(s);
   ii. liver maps to the T8, T9, T10 and/or T11 dermatome(s) (preferably the T8, T9 and/or T10 dermatome(s));
   iii. gall bladder maps to the T8, T9, T10 and/or T11 dermatome(s) (preferably the T8, T9 and/or T10 dermatome(s));
   iv. pancreas maps to the T7, T8, T9, T10, T11 and/or T12 dermatome(s);
   v. small intestine maps to the T10, T11 and/or T12 dermatome(s);
   vi. colon maps to the T11, S1, S2, S3 and/or S4 dermatome(s) (preferably the S1, S2, S3 and/or S4 dermatome(s));
   vii. kidney maps to the T10, T11, T12 and/or L1 dermatome(s) (preferably the T11, T12 and/or L1 dermatome(s));
   viii. ureter maps to the T11, T12 and/or L1 dermatome(s);
   ix. urinary bladder maps to the T12, L1, S2, S3 and/or S4 dermatome(s);
   x. ovary maps to the T11 dermatome(s);
   xi. fallopian tube(s) maps to the T11 dermatome(s);
   xii. uterus maps to the T12 and/or L1 dermatome(s);
   xiii. cervix maps to the S2, S3 and/or S4 dermatome(s);
   xiv. testes maps to the T10, T11, L1, L2, S1, S2, S3 and/or S4 dermatome(s) (preferably the T11, L1, L2, S1, S2, S3 and/or S4 dermatome(s));
   xv. epididymis maps to the T10, T11, L1, L2, S1, S2, S3 and/or S4 dermatome(s) (preferably the T11, L1, L2, S1, S2, S3 and/or S4 dermatome(s)); and
c. administering a clostridial neurotoxin at a site proximal to the same one or more dermatome nerve that map(s) to the organ identified as contributing to the visceral pain, and wherein following administration of said clostridial neurotoxin to the patient, the clostridial neurotoxin is transported via retrograde axonal transport to the spinal cord (e.g. the dorsal horn(s)) and suppresses the underlying visceral pain integration.

One aspect provides use of a clostridial neurotoxin in the manufacture of a medicament for suppressing visceral pain in a patient, wherein suppressing visceral pain comprises:
a. identifying an organ in the patient that is contributing to said visceral pain;
b. mapping said organ to a specific dermatome nerve selected from the following combinations:
   i. stomach maps to the T8, T9 and/or T10 dermatome(s);
   ii. liver maps to the T8, T9, T10 and/or T11 dermatome(s) (preferably the T8, T9 and/or T10 dermatome(s));
   iii. gall bladder maps to the T8, T9, T10 and/or T11 dermatome(s) (preferably the T8, T9 and/or T10 dermatome(s));
   iv. pancreas maps to the T7, T8, T9, T10, T11 and/or T12 dermatome(s);
   v. small intestine maps to the T10, T11 and/or T12 dermatome(s);
   vi. colon maps to the T11, S1, S2, S3 and/or S4 dermatome(s) (preferably the S1, S2, S3 and/or S4 dermatome(s));
   vii. kidney maps to the T10, T11, T12 and/or L1 dermatome(s) (preferably the T11, T12 and/or L1 dermatome(s));
   viii. ureter maps to the T11, T12 and/or L1 dermatome(s);
   ix. urinary bladder maps to the T12, L1, S2, S3 and/or S4 dermatome(s);
   x. ovary maps to the T11 dermatome(s);
   xi. fallopian tube(s) maps to the T11 dermatome(s);
   xii. uterus maps to the T12 and/or L1 dermatome(s);
   xiii. cervix maps to the S2, S3 and/or S4 dermatome(s);
   xiv. testes maps to the T10, T11, L1, L2, S1, S2, S3 and/or S4 dermatome(s) (preferably the T11, L1, L2, S1, S2, S3 and/or S4 dermatome(s));
   xv. epididymis maps to the T10, T11, L1, L2, S1, S2, S3 and/or S4 dermatome(s) (preferably the T11, L1, L2, S1, S2, S3 and/or S4 dermatome(s)); and
c. administering a clostridial neurotoxin at a site proximal to the same one or more dermatome nerve that map(s) to the organ identified as contributing to the visceral pain, and wherein following administration of said clostridial neurotoxin to the patient, the clostridial neurotoxin is transported via retrograde axonal transport to the spinal cord (e.g. the dorsal horn(s)) and suppresses the underlying visceral pain integration.

As mentioned above, it is believed that upon administration of clostridial neurotoxin to a dermatome nerve (e.g. to a dermatome), retrograde axonal transport of the neurotoxin (more particularly, via a spinal nerve afferent) allows for delivery of the neurotoxin to the spinal cord to act at or target the same area in the spinal cord in which visceral pain is integrated. For example, as evidenced in the examples, delivery of clostridial neurotoxin to the spinal cord (following administration to a dermatome) can cause SNARE protein (e.g. SNAP-25) cleavage. This is because, following administration of said clostridial neurotoxin to the patient (wherein the clostridial neurotoxin is transported via retrograde axonal transport to the spinal cord), the clostridial neurotoxin may bind sensory afferent nerve cells, enter said cells (e.g. by receptor-mediated endocytosis) and suppress neurotransmitter release therefrom e.g. by cleaving a SNARE protein, thereby suppressing said visceral pain.

For example, through a protease domain in the L-chain of the clostridial, the clostridial neurotoxin may cleave a SNARE protein (thus suppress neurotransmitter release, thereby suppressing said visceral pain). Examples of SNARE proteins include synaptobrevin (otherwise known as vesicle-associated membrane protein (VAMP)), SNAP-25 and syntaxin. For example, BoNT/B, BoNT/D, BoNT/F, BoNT/G, and TeNT cleave synaptobrevin, otherwise known as vesicle-associated membrane protein (VAMP); BoNT/A, BoNT/C, and BoNT/E cleave the synaptosomal-associated protein of 25 kDa (“SNAP-25”); BoNT/C cleaves syntaxin.

SNARE proteins are associated either with the membrane of a secretory vesicle or with a cell membrane and facilitate exocytosis of molecules by mediating the fusion of the secretory vesicle with the cell membrane, thus allowing for the contents of the vesicle to be expelled outside the cell. The cleavage of such SNARE proteins inhibits such exocytosis and thus inhibits the release of neurotransmitter from such neurons. As a result thereof, integration of visceral pain into the central nervous system can be suppressed; in other words, visceral pain may be suppressed.

“Retrograde axonal transport” is a form of axonal transport (aka. axoplasmic transport or axoplasmic flow), a cellular process normally responsible for movement of mitochondria, lipids, synaptic vesicles, proteins, and other organelles to and from a neuron's cell body, through the cytoplasm of its axon called the axoplasm. Axons are on the order of meters long, such that neurons cannot rely on diffusion to carry products of the nucleus and organelles to the end of their axons, hence the use of axonal transport. Axonal transport is also responsible for moving molecules destined for degradation from the axon back to the cell body, where they are broken down by lysosomes. Movement toward the cell body is called “retrograde transport” and movement toward the synapse is called “anterograde transport”. The present invention takes advantage of vesicular retrograde transport to move a clostridial neurotoxin to the spinal cord to exert a central effect.

Thus, the term “retrograde axonal transport to the spinal cord” may be defined as axonal transport of the clostridial neurotoxin toward the nerve cell body that is positioned in the proximity of the spinal cord.

The term “dermatome nerve” may be used synonymously with the term “spinal nerve” and/or “spinal nerve afferent”. Spinal nerves innervate particular dermatomes (e.g. to provide dermatome nerves), and further details on the spinal nerves is provided below. As will be explained in more detail below, a “dermatome” may be defined as an area of skin that is supplied by any given spinal nerve. For convenience, the nerve(s) targeted by the present invention may be referred to as “dermatome nerve(s)”, to illustrate the connection between the targeted nerve(s) and the dermatome(s). That being said, it should be noted that throughout this disclosure, a “dermatome nerve” may also (e.g. alternatively) be referred to as a “spinal nerve” and/or “spinal nerve afferent”. For example, “the T10 dermatome nerve” may be referred to as “the T10 spinal nerve”, and so on for any dermatome nerve described herein.

The clostridial neurotoxin may be administered by any route that allows for administration of the clostridial neurotoxin to a dermatome nerve (e.g. spinal nerve) and subsequent retrograde axonal transport of the clostridial neurotoxin to the spinal cord. Examples of suitable administration routes include intradermal and intrathecal administration.

In a preferable embodiment, the clostridial neurotoxin is administered by intradermal administration (e.g. to a dermatome). In this way, a clostridial neurotoxin may be administered in a minimally invasive manner while targeting pain caused by an insult at an internal organ, which would otherwise require access to internal regions of a patient's body. In addition to the convenience provided by intradermal administration, it is further believed that intradermal administration provides an enhanced effect when compared with alternative administration routes (e.g. intramuscular or subcutaneous administration). In a particularly preferred embodiment, the clostridial neurotoxin is administered by intradermal administration to a dermatome. In other words, it is preferred that the clostridial neurotoxin is administered by intradermal administration to a dermatome having a dermatome nerve that maps to the viscera contributing to visceral pain.

The clostridial neurotoxin may be administered (e.g., by intradermal injection) at up to 20 or 15 injection sites per treatment session. Preferably, the clostridial neurotoxin may be administered (e.g., by intradermal injection) at up to 10 injection sites per treatment session, for example up to 9, 8, 7, 6, 5, 4, 3 or 2 sites per treatment session. In one embodiment a clostridial neurotoxin may be administered at 1-10, 3-10, 5-10 or 7-10 injection sites per treatment session.

A “dermatome” (aka “peripheral nerve field”) may be defined as an area of skin that is mainly supplied by afferent nerve fibres from the dorsal root of any given spinal nerve. The dermatomes follow the pattern of spinal nerve innervation of the skin. For example, the dermatomes of the thorax and abdomen present as a stack of discs forming an individual.

Along the arms and legs, the dermatomes run longitudinally. The general pattern of the dermatomes is shared amongst all individuals. Because each segment (nerve) of the spinal cord innervates a different region of the body, dermatomes can be precisely mapped on the body surface. A typical map of the dermatomes of a human subject that is used clinically is outlined in the American Spinal Injury Association's (ASIA) worksheet produced as the International Standards for Neurological Classification of Spinal Cord Injury (ISNCSCI). At the time of writing, the 2019 edition of said worksheet is obtainable here: https://asia-spinalinjury.org/wp-content/uploads/2019/10/ASIA-ISCOS-Worksheet_10.2019_PRINT-Page-1-2.pdf. Dermatome maps are also described in textbooks such as in Wall and Melzack's “Textbook of Pain” (edited by McMahon et al.; 6th edition)—see chapter 53, and Figure 53-1 (partially adapted herein as FIG. 1B). This figure illustrates the segmental level of visceral innervation and the corresponding dermatomes of various viscera e.g. the dermatomes that ‘map’ to the viscera.

Each dermatome is typically supplied by a single spinal nerve. Thus, a “dermatome” may be defined as an area of skin that is supplied by any given spinal nerve which, as mentioned above, is also referred to as a dermatome nerve herein. More particularly, as mentioned above, a “dermatome” may refer to an area of skin that is supplied by afferent nerve fibres from the dorsal root of any given spinal nerve. In humans, there are 30 dermatomes (one less than the number of spinal nerves because the C1 spinal nerve typically doesn't have a sensory root; as a result, dermatomes begin with spinal nerve C2).

Throughout this disclosure, the name given to a dermatome conveniently corresponds to the name of the particular spinal nerve (also referred to a dermatome nerve herein) that innervates said dermatome. For example, a dermatome innervated by the C3 spinal nerve (also referred to the C3 dermatome nerve) is conveniently referred to as the “C3 dermatome”. Similarly, a dermatome innervated by the T1 spinal nerve (also referred to the T1 dermatome nerve) is conveniently referred to as the “T1 dermatome”.

The viscera typically transmit visceral nociceptive information to the central nervous system (e.g. the spinal cord) via visceral afferent fibres following stimulation of nociceptive receptors in the viscera. Visceral afferent nerve innervation of the spinal cord is distributed from the cervical to the sacral spinal segments. Second-order spinal neurons receive visceral afferent input to integrate the visceral pain. Said second-order neurons in the spinal cord that receive visceral afferent input (aka “visceroceptive neurons”) are located principally in the superficial spinal laminae, deeper in lamina V (including the intermediolateral cell column in the TL spinal cord and the sacral parasympathetic nucleus), and in the medial LS spinal cord dorsal to the central canal (lamina X).

Each viscera comprises a corresponding dermatome to which visceral pain (contributed to by said viscera) may be referred. This is because visceral afferent input (e.g. sensory input) to the spinal cord is also characterized by convergence. That is, the second-order spinal neurons that receive visceral input (afferent/sensory input) also receive convergent somatic (or cutaneous) input from skin/a dermatome, which provides an explanation for referral of visceral sensations to somatic sites (e.g., deep retrosternal pain that radiates to the neck, shoulder, or jaw with angina). Indeed, the distribution of referred pain from the viscera can be predicted by the dermatomal distribution of the somatic afferents that enter the spinal cord at the same level as the visceral afferents. The convergence of somatic (e.g. dermatome nerve) and visceral nerves at the spinal cord may be referred to as “somatovisceral convergence”.

Without wishing to be bound by theory, it is believed that visceral pain suppression achieved by the inventors herein takes advantage of such somatovisceral convergence onto second-order spinal neurons, by employing dermatome nerves (e.g. spinal nerves/afferents) to deliver a clostridial neurotoxin (by retrograde transport) to a region of the spinal cord where said dermatome nerve converges with a visceral afferent. Thus, it is believed that the clostridial neurotoxin can exert an effect in the spinal cord (as evidenced by SNAP-25 cleavage identified in the dorsal horns) to inhibit signal transmission (e.g. via neurotransmitter secretion) from the visceral afferent to the second-order spinal neurons, thus inhibiting visceral pain integration into the central nervous system.

It is understood that visceral afferent fibers have their cell bodies in dorsal root ganglia (DRG) and terminate in the spinal dorsal horn, from which visceral sensory information is transmitted rostrally in the contralateral spinothalamic tract (STT) or ipsilateral dorsal column (DC) to supraspinal sites (the brain). In other words, there is ‘ascending transmission’. Because of this rostral (or ‘ascending’) transmission, it is believed that suppression of visceral pain at any given segment of the spinal cord may also suppress visceral pain integration at more rostral segments. By way of example, by administering a clostridial neurotoxin to the L1 dermatome to provide suppression of visceral pain integration at the L1 segment of the dermatome, suppression of visceral pain integration at the T12 segment (which is rostral to L1) may also be provided.

The neurons that receive visceral afferent input may be referred to as “visceroceptive spinal neurons”.

Thus, the term “one or more specifically designated dermatome nerve(s)” may refer to a dermatome nerve (e.g. spinal nerve afferent) that converges, in the spinal cord, with a visceral afferent from the organ/viscera identified as contributing to (e.g. causing) the visceral pain. The clostridial neurotoxin may be administered to a “dermatome” toward administering the neurotoxin to a “dermatome nerve”; in which case, the method may comprise mapping said organ to a specific dermatome according to instructions outlined in the claims, thereby matching said organ to one or more specifically designated dermatome(s). The term “one or more specifically designated dermatome(s)” may refer to a dermatome innervated by a spinal nerve that converges, in the spinal cord, with a visceral afferent from the organ/viscera identified as contributing to the visceral pain.

As indicated above, convergence of afferent input is characteristic of visceroceptive spinal neurons. It is believed that typically all visceroceptive spinal neurons receive convergent somatic input. The dermatome(s)/dermatome nerve(s) corresponding to (e.g. mapping to) any given viscera have been mapped out, for example via studies into the phenomenon of “referred pain”. Indeed, the distribution of referred pain from the viscera can be predicted by the dermatomal distribution of the somatic afferents that enter the spinal cord at the same level as the visceral afferents. The present inventors have thus taken advantage of such dermatomal mapping to target and suppress visceral pain integration via administration of a clostridial neurotoxin to an appropriate dermatome/dermatome nerve.

The term “one or more dermatome nerve that map(s) to the organ identified as contributing to the visceral pain” may refer to a dermatome nerve (e.g. spinal nerve afferent) that converges, in the spinal cord, with a visceral afferent from the organ/viscera identified as contributing to (e.g. causing) the visceral pain. In other words, the term “one or more dermatome that map(s) to the organ identified as contributing to the visceral pain” may refer to a dermatome innervated by a spinal nerve that converges, in the spinal cord, with a visceral afferent from the organ/viscera identified as contributing to the visceral pain. As indicated above, convergence of afferent input is characteristic of visceroceptive spinal neurons. It is believed that typically all visceroceptive spinal neurons receive convergent somatic input. The dermatome(s) corresponding to any given viscera have been mapped out via studies into the phenomenon of “referred pain”. Indeed, the distribution of referred pain from the viscera can be predicted by the dermatomal distribution of the somatic afferents that enter the spinal cord at the same level as the visceral afferents. The present inventors have thus taken advantage of such dermatomal mapping to target and suppress visceral pain integration via administration of a clostridial neurotoxin to an appropriate dermatome.

The term “underlying visceral pain integration” may refer to the activation of second-order spinal neurons (aka. visceroceptive neurons) that receive visceral afferent input. A nociceptive stimulus is detected at the viscera via nociceptive receptors, and nociceptive information is transmitted to the central nervous system which processes the information into an unpleasant sensation perceived as pain. As outlined above, second-order spinal neurons (aka. visceroceptive neurons) are activated by visceral afferent input to integrate the visceral pain. It is believed that the pain suppression demonstrated by the inventors (see the Examples) is achieved by suppressing signal transfer from a visceral afferent to a visceroceptive neuron in the spinal cord, for example by inhibiting neurotransmitter secretion from the visceral afferent that would otherwise cross the synapse between said visceral afferent and visceroceptive neuron to bind a corresponding receptor on the visceroceptive neuron. Indeed, SNARE protein (more particularly, SNAP-25) cleavage has been detected in the spinal cord following administration of clostridial neurotoxin to a dermatome, said cleavage correlating with visceral pain suppression.

Because of their involvement in defining any given dermatome, a discussion of the spinal nerves (also referred to as dermatome nerves throughout this disclosure) is now provided.

Spinal nerves are part of the peripheral nervous system (PNS) that connects the CNS with the rest of the body. In humans, there are 31 pairs of spinal nerves. They form from nerve roots that branch from the spinal cord. Spinal nerves are named and grouped by the region of the spine that they are associated with. There are five groups of spinal nerves, namely:
Cervical nerves. There are eight pairs of these cervical nerves, numbered C1-C8. They originate from the neck (e.g. spine at the neck);
Thoracic nerves. There are 12 pairs of thoracic nerves, numbered T1-T12. They originate in the part of the spine that makes up the torso;
Lumbar nerves. There are five pairs of lumbar spinal nerves, numbered L1-L5. They come from the part of the spine that makes up your lower back;
Sacral nerves. There are five pairs of sacral spinal nerves, numbered S1-S5. They are associated with the sacrum, one of the bones found in the pelvis;
Coccygeal nerves. There is a single pair of coccygeal spinal nerves. This pair of nerves originates from the area of the coccyx, or tailbone.

Each of these nerves relays sensation (including pain) from a particular region of skin (i.e. a dermatome) to the brain. Along the thorax and abdomen, the dermatomes are like a stack of discs forming a human, each supplied by a different spinal nerve. Along the arms and the legs, the pattern is different: the dermatomes run longitudinally along the limbs. The general pattern is similar in all people.

The term “at a site proximal to said one or more specifically designated dermatome nerve(s)” may mean a site that is located within (and including) 5 mm, 4 mm, 3 mm, 2 mm or 1 mm of said specifically designated dermatome nerve(s). Preferably, “at a site proximal to said one or more specifically designated dermatome nerve(s)” means the clostridial neurotoxin is administered to said specifically designated dermatome nerve.

The term “at a site proximal to said one or more specifically designated dermatome nerve(s)” may mean a site that is located within (and including) 5 mm, 4 mm, 3 mm, 2 mm or 1 mm of the dermatome of said one or more specifically designated dermatome nerve(s). Preferably, “at a site proximal to said one or more specifically designated dermatome nerve(s)” means the clostridial neurotoxin is administered to the dermatome of said specifically designated dermatome nerve.

The term “at a site proximal to said one or more specifically designated dermatome(s)” may mean a site that is located within (and including) 5 mm, 4 mm, 3 mm, 2 mm or 1 mm of said one or more specifically designated dermatome(s). Preferably, “at a site proximal to said one or more specifically designated dermatome nerve(s)” means the clostridial neurotoxin is administered to said one or more specifically designated dermatome nerve(s).

The following is a list of spinal nerves (also known as dermatome nerves herein), together with the location on the body belonging to the dermatome of any given nerve:
C2—at least one cm lateral to the occipital protuberance at the base of the skull.

Alternately, a point at least 3 cm behind the ear.

C3—In the supraclavicular fossa, at the midclavicular line.

C4—Over the acromioclavicular joint.

C5—On the lateral (radial) side of the antecubital fossa, just proximally to the elbow.

C6—On the dorsal surface of the proximal phalanx of the thumb.

C7—On the dorsal surface of the proximal phalanx of the middle finger.

C8—On the dorsal surface of the proximal phalanx of the little finger.

T1—On the medial (ulnar) side of the antecubital fossa, just proximally to the medial epicondyle of the humerus.
T2—At the apex of the axilla.
T3—Intersection of the midclavicular line and the third intercostal space.
T4—Intersection of the midclavicular line and the fourth intercostal space, located at the level of the nipples.
T5—Intersection of the midclavicular line and the fifth intercostal space, horizontally located midway between the level of the nipples and the level of the xiphoid process.
T6—Intersection of the midclavicular line and the horizontal level of the xiphoid process.
T7—Intersection of the midclavicular line and the horizontal level at one quarter the distance between the level of the xiphoid process and the level of the umbilicus.
T8—Intersection of the midclavicular line and the horizontal level at one half the distance between the level of the xiphoid process and the level of the umbilicus.
T9—Intersection of the midclavicular line and the horizontal level at three quarters of the distance between the level of the xiphoid process and the level of the umbilicus.
T10—Intersection of the midclavicular line, at the horizontal level of the umbilicus.
T11—Intersection of the midclavicular line, at the horizontal level midway between the level of the umbilicus and the inguinal ligament.
T12—Intersection of the midclavicular line and the midpoint of the inguinal ligament.
L1—Midway between the key sensory points for T12 and L2.
L2—On the anterior medial thigh, at the midpoint of a line connecting the midpoint of the inguinal ligament and the medial epicondyle of the femur.
L3—At the medial epicondyle of the femur.
L4—Over the medial malleolus.
L5—On the dorsum of the foot at the third metatarsophalangeal joint.
S1—On the lateral aspect of the calcaneus.
S2—At the midpoint of the popliteal fossa.
S3—Over the tuberosity of the ischium or infragluteal fold.
S4 and S5—In the perianal area, less than one cm lateral to the mucocutaneous zone.

Thus, to administer a clostridial neurotoxin to the:
i. C2 dermatome, the clostridial neurotoxin may be administered in the proximity of at least one cm lateral to the occipital protuberance at the base of the skull (additionally or alternatively, in the proximity of a point at least 3 cm behind the ear);
ii. C3 dermatome, the clostridial neurotoxin may be administered in the proximity of the supraclavicular fossa, at the midclavicular line;
iii. C4 dermatome, the clostridial neurotoxin may be administered in the proximity of over the acromioclavicular joint;
iv. C5 dermatome, the clostridial neurotoxin may be administered in the proximity of the lateral (radial) side of the antecubital fossa, just proximally to the elbow;
v. C6 dermatome, the clostridial neurotoxin may be administered in the proximity of the dorsal surface of the proximal phalanx of the thumb;
vi. C7 dermatome, the clostridial neurotoxin may be administered in the proximity of the dorsal surface of the proximal phalanx of the middle finger;
vii. C8 dermatome, the clostridial neurotoxin may be administered in the proximity of the dorsal surface of the proximal phalanx of the little finger;
viii. T1 dermatome, the clostridial neurotoxin may be administered in the proximity of the medial (ulnar) side of the antecubital fossa, just proximally to the medial epicondyle of the humerus;
ix. T2 dermatome, the clostridial neurotoxin may be administered in the proximity of the apex of the axilla;
x. T3 dermatome, the clostridial neurotoxin may be administered in the proximity of the intersection of the midclavicular line and the third intercostal space;
xi. T4 dermatome, the clostridial neurotoxin may be administered in the proximity of the intersection of the midclavicular line and the fourth intercostal space, located at the level of the nipples;
xii. T5 dermatome, the clostridial neurotoxin may be administered in the proximity of the intersection of the midclavicular line and the fifth intercostal space, horizontally located midway between the level of the nipples and the level of the xiphoid process;
xiii. T6 dermatome, the clostridial neurotoxin may be administered in the proximity of the intersection of the midclavicular line and the horizontal level of the xiphoid process;
xiv. T7 dermatome, the clostridial neurotoxin may be administered in the proximity of the intersection of the midclavicular line and the horizontal level at one quarter the distance between the level of the xiphoid process and the level of the umbilicus;
xv. T8 dermatome, the clostridial neurotoxin may be administered in the proximity of the intersection of the midclavicular line and the horizontal level at one half the distance between the level of the xiphoid process and the level of the umbilicus;
xvi. T9 dermatome, the clostridial neurotoxin may be administered in the proximity of the intersection of the midclavicular line and the horizontal level at three quarters of the distance between the level of the xiphoid process and the level of the umbilicus;
xvii. T10 dermatome, the clostridial neurotoxin may be administered in the proximity of the intersection of the midclavicular line, at the horizontal level of the umbilicus;
xviii. T11 dermatome, the clostridial neurotoxin may be administered in the proximity of the intersection of the midclavicular line, at the horizontal level midway between the level of the umbilicus and the inguinal ligament;
xix. T12 dermatome, the clostridial neurotoxin may be administered in the proximity of the intersection of the midclavicular line and the midpoint of the inguinal ligament;
xx. L1 dermatome, the clostridial neurotoxin may be administered in the proximity of the midway between the key sensory points for T12 and L2;
xxi. L2 dermatome, the clostridial neurotoxin may be administered in the proximity of the anterior medial thigh, at the midpoint of a line connecting the midpoint of the inguinal ligament and the medial epicondyle of the femur;
xxii. L3 dermatome, the clostridial neurotoxin may be administered in the proximity of the medial epicondyle of the femur;
xxiii. L4 dermatome, the clostridial neurotoxin may be administered in the proximity of over the medial malleolus;
xxiv. L5 dermatome, the clostridial neurotoxin may be administered in the proximity of the dorsum of the foot at the third metatarsophalangeal joint;
xxv. S1 dermatome, the clostridial neurotoxin may be administered in the proximity of the lateral aspect of the calcaneus;
xxvi. S2 dermatome, the clostridial neurotoxin may be administered in the proximity of the midpoint of the popliteal fossa;
xxvii. S3 dermatome, the clostridial neurotoxin may be administered in the proximity of over the tuberosity of the ischium or infragluteal fold;
xxviii. S4 and/or S5 dermatome, the clostridial neurotoxin may be administered in the proximity of the perianal area, less than one cm lateral to the mucocutaneous zone.

To administer a clostridial neurotoxin to the:
i. C2 dermatome nerve, the clostridial neurotoxin may be administered in the proximity of at least one cm lateral to the occipital protuberance at the base of the skull (additionally or alternatively, in the proximity of a point at least 3 cm behind the ear);
ii. C3 dermatome nerve, the clostridial neurotoxin may be administered in the proximity of the supraclavicular fossa, at the midclavicular line;
iii. C4 dermatome nerve, the clostridial neurotoxin may be administered in the proximity of over the acromioclavicular joint;
iv. C5 dermatome nerve, the clostridial neurotoxin may be administered in the proximity of the lateral (radial) side of the antecubital fossa, just proximally to the elbow;
v. C6 dermatome nerve, the clostridial neurotoxin may be administered in the proximity of the dorsal surface of the proximal phalanx of the thumb;
vi. C7 dermatome nerve, the clostridial neurotoxin may be administered in the proximity of the dorsal surface of the proximal phalanx of the middle finger;
vii. C8 dermatome nerve, the clostridial neurotoxin may be administered in the proximity of the dorsal surface of the proximal phalanx of the little finger;
viii. T1 dermatome nerve, the clostridial neurotoxin may be administered in the proximity of the medial (ulnar) side of the antecubital fossa, just proximally to the medial epicondyle of the humerus;
ix. T2 dermatome nerve, the clostridial neurotoxin may be administered in the proximity of the apex of the axilla;
x. T3 dermatome nerve, the clostridial neurotoxin may be administered in the proximity of the intersection of the midclavicular line and the third intercostal space;
xi. T4 dermatome nerve, the clostridial neurotoxin may be administered in the proximity of the intersection of the midclavicular line and the fourth intercostal space, located at the level of the nipples;
xii. T5 dermatome nerve, the clostridial neurotoxin may be administered in the proximity of the intersection of the midclavicular line and the fifth intercostal space, horizontally located midway between the level of the nipples and the level of the xiphoid process;
xiii. T6 dermatome nerve, the clostridial neurotoxin may be administered in the proximity of the intersection of the midclavicular line and the horizontal level of the xiphoid process;
xiv. T7 dermatome nerve, the clostridial neurotoxin may be administered in the proximity of the intersection of the midclavicular line and the horizontal level at one quarter the distance between the level of the xiphoid process and the level of the umbilicus;
xv. T8 dermatome nerve, the clostridial neurotoxin may be administered in the proximity of the intersection of the midclavicular line and the horizontal level at one half the distance between the level of the xiphoid process and the level of the umbilicus;
xvi. T9 dermatome nerve, the clostridial neurotoxin may be administered in the proximity of the intersection of the midclavicular line and the horizontal level at three quarters of the distance between the level of the xiphoid process and the level of the umbilicus;
xvii. T10 dermatome nerve, the clostridial neurotoxin may be administered in the proximity of the intersection of the midclavicular line, at the horizontal level of the umbilicus;
xviii. T11 dermatome nerve, the clostridial neurotoxin may be administered in the proximity of the intersection of the midclavicular line, at the horizontal level midway between the level of the umbilicus and the inguinal ligament;
xix. T12 dermatome nerve, the clostridial neurotoxin may be administered in the proximity of the intersection of the midclavicular line and the midpoint of the inguinal ligament;
xx. L1 dermatome nerve, the clostridial neurotoxin may be administered in the proximity of the midway between the key sensory points for T12 and L2;
xxi. L2 dermatome nerve, the clostridial neurotoxin may be administered in the proximity of the anterior medial thigh, at the midpoint of a line connecting the midpoint of the inguinal ligament and the medial epicondyle of the femur;
xxii. L3 dermatome nerve, the clostridial neurotoxin may be administered in the proximity of the medial epicondyle of the femur;
xxiii. L4 dermatome nerve, the clostridial neurotoxin may be administered in the proximity of over the medial malleolus;
xxiv. L5 dermatome nerve, the clostridial neurotoxin may be administered in the proximity of the dorsum of the foot at the third metatarsophalangeal joint;
xxv. S1 dermatome nerve, the clostridial neurotoxin may be administered in the proximity of the lateral aspect of the calcaneus;
xxvi. S2 dermatome nerve, the clostridial neurotoxin may be administered in the proximity of the midpoint of the popliteal fossa;
xxvii. S3 dermatome nerve, the clostridial neurotoxin may be administered in the proximity of over the tuberosity of the ischium or infragluteal fold;
xxviii. S4 and/or S5 dermatome nerve, the clostridial neurotoxin may be administered in the proximity of the perianal area, less than one cm lateral to the mucocutaneous zone.

The term “at a site proximal to said one or more specifically designated dermatome nerve(s)” may mean a site that is located within (and including) 5 mm, 4 mm, 3 mm, 2 mm or 1 mm of said location on the body belonging to the dermatome of said one or more specifically designated dermatome nerve.

The term “at a site proximal to said one or more specifically designated dermatome(s)” may mean a site that is located within (and including) 5 mm, 4 mm, 3 mm, 2 mm or 1 mm of said location on the body belonging to said one or more specifically designated dermatome.

As mentioned above, the association between viscera and the dermatome(s) has been widely mapped out, for example from studies into the phenomenon of “referred pain”. In referred pain, sensory nerve fibers from dermatomes may come together at the same spinal cord level as the general visceral afferent fibers, such as that from the heart. When the general visceral sensory fiber is simulated, the central nervous system does not clearly discern whether the pain is coming from the body wall or from the viscera, so it perceives the pain as coming from somewhere on the body wall, e.g. left arm/hand pain, jaw pain. So the pain is “referred to” the related dermatomes of the same spinal segment.

Thus, it is possible to identify a suitable dermatome/dermatome nerve for receiving administration of a clostridial neurotoxin toward suppressing pain in a given viscera/organ. An illustration of the interaction between the dermatomes and various viscera is provided in FIG. 1.

For example, administration (of clostridial neurotoxin) to:
one or more of the T8, T9, or T10 dermatome(s) may suppress visceral pain, wherein the stomach is contributing to the visceral pain;
one or more of the T8, T9, T10 and/or T11 dermatome(s) (preferably the T8, T9, and/or T10 dermatome(s)) may suppress visceral pain, wherein the liver is contributing to the visceral pain;
one or more of the T8, T9, T10 and/or T11 dermatome(s) (preferably the T8, T9, and/or T10 dermatome(s)) may suppress visceral pain, wherein the gall bladder is contributing to the visceral pain;
one or more of the T7, T8, T9, T10, T11 and/or T12 dermatome(s) may suppress visceral pain, wherein the pancreas is contributing to the visceral pain;
one or more of the T10, T11 and/or T12 dermatome(s) may suppress visceral pain, wherein the small intestine is contributing to the visceral pain;
one or more of the T11, S1, S2, S3 and/or S4 dermatome(s) (preferably the S1, S2, S3 and/or S4 dermatome(s)) may suppress visceral pain, wherein the colon is contributing to the visceral pain;
one or more of the T11, S1, S2, S3 and/or S4 dermatome(s) (preferably the S1, S2, S3 and/or S4 dermatome(s)) may suppress visceral pain, wherein the rectum is contributing to the visceral pain;
one or more of the T10, T11, T12 and/or L1 dermatome(s) (preferably the T11, T12 and/or L1 dermatome(s)) may suppress visceral pain, wherein the kidney is contributing to the visceral pain;
one or more of the T11, T12 and/or L1 dermatome(s) may suppress visceral pain, wherein the ureter is contributing to the visceral pain;
one or more of the T11, T12, L1, S1, S2, S3 and/or S4 dermatome(s) (preferably the T12, L1, S1, S2, S3 and/or S4 dermatome(s)) may suppress visceral pain, wherein the bladder (urinary bladder) is contributing to the visceral pain;
the T11 dermatome may suppress visceral pain, wherein the ovary is contributing to the visceral pain;
the T11 dermatome may suppress visceral pain, wherein a fallopian tube(s) is contributing to the visceral pain;
one or more of the T12 and/or L1 dermatome(s) may suppress visceral pain, wherein the uterus is contributing to the visceral pain;
one or more of the S2, S3 and/or S4 dermatome(s) may suppress visceral pain, wherein the cervix is contributing to the visceral pain;
one or more of the T10, T11, L1, L2, S1, S2, S3 and/or S4 dermatome(s) (preferably the T11, L1, L2, S1, S2, S3 and/or S4 dermatome(s)) may suppress visceral pain, wherein the testes is contributing to the visceral pain;
one or more of the T10, T11, L1, L2, S1, S2, S3 and/or S4 dermatome(s) (preferably the T11, L1, L2, S1, S2, S3 and/or S4 dermatome(s)) may suppress visceral pain, wherein the epididymis is contributing to the visceral pain;
one or more of the T3 and/or T4 dermatome(s) may suppress visceral pain, wherein the heart is contributing to the visceral pain;
one or more of the the T4 and/or T5 dermatome(s) may suppress visceral pain, wherein the esophagus is contributing to the visceral pain.

For example, administration (of clostridial neurotoxin) to:
one or more of the T8, T9, or T10 dermatome nerve(s) may suppress visceral pain, wherein the stomach is contributing to the visceral pain;
one or more of the T8, T9, T10 and/or T11 dermatome nerve(s) (preferably the T8, T9, and/or T10 dermatome nerve(s)) may suppress visceral pain, wherein the liver is contributing to the visceral pain;
one or more of the T8, T9, T10 and/or T11 dermatome nerve(s) (preferably the T8, T9, and/or T10 dermatome nerve(s)) may suppress visceral pain, wherein the gall bladder is contributing to the visceral pain;
one or more of the T7, T8, T9, T10, T11 and/or T12 dermatome nerve(s) may suppress visceral pain, wherein the pancreas is contributing to the visceral pain;
one or more of the T10, T11 and/or T12 dermatome nerve(s) may suppress visceral pain, wherein the small intestine is contributing to the visceral pain;
one or more of the T11, S1, S2, S3 and/or S4 dermatome nerve(s) (preferably the S1, S2, S3 and/or S4 dermatome nerve(s)) may suppress visceral pain, wherein the colon is contributing to the visceral pain;
one or more of the T11, S1, S2, S3 and/or S4 dermatome nerve(s) (preferably the S1, S2, S3 and/or S4 dermatome(s)) may suppress visceral pain, wherein the rectum is contributing to the visceral pain;
one or more of the T10, T11, T12 and/or L1 dermatome nerve(s) (preferably the T11, T12 and/or L1 dermatome nerve(s)) may suppress visceral pain, wherein the kidney is contributing to the visceral pain;
one or more of the T11, T12 and/or L1 dermatome nerve(s) may suppress visceral pain, wherein the ureter is contributing to the visceral pain;
one or more of the T11, T12, L1, S1, S2, S3 and/or S4 dermatome nerve(s) (preferably the T12, L1, S1, S2, S3 and/or S4 dermatome nerve(s)) may suppress visceral pain, wherein the bladder (urinary bladder) is contributing to the visceral pain;
the T11 dermatome nerve may suppress visceral pain, wherein the ovary is contributing to the visceral pain;
the T11 dermatome nerve may suppress visceral pain, wherein a fallopian tube(s) is contributing to the visceral pain;
one or more of the T12 and/or L1 dermatome nerve(s) may suppress visceral pain, wherein the uterus is contributing to the visceral pain;
one or more of the S2, S3 and/or S4 dermatome nerve(s) may suppress visceral pain, wherein the cervix is contributing to the visceral pain;
one or more of the T10, T11, L1, L2, S1, S2, S3 and/or S4 dermatome nerve(s) (preferably the T11, L1, L2, S1, S2, S3 and/or S4 dermatome nerve(s)) may suppress visceral pain, wherein the testes is contributing to the visceral pain;
one or more of the T10, T11, L1, L2, S1, S2, S3 and/or S4 dermatome nerve(s) (preferably the T11, L1, L2, S1, S2, S3 and/or S4 dermatome nerve(s)) may suppress visceral pain, wherein the epididymis is contributing to the visceral pain;
one or more of the T3 and/or T4 dermatome nerve(s) may suppress visceral pain, wherein the heart is contributing to the visceral pain;
one or more of the the T4 and/or T5 dermatome nerve(s) may suppress visceral pain, wherein the esophagus is contributing to the visceral pain.

It is worth noting that a number of dermatomes/dermatome nerves may map to any given viscera, and there may be overlap in the dermatomes which map to different viscera. This is because spinal innervation of the viscera (e.g. the visceral afferents) is distributed throughout the spine from the cervical to the sacral spinal segments. Indeed, spinal visceral afferent input has been documented to spread to several segments rostral and caudal from the spinal segment of entry. By way of example, visceral afferents from the stomach (which relay nociceptive signals from the stomach to the spinal cord) may innervate each of the T8, T9 and T10 segments of the spinal cord. Thus, a dermatome that maps (via a nerve fiber, more particularly a spinal nerve afferent) to any one of the T8, T9 and T10 segments may be employed in the present invention for administering a clostridial neurotoxin to suppress integration of pain from the stomach's visceral afferents into the central nervous system (more particularly the spinal cord). Visceral afferent fibers from the liver also innervate the T8, T9 and T10 segments, hence dermatomes that map to one viscera may also map to another.

In one embodiment, the clostridial neurotoxin is administered to (or proximal to) the T8, T9 and/or T10 dermatome (or dermatome nerve). For example, the clostridial neurotoxin may be administered to (or proximal to) the T8 dermatome (or dermatome nerve). Additionally or alternatively, the clostridial neurotoxin may be administered to (or proximal to) the T9 dermatome (or dermatome nerve). Additionally or alternatively, the clostridial neurotoxin may be administered to (or proximal to) the T10 dermatome (or dermatome nerve). The clostridial neurotoxin may be administered to (or proximal to) at least two dermatomes (or dermatome nerves) selected from the T8, T9 and T10 dermatome. For example, the clostridial neurotoxin may be administered to (or proximal to) each of the T8, T9 and T10 dermatome (or dermatome nerve). Said dermatome(s) map to the stomach.

In one embodiment, the clostridial neurotoxin is administered to (or proximal to) the T8, T9, T10 and/or T11 (e.g. T8, T9, and/or T10) dermatome (or dermatome nerve). For example, the clostridial neurotoxin may be administered to (or proximal to) the T8 dermatome (or dermatome nerve). Additionally or alternatively, the clostridial neurotoxin may be administered to (or proximal to) the T9 dermatome (or dermatome nerve). Additionally or alternatively, the clostridial neurotoxin may be administered to (or proximal to) the T10 dermatome (or dermatome nerve). Additionally or alternatively, the clostridial neurotoxin may be administered to (or proximal to) the T11 dermatome (or dermatome nerve). The clostridial neurotoxin may be administered to (or proximal to) at least two or three dermatomes (or dermatome nerves) selected from the T8, T9, T10 and T11 (e.g. T8, T9, and/or T10) dermatome (or dermatome nerve). For example, the clostridial neurotoxin may be administered to (or proximal to) each of the T8, T9, T10 and T11 (e.g. T8, T9, and/or T10) dermatome (or dermatome nerve). Said dermatome(s) (or dermatome nerve(s)) map to the liver. Additionally or alternatively, said dermatome(s) map to the gall bladder.

In one embodiment, the clostridial neurotoxin is administered to (or proximal to) the T7, T8, T9, T10, T11 and/or T12 dermatome (or dermatome nerve). For example, the clostridial neurotoxin may be administered to (or proximal to) the T7 dermatome (or dermatome nerve). Additionally or alternatively, the clostridial neurotoxin may be administered to (or proximal to) the T8 dermatome (or dermatome nerve). Additionally or alternatively, the clostridial neurotoxin may be administered to (or proximal to) the T9 dermatome (or dermatome nerve). Additionally or alternatively, the clostridial neurotoxin may be administered to (or proximal to) the T10 dermatome (or dermatome nerve). Additionally or alternatively, the clostridial neurotoxin may be administered to (or proximal to) the T11 dermatome (or dermatome nerve). Additionally or alternatively, the clostridial neurotoxin may be administered to (or proximal to) the T12 dermatome (or dermatome nerve). The clostridial neurotoxin may be administered to (or proximal to) at least two dermatomes selected from the T7, T8, T9, T10, T11 and T12 dermatome (or dermatome nerve). For example, the clostridial neurotoxin may be administered to (or proximal to) each of the T7, T8, T9, T10, T11 and T12 dermatomes (or dermatome nerves). Said dermatome(s) (or dermatome nerve(s)) map to the pancreas.

In one embodiment, the clostridial neurotoxin is administered to (or proximal to) the T10, T11 and/or T12 dermatome (or dermatome nerve). For example, the clostridial neurotoxin may be administered to (or proximal to) the T10 dermatome (or dermatome nerve). Additionally or alternatively, the clostridial neurotoxin may be administered to (or proximal to) the T11 dermatome (or dermatome nerve). Additionally or alternatively, the clostridial neurotoxin may be administered to (or proximal to) the T12 dermatome (or dermatome nerve). The clostridial neurotoxin may be administered to (or proximal to) at least two dermatomes (or dermatome nerves) selected from the T10, T11 and T12 dermatome (or dermatome nerve). For example, the clostridial neurotoxin may be administered to (or proximal to) each of the T10, T11 and T12 dermatomes (or dermatome nerves). Said dermatome(s) (or dermatome nerves) map to the small intestine.

In one embodiment, the clostridial neurotoxin is administered to (or proximal to) the T11, S1, S2, S3 and/or S4 dermatome (or dermatome nerve) (preferably the S1, S2, S3 and/or S4 dermatome (or dermatome nerve)). For example, the clostridial neurotoxin may be administered to (or proximal to) the T11 dermatome (or dermatome nerve). Additionally or alternatively, the clostridial neurotoxin may be administered to (or proximal to) the S1 dermatome (or dermatome nerve). Additionally or alternatively, the clostridial neurotoxin may be administered to (or proximal to) the S2 dermatome (or dermatome nerve). Additionally or alternatively, the clostridial neurotoxin may be administered to (or proximal to) the S3 dermatome (or dermatome nerve). Additionally or alternatively, the clostridial neurotoxin may be administered to (or proximal to) the S4 dermatome (or dermatome nerve). The clostridial neurotoxin may be administered to (or proximal to) at least two, three or four dermatomes (or dermatome nerves) selected from the T11, S1, S2, S3 and S4 (e.g. S1, S2, S3 and S4) dermatome (or dermatome nerve). For example, the clostridial neurotoxin may be administered to (or proximal to) each of the T11, S1, S2, S3 and S4 (e.g. S1, S2, S3 and S4) dermatomes (or dermatome nerves). Said dermatome(s) (or dermatome nerves) map to the colon.

In one embodiment, the clostridial neurotoxin is administered to (or proximal to) the T10, T11, T12 and/or L1 (e.g. T11, T12 and/or L1) dermatome (or dermatome nerve). For example, the clostridial neurotoxin may be administered to (or proximal to) the T10 dermatome (or dermatome nerve). Additionally or alternatively, the clostridial neurotoxin may be administered to (or proximal to) the T11 dermatome (or dermatome nerve). Additionally or alternatively, the clostridial neurotoxin may be administered to (or proximal to) the T12 dermatome (or dermatome nerve). Additionally or alternatively, the clostridial neurotoxin may be administered to (or proximal to) the L1 dermatome (or dermatome nerve). The clostridial neurotoxin may be administered to (or proximal to) at least two or three dermatomes selected from the T10, T11, T12 and L1 (e.g. T11, T12 and/or L1) dermatome (or dermatome nerve). For example, the clostridial neurotoxin may be administered to (or proximal to) each of the T10, T11, T12 and L1 (e.g. T11, T12 and L1) dermatomes (or dermatome nerves). Said dermatome(s) (or dermatome nerve(s)) map to the kidney(s).

In one embodiment, the clostridial neurotoxin is administered to (or proximal to) the T11, T12, L1, S1, S2, S3 and/or S4 (e.g. T12, L1, S1, S2, S3 and/or S4) dermatome (or dermatome nerves). For example, the clostridial neurotoxin may be administered to (or proximal to) the T11 dermatome (or dermatome nerve). Additionally or alternatively, the clostridial neurotoxin may be administered to (or proximal to) the T12 dermatome (or dermatome nerve). Additionally or alternatively, the clostridial neurotoxin may be administered to (or proximal to) the L1 dermatome (or dermatome nerve). Additionally or alternatively, the clostridial neurotoxin may be administered to (or proximal to) the S1 dermatome (or dermatome nerve). Additionally or alternatively, the clostridial neurotoxin may be administered to (or proximal to) the S2 dermatome (or dermatome nerve). Additionally or alternatively, the clostridial neurotoxin may be administered to (or proximal to) the S3 dermatome (or dermatome nerve). Additionally or alternatively, the clostridial neurotoxin may be administered to (or proximal to) the S4 dermatome (or dermatome nerve). The clostridial neurotoxin may be administered to (or proximal to) at least two, three or four or five dermatomes (or dermatome nerves) selected from the T11, T12, L1, S1, S2, S3 and S4 (e.g. T12, L1, S1, S2, S3 and/or S4) dermatome (or dermatome nerve). For example, the clostridial neurotoxin may be administered to (or proximal to) each of the T11, T12, L1, S1, S2, S3 and S4 (e.g. T12, L1, S1, S2, S3 and S4) dermatomes (or dermatome nerves). Said dermatome(s) (or dermatome nerve(s)) map to the bladder (urinary bladder).

In one embodiment, the clostridial neurotoxin is administered to (or proximal to) the T11 dermatome (or dermatome nerve). Said dermatome (or dermatome nerve) maps to the ovary/ovaries. Additionally or alternatively, said dermatome (or dermatome nerve) maps to the fallopian tube(s).

In one embodiment, the clostridial neurotoxin is administered to (or proximal to) the T12, and/or L1 dermatome(s) (or dermatome nerve(s)). For example, the clostridial neurotoxin may be administered to (or proximal to) the T12 dermatome (or dermatome nerve). Additionally or alternatively, the clostridial neurotoxin may be administered to (or proximal to) the L1 dermatome (or dermatome nerve). The clostridial neurotoxin may be administered to (or proximal to) each of the T12, and L1 dermatomes (or dermatome nerves). Said dermatome(s) (or dermatome nerve(s)) map to the uterus.

In one embodiment, the clostridial neurotoxin is administered to (or proximal to) the S2, S3 and/or S4 dermatome (or dermatome nerve). For example, the clostridial neurotoxin may be administered to (or proximal to) the S2 dermatome (or dermatome nerve). Additionally or alternatively, the clostridial neurotoxin may be administered to (or proximal to) the S3 dermatome (or dermatome nerve). Additionally or alternatively, the clostridial neurotoxin may be administered to (or proximal to) the S4 dermatome (or dermatome nerve). The clostridial neurotoxin may be administered to (or proximal to) at least two dermatomes (or dermatome nerves) selected from the S2, S3 and S4 dermatome (or dermatome nerve). For example, the clostridial neurotoxin may be administered to (or proximal to) each of the S2, S3 and S4 dermatomes (or dermatome nerves). Said dermatome(s) (or dermatome nerves) map to the cervix.

In one embodiment, the clostridial neurotoxin is administered to (or proximal to) the T10, T11, L1, L2, S1, S2, S3 and/or S4 (e.g. T11, L1, L2, S1, S2, S3 and/or S4) dermatome (or dermatome nerve). For example, the clostridial neurotoxin may be administered to (or proximal to) the T10 dermatome (or dermatome nerve). Additionally or alternatively, the clostridial neurotoxin may be administered to (or proximal to) the T11 dermatome (or dermatome nerve). Additionally or alternatively, the clostridial neurotoxin may be administered to (or proximal to) the L1 dermatome (or dermatome nerve). Additionally or alternatively, the clostridial neurotoxin may be administered to (or proximal to) the L2 dermatome (or dermatome nerve). Additionally or alternatively, the clostridial neurotoxin may be administered to (or proximal to) the S1 dermatome (or dermatome nerve). Additionally or alternatively, the clostridial neurotoxin may be administered to (or proximal to) the S2 dermatome (or dermatome nerve). Additionally or alternatively, the clostridial neurotoxin may be administered to (or proximal to) the S3 dermatome (or dermatome nerve). Additionally or alternatively, the clostridial neurotoxin may be administered to (or proximal to) the S4 dermatome (or dermatome nerve). The clostridial neurotoxin may be administered to (or proximal to) at least two, three, four, five, six or seven dermatomes (or dermatome nerves) selected from the T10, T11, L1, L2, S1, S2, S3 and S4 (e.g. T11, L1, L2, S1, S2, S3 and/or S4) dermatome (or dermatome nerve). For example, the clostridial neurotoxin may be administered to (or proximal to) each of the T10, T11, L1, L2, S1, S2, S3 and S4 (e.g. the T11, L1, L2, S1, S2, S3 and S4) dermatomes (or dermatome nerves). Said dermatome(s) (or dermatome nerve(s)) map to the testes. Additionally or alternatively, said dermatomes(s) (or dermatome nerve(s)) map to the epididymis.

Thus, visceral pain may be suppressed in a number of organs identified as contributing to the visceral pain.

A number of diseases/conditions cause injury or insult to internal organs, leading to the unpleasant sensation of visceral pain that can often be chronic and important to manage (e.g. as the disease itself may take time to heal). For example, in Crohn's disease, parts of the digestive system such as the stomach and intestine become inflamed, leading to the emergence of pain from said organs. It follows, therefore, that the present invention finds advantageous utility in suppressing the visceral pain symptoms caused by a variety of different diseases/conditions.

In one embodiment, visceral pain may be bladder pain syndrome (interstitial cystitis), chronic functional abdominal pain (CFAP), functional constipation, functional dyspepsia, a non-cardiac chest pain (NCCP), a chronic abdominal pain, a gastritis, an inflammatory bowel disease, like, e.g., a Crohn's disease, an ulcerative colitis, a microscopic colitis, a diverticulitis and a gastroenteritis; an interstitial cystitis; an intestinal ischemia; a cholecystitis; an appendicitis; a gastroesophageal reflux; an ulcer, a nephrolithiasis, a urinary tract infection, a pancreatitis, a hernia, autoimmune pain including, for example, a sarcoidosis and a vasculitis; organic visceral pain including, for example, pain resulting from a traumatic, inflammatory or degenerative lesion of the gut or produced by a tumour impinging on sensory innervation; treatment-induced visceral pain includes, for example, a pain attendant to chemotherapy therapy or a pain attendant to radiation therapy; Crohn's disease, ulcerative colitis, cirrhosis, irritable bowel syndrome, coeliac disease, endometriosis, gastric ulcers, kidney stones, bladder stones, gallbladder stones, and/or peritonitis.

In a preferred embodiment, visceral pain is caused by a condition selected from bladder pain syndrome (interstitial cystitis), cirrhosis, Crohn's disease, ulcerative colitis, irritable bowel syndrome, coeliac disease, endometriosis, gastric ulcers, kidney stones, bladder stones, gallbladder stones, pancreatitis and peritonitis.

In other words, the visceral pain may be interstitial cystitis pain, cirrhosis pain, Crohn's disease pain, ulcerative colitis pain, irritable bowel syndrome pain, coeliac disease pain, endometriosis pain, gastric ulcer pain, kidney stone pain, bladder stone pain, gallbladder stone pain, pancreatitis pain and/or peritonitis pain.

The visceral pain may be caused by or associated with a vascular disorder.

Examples of visceral pain include the following:

### A. Functional Visceral Pain

Functional visceral pain includes, for example, an irritable bowel syndrome and a chronic functional abdominal pain (CFAP), a functional constipation and a functional dyspepsia, a non-cardiac chest pain (NCCP) and a chronic abdominal pain.

### B. Chronic Gastrointestinal Inflammation

Chronic gastrointestinal inflammation includes, for example, a gastritis, an inflammatory bowel disease, like, e.g., a Crohn's disease, an ulcerative colitis, a microscopic colitis, a diverticulitis and a gastroenteritis; an interstitial cystitis; an intestinal ischemia; a cholecystitis; an appendicitis; a gastroesophageal reflux; an ulcer, a nephrolithiasis, a urinary tract infection, a pancreatitis and a hernia.

### C. Autoimmune Pain

Autoimmune pain includes, for example, a sarcoidosis and a vasculitis.

### D. Organic Visceral Pain

Organic visceral pain includes, for example, pain resulting from a traumatic, inflammatory or degenerative lesion of the gut or produced by a tumour impinging on sensory innervation.

### E. Treatment-Induced Visceral Pain

Treatment-induced visceral pain includes, for example, a pain attendant to chemotherapy therapy or a pain attendant to radiation therapy.

The visceral pain may be selected from: endometriosis pain, pancreatitis pain, gastrointestinal pain, and visceral pain caused by or associated with a vascular disorder (more preferably endometriosis pain).

For example, the visceral pain may be caused by bladder pain syndrome (interstitial cystitis). In this case, the clostridial neurotoxin may be administered to the T12, L1, S2, S3 and/or S4 dermatome (or dermatome nerve). In other words, to suppress visceral pain caused by bladder pain syndrome (interstitial cystitis), a method of the invention may comprise (a) identifying the bladder as an organ in the patient that is contributing to said visceral pain; (b) mapping said organ to a specific dermatome nerve according to the following instructions, thereby matching said organ to one or more specifically designated dermatome nerve(s): urinary bladder maps to the T11, T12, L1, S1, S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by cirrhosis (liver cirrhosis). In this case, the clostridial neurotoxin may be administered to the T8, T9, T10 and/or T11 dermatome nerve(s). In other words, to suppress visceral pain caused by cirrhosis, a method of the invention may comprise (a) identifying the liver as an organ in the patient that is contributing to said visceral pain; (b) mapping said organ to a specific dermatome nerve according to the following instructions, thereby matching said organ to one or more specifically designated dermatome nerve(s): liver maps to the T8, T9, T10 and/or T11 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by gall bladder stones. In this case, the clostridial neurotoxin may be administered to the T8, T9, T10 and/or T11 dermatome nerve(s). In other words, to suppress visceral pain caused by cirrhosis, a method of the invention may comprise (a) identifying the liver as an organ in the patient that is contributing to said visceral pain; (b) mapping said organ to a specific dermatome nerve according to the following instructions, thereby matching said organ to one or more specifically designated dermatome nerve(s): gall bladder maps to the T8, T9, T10 and/or T11 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by Crohn's disease. In this case, the clostridial neurotoxin may be administered to the T8, T9, T10, T11, T12, S1, S2, S3 and/or S4 dermatome. In other words, to suppress visceral pain caused by Crohn's disease, a method of the invention may comprise (a) identifying the stomach, small intestine and/or colon as an organ in the patient that is contributing to said visceral pain; (b) mapping said organ to a specific dermatome nerve according to the following instructions, thereby matching said organ to one or more specifically designated dermatome nerve(s): stomach maps to the T8, T9 and/or T10 dermatome nerve(s), small intestine maps to the T10, T11 and/or T12 dermatome nerve(s), colon maps to the T11, S1, S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

It should be noted that five main types of Crohn's disease exist, with each typically being defined by the location of inflammation in the gastrointestinal tract. The types of Crohn's disease include ileocolitis (e.g. inflames the end of the small intestine/ileum and a portion of the large intestine/colon), ileitis (e.g. inflames the last section of the small intestine/ileum); gastroduodenal Crohn's (e.g. inflames the stomach and the start of the small intestine/duodenum), jejunoileitis (e.g. inflames the middle part of the small intestine/jejunum) and Crohn's (granulomatous) colitis (e.g. inflames the colon).

Thus, to suppress visceral pain caused by ileocolitis, a method of the invention may comprise (a) identifying the small intestine and/or colon as an organ in the patient that is contributing to said visceral pain; (b) mapping said organ to a specific dermatome nerve according to the following instructions, thereby matching said organ to one or more specifically designated dermatome nerve(s): small intestine maps to the T10, T11 and/or T12 dermatome nerve(s), colon maps to the T11, S1, S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

To suppress visceral pain caused by ileitis, a method of the invention may comprise (a) identifying the small intestine as an organ in the patient that is contributing to said visceral pain; (b) mapping said organ to a specific dermatome nerve according to the following instructions, thereby matching said organ to one or more specifically designated dermatome nerve(s): small intestine maps to the T10, T11 and/or T12 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

To suppress visceral pain caused by gastroduodenal Crohn's, a method of the invention may comprise (a) identifying the stomach and/or small intestine as an organ in the patient that is contributing to said visceral pain; (b) mapping said organ to a specific dermatome nerve according to the following instructions, thereby matching said organ to one or more specifically designated dermatome nerve(s): stomach maps to the T8, T9 and/or T10 dermatome nerve(s), small intestine maps to the T10, T11 and/or T12 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

To suppress visceral pain caused by jejunoileitis, a method of the invention may comprise (a) identifying the small intestine as an organ in the patient that is contributing to said visceral pain; (b) mapping said organ to a specific dermatome nerve according to the following instructions, thereby matching said organ to one or more specifically designated dermatome nerve(s): small intestine maps to the T10, T11 and/or T12 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

To suppress visceral pain caused by Crohn's (granulomatous) colitis, a method of the invention may comprise (a) identifying the colon as an organ in the patient that is contributing to said visceral pain; (b) mapping said organ to a specific dermatome nerve according to the following instructions, thereby matching said organ to one or more specifically designated dermatome nerve(s): colon maps to the T11, S1, S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by ulcerative colitis. In this case, the clostridial neurotoxin may be administered to the S1, S2, S3 and/or S4 dermatome. In other words, to suppress visceral pain caused by ulcerative colitis, a method of the invention may comprise (a) identifying the colon and/or rectum as an organ in the patient that is contributing to said visceral pain; (b) mapping said organ to a specific dermatome nerve according to the following instructions, thereby matching said organ to one or more specifically designated dermatome nerve(s): colon maps to the T11, S1, S2, S3 and/or S4 dermatome nerve(s), rectum maps to the T11, S1, S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by irritable bowel syndrome. In this case, the clostridial neurotoxin may be administered to the S1, S2, S3 and/or S4 dermatome. In other words, a method of the invention may comprise (a) identifying the large intestine as an organ in the patient that is contributing to said visceral pain; (b) mapping said organ to a specific dermatome nerve according to the following instructions, thereby matching said organ to one or more specifically designated dermatome nerve(s): large intestine maps to the T11, S1, S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by coeliac disease. In this case, the clostridial neurotoxin may be administered to the T10, T11, and/or T12 dermatome. In other words, to suppress visceral pain caused by coeliac disease, a method of the invention may comprise (a) identifying the small intestine as an organ in the patient that is contributing to said visceral pain; (b) mapping said organ to a specific dermatome nerve according to the following instructions, thereby matching said organ to one or more specifically designated dermatome nerve(s): small intestine maps to the T10, T11 and/or T12 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by gastric ulcers. In this case, the clostridial neurotoxin may be administered to the T8, T9 and/or T10 dermatome. In other words, to suppress visceral pain caused by gastric ulcers, a method of the invention may comprise (a) identifying the stomach as an organ in the patient that is contributing to said visceral pain; (b) mapping said organ to a specific dermatome nerve according to the following instructions, thereby matching said organ to one or more specifically designated dermatome nerve(s): stomach maps to the T8, T9 and/or T10 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by pancreatitis. In this case, the clostridial neurotoxin may be administered to the T7, T8, T9, T10, T11 and/or T12 dermatome. In other words, to suppress visceral pain caused by pancreatitis, a method of the invention may comprise (a) identifying the pancreas as an organ in the patient that is contributing to said visceral pain; (b) mapping said organ to a specific dermatome nerve according to the following instructions, thereby matching said organ to one or more specifically designated dermatome nerve(s): pancreas maps to the T7, T8, T9, T10, T11 and/or T12 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may be caused by peritonitis (e.g. peritonitis of visceral peritoneum).

Peritonitis is inflammation of the peritoneum, a membrane that lines the inner abdominal wall and abdominal organs (viscera). The peritoneum and the organ (viscera) it lines share the same sensory ganglion and spinal cord segment. Thus, the appropriate dermatome to target for suppressing visceral pain that is caused by peritonitis depends on the organ that the (inflamed) peritoneum lines. For example, where the (inflamed) peritoneum lines the stomach: a method of the invention may comprise (a) identifying the stomach as an organ in the patient that is contributing to said visceral pain (e.g. due to inflammation of the peritoneum); (b) mapping said organ to a specific dermatome nerve according to the following instructions, thereby matching said organ to one or more specifically designated dermatome nerve(s): stomach maps to the T8, T9 and/or T10 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In a preferred embodiment, the visceral pain is caused by endometriosis. For example, endometriosis that is associated with a symptom selected from dysmenorrhea, chronic pelvic pain (CPP), dyspareunia, constipation, dyschezia, and chronic back pain.

Endometriosis is a chronic and debilitating condition characterized by chronic pelvic pain and infertility. Endometriosis has a large clinical burden, affecting approximately 1 in 10 women globally. Endometriosis is associated with significant societal and economic burden that costs the US economy $22 billion annually in lost productivity and direct healthcare costs. Endometriosis may be defined as the presence of endometrial glands and stroma in an abnormal or ectopic location outside the uterine cavity, for example ectopic positions such as the ovaries and fallopian tubes. Endometriosis occurs in 20-50% in women with infertility and 71-87% in women with chronic pelvic pain.

Endometriosis is a disease of the female reproductive system in which cells similar to those in the endometrium, the layer of tissue that normally covers the inside of the uterus, grow outside the uterus. Most often this is on the ovaries, fallopian tubes, and tissue around the uterus and ovaries; however, it may also occur in other parts of the body. For example, ectopic ‘endometrium’ could grow in very different areas of the visceral cavity, including the bladder, the pelvic ligaments, up to the diaphragm; and the associated pain can be felt outside of the menstruation period. Endometriosis is thus a pathology where various dermatomes could be of interest depending on the site of endometriotic growth.

The most common symptoms of endometriosis are dysmenorrhea, chronic pelvic pain (CPP), dyspareunia, constipation, dyschezia, and chronic back pain. In this regard, clostridial neurotoxin treatment (BoNT/A) has previously been tested for alleviating pain (e.g. caused by endometriosis) associated with acute dysmenorrhoea (menstrual cramps) and pelvic pain syndrome of uterine origin, allowing from improvement of quality of life scores in the majority of patients. However, such treatment required injection of botulinum toxin directly into the myometrial wall under hysteroscopic control. This is highly invasive and inconvenient, particularly since repeated administration may be required over the long term in such incurable disease scenarios. Thus, the present invention represents a particularly advantageous pain relief strategy for alleviating visceral pain that would otherwise require invasive administration of medicament (e.g. as in the case of visceral pain caused by endometriosis).

As mentioned above, endometriosis pain can effect a number of different organs, and thus represents a pathology where various dermatomes can advantageously be utilised as an administration route depending on the site of endometriotic growth, thus the organ contributing to the visceral pain. For example, where a patient suffers endometriosis, the bladder may be identified as the organ contributing to visceral pain, and the clostridial neurotoxin may thus be administered to a specific dermatome nerve that maps to the bladder.

In embodiments directed to suppressing visceral pain caused by endometriosis, the clostridial neurotoxin may be administered to the T11, T12, L1, S2, S3 and/or S4 dermatome. In other words, to suppress visceral pain caused by endometriosis, a method of the invention may comprise (a) identifying an ovary, fallopian tube, uterus and/or cervix as an organ in the patient that is contributing to said visceral pain; (b) mapping said organ to a specific dermatome nerve according to the following instructions, thereby matching said organ to one or more specifically designated dermatome nerve(s): ovary maps to the T11 dermatome nerve(s), fallopian tube(s) maps to the T11 dermatome nerve(s), uterus maps to the T12 and/or L1 dermatome nerve(s), cervix maps to the S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

It should be noted that, in endometriosis, the visceral pain may be contributed to by a particular organ of the female reproductive system (e.g. one or more of ovary, fallopian tube, uterus and/or cervix).

To suppress visceral pain caused by endometriosis in an ovary, a method of the invention may comprise (a) identifying an ovary as an organ in the patient that is contributing to said visceral pain; (b) mapping said organ to a specific dermatome nerve according to the following instructions, thereby matching said organ to one or more specifically designated dermatome nerve(s): ovary maps to the T11 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

To suppress visceral pain caused by endometriosis in a fallopian tube, a method of the invention may comprise (a) identifying a fallopian tube as an organ in the patient that is contributing to said visceral pain; (b) mapping said organ to a specific dermatome nerve according to the following instructions, thereby matching said organ to one or more specifically designated dermatome nerve(s): fallopian tube(s) maps to the T11 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

To suppress visceral pain caused by endometriosis in the uterus, a method of the invention may comprise (a) identifying the uterus as an organ in the patient that is contributing to said visceral pain; (b) mapping said organ to a specific dermatome nerve according to the following instructions, thereby matching said organ to one or more specifically designated dermatome nerve(s): uterus maps to the T12 and/or L1 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

To suppress visceral pain caused by endometriosis in the cervix, a method of the invention may comprise (a) identifying the cervix as an organ in the patient that is contributing to said visceral pain; (b) mapping said organ to a specific dermatome nerve according to the following instructions, thereby matching said organ to one or more specifically designated dermatome nerve(s): cervix maps to the S2, S3 and/or S4 dermatome nerve(s); and (c) administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain.

In one embodiment, the visceral pain may comprise or consist of allodynia. In other words, the visceral pain may be allodynia.

Allodynia means “other pain”. It is a pain that results from a stimulus that is not normally painful. By way of example, a subject suffering from bad sunburn may experience intense pain even to light touch. In more detail, sun exposure can overly sensitize the skin such that wearing a shirt or taking a shower can be very painful. Thus, a sufferer of tactile allodynia (aka static tactile allodynia or mechanical allodynia) may experience pain to touch, such as with resting one's head on a pillow, or with wearing a hat, earrings, or necklace. Similarly, a sufferer of dynamic allodynia may experience pain from lightly brushing one's hair, or from shaving one's face. Allodynia is a condition that is distinct from hyperalgesia, which is a pain stimulus that is more painful than usual. Indeed, as mentioned above, allodynia is by its very definition “pain due to a stimulus that does not usually provoke pain”, as opposed to hyperalgesia (increased pain from a stimulus that does usually provoke pain). Allodynia may also be referred to as “hypersensitivity” herein.

With regard to visceral pain, allodynia often occurs in particular as a result of gastrointestinal disorder, such as oesophagitis, gastro-oesophageal reflux disease, non-ulcer dyspepsia, gastroparesis, and irritable bowel syndrome (IBS). Visceral allodynia/hypersensitivity may also occur in non-gastrointestinal disorders such as interstitial cystitis and ureteric colic (e.g. urinary stone). Visceral allodynia/hypersensitivity may be defined as an increased intensity of sensations and lowered thresholds for visceral pain in a patient.

The present inventors have demonstrated that the present invention may be used to suppress allodynia in a distinct manner from suppressing hyperalgesia. This is demonstrated in the accompanying Examples, in which experiments were designed to test the effect of dermatome injection of clostridial neurotoxin in alleviating both allodynia and hyperalgesia types of visceral pain. In more detail, in the rodent model of visceral pain (in the bladder), visceral pain was evaluated in a blinded manner by applying to the lower abdomen (close to the urinary bladder) a set of 8 calibrated von Frey filaments of increasing forces (1, 2, 4, 6, 8, 10, 15 and 26 g) with an interstimulus interval of 5 seconds. Forces of 1-6 g are considered low (e.g. as they would not normally cause pain in a healthy rodent) and thus emulate the non-noxious stimuli that lead to pain perception in allodynia. The forces above 6 g were used to invoke pain consistent with hyperalgesia. As demonstrated in FIG. 5, clostridial neurotoxin (e.g. Dysport) administration led to a marked decrease in the nociceptive scores (indicative of reduced pain perception) in the ‘allodynia range’ of forces (e.g. 1-6 g), with pain suppression being less marked in the ‘hyperalgesia range’ of forces (e.g. 9-32 g).

One particular of advantage of targeting allodynia, in particular, is the ability to treat chronic pain while sparing (‘acute’) nociception. This is because the ability to feel acute pain is a physiological process involved in alerting an individual to certain dangers and protects us from injury, thus it may be preferable to retain (at least a certain level) of general acute pain perception even as part of an analgesic-treatment strategy.

Thus, in one embodiment, allodynia is suppressed, and hyperalgesia is not suppressed. In one embodiment, chronic pain is suppressed to a greater extent than acute pain.

Furthermore, the inventors have demonstrated that administration of a clostridial neurotoxin may suppress visceral pain over an extended period of time, taking advantage of the long-term effect achievable by administering a clostridial neurotoxin such as botulinum neurotoxin. Such effect is particularly advantageous for alleviating chronic pain. The visceral pain may be suppressed for at least 4, 8, 12, 16, 20, 24, 28, 32, 36, 40, 44, 48, 52, 56, or 60 days following administration of the clostridial neurotoxin. For example, said visceral pain may be suppressed for at least 60, 64, 68, 72, 76, 80, 84, or 88 days following administration of the clostridial neurotoxin.

The visceral pain may be suppressed for at least 1, 2, 3, 4, 5, 7, 8, or 9 months following administration of the clostridial neurotoxin. For example, said visceral pain may be suppressed for at up to an including 9 months following administration of the clostridial neurotoxin.

Said visceral pain may be reduced for at least 10 days following administration of the clostridial neurotoxin. Preferably, visceral pain may be reduced for at least 30 days following administration of the clostridial neurotoxin. More preferably, said visceral pain may be reduced for at least 50 days following administration of the clostridial neurotoxin.

Visceral pain may be suppressed within at least 1 day following administration of the clostridial neurotoxin; for example within 1, 2, 3, 4, 5, 6, 7, 8, 9, 10, 11 or 12 days following administration of the clostridial neurotoxin. Preferably, visceral pain may be suppressed within at least 7 days following administration of the clostridial neurotoxin. More preferably, visceral pain may be suppressed within at least 10 days following administration of the clostridial neurotoxin.

Certain terms described above will now be discussed in more detail.

A “patient” (which may be used synonymously with the term “subject”) as used herein may be a mammal, such as a human or other mammal. Preferably “patient” means a human subject. The patient may be a patient having a lower than average (basal) neuroimmune response, e.g. a patient receiving an alternative therapy, such as chemotherapy, which may suppress the immune system.

The term “disorder” as used herein also encompasses a “disease”. In one embodiment the disorder is a disease.

The term “suppressing” may be used synonymously with the term “treating” herein. Thus, the present invention embraces a method for treating visceral pain in a patient, said method comprising administration of a therapeutically effective amount of a clostridial to a patient suffering from visceral pain. The present invention also embraces a corresponding therapeutic use, namely a clostridial neurotoxin for use in treating visceral pain in a patient.

The term “suppress” or “suppressing”, or “treat” or “treating” as used herein encompasses prophylactic suppression and treatment (e.g. to prevent onset of visceral pain) as well as corrective suppression and treatment (suppression and treatment of a subject already suffering from visceral pain). In a preferable embodiment, the term “suppress” or “suppressing” as used herein means corrective treatment. In a preferable embodiment, the term “treat” or “treating” as used herein means corrective treatment. The term “suppress” or “suppressing”, or “treat” or “treating” encompasses suppressing and treating both the visceral pain and a symptom thereof. In some embodiments the term “suppress” or “suppressing”, or “treat” or “treating” refers to a symptom of visceral pain.

In one embodiment, a clostridial neurotoxin of the invention suppresses a visceral pain by at least 10%, or at least 20%, or at least 30%, or at least 40%, or at least 50%, or at least 60%, or at least 70%, or at least 80%, or at least 90% or by 100% greater that an otherwise identical administration lacking a clostridial neurotoxin (e.g. a vehicle-only administration).

In one embodiment, a clostridial neurotoxin of the invention suppresses a visceral pain by at least 10%, or at least 20%, or at least 30%, or at least 40%, or at least 50%, or at least 60%, or at least 70%, or at least 80%, or at least 90% or by 100% compared to visceral pain perceived by the patient pre-administration of the clostridial neurotoxin.

Suppressing (or treating) pain may be used synonymously with “reducing pain”. In other words, administration of a clostridial neurotoxin of the invention may reduce visceral pain in a subject.

In more detail, reference to “reduced” or “reducing” (in terms of visceral pain) preferably means a lower level of visceral pain is perceived by the subject after administration with a clostridial neurotoxin of the invention (post-administration) when compared with a level of visceral pain perceived by the subject prior to administration (pre-administration). For example, the level of visceral pain perceived may be reduced by at least 15%, 25%, 35%, 45%, 55%, 65%, 75%, 85% or 95% post-administration relative to pre-administration. For example, the level of visceral pain perceived may be reduced by at least 75%; preferably at least 85%; more preferably at least 95% post-administration.

A variety of means for assessing pain perception are known to those skilled in the art. For example, evaluation of mechanical allodynia (either static or dynamic) is routinely used in human pain studies as described in Pogatzki-Zahn et. al. (Pain Rep. 2017 March; 2(2): e588), incorporated herein by reference.

A suitable (albeit non-limiting) method for assessing pain perception in a subject includes the following: Numerical Rating Scale (NRS) score; although the skilled person is aware of other methods which may be used additionally or alternatively such as sensory threshold, pain perception threshold, static mechanical allodynia, dynamic mechanical allodynia, temporal summation, pressure pain threshold, conditioned pain modulation, and temperature threshold.

Other non-limiting examples of pain perception measures include: change from baseline in SF-36 scores at each scheduled time point; amount of rescue medication taken during the study and time to first intake of rescue medication. These may be considered “exploratory” endpoints or pain perception assessment measures.

Thus, in a preferred embodiment, following the administration of a clostridial neurotoxin of the invention, pain perception may be assessed by one or more of: (a) a Numerical Rating Scale (NRS); (b) a stimulus-evoked NRS; (c) temperature of the painful area; (d) size of the painful area; (e) time to onset of analgesic effect; (f) peak analgesic effect; (g) time to peak analgesic effect; (h) duration of analgesic effect; and (i) an SF-36 quality of life assessment.

The skilled person is aware of such methods for assessing pain perception. For convenience, further description of the Numerical Rating Score and Quality of Life questionnaire Short Form-36 are provided below.

Numerical Rating Scale (NRS): Typically pain perception according to the present invention uses the Numerical Rating Scale (NRS). The NRS is an 11-point scale to assess subject pain perception. Subjects are asked to give a number between 0 and 10 that fits best to their pain intensity. Zero represents ‘no pain at all’ whereas the upper limit, 10, represents ‘the worst pain possible’.

The NRS can be used to assess numerous facets of pain, including spontaneous average pain, spontaneous worst pain, and spontaneous current pain. Spontaneous average pain is assessed by asking a subject to select a number that best describes the subject's average pain (e.g. perceived pain) over a period of time, for example at least 6 hours, 12 hours, 24 hours, or at least 48 hours. Spontaneous worst pain is assessed by asking a subject to select a number that best describes the subject's pain at its worst during a specified period, e.g. at least the previous 6 hours, 12 hours, 24 hours or previous 48 hours. Spontaneous current pain is assessed by asking a subject to select a number that best describes how much pain the subject is in at the time of assessment.

The NRS can also be used to assess a subject's pain perception in response to a variety of different stimuli. To assess pain perception in response to a stimulus, the subject will be submitted to stimuli of various nature applied to the painful area. Subjects will be asked what are their current NRS scores pre-dose and post-stimulus.

Examples of stimuli used include: (i) light touch (which can be assessed by measuring pain on the surface of the painful area on radial spokes following application of a von Frey filament as described herein); (ii) pressure (pressure pain threshold), which can be assessed by asking the subject to give a NRS score as increasing pressure is applied using a pressure algometer; and (iii) temperature (which can be assessed by asking the subject for an NRS score for warm, cold and hot stimulation using a thermode applied to the painful area).

Preferably, administration of a clostridial neurotoxin of the invention reduces the subject's NRS score post-administration (e.g. from a rating of ≥7 to a rating of ≤6) when compared with the subject's NRS score pre-administration.

Quality of Life questionnaire Short Form-36 (SF-36): The SF-36 quality of life questionnaire may be used to assess a subject's pain perception. The SF-36 is a 36-item, subject-reported survey of subject health. The SF-36 consists of eight scaled scores (vitality, physical functioning, bodily pain, general health perceptions, physical role functioning, emotional role functioning, social role functioning and mental health). Each scale is directly transformed into a 0-100 scale on the assumption that each question carries equal weight. The higher the score recorded in the SF-36, the less disability.

Relevant parameters commonly tested in clinical trials for the treatment of pain are known in the art and could be readily selected by one of ordinary skill in the art. Examples of such parameters include, but are not limited to NRS; stimulus-evoked NRS; temperature of the painful area; size of the painful area; time to onset of analgesic effect; peak analgesic effect; time to peak analgesic effect; duration of analgesic effect; and/or SF-36 quality of life as described herein. Methods for assessing these parameters are also known in the art and can be carried out by one of ordinary skill using routine methods and procedures.

Preferably, administration of a clostridial neurotoxin of the invention increases the subject's SF-36 score post-administration (e.g. from a score of ≤50 to a score of ≥50) when compared with the subject's SF-36 score pre-administration.

A clostridial neurotoxin may be administered to a subject in a therapeutically effective amount or a prophylactically effective amount.

A “therapeutically effective amount” is any amount of the clostridial neurotoxin, which when administered alone or in combination to a subject for suppressing/treating visceral pain (or a symptom thereof) is sufficient to effect such suppression/treatment of the visceral pain, or symptom thereof.

A “prophylactically effective amount” is any amount of the clostridial neurotoxin that, when administered alone or in combination to a subject inhibits or delays the onset or reoccurrence of visceral pain (or a symptom thereof). In some embodiments, the prophylactically effective amount prevents the onset or reoccurrence of visceral pain entirely. “Inhibiting” the onset means either lessening the likelihood of visceral pain onset (or symptom thereof), or preventing the onset entirely.

### Clostridial Neurotoxins

Clostridial neurotoxins of the invention will now be described in more detail, together with details of how they may be formulated e.g. into pharmaceutical compositions.

The clostridial neurotoxins of the invention may be formulated in any suitable manner for administration to a subject, for example as part of a pharmaceutical composition. Thus, in one aspect, the invention provides a pharmaceutical composition for use in any method of treatment described herein, the pharmaceutical composition comprising a clostridial neurotoxin of the invention and a pharmaceutically acceptable carrier, excipient, adjuvant, propellant and/or salt.

In some embodiments, the clostridial neurotoxin of the invention may be in a single-chain form, while in other embodiments the clostridial neurotoxin may be in a di-chain form, e.g. where the two chains are linked by a di-sulphide bridge. Preferably the clostridial neurotoxin is in a di-chain form.

The clostridial neurotoxins of the present invention may preferably be formulated for injection to the subject, for example via intradermal injection. Compositions suitable for injection may be in the form of solutions, suspensions or emulsions, or dry powders which are dissolved or suspended in a suitable vehicle prior to use.

The dosage ranges for administration of the clostridial neurotoxins of the present invention are those to produce the desired therapeutic and/or prophylactic effect. It will be appreciated that the dosage range required depends on the precise nature of the clostridial neurotoxin or composition, the route of administration, the nature of the formulation, the age of the subject, the nature, extent or severity of the subject's condition, contraindications, if any, and the judgement of the attending physician. Variations in these dosage levels can be adjusted using standard empirical routines for optimisation.

In one embodiment a dosage of the clostridial neurotoxin is a flat dose. A flat dose may be in the range of 50 pg to 250 μg, preferably 100 pg to 100 μg. In one embodiment a flat dose may be at least 50 pg, 100 pg, 500 pg, 1 ng, 50 ng, 100 ng, 500 ng, 1 μg or 50 μg. Said dose may be a single flat dose.

For convenience of the physician, a clostridial neurotoxin may be administered by way of a unit dose. Said unit dose may be administered at a single site or, alternatively, less than a unit dose may be administered at an administration site (e.g. where there are two or more administration sites and the dose is divided (equally or unequally) between said sites). In one embodiment, a single unit dose may be administered per dermatome injected when carrying out the present invention.

A suitable unit dose may be 5 pg to 17,000 pg of the clostridial neurotoxin. An upper limit of the unit dose range may be 16,500, 15,500, 14,500, 13,500, 12,500, 11,500, 10,500, 9,500, 8,500, 7,500, 6,500, 5,500, 4,500, 3,500, 2,500, 1,500 or 500 pg of clostridial neurotoxin, preferably the upper limit is 16,000 pg. A lower limit of the unit dose range may be 10, 20, 30, 50, 100, 200, 250, 350, 450, 550, 650, 750, 850, 950, 1,000, 1,500, 2,000, 2,500, 3,000, 3,500, 4,000, 4,500 or 5,000 pg of clostridial neurotoxin, preferably the lower limit is 1,000 pg. Preferably, the unit dose is 750 pg to 17,000 pg of clostridial neurotoxin. More preferably, the unit dose of clostridial neurotoxin is 1,000 pg to 16,000 pg of (e.g. modified) BoNT/A, e.g. 4,000 pg to 6,000 pg.

A total dose administered per treatment session may be up to 255,000 pg of the clostridial neurotoxin. This may correspond to 15× the unit dose. In other words, the total amount of clostridial neurotoxin administered at a given treatment session may be up to 255,000 pg. The total dose may be up to 240,000, 220,000, 200,000, 180,000, 160,000, 140,000, 110,000, 100,000, 90,000, 80,000, 70,000, 60,000, 50,000, 40,000, 30,000, 20,000, 10,000 or 5,000 pg. Preferably, the total dose may be up to 240,000 pg of clostridial neurotoxin. The total dose may be at least 900, 1,000, 2,000, 3,000, 4,000, 5,000, 7,500, 10,000, 12,500, 15,000, 20,000, 30,000, 40,000, 50,000, 60,000, 70,000, 80,000, 90,000, 100,000, 120,000, 150,000, 175,000, 200,000 or 220,000 pg. Preferably, the total dose may be at least 1,500 pg, more preferably at least 2,000 pg of clostridial neurotoxin, e.g. at least 12,000 pg. The total dose may be 2,000-240,000 pg, preferably 128,000-240,000 pg. More preferably, the total dose administered is 15,000-240,000 pg.

The total number of unit doses administered in a given treatment may be up to 15× the unit dose. For example, the total number of unit doses administered may be up to 14×, 13×, 12×, 11×, 10×, 9×, 8× or 7×. The total number of unit doses administered may be at least 2×, 3×, 4×, 5×, 6×, 7× the unit dose, preferably at least 2×. The total number of unit doses administered may be 2× to 15×, 7× to 15× or 10× to 14×. Preferably, the number of unit doses administered is 15×.

The term “up to” when used in reference to a value (e.g. up to 255,000 pg) means up to and including the value recited. Thus, as an example, reference to administering “up to 255,000 pg” of clostridial neurotoxin encompasses administration of 255,000 pg of (e.g. modified) BoNT/A as well as administration of less than 255,000 pg of clostridial neurotoxin.

The clostridial neurotoxin of the invention may be administered iteratively (e.g. up to 5, 10, 15 or 20 times) as part of a treatment regimen (such as on different days, e.g. with at least 1 day between successive treatments). Iterative administration means administration at least two times, e.g. at least 5, 10, 15 or 20 times. Thus, in one embodiment, a clostridial neurotoxin of the invention may be administered two or more times to suppress/treat the visceral pain of a subject. This is particularly pertinent for the treatment of chronic conditions, such as chronic pain, where ongoing treatment is typically necessary. In one embodiment a clostridial neurotoxin of the invention may be administered weekly, twice monthly, monthly, every two months, every six months or annually, preferably at least twice annually or annually. In one embodiment, a clostridial neurotoxin of the invention is administered two or more times in a period of 10 years, 5 years, 2 years or 1 year. Preferably, a clostridial neurotoxin of the invention is administered two or more times in a period of 1 year. Treatment may continue for at least 6 months, 1 year, 2 years, 3 years, 5 years, 10 years, 15 years, 20 years, 25 years or 30 years.

It is preferred that the clostridial neurotoxin is not administered together with a further therapeutic or diagnostic agent (e.g. a nucleic acid, protein, peptide or small molecule therapeutic or diagnostic agent) additional to the light-chain and heavy-chain. For example, in one embodiment the clostridial neurotoxin is not administered with a further analgesic. In one embodiment a clostridial neurotoxin of the invention is not administered together with a covalently associated therapeutic agent. In one embodiment a clostridial neurotoxin of the invention is not administered together with a non-covalently associated therapeutic agent.

Fluid dosage forms are typically prepared utilising the clostridial neurotoxin and a pyrogen-free sterile vehicle. The clostridial neurotoxin, depending on the vehicle and concentration used, can be either dissolved or suspended in the vehicle. In preparing solutions the clostridial neurotoxin can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

The clostridial neurotoxin may be administered prior to, simultaneous or subsequent to the onset of visceral pain. The clostridial neurotoxin may be administered prior to, simultaneous or subsequent to the onset of a condition/disease that cause visceral pain. For example, where the visceral pain is caused by an infection, the clostridial neurotoxin may be administered subsequent to detection of the infection to suppress visceral pain.

As explained in more detail elsewhere in this disclosure, the term “clostridial neurotoxin” embraces a clostridial neurotoxin fragment thereof.

Further information on clostridial neurotoxins, and suitable neurotoxins for use in the invention, is now provided below.

The term “clostridial neurotoxin” embraces toxins produced by C. botulinum (botulinum neurotoxin serotypes A, B, C1, D, E, F, G, and X), C. tetani (tetanus neurotoxin), C. butyricum (botulinum neurotoxin serotype E), and C. baratii (botulinum neurotoxin serotype F), as well as modified clostridial neurotoxins or derivatives derived from any of the foregoing.

Botulinum neurotoxin (BoNT) is produced by C. botulinum in the form of a large protein complex, consisting of BoNT itself complexed to a number of accessory proteins. There are at present eight different classes of botulinum neurotoxin, namely: botulinum neurotoxin serotypes A, B, C1, D, E, F, G, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1.

Tetanus toxin is produced in a single serotype by C. tetani. C. butyricum produces BoNT/E, while C. baratii produces BoNT/F.

A clostridial neurotoxin may be selected from BoNT/A, BoNT/B, BoNT/C, BoNT/D, BoNT/E, BoNT/F, BoNT/G, BoNT/X, and TeNT (tetanus neurotoxin). Preferably, a clostridial neurotoxin is a botulinum neurotoxin, such as a botulinum neurotoxin selected from BoNT/A, BoNT/B, BoNT/C, BoNT/D, BoNT/E, BoNT/F, BoNT/G, and BoNT/X.

In one embodiment the clostridial neurotoxin may be BoNT/A. A reference BoNT/A sequence is shown as SEQ ID NO: 51 (additionally or alternatively, reference BoNT/A sequence may be that shown as SEQ ID NO: 62). In another embodiment the clostridial neurotoxin may be BoNT/B. A reference BoNT/B sequence is shown as SEQ ID NO: 52. In another embodiment the clostridial neurotoxin may be BoNT/C. A reference BoNT/C sequence is shown as SEQ ID NO: 53. In another embodiment the clostridial neurotoxin may be BoNT/D. A reference BoNT/D sequence is shown as SEQ ID NO: 54. In another embodiment the clostridial neurotoxin may be BoNT/E. A reference BoNT/E sequence is shown as SEQ ID NO: 55. In another embodiment the clostridial neurotoxin may be BoNT/F. A reference BoNT/F sequence is shown as SEQ ID NO: 56. In another embodiment the clostridial neurotoxin may be BoNT/G. A reference BoNT/G sequence is shown as SEQ ID NO: 57. In another embodiment the clostridial neurotoxin may be TeNT. A reference TeNT sequence is shown as SEQ ID NO: 58. In another embodiment the clostridial neurotoxin may be BoNT/X. A reference BoNT/X sequence is shown as SEQ ID NO: 59.

The term “clostridial neurotoxin” is intended to encompass both full length neurotoxins (comprising a clostridial neurotoxin L-chain and a clostridial neurotoxin H-chain), as well as fragments thereof. For example, the “clostridial neurotoxin” may refer to a polypeptide that comprises or consists of a clostridial neurotoxin L-chain, a clostridial neurotoxin translocation domain (H_{N}) and/or a clostridial neurotoxin receptor binding domain (H_{C}) domain. The “clostridial neurotoxin” may refer to a polypeptide that comprises or consists of a clostridial neurotoxin L-chain, a clostridial neurotoxin translocation domain (H_{N}) and/or a clostridial neurotoxin receptor binding domain (H_{C}) domain, wherein when the polypeptide comprises a clostridial neurotoxin L-chain, the L-chain is a catalytically inactive. In other words, the present invention may encompass the use of full-length clostridial neurotoxins comprising a clostridial neurotoxin L-chain and a clostridial neurotoxin H-chain, optionally with the proviso that said clostridial neurotoxin L-chain is catalytically inactive.

Advantageously, the use of a clostridial neurotoxin fragment allows for the use of non-toxic (or substantially non-toxic) fragments of clostridial neurotoxins, which given the smaller size (compared to the full-length H-chain or full-length clostridial neurotoxin), are less likely to provoke an adverse immune response (against the fragment) in a subject administered said fragments. Moreover, the non-toxic (or substantially non-toxic) fragments are less expensive and/or less complex to manufacture than full-length clostridial neurotoxins. Additionally, the non-toxic (or substantially non-toxic) fragments constitute a more well-defined therapeutic than the full-length clostridial toxins, and given the shorter length of the polypeptides there is a reduced probability of, for example, cysteine shuffling between domains.

In embodiments which refer to a particular domain of a clostridial neurotoxin, the molecule may conveniently be referred to as a “polypeptide”. That is, the term “clostridial neurotoxin” may be substituted for the term “polypeptide”.

Thus, the clostridial neurotoxin referred to herein may be a polypeptide that comprises (or consists of) a clostridial neurotoxin light-chain (L-chain), a clostridial neurotoxin translocation domain (H_{N} domain) and/or a clostridial neurotoxin receptor binding domain (H_{C} domain). The clostridial neurotoxin referred to herein may be a polypeptide that comprises (or consists of) a clostridial neurotoxin light-chain (L-chain), a clostridial neurotoxin translocation domain (H_{N} domain) and/or a clostridial neurotoxin receptor binding domain (H_{C} domain), wherein when the polypeptide comprises a clostridial neurotoxin L-chain, the L-chain is catalytically inactive.

It follows, therefore, that aspects of the invention provide any of the following:
a polypeptide for use in a method of suppressing visceral pain;
a method of suppressing visceral pain in a patient, the method comprising administering a polypeptide to the patient;
use of a polypeptide in the manufacture of a medicament to suppress visceral pain in a patient;
wherein said peptide comprises (or consists of) a clostridial neurotoxin light-chain (L-chain), a clostridial neurotoxin translocation domain (H_{N} domain) and/or a clostridial neurotoxin receptor binding domain (H_{C} domain); optionally wherein when the polypeptide comprises a clostridial neurotoxin L-chain, the L-chain is catalytically inactive.

In one embodiment, a clostridial neurotoxin of the invention may be encoded by a nucleotide sequence having at least 70% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60. In one embodiment, a clostridial neurotoxin of the invention may be encoded by a nucleotide sequence having at least 80%, 90%, 95% or 98% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60. Preferably, a clostridial neurotoxin of the invention may be encoded by a nucleotide sequence comprising any one of SEQ ID NOs: 1, 3, 5, 7, 9, 11, 13, 15, 17, 19, 21, 23, 25, 27, 29, 31, 33, 35, 37, 39, 41, 43, 45, 47, 49, or 60.

In one embodiment a clostridial neurotoxin of the invention may comprise a clostridial neurotoxin sequence having at least 70% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65. In one embodiment a clostridial neurotoxin of the invention may comprise a clostridial neurotoxin sequence having at least 80%, 90%, 95% or 98% sequence identity to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65. Preferably, a clostridial neurotoxin of the invention may comprise a clostridial neurotoxin sequence of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 51, 52, 53, 54, 55, 56, 57, 58, 59, 61, 62, 63, 64 or 65.

In one embodiment a clostridial neurotoxin of the invention may comprise a clostridial neurotoxin sequence having at least 70% sequence identity to any one of SEQ ID NOs: 51, 52, 53, 54, 55, 56, 57, 58, or 59 (preferably SEQ ID NOs: 51, 52, 53, 54, 55, 56, 57, or 59). In one embodiment a clostridial neurotoxin of the invention may comprise a clostridial neurotoxin sequence having at least 80%, 90%, 95% or 98% sequence identity to any one of SEQ ID NOs: 51, 52, 53, 54, 55, 56, 57, 58, or 59 (preferably SEQ ID NOs: 51, 52, 53, 54, 55, 56, 57, or 59). Preferably, a clostridial neurotoxin of the invention may comprise a clostridial neurotoxin sequence of any one of SEQ ID NOs: 51, 52, 53, 54, 55, 56, 57, 58, or 59 (preferably SEQ ID NOs: 51, 52, 53, 54, 55, 56, 57, or 59). In one embodiment a clostridial neurotoxin of the invention may comprise a clostridial neurotoxin sequence having at least 70% sequence identity to SEQ ID NO: 51. In one embodiment a clostridial neurotoxin of the invention may comprise a clostridial neurotoxin sequence having at least 80%, 90%, 95% or 98% sequence identity to SEQ ID NO: 51. Preferably, a clostridial neurotoxin of the invention may comprise a clostridial neurotoxin sequence of SEQ ID NO: 51.

The term “clostridial neurotoxin” is also intended to embrace modified clostridial neurotoxins and derivatives thereof, including but not limited to those described below. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, in one embodiment, the clostridial neurotoxin of the invention is a modified clostridial neurotoxin, or a modified clostridial neurotoxin derivative, or a clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL, xExxxLL, xExxxlL, and xExxxLM (wherein x is any amino acid)—e.g. SEQ ID Nos: 66-69. Suitable tyrosine-based motifs include Y-x-x-Hy (wherein Hy is a hydrophobic amino acid)—e.g. SEQ ID NO: 70. Examples of modified clostridial neurotoxins comprising leucine- and tyrosine-based motifs are described in WO 2002/08268, which is hereby incorporated by reference in its entirety.

As described above, a modified clostridial neurotoxin (or clostridial neurotoxin fragment) may be one that comprises one or more modifications that increases the isoelectric point of the clostridial neurotoxin when compared to an equivalent unmodified clostridial neurotoxin lacking said one or more modifications. Suitable modified clostridial neurotoxins are described above and in WO 2015/004461 A1 and WO 2016/110662 A1, which are incorporated herein by reference. Exemplary sequences include SEQ ID NOs: 61 and 42 described herein.

A modified clostridial neurotoxin may preferably comprise or consist of a sequence of SEQ ID No: 61.

The term “clostridial neurotoxin” is intended to embrace hybrid and chimeric clostridial neurotoxins. A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may contain the entire light chain of a light chain from one clostridial neurotoxin subtype and the heavy chain from another clostridial neurotoxin subtype. In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to subjects who are immunologically resistant to a given clostridial neurotoxin subtype, to subjects who may have a lower than average concentration of receptors to a given clostridial neurotoxin heavy chain binding domain, or to subjects who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, in one embodiment, the clostridial neurotoxin (or fragment thereof) of the invention is a hybrid clostridial neurotoxin, or a chimeric clostridial neurotoxin.

In a particularly preferred embodiment, a polypeptide of the invention may be a chimeric clostridial neurotoxin comprising (preferably consisting of) a BoNT/A light-chain and translocation domain, and a BoNT/B receptor binding domain (H_{C} domain) or a portion thereof (e.g. BoNT/A LH_{N}-BoNT/B H_{C}). A suitable chimeric and/or hybrid clostridial neurotoxin may be one taught in WO 2017/191315 A1, which is incorporated herein by reference. Such preferred sequences include SEQ ID NOs: 44, 63, and 64.

For example, a chimeric clostridial neurotoxin may comprise (preferably consist of) a sequence of SEQ ID NO: 64 (which may be said to comprise a catalytically inactive L-chain).

In a preferable embodiment, a chimeric clostridial neurotoxin may comprise (preferably consist of) a sequence of SEQ ID NO: 63.

The BoNT/A LH_{N} domain may be covalently linked to the BoNT/B H_{C} domain. Said chimeric BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

Reference herein to the “first amino acid residue of the 3₁₀ helix separating the LH_{N} and He domains of BoNT/A” means the N-terminal residue of the 3₁₀ helix separating the LH_{N} and He domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å(=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a S-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureId=3BTA) and 1EPW (http://www.rcsb.org/pdb/explore/explore.do?structureId=1EPW) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular that the region around the “H_{N}/H_{CN}” junction is structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) was used to obtain a predicted structure of other BoNT serotypes based on the BoNT/A1 crystal structure (3BTA.pdb);
2. The structural (pdb) files thus obtained were edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) was used to superpose each serotype onto the 3BTA.pdb structure;
4. The superposed pdb files were inspected to locate the 3₁₀ helix at the start of the H_{C} domain of BoNT/A1, and corresponding residues in the other serotype were then identified;
5. The other BoNT serotype sequences were aligned with Clustal Omega in order to check that corresponding residues were correct.

Examples of LH_{N}, H_{C} and 3₁₀ helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | (e.g. SEQ ID |
| NO: 62) | | | | NO: 71) |
| BoNT/A2 | X73423.3 | 1-872 | 873-1296 | ⁸⁷²NIVNTS⁸⁷⁷ |
| | | | | (e.g. SEQ ID |
| | | | | NO: 72) |
| BoNT/A3 | DQ185900.1 (aka | 1-872 | 873-1292 | ⁸⁷²NIVNTS⁸⁷⁷ |
| | Q3LRX9.1) | | | (e.g. SEQ ID |
| | | | | NO: 72) |
| BoNT/A4 | EU341307.1 (aka | 1-872 | 873-1296 | ⁸⁷²NITNAS⁸⁷⁷ |
| | Q3LRX8.1) | | | (e.g. SEQ ID |
| | | | | NO: 73) |
| BoNT/A5 | EU679004.1 (aka | 1-872 | 873-1296 | ⁸⁷²NIINTS⁸⁷⁷ |
| | C1IPK2.1) | | | (e.g. SEQ ID |
| | | | | NO: 71) |
| BoNT/A6 | FJ981696.1 | 1-872 | 873-1296 | ⁸⁷²NIINTS⁸⁷⁷ |
| | | | | (e.g. SEQ ID |
| | | | | NO: 71) |
| BoNT/A7 | JQ954969.1 (aka | 1-872 | 873-1296 | ⁸⁷²NIINTS⁸⁷⁷ |
| | K4LN57.1) | | | (e.g. SEQ ID |
| | | | | NO: 71) |
| BoNT/A8 | KM233166.1 | 1-872 | 873-1297 | ⁸⁷²NITNTS⁸⁷⁷ |
| | | | | (e.g. SEQ ID |
| | | | | NO: 74) |
| BoNT/B1 | B1INP5.1 | 1-859 | 860-1291 | ⁸⁵⁹EILNNI⁸⁶⁴ |
| (a.k.a. SEQ | | | | (e.g. SEQ ID |
| ID NO: 52) | | | | NO: 75) |
| BoNT/B2 | AB084152.1 (aka | 1-859 | 860-1291 | ⁸⁵⁹EILNNI⁸⁶⁴ |
| | Q8GR96.1) | | | (e.g. SEQ ID |
| | | | | NO: 75) |
| BoNT/B3 | EF028400.1 (aka | 1-859 | 860-1291 | ⁸⁵⁹EILNNI⁸⁶⁴ |
| | A2I2S2.1) | | | (e.g. SEQ ID |
| | | | | NO: 75) |
| BoNT/B4 | EF051570.1 (aka | 1-859 | 860-1291 | ⁸⁵⁹EILNNI⁸⁶⁴ |
| | A2I2W0.1) | | | (e.g. SEQ ID |
| | | | | NO: 75) |
| BoNT/B5 | EF033130.1 (aka | 1-859 | 860-1291 | ⁸⁵⁹DILNNI⁸⁶⁴ |
| | A2I2U6.1) | | | (e.g. SEQ ID |
| | | | | NO: 76) |
| BoNT/B6 | AB302852.1 (aka | 1-859 | 860-1291 | ⁸⁵⁹EILNNI⁸⁶⁴ |
| | A8R089.1) | | | (e.g. SEQ ID |
| | | | | NO: 75) |
| BoNT/B7 | JQ354985.1 (aka | 1-859 | 860-1291 | ⁸⁵⁹EILNNI⁸⁶⁴ |
| | H9CNK9.1) | | | (e.g. SEQ ID |
| | | | | NO: 75) |
| BoNT/B8 | JQ964806.1 (aka | 1-859 | 860-1292 | ⁸⁵EILNNI⁸⁶⁴ |
| | I6Z8G9.1) | | | (e.g. SEQ ID |
| | | | | NO: 75) |

Using structural analysis and sequence alignments, it was found that the p-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a He domain from BoNT/B,
wherein the C-terminal amino acid residue of the LH_{N} domain corresponds to the eighth amino acid residue N-terminally to the p-strand located at the beginning (N-term) of the H_{C} domain of BoNT/A, and
wherein the N-terminal amino acid residue of the H_{C} domain corresponds to the seventh amino acid residue N-terminally to the p-strand located at the beginning (N-term) of the H_{C} domain of BoNT/B.

A BoNT/AB chimera may comprise an LH_{N} domain from BoNT/A covalently linked to a He domain from BoNT/B,
wherein the C-terminal amino acid residue of the LH_{N} domain corresponds to the C-terminal amino acid residue of the α-helix located at the end (C-term) of LH_{N} domain of BoNT/A, and
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity.

The LH_{N} domain from BoNT/A may correspond to amino acid residues 1 to 872 of SEQ ID NO: 62, or a polypeptide sequence having at least 70% sequence identity thereto. The LH_{N} domain from BoNT/A may correspond to amino acid residues 1 to 872 of SEQ ID NO: 62, or a polypeptide sequence having at least 80%, 90% or 95% sequence identity thereto. Preferably, the LH_{N} domain from BoNT/A corresponds to amino acid residues 1 to 872 of SEQ ID NO: 62.

The H_{C} domain from BoNT/B may correspond to amino acid residues 860 to 1291 of SEQ ID NO: 52, or a polypeptide sequence having at least 70% sequence identity thereto. The He domain from BoNT/B may correspond to amino acid residues 860 to 1291 of SEQ ID NO: 52, or a polypeptide sequence having at least 80%, 90% or 95% sequence identity thereto. Preferably, the H_{C} domain from BoNT/B corresponds to amino acid residues 860 to 1291 of SEQ ID NO: 52.

Preferably, the BoNT/AB chimera comprises a BoNT/A LH_{N} domain and a BoNT/B He domain. More preferably, the LH_{N} domain corresponds to amino acid residues 1 to 872 of BoNT/A (SEQ ID NO: 62) and the H_{C} domain corresponds to amino acid residues 860 to 1291 of BoNT/B (SEQ ID NO: 52).

Preferably, a BoNT/B H_{C} domain further comprises at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain have been disclosed in WO 2013/180799 and in WO 2016/154534 (both herein incorporated by reference).

Suitable amino acid residue substitution, addition or deletion in the BoNT/B H_{CC} subdomain include substitution mutations selected from the group consisting of: V1118M; Y1183M; E1191M; E1191I; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

Suitable amino acid residue substitution, addition or deletion in the BoNT/B H_{CC} subdomain further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

Suitable amino acid residue substitution, addition or deletion in the BoNT/B H_{CC} subdomain also include a combination of three substitution mutations which are E1191M, S1199W and W1178Q.

Preferably, the suitable amino acid residue substitution, addition or deletion in the BoNT/B H_{CC} subdomain includes a combination of two substitution mutations which are E1191M and S1199Y.

The modification may be a modification when compared to unmodified BoNT/B shown as SEQ ID NO: 52, wherein the amino acid residue numbering is determined by alignment with SEQ ID NO: 52. As the presence of a methionine residue at position 1 of SEQ ID NO: 52 is optional, the skilled person will take the presence/absence of the methionine residue into account when determining amino acid residue numbering. For example, where SEQ ID NO: 52 includes a methionine, the position numbering will be as defined above (e.g. E1191 will be E1191 of SEQ ID NO: 52). Alternatively, where the methionine is absent from SEQ ID NO: 52 the amino acid residue numbering should be modified by −1 (e.g. E1191 will be E1190 of SEQ ID NO: 52). Similar considerations apply when the methionine at position 1 of the other polypeptide sequences described herein is present/absent, and the skilled person will readily determine the correct amino acid residue numbering using techniques routine in the art.

Thus, in one aspect, the invention provides a clostridial neurotoxin for use in treating a brain disorder by promoting a neuroimmune response in a subject, wherein the clostridial neurotoxin comprises a polypeptide sequence having at least 70% sequence identity to SEQ ID NO: 63 or 64 (preferably wherein the clostridial neurotoxin comprises a polypeptide sequence having at least 70% sequence identity to SEQ ID NO: 63).

In a related aspect, there is provided a method for suppressing visceral pain in a subject, the method comprising administering a clostridial neurotoxin to the subject, wherein the clostridial neurotoxin comprises a polypeptide sequence having at least 70% sequence identity to SEQ ID NO: 63 or 64 (preferably wherein the clostridial neurotoxin comprises a polypeptide sequence having at least 70% sequence identity to SEQ ID NO: 63).

In a related aspect, there is provided a method for suppressing visceral pain in a subject, the method comprising administering a clostridial neurotoxin to the subject, wherein the clostridial neurotoxin comprises a polypeptide sequence having at least 70% sequence identity to SEQ ID NO: 63 or 64 (preferably wherein the clostridial neurotoxin comprises a polypeptide sequence having at least 70% sequence identity to SEQ ID NO: 63).

In one embodiment a clostridial neurotoxin for use according to the invention comprises a polypeptide sequence having at least 80%, 90%, 95% or 98% sequence identity to SEQ ID NO: 63 or 64. Preferably, a clostridial neurotoxin for use according to the invention comprises (more preferably consists of) a polypeptide sequence shown as SEQ ID NO: 63 or 64.

For example, a clostridial neurotoxin for use according to the invention may comprise a polypeptide sequence having at least 80%, 90%, 95% or 98% sequence identity to SEQ ID NO: 64. A clostridial neurotoxin may comprises (more preferably consist of) a polypeptide sequence shown as SEQ ID NO: 64.

In a preferred embodiment, a clostridial neurotoxin for use according to the invention may comprise a polypeptide sequence having at least 80%, 90%, 95% or 98% sequence identity to SEQ ID NO: 64. For example, a particularly suitable clostridial neurotoxin may comprises (more preferably consist of) a polypeptide sequence shown as SEQ ID NO: 63.

Preferably, the clostridial neurotoxin comprising a polypeptide sequence having at least 70% sequence identity to SEQ ID NO: 63 comprises a catalytically-inactive L-chain, such as SEQ ID NO: 64.

A chimeric and/or hybrid clostridial neurotoxin for use in the present invention may comprise a portion of a BoNT/A polypeptide and a portion of a BoNT/B polypeptide, an example of which includes the polypeptide described herein as SEQ ID NO: 44.

Suitable chimeric clostridial neurotoxins may include BoNT/FA. Indeed, in a particularly preferred embodiment, a clostridial neurotoxin (e.g. chimeric clostridial neurotoxin) of the invention may comprise BoNT/FA or a fragment thereof. Catalytically inactive forms of BoNT/FA are described herein as SEQ ID NOs: 26 and 34. Suitable fragments of BoNT/FA are also described herein as SEQ ID NOs: 28, 30, and 32.

The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OT022244.1), which cleaves VAMP2 and SNAP25, and the Chryseobacterium pipero encoded toxin (NCBI Ref.Seq: WP_034687872.1).

The clostridial neurotoxin of the present invention may lack a functional H_{C} domain of a clostridial neurotoxin and also lack any functionally equivalent exogenous ligand Targeting Moiety (TM).

The clostridial neurotoxin of the invention may be a re-targeted clostridial neurotoxin. In an alternative embodiment, a clostridial neurotoxin of the invention is not a re-targeted clostridial neurotoxin. In a re-targeted clostridial neurotoxin, the clostridial neurotoxin is modified to include an exogenous ligand known as a Targeting Moiety (TM). The TM is selected to provide binding specificity for a desired target cell, and as part of the re-targeting process the native binding portion of the clostridial neurotoxin (e.g. the H_{C} domain, or the H_{CC} domain) may be removed. Re-targeting technology is described, for example, in: EP-B-0689459; WO 1994/021300; EP-B-0939818; U.S. Pat. Nos. 6,461,617; 7,192,596; WO 1998/007864; EP-B-0826051; U.S. Pat. Nos. 5,989,545; 6,395,513; 6,962,703; WO 1996/033273; EP-B-0996468; U.S. Pat. No. 7,052,702; WO 1999/017806; EP-B-1107794; U.S. Pat. No. 6,632,440; WO 2000/010598; WO 2001/21213; WO 2006/059093; WO 2000/62814; WO 2000/04926; WO 1993/15766; WO 2000/61192; and WO 1999/58571; all of which are hereby incorporated by reference in their entirety.

As discussed above, (full-length) clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

In one embodiment a clostridial neurotoxin of the invention comprises a fragment of a BoNT/A or a fragment of a BoNT/F. In another embodiment, the clostridial neurotoxin of the invention comprises a catalytically inactive L-chain of BoNT/A or BoNT/F. For example, the clostridial neurotoxin of the invention may comprise a catalytically inactive L-chain of BoNT/A.

In embodiments where a clostridial neurotoxin described herein has a tag for purification (e.g. a His-tag) and/or a linker, said tag and/or linker are optional.

Suitable full-length clostridial neurotoxins are described herein.

In one embodiment a clostridial neurotoxin of the invention may comprise a polypeptide sequence having at least 70% sequence identity to any one of SEQ ID NOs: 2, 10, 12, 14, 16, 18, 26, 34, 51, 52, 53, 54, 55, 56, 57, 58, 59, 61, 62, 63, 64 or 65, preferably with the proviso that a clostridial neurotoxin L-chain of said clostridial neurotoxin is catalytically inactive. In one embodiment a clostridial neurotoxin of the invention may comprise a polypeptide sequence having at least 80%, 90%, 95% or 98% sequence identity to any one of SEQ ID NOs: 2, 10, 12, 14, 16, 18, 26, 34, 51, 52, 53, 54, 55, 56, 57, 58, 59, 61, 62, 63, 64 or 65, preferably with the proviso that a clostridial neurotoxin L-chain of said clostridial neurotoxin is catalytically inactive. Preferably, a clostridial neurotoxin of the invention may comprise a polypeptide sequence comprising any one of SEQ ID NOs: 2, 10, 12, 14, 16, 18, 26, 34, 51, 52, 53, 54, 55, 56, 57, 58, 59, 61, 62, 63, 64 or 65, preferably with the proviso that a clostridial neurotoxin L-chain of said clostridial neurotoxin is catalytically inactive.

In one embodiment a clostridial neurotoxin of the invention may be one encoded by a nucleotide sequence having at least 70% sequence identity to any one of SEQ ID NOs: 1, 9, 11, 13, 15, 17, 25, 33, or 60, preferably with the proviso that the clostridial neurotoxin L-chain of said clostridial neurotoxin is catalytically inactive. In one embodiment a clostridial neurotoxin of the invention is one encoded by a nucleotide sequence having at least 80%, 90%, 95% or 98% sequence identity to any one of SEQ ID NOs: 1, 9, 11, 13, 15, 17, 25, 33, or 60, preferably with the proviso that the clostridial neurotoxin L-chain of said clostridial neurotoxin is catalytically inactive. Preferably, a clostridial neurotoxin of the invention is one encoded by a nucleotide sequence comprising any one of SEQ ID NOs: 1, 9, 11, 13, 15, 17, 25, 33, or 60, preferably with the proviso that the clostridial neurotoxin L-chain of said clostridial neurotoxin is catalytically inactive.

In one embodiment a clostridial neurotoxin of the invention may comprise a polypeptide sequence having at least 70% sequence identity to any one of SEQ ID NOs: 2, 10, 12, 14, 16, 18, 26, 34, 64 or 65, preferably with the proviso that the clostridial neurotoxin L-chain of said clostridial neurotoxin is catalytically inactive. In one embodiment a clostridial neurotoxin of the invention comprises a polypeptide sequence having at least 80%, 90%, 95% or 98% sequence identity to any one of SEQ ID NOs: 2, 10, 12, 14, 16, 18, 26, 34, 64 or 65, preferably with the proviso that the clostridial neurotoxin L-chain of said clostridial neurotoxin is catalytically inactive. Preferably, a clostridial neurotoxin of the invention comprises any one of SEQ ID NOs: 2, 10, 12, 14, 16, 18, 26, 34, 64 or 65, preferably with the proviso that the clostridial neurotoxin L-chain of said clostridial neurotoxin is catalytically inactive.

In one embodiment a clostridial neurotoxin of the invention is a full-length clostridial neurotoxin selected from BoNT/A, BoNT/B, BoNT/C, BoNT/D, BoNT/E, BoNT/F, BoNT/G, BoNT/X, and TeNT.

In one embodiment a clostridial neurotoxin of the invention is a full-length clostridial neurotoxin selected from BoNT/B, BoNT/C, BoNT/D, BoNT/E, BoNT/F, BoNT/G, BoNT/X, and TeNT.

In one embodiment a clostridial neurotoxin of the invention may comprise a polypeptide sequence having at least 70% sequence identity to any one of SEQ ID NOs: 51-59, 61 or 63.

In one embodiment a clostridial neurotoxin of the invention may comprise a polypeptide sequence having at least 80%, 90%, 95% or 98% sequence identity to any one of SEQ ID NOs: 51-59, 61 or 63. In one embodiment a clostridial neurotoxin of the invention may comprise a polypeptide sequence having at least 99% or 99.9% sequence identity to any one of SEQ ID NOs: 51-59, 61 or 63. Preferably, a clostridial neurotoxin of the invention may comprise (more preferably consist of) a polypeptide sequence comprising any one of SEQ ID NOs: 51-59, 61 or 63.

In one embodiment a clostridial neurotoxin of the invention may comprise a polypeptide sequence having at least 70% sequence identity to any one of SEQ ID NOs: 52-59, 61 or 63. In one embodiment a clostridial neurotoxin of the invention may comprise a polypeptide sequence having at least 80%, 90%, 95% or 98% sequence identity to any one of SEQ ID NOs: 52-59, 61 or 63. In one embodiment a clostridial neurotoxin of the invention may comprise a polypeptide sequence having at least 99% or 99.9% sequence identity to any one of SEQ ID NOs: 52-59, 61 or 63. Preferably, a clostridial neurotoxin of the invention may comprise (more preferably consist of) a polypeptide sequence comprising any one of SEQ ID NOs: 52-59, 61 or 63.

In one embodiment a clostridial neurotoxin of the invention is not a full-length catalytically active clostridial neurotoxin, e.g. is not full-length catalytically active BoNT/A.

The clostridial neurotoxin of the present invention may comprise (or consist of) a fragment of a clostridial neurotoxin, e.g. a fragment of any full-length clostridial neurotoxin described herein.

In one embodiment a clostridial neurotoxin of the invention may comprise a fragment of a polypeptide sequence having at least 70% sequence identity to any one of SEQ ID NOs: 2, 10, 12, 14, 16, 18, 26, 34, 51, 52, 53, 54, 55, 56, 57, 58, 59, 61, 62, 63, 64 or 65. In one embodiment a clostridial neurotoxin of the invention may comprise a fragment of a polypeptide sequence having at least 80%, 90%, 95% or 98% sequence identity to any one of SEQ ID NOs: 2, 10, 12, 14, 16, 18, 26, 34, 51, 52, 53, 54, 55, 56, 57, 58, 59, 61, 62, 63, 64 or 65. Preferably, a clostridial neurotoxin of the invention may comprise a fragment of a polypeptide sequence comprising any one of SEQ ID NOs: 2, 10, 12, 14, 16, 18, 26, 34, 51, 52, 53, 54, 55, 56, 57, 58, 59, 61, 62, 63, 64 or 65.

In one embodiment a clostridial neurotoxin of the invention comprises (or consists of) a clostridial neurotoxin L-chain or fragment thereof. A fragment of a clostridial neurotoxin L-chain may have ≤400, ≤350, ≤300, ≤250, ≤200, ≤150, ≤100 or ≤50 amino acid residues of a clostridial neurotoxin L-chain. In one embodiment, a fragment of a clostridial neurotoxin L-chain has at least 20, 30, 40, 50, 60, 70, 80, 90, 100, 120, 150 or 200 amino acid residues of a clostridial neurotoxin L-chain. For example, a fragment of a clostridial neurotoxin L-chain may have 20-400, 50-300 or 100-200 amino acid residues of a clostridial neurotoxin L-chain.

Examples of L-chain reference sequences include:
Botulinum type A neurotoxin: amino acid residues 1-448
Botulinum type B neurotoxin: amino acid residues 1-440
Botulinum type C1 neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently-identified BoNT/X, the L-chain has been reported as corresponding to amino acids 1-439 thereof, with the L-chain boundary potentially varying by approximately 25 amino acids (e.g. 1-414 or 1-464).

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C1 neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

Suitable clostridial neurotoxin L-chains are described herein.

A clostridial neurotoxin L-chain may comprise a polypeptide sequence having at least 70% sequence identity to any one of SEQ ID NOs: 6, 24, 32 or 40 or a fragment thereof. In one embodiment a clostridial neurotoxin L-chain comprises a polypeptide sequence having at least 80%, 90%, 95% or 98% sequence identity to any one of SEQ ID NOs: 6, 24, 32 or 40 or a fragment thereof. Preferably, a clostridial neurotoxin L-chain comprises (more preferably consists of) a polypeptide sequence comprising any one of SEQ ID NOs: 6, 24, 32 or 40 or a fragment thereof.

A clostridial neurotoxin L-chain may be one encoded by a nucleotide sequence having at least 70% sequence identity to any one of SEQ ID NOs: 5, 23, 31 or 39 or a fragment thereof. In one embodiment a clostridial neurotoxin L-chain is one encoded by a nucleotide sequence having at least 80%, 90%, 95% or 98% sequence identity to any one of SEQ ID NOs: 5, 23, 31 or 39 or a fragment thereof. Preferably, a clostridial neurotoxin L-chain is one encoded by a nucleotide sequence comprising any one of SEQ ID NOs: 5, 23, 31 or 39 or a fragment thereof.

In one embodiment a clostridial neurotoxin of the invention comprises (or consists of) a fragment of a clostridial neurotoxin H-chain. A fragment of a clostridial neurotoxin H-chain may have ≤800, ≤700, ≤600, ≤500, ≤400, ≤350, ≤300, ≤250, ≤200, ≤150, ≤100 or ≤50 amino acid residues of a clostridial neurotoxin H-chain. In one embodiment, a fragment of a clostridial neurotoxin H-chain has at least 20, 30, 40, 50, 60, 70, 80, 90, 100, 120, 150 or 200 amino acid residues of a clostridial neurotoxin H-chain. For example, a fragment of a clostridial neurotoxin H-chain may have 20-800, 30-600, 40-400, 50-300 or 100-200 amino acid residues of a clostridial neurotoxin H-chain.

A clostridial neurotoxin H-chain comprises two structural/functional domains: the translocation domain (H_{N}) and receptor binding domain (H_{C}).

In one embodiment a clostridial neurotoxin of the invention comprises (or consists of) a clostridial neurotoxin translocation domain or a fragment thereof. A fragment of a clostridial neurotoxin translocation domain may have ≤400, ≤350, ≤300, ≤250, ≤200, ≤150, ≤100 or ≤50 amino acid residues of a clostridial neurotoxin translocation domain. In one embodiment, a fragment of a clostridial neurotoxin translocation domain has at least 20, 30, 40, 50, 60, 70, 80, 90, 100, 120, 150 or 200 amino acid residues of a clostridial neurotoxin translocation domain. For example, a fragment of a clostridial neurotoxin translocation domain may have 20-400, 50-300 or 100-200 amino acid residues of a clostridial neurotoxin translocation domain.

The translocation domain is a fragment of the H-chain of a clostridial neurotoxin approximately equivalent to the amino-terminal half of the H-chain, or the domain corresponding to that fragment in the intact H-chain. In one embodiment the He function of the H-chain may be removed by deletion of the He amino acid sequence (either at the DNA synthesis level, or at the post-synthesis level by nuclease or protease treatment).

Alternatively, the He function may be inactivated by chemical or biological treatment. Thus, in some embodiments the H-chain may be incapable of binding to the Binding Site on a target cell to which native clostridial neurotoxin (i.e. holotoxin) binds.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (S447-S863)
Tetanus neurotoxin—amino acid residues (S458-V879)

In the context of the present invention, a variety of clostridial neurotoxin H_{N} regions comprising a translocation domain can be useful in aspects of the present invention. In one embodiment these active fragments can facilitate the release of a non-cytotoxic protease (e.g. a clostridial L-chain) from intracellular vesicles into the cytoplasm of the target cell and thus participate in executing the overall cellular mechanism whereby a clostridial neurotoxin proteolytically cleaves a substrate. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, aspects of this embodiment can include clostridial neurotoxin H_{N} regions comprising a translocation domain having a length of, for example, at least 350 amino acids, at least 375 amino acids, at least 400 amino acids and at least 425 amino acids. Other aspects of this embodiment can include clostridial neurotoxin H_{N} regions comprising a translocation domain having a length of, for example, at most 350 amino acids, at most 375 amino acids, at most 400 amino acids and at most 425 amino acids.

For further details on the genetic basis of toxin production in Clostridium botulinum and C. tetani, see Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues. In one embodiment said modified H_{N} portions still demonstrate the above-mentioned translocation function.

In a preferred embodiment a clostridial neurotoxin of the invention comprises (or consists of) a clostridial neurotoxin receptor binding domain (H_{C}) or a fragment thereof. A fragment of a clostridial neurotoxin receptor binding domain (H_{C}) may have ≤350, ≤300, ≤250, ≤200, ≤150, ≤100 or ≤50 amino acid residues of a clostridial neurotoxin receptor binding domain (He). In one embodiment, a fragment of a clostridial neurotoxin receptor binding domain (H_{C}) has at least 20, 30, 40, 50, 60, 70, 80, 90, 100, 120, 150 or 200 amino acid residues of a clostridial neurotoxin receptor binding domain (He). For example, a fragment of a clostridial neurotoxin receptor binding domain (H_{C}) may have 20-350, 50-300 or 100-200 amino acid residues of a clostridial neurotoxin receptor binding domain (He).

Examples of clostridial neurotoxin receptor binding domain (H_{C}) reference sequences include:
BoNT/A—N872-L1296
BoNT/B—E859-E1291
BoNT/C1—N867-E1291
BoNT/D—S863-E1276
BoNT/E—R846-K1252
BoNT/F—K865-E1274
BoNT/G—N864-E1297
TeNT—1880-D1315

For recently-identified BoNT/X, the H_{C} domain has been reported as corresponding to amino acids 893-1306 thereof, with the domain boundary potentially varying by approximately 25 amino acids (e.g. 868-1306 or 918-1306).

A clostridial neurotoxin H-chain may further comprise a translocation facilitating domain. Said domain facilitates delivery of the L-chain into the cytosol of the target cell and are described, for example, in WO 08/008803 and WO 08/008805, each of which is herein incorporated by reference thereto.

By way of example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{CN} translocation facilitating domain preferably has a length of at most 200 amino acids, at most 225 amino acids, at most 250 amino acids, or at most 275 amino acids.

Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Tetanus neurotoxin—amino acid residues (880-1127)

The above sequence positions may vary a little according to serotype/sub-type, and further examples of suitable (reference) clostridial neurotoxin H_{CN} domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Suitable clostridial neurotoxin H_{C} domains are described herein.

A clostridial neurotoxin H_{C} domain may comprise a polypeptide sequence having at least 70% sequence identity to any one of SEQ ID NOs: 8, 22, 30, 38, 42, 44, 46, 48 or 50 or a fragment thereof. In one embodiment a clostridial neurotoxin H_{C} domain comprises a polypeptide sequence having at least 80%, 90%, 95% or 98% sequence identity to any one of SEQ ID NOs: 8, 22, 30, 38, 42, 44, 46, 48 or 50 or a fragment thereof. Preferably, a clostridial neurotoxin H_{C} domain comprises (more preferably consists of) a polypeptide sequence comprising any one of SEQ ID NOs: 8, 22, 30, 38, 42, 44, 46, 48 or 50 or a fragment thereof.

A clostridial neurotoxin H_{C} domain may be one encoded by a nucleotide sequence having at least 70% sequence identity to any one of SEQ ID NOs: 7, 21, 29, 37, 41, 43, 45, 47 or 49 or a fragment thereof. In one embodiment a clostridial neurotoxin H_{C} domain is one encoded by a nucleotide sequence having at least 80%, 90%, 95% or 98% sequence identity to any one of SEQ ID NOs: 7, 21, 29, 37, 41, 43, 45, 47 or 49 or a fragment thereof. Preferably, a clostridial neurotoxin H_{C} domain is one encoded by a nucleotide sequence comprising any one of SEQ ID NOs: 7, 21, 29, 37, 41, 43, 45, 47 or 49 or a fragment thereof.

In one embodiment a clostridial neurotoxin H_{C} domain for use in the invention is a variant BoNT/A H_{C} domain. Said variant BoNT/A H_{C} domain may comprise a modification of one or more amino acids residues selected from Y1117, F1252, H1253, and L1278. For example, a variant BoNT/A H_{C} domain may comprise one or more (preferably two or more) of the following modifications Y1117V, F1252Y, H1253K, and L1278F or L1278H.

In one embodiment a variant BoNT/A H_{C} domain comprises the following modifications: Y1117V and H1253K; or Y1117V, F1252Y, H1253K, and L1278F; or Y1117V, F1252Y, H1253K, and L1278H.

Preferably, a variant BoNT/A H_{C} domain comprises the following modifications: Y1117V and H1253K; or Y1117V, F1252Y, H1253K, and L1278H.

The modification may be a modification when compared to unmodified BoNT/A shown as SEQ ID NO: 62, wherein the amino acid residue numbering is determined by alignment with SEQ ID NO: 62. As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. Y1117 will align against Y1117 of SEQ ID NO: 62). Alternatively, where the methionine is absent from SEQ ID NO: 62 the amino acid residue numbering should be modified by −1 (e.g. Y1117 will align against Y1116 of SEQ ID NO: 52). Similar considerations apply when the methionine at position 1 of the other polypeptide sequences described herein is present/absent, and the skilled person will readily determine the correct amino acid residue numbering using techniques routine in the art.

A variant BoNT/A H_{C} domain may comprise a polypeptide sequence having at least 70% sequence identity to any one of SEQ ID NOs: 46, 48 or 50 or a fragment thereof with the proviso that the variant BoNT/A H_{C} domain comprises a modification as described above. In one embodiment a variant BoNT/A H_{C} domain comprises a polypeptide sequence having at least 80%, 90%, 95% or 98% sequence identity to any one of SEQ ID NOs: 46, 48 or 50 or a fragment thereof with the proviso that the variant BoNT/A H_{C} domain comprises a modification as described above. In one embodiment a variant BoNT/A H_{C} domain comprises a polypeptide sequence having at least 99% or 99.9% sequence identity to any one of SEQ ID NOs: 46, 48 or 50 or a fragment thereof with the proviso that the variant BoNT/A He domain comprises a modification as described above. Preferably, a variant BoNT/A He domain comprises (more preferably consists of) a polypeptide sequence comprising any one of SEQ ID NOs: 46, 48 or 50 or a fragment thereof.

A variant BoNT/A H_{C} domain may comprise a polypeptide sequence having at least 70% sequence identity to any one of SEQ ID NOs: 46 or 50 or a fragment thereof with the proviso that the variant BoNT/A H_{C} domain comprises a modification as described above. In one embodiment a variant BoNT/A H_{C} domain comprises a polypeptide sequence having at least 80%, 90%, 95% or 98% sequence identity to any one of SEQ ID NOs: 46 or 50 or a fragment thereof with the proviso that the variant BoNT/A H_{C} domain comprises a modification as described above. In one embodiment a variant BoNT/A H_{C} domain comprises a polypeptide sequence having at least 99% or 99.9% sequence identity to any one of SEQ ID NOs: 46 or 50 or a fragment thereof with the proviso that the variant BoNT/A H_{C} domain comprises a modification as described above. Preferably, a variant BoNT/A H_{C} domain comprises (more preferably consists of) a polypeptide sequence comprising any one of SEQ ID NOs: 46 or 50 or a fragment thereof.

A variant BoNT/A H_{C} domain may be one encoded by a nucleotide sequence having at least 70% sequence identity to any one of SEQ ID NOs: 45, 47 or 49 or a fragment thereof with the proviso that the variant BoNT/A H_{C} domain comprises a modification as described above.

In one embodiment a variant BoNT/A H_{C} domain be one encoded by a nucleotide sequence having at least 80%, 90%, 95% or 98% sequence identity to any one of SEQ ID NOs: 45, 47 or 49 or a fragment thereof with the proviso that the variant BoNT/A H_{C} domain comprises a modification as described above. In one embodiment a variant BoNT/A H_{C} domain be one encoded by a nucleotide sequence having at least 99% or 99.9% sequence identity to any one of SEQ ID NOs: 45, 47 or 49 or a fragment thereof with the proviso that the variant BoNT/A H_{C} domain comprises a modification as described above. Preferably, a variant BoNT/A H_{C} domain be one encoded by any one of SEQ ID NOs: 45, 47 or 49 or a fragment thereof.

A variant BoNT/A H_{C} domain may be one encoded by a nucleotide sequence having at least 70% sequence identity to any one of SEQ ID NOs: 45 or 49 or a fragment thereof with the proviso that the variant BoNT/A H_{C} domain comprises a modification as described above. In one embodiment a variant BoNT/A H_{C} domain be one encoded by a nucleotide sequence having at least 80%, 90%, 95% or 98% sequence identity to any one of SEQ ID NOs: 45 or 49 or a fragment thereof with the proviso that the variant BoNT/A H_{C} domain comprises a modification as described above. In one embodiment a variant BoNT/A H_{C} domain be one encoded by a nucleotide sequence having at least 99% or 99.9% sequence identity to any one of SEQ ID NOs: 45 or 49 or a fragment thereof with the proviso that the variant BoNT/A H_{C} domain comprises a modification as described above. Preferably, a variant BoNT/A He domain be one encoded by any one of SEQ ID NOs: 45 or 49 or a fragment thereof.

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide. Alternatively, a clostridial neurotoxin H_{CN} translocation facilitating domain may be combined with a non-clostridial translocation domain peptide. Alternatively, a clostridial neurotoxin H_{CN} facilitating domain may be combined with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

In some embodiments the clostridial neurotoxins of the present invention may lack a functional H_{C} domain of a clostridial neurotoxin. In one embodiment, the clostridial neurotoxins preferably lack the last 50 C-terminal amino acids of a clostridial neurotoxin holotoxin. In another embodiment, the clostridial neurotoxins preferably lack the last 100, preferably the last 150, more preferably the last 200, particularly preferably the last 250, and most preferably the last 300 C-terminal amino acid residues of a clostridial neurotoxin holotoxin. Alternatively, the He binding activity may be negated/reduced by mutagenesis—by way of example, referring to BoNT/A for convenience, modification of one or two amino acid residue mutations (W1266 to L and Y1267 to F) in the ganglioside binding pocket causes the He region to lose its receptor binding function. Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of He receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

The He peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the H_{CC} peptide or domain). This fact is confirmed by the following publications, each of which is herein incorporated in its entirety by reference thereto: Umland TC (1997) Nat. Struct. Biol. 4: 788-792; Herreros J (2000) Biochem. J. 347: 199-204; Halpern J (1993) J. Biol. Chem. 268: 15, pp. 11188-11192; Rummel A (2007) PNAS 104: 359-364; Lacey DB (1998) Nat. Struct. Biol. 5: 898-902; Knapp (1998) Am. Cryst. Assoc. Abstract Papers 25: 90; Swaminathan and Eswaramoorthy (2000) Nat. Struct. Biol. 7: 1751-1759; and Rummel A (2004) Mol. Microbiol. 51(3), 631-643. Moreover, it has been well documented that the C-terminal region (H_{CC}), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking a functional heavy chain He peptide (or domain) such that the heavy-chain is incapable of binding to cell surface receptors to which a native clostridial neurotoxin binds means that the clostridial heavy-chain simply lacks a functional H_{CC} peptide. In other words, the H_{CC} peptide region may be either partially or wholly deleted, or otherwise modified (e.g. through conventional chemical or proteolytic treatment) to reduce its native binding ability for nerve terminals at the neuromuscular junction.

Thus, in one embodiment, a clostridial neurotoxin H_{N} peptide of the present invention lacks part of a C-terminal peptide portion (H_{CC}) of a clostridial neurotoxin and thus lacks the He binding function of native clostridial neurotoxin. By way of example, in one embodiment, the C-terminally extended clostridial H_{N} peptide lacks the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. In another embodiment, the clostridial H_{N} peptide of the present invention lacks the entire C-terminal peptide portion (H_{CC}) of a clostridial neurotoxin and thus lacks the He binding function of native clostridial neurotoxin. By way of example, in one embodiment, the clostridial H_{N} peptide lacks the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention lacks a clostridial H_{CC} reference sequence selected from the group consisting of:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K1252)
Botulinum type F neurotoxin—amino acid residues (Y1106-E1274)
Botulinum type G neurotoxin—amino acid residues (Y1106-E1297)
Tetanus neurotoxin—amino acid residues (Y1128-D1315).

The above-identified reference sequences should be considered a guide as slight variations may occur according to sub-serotypes.

In a preferred embodiment a clostridial neurotoxin of the invention comprises (or consists of) a clostridial neurotoxin L-chain or fragment thereof and a fragment of a clostridial neurotoxin H-chain. For example, a clostridial neurotoxin may comprise (or consist of) a clostridial neurotoxin L-chain or fragment thereof and a clostridial neurotoxin translocation domain (H_{N}). Preferably, the clostridial neurotoxin does not further comprise a clostridial neurotoxin receptor binding domain (H_{C}) or at least the C-terminal portion of a clostridial neurotoxin receptor binding domain (H_{CC}). Thus, in one embodiment a clostridial neurotoxin of the present invention lacks a C-terminal portion of a clostridial neurotoxin receptor binding domain (H_{CC}). Advantageously, such clostridial neurotoxins lack the endogenous clostridial neurotoxin receptor binding capabilities and thus exhibit fewer off-target effects in a subject administered said clostridial neurotoxin.

In one embodiment a clostridial neurotoxin of the invention consists essentially of a clostridial neurotoxin L-chain or fragment thereof and/or a fragment of a clostridial neurotoxin H-chain. The term “consists essentially of” as used in this context means that the clostridial neurotoxin does not further comprise one or more amino acid residues that confer additional functionality to the clostridial neurotoxin, e.g. when administered to a subject. In other words, a clostridial neurotoxin that “consists essentially of” a clostridial neurotoxin L-chain or fragment thereof and/or a fragment of a clostridial neurotoxin H-chain may further comprise one or more amino acid residues (to those of the clostridial neurotoxin L-chain or fragment thereof and/or fragment of a clostridial neurotoxin H-chain) but said one or more further amino acid residues do not confer additional functionality to the clostridial neurotoxin, e.g. when administered to a subject. Additional functionality may include enzymatic activity, binding activity and/or any physiological activity whatsoever.

In one embodiment a clostridial neurotoxin may comprise non-clostridial neurotoxin sequences in addition to any clostridial neurotoxin sequences. The non-clostridial neurotoxin sequences preferably do not disrupt the ability of a clostridial neurotoxin of the invention to promote a neuroimmune response. Preferably, the non-clostridial neurotoxin sequence is not one having catalytic activity, e.g. enzymatic activity. Preferably, the non-clostridial sequence is not one that binds to a cellular receptor. In other words, it is most preferred that the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp #db_reports. Non-clostridial neurotoxin sequences may include tags to aid in purification, such as His-tags. It is preferred that any clostridial neurotoxin sequences comprised in said clostridial neurotoxin consist of a clostridial neurotoxin L-chain or fragment thereof and/or a fragment of a clostridial neurotoxin H-chain. In one embodiment, the clostridial neurotoxin sequence comprised in said clostridial neurotoxin may consist of a clostridial neurotoxin L-chain. In one embodiment, the clostridial neurotoxin sequence comprised in said clostridial neurotoxin may consist of a clostridial neurotoxin translocation domain. In one embodiment, the clostridial neurotoxin sequence comprised in said clostridial neurotoxin may consist of a clostridial neurotoxin receptor binding domain. In one embodiment, the clostridial neurotoxin sequence comprised in said clostridial neurotoxin may consist of a clostridial neurotoxin L-chain and a clostridial neurotoxin translocation domain.

Suitable clostridial neurotoxins comprising (or consisting of) a clostridial neurotoxin L-chain and translocation domain are described herein.

A clostridial neurotoxin comprising (or consisting of) a clostridial neurotoxin L-chain and translocation domain may comprise a polypeptide sequence having at least 70% sequence identity to any one of SEQ ID NOs: 4, 20, 28 or 36 or a fragment thereof. In one a clostridial neurotoxin comprising (or consisting of) a clostridial neurotoxin L-chain and translocation domain comprises a polypeptide sequence having at least 80%, 90%, 95% or 98% sequence identity to any one of SEQ ID NOs: 4, 20, 28 or 36 or a fragment thereof. Preferably, a clostridial neurotoxin comprising (or consisting of) a clostridial neurotoxin L-chain and translocation domain comprises (more preferably consists of) a polypeptide sequence comprising any one of SEQ ID NOs: 4, 20, 28 or 36 or a fragment thereof.

A clostridial neurotoxin comprising (or consisting of) a clostridial neurotoxin L-chain and translocation domain may be one encoded by a nucleotide sequence having at least 70% sequence identity to any one of SEQ ID NOs: 3, 19, 27 or 35 or a fragment thereof. In one embodiment a clostridial neurotoxin comprising (or consisting of) a clostridial neurotoxin L-chain and translocation domain is one encoded by a nucleotide sequence having at least 80%, 90%, 95% or 98% sequence identity to any one of SEQ ID NOs: 3, 19, 27 or 35 or a fragment thereof. Preferably, a clostridial neurotoxin comprising (or consisting of) a clostridial neurotoxin L-chain and translocation domain is one encoded by a nucleotide sequence comprising any one of SEQ ID NOs: 3, 19, 27 or 35 or a fragment thereof.

The clostridial neurotoxin of the present invention may be free from the complexing proteins that are present in a naturally occurring clostridial neurotoxin complex.

The clostridial neurotoxins of the present invention can be produced using recombinant nucleic acid technologies. Thus, in one embodiment, a clostridial neurotoxin (as described above) is a recombinant clostridial neurotoxin.

In one embodiment a clostridial neurotoxin of the invention comprises a clostridial neurotoxin L-chain. It is preferred that the L-chain is catalytically inactive.

Active clostridial neurotoxin L-chain has non-cytotoxic protease activity. Specifically, active clostridial neurotoxin L-chain has endopeptidase activity and is capable of cleaving a protein of the exocytic fusion apparatus in a target cell. A protein of the exocytic fusion apparatus is preferably a SNARE protein, such as SNAP-25, synaptobrevinNAMP, or syntaxin.

The term “catalytically inactive” as used herein in respect of a clostridial neurotoxin L-chain means that said L-chain exhibits substantially no non-cytotoxic protease activity, preferably the term “catalytically inactive” as used herein in respect of a clostridial neurotoxin L-chain means that said L-chain exhibits no non-cytotoxic protease activity. In one embodiment, a catalytically inactive clostridial neurotoxin L-chain is one that does not cleave a protein of the exocytic fusion apparatus in a target cell. The term “substantially no non-cytotoxic protease activity” means that the clostridial neurotoxin L-chain has less than 5% of the non-cytotoxic protease activity of a catalytically active clostridial neurotoxin L-chain (preferably an L-chain of native BoNT/A shown as SEQ ID NO: 6), for example less than 2%, 1% or preferably less than 0.1% of the non-cytotoxic protease activity of a catalytically active clostridial neurotoxin L-chain. Non-cytotoxic protease activity can be determined in vitro by incubating a test clostridial neurotoxin L-chain with a SNARE protein and comparing the amount of SNARE protein cleaved by the test clostridial neurotoxin L-chain when compared to the amount of SNARE protein cleaved by a catalytically active clostridial neurotoxin L-chain under the same conditions. Routine techniques, such as SDS-PAGE and Western blotting can be used to quantify the amount of SNARE protein cleaved. Suitable in vitro assays are described in WO 2019/145577 A1, which is incorporated herein by reference.

Cell-based and in vivo assays may also be used to determine if a clostridial neurotoxin comprising an L-chain and a functional cell binding and translocation domain has non-cytotoxic protease activity. Assays such as the Digit Abduction Score (DAS), the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Donald et al (2018), Pharmacol Res Perspect, e00446, 1-14, which is incorporated herein by reference.

A catalytically inactive L-chain may have one or more mutations that inactivate said catalytic activity. For example, a catalytically inactive BoNT/A L-chain may comprise a mutation of an active site residue, such as His223, Glu224, His227, Glu262, and/or Tyr366. The position numbering corresponds to the amino acid positions of SEQ ID NO: 62 and can be determined by aligning a polypeptide with SEQ ID NO: 62. As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. His223 will be His223 of SEQ ID NO: 62). Alternatively, where the methionine is absent from SEQ ID NO: 62 the amino acid residue numbering should be modified by −1 (e.g. His223 will be His222 of SEQ ID NO: 62). Similar considerations apply when the methionine at position 1 of the other polypeptide sequences described herein is present/absent, and the skilled person will readily determine the correct amino acid residue numbering using techniques routine in the art.

In a particularly preferred embodiment, a clostridial neurotoxin of the invention may comprise a modified BoNT/A or fragment thereof (preferably a BoNT/A H_{C} domain or fragment thereof). The modified BoNT/A or fragment thereof may be one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277. Such a modified BoNT/A or fragment thereof may demonstrate a reduction in, or absence of, side effects compared to the use of known BoNT/A. The increased tissue retention properties of the modified BoNT/A of the invention may also provide increased potency and/or duration of action and can allow for reduced dosages to be used compared to known clostridial toxin therapeutics (or increased dosages without any additional adverse effects), thus providing further advantages.

The modification may be a modification when compared to unmodified BoNT/A shown as SEQ ID NO: 62, wherein the amino acid residue numbering is determined by alignment with SEQ ID NO: 62. As the presence of a methionine residue at position 1 of SEQ ID NO: 62 (as well as the SEQ ID NOs corresponding to modified BoNT/A polypeptides or fragments thereof described herein) is optional, the skilled person will take the presence/absence of the methionine residue into account when determining amino acid residue numbering. For example, where SEQ ID NO: 62 includes a methionine, the position numbering will be as defined above (e.g. ASN 886 will be ASN 886 of SEQ ID NO: 62). Alternatively, where the methionine is absent from SEQ ID NO: 62 the amino acid residue numbering should be modified by −1 (e.g. ASN 886 will be ASN 885 of SEQ ID NO: 62). Similar considerations apply when the methionine at position 1 of the other polypeptide sequences described herein is present/absent, and the skilled person will readily determine the correct amino acid residue numbering using techniques routine in the art.

The amino acid residue(s) indicated for modification above are surface exposed amino acid residue(s).

A modified BoNT/A or fragment thereof may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277.

The term “one or more amino acid residue(s)” when used in the context of modified BoNT/A or fragment thereof preferably means at least 2, 3, 4, 5, 6 or 7 of the indicated amino acid residue(s). Thus, a modified BoNT/A may comprise at least 2, 3, 4, 5, 6 or 7 (preferably 7) modifications at the indicated amino acid residue(s). A modified BoNT/A or fragment thereof may comprise 1-30, 3-20, or 5-10 amino acid modifications. More preferably, the term “one or more amino acid residue(s)” when used in the context of modified BoNT/A or fragment thereof means all of the indicated amino acid residue(s).

Preferably, beyond the one or more amino acid modification(s) at the indicated amino acid residue(s), the modified BoNT/A or fragment thereof does not contain any further amino acid modifications when compared to SEQ ID NO: 62.

The modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A modification as indicated above results in a modified BoNT/A or fragment thereof that has an increased positive surface charge and increased isoelectric point when compared to the corresponding unmodified BoNT/A or fragment thereof.

The isoelectric point (pI) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pI). The isoelectric point is a standard concept in protein biochemistry with which the skilled person would be familiar.

The isoelectric point (pI) is therefore defined as the pH value at which a protein displays a net charge of zero. An increase in pI means that a higher pH value is required for the protein to display a net charge of zero. Thus, an increase in pI represents an increase in the net positive charge of a protein at a given pH. Conversely, a decrease in pI means that a lower pH value is required for the protein to display a net charge of zero. Thus, a decrease in pI represents a decrease in the net positive charge of a protein at a given pH.

Methods of determining the pI of a protein are known in the art and would be familiar to a skilled person. By way of example, the pI of a protein can be calculated from the average pKa values of each amino acid present in the protein (“calculated pI”). Such calculations can be performed using computer programs known in the art, such as the Compute pI/MW Tool from ExPASy (https://web.expasy.org/compute_pi/), which is the preferred method for calculating pI in accordance with the present invention. Comparisons of pI values between different molecules should be made using the same calculation technique/program.

Where appropriate, the calculated pI of a protein can be confirmed experimentally using the technique of isoelectric focusing (“observed pI”). This technique uses electrophoresis to separate proteins according to their pI. Isoelectric focusing is typically performed using a gel that has an immobilised pH gradient. When an electric field is applied, the protein migrates through the pH gradient until it reaches the pH at which it has zero net charge, this point being the pI of the protein. Results provided by isoelectric focusing are typically relatively low-resolution in nature, and thus the present inventors believe that results provided by calculated pI (as described above) are more appropriate to use.

Throughout the present specification, “pI” means “calculated pI” unless otherwise stated. The pI of a protein may be increased or decreased by altering the number of basic and/or acidic groups displayed on its surface. This can be achieved by modifying one or more amino acids of the protein. For example, an increase in pI may be provided by reducing the number of acidic residues, or by increasing the number of basic residues.

A modified BoNT/A or fragment thereof of the invention may have a pI value that is at least 0.2, 0.4, 0.5 or 1 pI units higher than that of an unmodified BoNT/A (e.g. SEQ ID NO: 62) or fragment thereof. Preferably, a modified BoNT/A or fragment thereof may have a pI of at least 6.6, e.g. at least 6.8.

The properties of the 20 standard amino acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence or fragment thereof, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A or fragment thereof. In an amino acid substitution, an amino acid residue that forms part of the BoNT/A polypeptide sequence or fragment thereof is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Preferably, a modified BoNT/A or fragment thereof for use in the invention comprises between 4 and 40 amino acid modifications located in the clostridial toxin H_{CN} domain. Said modified BoNT/A or fragment thereof preferably also has pI of at least 6.6. Said modified BoNT/A preferably comprises modifications of at least 4 amino acids selected from: ASN 886, ASN 930, ASN 954, SER 955, GLN 991, ASN 1025, ASN 1026, and ASN 1052, wherein said modification comprises substitution of the amino acids with a lysine residue or an arginine residue. For example, said modified BoNT/A or fragment thereof may comprise modifications of at least 5 amino acids selected from: ASN 886, ASN 930, ASN 954, SER 955, GLN 991, ASN 1025, ASN 1026, ASN 1052, and GLN 1229, wherein said modification comprises substitution of the amino acids with a lysine residue or an arginine residue.

Methods for modifying proteins by substitution, insertion or deletion of amino acid residues are known in the art. By way of example, amino acid modifications may be introduced by modification of a DNA sequence encoding a polypeptide (e.g. encoding unmodified BoNT/A or a fragment thereof). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

In one embodiment a clostridial neurotoxin for use according to the invention comprises a polypeptide sequence having at least 80%, 90%, 95% or 98% sequence identity to SEQ ID NO: 42. Preferably, a clostridial neurotoxin for use according to the invention comprises a polypeptide sequence shown as SEQ ID NO: 42.

In one embodiment a clostridial neurotoxin for use according to the invention comprises a polypeptide sequence that is encoded by a nucleotide sequence having at least 80%, 90%, 95% or 98% sequence identity to SEQ ID NO: 41. Preferably, a clostridial neurotoxin for use according to the invention comprises a polypeptide sequence that is encoded by a nucleotide sequence shown as SEQ ID NO: 41.

In one embodiment a clostridial neurotoxin for use according to the invention (e.g. comprising SEQ ID NO: 42 or encoded by SEQ ID NO: 41) may be a portion of a polypeptide having at least 70% sequence identity to SEQ ID NO: 61 or 65. Thus, in one embodiment a clostridial neurotoxin for use according to the invention may comprise a polypeptide sequence having at least 80%, 90%, 95% or 98% sequence identity to SEQ ID NO: 61 or 65. Preferably, a clostridial neurotoxin for use according to the invention may comprise (more preferably consist of) SEQ ID NO: 61 or 65. In one embodiment the clostridial neurotoxin comprises a catalytically-inactive L-chain (e.g. as per SEQ ID NO: 65).

In one embodiment a clostridial neurotoxin for use according to the invention (e.g. comprising SEQ ID NO: 42 or encoded by SEQ ID NO: 41) may be encoded by a nucleotide sequence having at least 70% sequence identity to SEQ ID NO: 60. Thus, in one embodiment a clostridial neurotoxin for use according to the invention may be encoded by a nucleotide sequence having at least 80%, 90%, 95% or 98% sequence identity to SEQ ID NO: 60. Preferably, a clostridial neurotoxin for use according to the invention may be encoded by a nucleotide sequence comprising (more preferably consisting of) SEQ ID NO: 60. In one embodiment the clostridial neurotoxin comprises a catalytically-inactive L-chain.

SEQ ID NO: 42 is an example of a modified BoNT/A fragment and SEQ ID NOs: 61 and 65 are examples of modified BoNT/A polypeptides that are catalytically active and inactive, respectively. Such modified BoNT/A polypeptides and fragments are particularly preferred for use in the present invention. The polypeptides shown as SEQ ID NO: 42, 61 and 65 have a number of amino acid modifications (e.g. substitutions) when compared to wild-type BoNT/A, which increase the isoelectric point of the polypeptide. Without wishing to be bound by theory, it is believed that the increased net positive charge promotes electrostatic interactions between the polypeptide and anionic extracellular components, thereby promoting binding between the polypeptide and cell surface thus increasing retention at a site of administration and/or duration of action. Thus, it is envisaged that neuroimmune response-promotion properties of SEQ ID NO: 42, 61 and 65 will be improved compared to equivalent polypeptides lacking said modifications.

For the catalytically active modified BoNT/A polypeptides described above (e.g. SEQ ID NO: 61), one way in which these advantageous properties (which represent an increase in the therapeutic index) may be defined is in terms of the Safety Ratio of the modified BoNT/A. In this regard, undesired effects of a clostridial neurotoxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial neurotoxin can be assessed experimentally by Digital Abduction Score (DAS) assay, a measurement of muscle paralysis. The DAS assay may be performed by injection of 20 μl of clostridial neurotoxin, formulated in Gelatin Phosphate Buffer, into the mouse gastrocnemius/soleus complex, followed by assessment of Digital Abduction Score using the method of Aoki (Aoki K R, Toxicon 39: 1815-1820; 2001). In the DAS assay, mice are suspended briefly by the tail in order to elicit a characteristic startle response in which the mouse extends its hind limbs and abducts its hind digits. Following clostridial neurotoxin injection, the varying degrees of digit abduction are scored on a five-point scale (0=normal to 4=maximal reduction in digit abduction and leg extension).

The Safety Ratio of a clostridial neurotoxin may then be expressed as the ratio between the amount of toxin required for a 10% drop in a bodyweight (measured at peak effect within the first seven days after dosing in a mouse) and the amount of toxin required for a DAS score of 2. High Safety Ratio scores are therefore desired and indicate a toxin that is able to effectively paralyse a target muscle with little undesired off-target effects. A catalytically active modified BoNT/A of the present invention may have a Safety Ratio that is higher than the Safety Ratio of an equivalent unmodified (native) botulinum toxin (e.g. SEQ ID NO: 62).

Thus, in one embodiment, a catalytically active modified BoNT/A of the present invention has a Safety Ratio of at least 8 (for example, at least 8, 9, 10, 15, 20, 25, 30, 35, 40, 45 or 50), wherein Safety Ratio is calculated as: dose of toxin required for −10% bodyweight change (pg/mouse) divided by DAS ED₅₀ (pg/mouse) [ED₅₀=dose required to produce a DAS score of 2].

In one embodiment, a catalytically active modified BoNT/A of the present invention has a Safety Ratio of at least 10. In one embodiment, a modified BoNT/A or fragment thereof of the present invention has a Safety Ratio of at least 15.

Clostridial neurotoxins comprising at least 70% sequence identity to SEQ ID NO: 61 are described in WO 2015/004461 A1, which is incorporated herein by reference in its entirety.

In one embodiment a clostridial neurotoxin comprising a polypeptide sequence having at least 70% sequence identity to SEQ ID NO: 42, 61 or 65 and/or comprising a polypeptide sequence that is encoded by a nucleotide sequence having at least 70% sequence identity to SEQ ID NO: 41 or 60 comprises a substitution at one or more (preferably two or more, three or more, four or more, five or more or six or more, more preferably at all) of positions 930, 955, 991, 1026, 1052, 1229, and 886. The position numbering corresponds to the positions of SEQ ID NO: 62 and can be determined by aligning the polypeptide sequence with SEQ ID NO: 62 (unmodified/wild-type BoNT/A). As the presence of a methionine residue at position 1 of SEQ ID NO: 62 is optional, the skilled person will take the presence/absence of the methionine residue into account when determining amino acid residue numbering. For example, where SEQ ID NO: 62 includes a methionine, the position numbering will be as defined above (e.g. position 886 will be ASN 886 of SEQ ID NO: 62). Alternatively, where the methionine is absent from SEQ ID NO: 62 the amino acid residue numbering should be modified by −1 (e.g. position 886 will be ASN 885 of SEQ ID NO: 62). Similar considerations apply when the methionine at position 1 of the other polypeptide sequences described herein is present/absent, and the skilled person will readily determine the correct amino acid residue numbering using techniques routine in the art.

Preferably, the clostridial neurotoxin comprising a polypeptide sequence having at least 70% sequence identity to SEQ ID NO: 42, 61 or 65 and/or comprising a polypeptide sequence that is encoded by a nucleotide sequence having at least 70% sequence identity to SEQ ID NO: 41 or 60 comprises lysine or arginine (more preferably lysine) at one or more of positions 930, 955, 991, 1026, 1052, 1229, and 886. In one embodiment, the clostridial neurotoxin comprises lysine or arginine (more preferably lysine) at least two, three, four, five, six or all of positions 930, 955, 991, 1026, 1052, 1229, and 886. Most preferably, the clostridial neurotoxin comprises lysine or arginine (more preferably lysine) at all of positions 930, 955, 991, 1026, 1052, 1229, and 886.

Embodiments related to the various therapeutic uses of the invention are intended to be applied equally to methods of treatment, polypeptides of the invention, and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}\begin{matrix}[ length of the longer sequence plus the \\ number of gaps introduced into the longer\end{matrix} \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such neurotoxin and reference to “the clostridial neurotoxin” includes reference to one or more clostridial neurotoxin(s) and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1. A, an illustration of the dermatomes (adapted Beilin, Daniel. “The Physiology of Dynamic Thermometric Analysis of the Skin in Visceral Health & Disease.” (2014)—see FIG. 3 thereof)—examples of certain dermatomes that map to different organs is shown here, many other mappings are known e.g. as per B; and (B) the viscera with which they are associated (adapted from Wall and Melzack's “Textbook of Pain” (edited by McMahon et al.; 6th edition)—see chapter 53, and Figure 53-1 thereof). C, an illustration the relationships between the dermatome and enterotome (and myotome); the invention utilises retrograde transport from the dermatome to target an area of visceral pain integration via administration of clostridial neurotoxin to a dermatome (adapted Beilin, Daniel. “The Physiology of Dynamic Thermometric Analysis of the Skin in Visceral Health & Disease.” (2014)—see Figure 1 thereof).

FIG. 2. Protocol design.

FIG. 3. Caudal ventral view of female rat, illustrating the area to which clostridial neurotoxin was injected (circle).

FIG. 4. Repeated CYP injections (40 mg/kg, i.p.) between DO and D6 induced visceral pain, which was assessed at D7 and D10. Nociceptive threshold in g (A), nociceptive scores in % (B) and AUC calculated between 1 and 6 g (C) or 6 and 26 g (D), before (D0, “Basal”) and post-CYP injection (D7 and D10) within the Vehicle group. Results are expressed as mean±SEM. Friedman test, Two-way RM ANOVA by both factors or One-way RM ANOVA with Dunn's, Dunnett's or Holm-Sidak's post-test vs Basal, ^{##}P<0.01 and ^{####}P<0.0001. Vehicle=saline, 20 μL, i.d., n=10, once at D-10.

FIG. 5. Dysport (25 U/kg, i.d.) diminished CYP-induced chronic allodynia. Nociceptive threshold in g, before (D0, “Basal”) and post-CYP injection (D7 and D10) in Dysport and Vehicle groups. Results are expressed as mean±SEM. ns P>0.05 and * P<0.05, Mann Whitney test or Unpaired t test. Vehicle=saline, 20 μL, i.d., n=10, once at D-10. Dysport=25 U/kg, i.d., n=10, once at D-10.

FIG. 6. Dysport (25 U/kg, i.d.) reduced CYP-induced chronic visceral pain (nociceptive scores). Nociceptive scores in %, before (DO, “Basal”) (A) and post-CYP injection; D7 (B) and D10 (C) in Dysport and Vehicle groups. Results are expressed as mean±SEM. ns P>0.05 and * P<0.05, Two-way RM ANOVA. Vehicle=saline, 20 μL, i.d., n=10, once at D-10. Dysport=25 U/kg, i.d., n=10, once at D-10.

FIG. 7. Dysport (25 U/kg, i.d.) decreased CYP-induced allodynia (AUC 1-6 g) and hyperalgesia (AUC 6-26 g). AUC calculated between 1 and 6 g (A) and 6 and 26 g (B), before (D0, “Basal”), and post-CYP injection (D7 and D10) in Dysport and Vehicle groups. Results are expressed as mean±SEM. ns P>0.05 and * P<0.05, Mann Whitney test or Unpaired t test. Vehicle=saline, 20 μL, i.d., n=10, once at D-10. Dysport=25 U/kg, i.d., n=10, once at D-10.

FIG. 8. c-SNAP25 IHC results in Thoracic spinal cord. IHC scoring from 0-20 (5 most intensely stained sections scored form 0-4 each). No c-SNAP25 staining was observed in saline-treated animals.

FIG. 9. c-SNAP25 IHC results in Lumbosacral spinal cord. IHC scoring from 0-20 (5 most intensely stained sections scored form 0-4 each). No c-SNAP25 staining was observed in saline-treated animals.

FIG. 10. Quantification of cleaved (a) SNAP-25 and (b) Glial fibrillary acidic protein (GFAP) immunostaining in the entire lumbar spinal cord (L1 to L6). In L3-L4 and L5-L6 segments of the lumbar spinal cord, a marked decrease in GFAP staining was noted which was associated with higher level of C-SNAP25 in the dorsal horns.

### SEQUENCE LISTING

Where an initial Met amino acid residue or a corresponding initial codon is indicated in any of the following SEQ ID NOs, said residue/codon is optional.
SEQ ID NO: 1—Nucleotide Sequence of Recombinant Catalytically Inactive BoNT/A (rBoNT/A(0))
SEQ ID NO: 2—Polypeptide Sequence of rBoNT/A(0)
SEQ ID NO: 3—Nucleotide Sequence of rLH_{N}/A (light-chain plus translocation domain only).

SEQ ID NO: 4—Polypeptide Sequence of rLH_{N}/A
SEQ ID NO: 5—Nucleotide Sequence of rL/A (light-chain only)
SEQ ID NO: 6—Polypeptide Sequence of rL/A
SEQ ID NO: 7—Nucleotide Sequence of rH_{C}/A
SEQ ID NO: 8—Polypeptide Sequence of rH_{C}/A
SEQ ID NO: 9—Nucleotide Sequence of rBoNT/B(0)
SEQ ID NO: 10—Polypeptide Sequence of rBoNT/B(0)
SEQ ID NO: 11—Nucleotide Sequence of rBoNT/C(0)
SEQ ID NO: 12—Polypeptide Sequence of rBoNT/C(0)
SEQ ID NO: 13—Nucleotide Sequence of rBoNT/E(0)
SEQ ID NO: 14—Polypeptide Sequence of rBoNT/E(0)
SEQ ID NO: 15—Nucleotide Sequence of rBoNT/F(0)
SEQ ID NO: 16—Polypeptide Sequence of rBoNT/F(0)
SEQ ID NO: 17—Nucleotide Sequence of rBoNT/A(0) (His-tagged)
SEQ ID NO: 18—Polypeptide Sequence of rBoNT/A(0) (His-tagged)
SEQ ID NO: 19—Nucleotide Sequence of rLH_{N}/A (His-tagged)
SEQ ID NO: 20—Polypeptide Sequence of rLH_{N}/A (His-tagged)
SEQ ID NO: 21—Nucleotide Sequence of rH_{C}/A (His-tagged)
SEQ ID NO: 22—Polypeptide Sequence of rH_{C}/A (His-tagged)
SEQ ID NO: 23—Nucleotide Sequence of rLC/A (His-tagged)
SEQ ID NO: 24—Polypeptide Sequence of rLC/A (His-tagged)
SEQ ID NO: 25—Nucleotide Sequence of rBoNT/FA(0) (His-tagged)
SEQ ID NO: 26—Polypeptide Sequence of rBoNT/FA(0) (His-tagged)
SEQ ID NO: 27—Nucleotide Sequence of rLH_{N}/FA (His-tagged)
SEQ ID NO: 28—Polypeptide Sequence of rLH_{N}/FA (His-tagged)
SEQ ID NO: 29—Nucleotide Sequence of rH_{C}/FA (His-tagged)
SEQ ID NO: 30—Polypeptide Sequence of rH_{C}/FA (His-tagged)
SEQ ID NO: 31—Nucleotide Sequence of rLC/FA (His-tagged)
SEQ ID NO: 32—Polypeptide Sequence of rLC/FA (His-tagged)
SEQ ID NO: 33—Nucleotide Sequence of rBoNT/F(0) (His-tagged)
SEQ ID NO: 34—Polypeptide Sequence of rBoNT/F(0) (His-tagged)
SEQ ID NO: 35—Nucleotide Sequence of rL_{H}N/F (His-tagged)
SEQ ID NO: 36—Polypeptide Sequence of rL_{H}N/F (His-tagged)
SEQ ID NO: 37—Nucleotide Sequence of rH_{C}/F (His-tagged)
SEQ ID NO: 38—Polypeptide Sequence of rH_{C}/F (His-tagged)
SEQ ID NO: 39—Nucleotide Sequence of rLC/F (His-tagged)
SEQ ID NO: 40—Polypeptide Sequence of rLC/F (His-tagged)
SEQ ID NO: 41—Nucleotide Sequence of Cationic rH_{C}/A (His-tagged)
SEQ ID NO: 42—Polypeptide Sequence of Cationic rH_{C}/A (His-tagged)
SEQ ID NO: 43—Nucleotide Sequence of rH_{C}/AB (His-tagged)
SEQ ID NO: 44—Polypeptide Sequence of rH_{C}/AB (His-tagged)
SEQ ID NO: 45—Nucleotide Sequence of rH_{C}/A Variant Y1117V H1253K (His-tagged)
SEQ ID NO: 46—Polypeptide Sequence of rH_{C}/A Variant Y1117V H1253K (His-tagged)
SEQ ID NO: 47—Nucleotide Sequence of rH_{C}/A Variant Y1117V F1252Y H1253K L1278F (His-tagged)
SEQ ID NO: 48—Polypeptide Sequence of rH_{C}/A Variant Y1117V F1252Y H1253K L1278F (His-tagged)
SEQ ID NO: 49—Nucleotide Sequence of rH_{C}/A Variant Y1117V F1252Y H1253K L1278H (His-tagged)
SEQ ID NO: 50—Polypeptide Sequence of rH_{C}/A Variant Y1117V F1252Y H1253K L1278H (His-tagged)
SEQ ID NO: 51—Polypeptide Sequence of BoNT/A—UniProt P10845
SEQ ID NO: 52—Polypeptide Sequence of BoNT/B—UniProt P10844
SEQ ID NO: 53—Polypeptide Sequence of BoNT/C—UniProt P18640
SEQ ID NO: 54—Polypeptide Sequence of BoNT/D—UniProt P19321
SEQ ID NO: 55—Polypeptide Sequence of BoNT/E—UniProt Q00496
SEQ ID NO: 56—Polypeptide Sequence of BoNT/F—UniProt A7GBG3
SEQ ID NO: 57—Polypeptide Sequence of BoNT/G—UniProt Q60393
SEQ ID NO: 58—Polypeptide Sequence of TeNT—UniProt P04958
SEQ ID NO: 59—Polypeptide Sequence of BoNT/X
SEQ ID NO: 60—Nucleotide Sequence of mrBoNT/A
SEQ ID NO: 61—Polypeptide Sequence of mrBoNT/A
SEQ ID NO: 62—Polypeptide Sequence of Unmodified BoNT/A1
SEQ ID NO: 63—Polypeptide Sequence of mrBoNT/AB
SEQ ID NO: 64—Polypeptide Sequence of mrBoNT/AB(0)
SEQ ID NO: 65—Polypeptide Sequence of mrBoNT/A(0)

SEQ ID NO: 1-Nucleotide Sequence of rBoNT/A(0)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATCAACTG
ATCtACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCAAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAACGCCATTGTCTACAACTCCATGTACGAGAATTTCTCCACCAGCTTCTGGATTCGCATCCCG
AAATACTTCAACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AACAGCAAGATTTACATCAATGGTCGCTTGATCGATCAGAAACCGATTAGCAACCTGGGTAATATCCACGCAAGC
AACAACATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACCAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 2-Polypeptide Sequence of rBoNT/A(0)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNEDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYENSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 3-Nucleotide Sequence of rLH_{N}/A
atggagttcgttaacaaacagttcaactataaagacccagttaacggtgttgacattgcttacatcaaaatcccg
aacgctggccagatgcagccggtaaaggcattcaaaatccacaacaaaatctgggttatcccggaacgtgatacc
tttactaacccggaagaaggtgacctgaacccgccaccggaagcgaaacaggtgccggtatcttactatgactcc
acctacctgtctaccgataacgaaaaggacaactacctgaaaggtgttactaaactgttcgagcgtatttactcc
accgacctgggccgtatgctgctgactagcatcgttcgcggtatcccgttctggggcggttctaccatcgatacc
gaactgaaagtaatcgacactaactgcatcaacgttattcagccggacggttcctatcgttccgaagaactgaac
ctggtgatcatcggcccgtctgctgatatcatccagttcgagtgtaagagctttggtcacgaagttctgaacctc
acccgtaacggctacggttccactcagtacatccgtttctctccggacttcaccttcggttttgaagaatccctg
gaagtagacacgaacccactgctgggcgctggtaaattcgcaactgatcctgcggttaccctggctcacgaactg
attcatgcaggccaccgcctgtacggtatcgccatcaatccgaaccgtgtcttcaaagttaacaccaacgcgtat
tacgagatgtccggtctggaagttagcttcgaagaactgcgtacttttggcggtcacgacgctaaattcatcgac
tctctgcaagaaaacgagttccgtctgtactactataacaagttcaaagatatcgcatccaccctgaacaaagcg
aaatccatcgtgggtaccactgcttctctccagtacatgaagaacgtttttaaagaaaaatacctgctcagcgaa
gacacctccggcaaattctctgtagacaagttgaaattcgataaactttacaaaatgctgactgaaatttacacc
gaagacaacttcgttaagttctttaaagttctgaaccgcaaaacctatctgaacttcgacaaggcagtattcaaa
atcaacatcgtgccgaaagttaactacactatctacgatggtttcaacctgcgtaacaccaacctggctgctaat
tttaacggccagaacacggaaatcaacaacatgaacttcacaaaactgaaaaacttcactggtctgttcgagttt
tacaagctgctgtgcGTCGACGGCATCATTACCTCCAAAACTAAATCTGACGATGACGATAAAAACAAAGCGCTG
AACCTGCAGtgtatcaaggttaacaactgggatttattcttcagcccgagtgaagacaacttcaccaacgacctg
aacaaaggtgaagaaatcacctcagatactaacatcgaagcagccgaagaaaacatctcgctggacctgatccag
cagtactacctgacctttaatttcgacaacgagccggaaaacatttctatcgaaaacctgagctctgatatcatc
ggccagctggaactgatgccgaacatcgaacgtttcccaaacggtaaaaagtacgagctggacaaatataccatg
ttccactacctgcgcgcgcaggaatttgaacacggcaaatcccgtatcgcactgactaactccgttaacgaagct
ctgctcaacccgtcccgtgtatacaccttcttctctagcgactacgtgaaaaaggtcaacaaagcgactgaagct
gcaatgttcttgggttgggttgaacagcttgtttatgattttaccgacgagacgtccgaagtatctactaccgac
aaaattgcggatatcactatcatcatcccgtacatcggtccggctctgaacattggcaacatgctgtacaaagac
gacttcgttggcgcactgatcttctccggtgcggtgatcctgctggagttcatcccggaaatcgccatcccggta
ctgggcacctttgctctggtttcttacattgcaaacaaggttctgactgtacaaaccatcgacaacgcgctgagc
aaacgtaacgaaaaatgggatgaagtttacaaatatatcgtgaccaactggctggctaaggttaatactcagatc
gacctcatccgcaaaaaaatgaaagaagcactggaaaaccaggcggaagctaccaaggcaatcattaactaccag
tacaaccagtacaccgaggaagaaaaaaacaacatcaacttcaacatcgacgatctgtcctctaaactgaacgaa
tccatcaacaaagctatgatcaacatcaacaagttcctgaaccagtgctctgtaagctatctgatgaactccatg
atcccgtacggtgttaaacgtctggaggacttcgatgcgtctctgaaagacgccctgctgaaatacatttacgac
aaccgtggcactctgatcggtcaggttgatcgtctgaaggacaaagtgaacaataccttatcgaccgacatccct
tttcagctcagtaaatatgtcgataaccaacgccttttgtccactctagaagcaCACCATCATCACcaccatcac
catcaccat
SEQ ID NO: 4-Polypeptide Sequence of rLH_{N}/A
MEFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVDGIITSKTKSDDDDKNKAL
NLQCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTLEAHHHHHHHHHH
SEQ ID NO: 5-Nucleotide Sequence of rL/A
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGggtctagaagcaCACCATCATCACcaccatcaccatcaccat
SEQ ID NO: 6-Polypeptide Sequence of rL/A
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLGLEAHHHHHHHHHH
SEQ ID NO: 7-Nucleotide Sequence of rH_{C}/A
ATGCATCATCACCATCACCACAAAAACATCATCAATACTAGCATTCTGAACCTGCGTTACGAGAGCAATCATCTG
ATTGATCTGAGCCGTTATGCAAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAACGCCATTGTCTACAACTCCATGTAC
GAGAATTTCTCCACCAGCTTCTGGATTCGCATCCCGAAATACTTCAACAGCATTAGCCTGAACAACGAGTATACT
ATCATCAACTGTATGGAGAACAACAGCGGTTGGAAGGTGTCTCTGAACTATGGTGAGATCATTTGGACCTTGCAG
GACACCCAAGAGATCAAGCAGCGCGTCGTGTTCAAGTACTCTCAAATGATCAACATTTCCGATTACATTAATCGT
TGGATCTTCGTGACCATTACGAATAACCGTCTGAATAACAGCAAGATTTACATCAATGGTCGCTTGATCGATCAG
AAACCGATTAGCAACCTGGGTAATATCCACGCAAGCAACAACATTATGTTCAAATTGGACGGTTGCCGCGATACC
CATCGTTATATCTGGATCAAGTATTTCAACCTGTTTGATAAAGAACTGAATGAGAAGGAGATCAAAGATTTGTAT
GACAACCAATCTAACAGCGGCATTTTGAAGGACTTCTGGGGCGATTATCTGCAATACGATAAGCCGTACTATATG
CTGAACCTGTATGATCCGAACAAATATGTGGATGTCAATAATGTGGGTATTCGTGGTTACATGTATTTGAAGGGT
CCGCGTGGCAGCGTTATGACGACCAACATTTACCTGAACTCTAGCCTGTACCGTGGTACGAAATTCATCATTAAG
AAATATGCCAGCGGCAACAAAGATAACATTGTGCGTAATAACGATCGTGTCTACATCAACGTGGTCGTGAAGAAT
AAAGAGTACCGTCTGGCGACCAACGCTTCGCAGGCGGGTGTTGAGAAAATTCTGAGCGCGTTGGAGATCCCTGAT
GTCGGTAATCTGAGCCAAGTCGTGGTTATGAAGAGCAAGAACGACCAGGGTATCACTAACAAGTGCAAGATGAAC
CTGCAAGACAACAATGGTAACGACATCGGCTTTATTGGTTTCCACCAGTTCAACAATATTGCTAAACTGGTAGCG
AGCAATTGGTACAATCGTCAGATTGAGCGCAGCAGCCGTACTTTGGGCTGTAGCTGGGAGTTTATCCCGGTCGAT
GATGGTTGGGGCGAACGTCCGCTG
SEQ ID NO: 8-Polypeptide Sequence of rH_{C}/A
MHHHHHHKNIINTSILNLRYESNHLIDLSRYASKINIGSKVNFDPIDKNQIQLFNLESSKIEVILKNAIVYNSMY
ENFSTSFWIRIPKYENSISLNNEYTIINCMENNSGWKVSLNYGEIIWTLQDTQEIKQRVVFKYSQMINISDYINR
WIFVTITNNRLNNSKIYINGRLIDQKPISNLGNIHASNNIMFKLDGCRDTHRYIWIKYFNLEDKELNEKEIKDLY
DNQSNSGILKDFWGDYLQYDKPYYMLNLYDPNKYVDVNNVGIRGYMYLKGPRGSVMTTNIYLNSSLYRGTKFIIK
KYASGNKDNIVRNNDRVYINVVVKNKEYRLATNASQAGVEKILSALEIPDVGNLSQVVVMKSKNDQGITNKCKMN
LQDNNGNDIGFIGFHQFNNIAKLVASNWYNRQIERSSRTLGCSWEFIPVDDGWGERPL
SEQ ID NO: 9-Nucleotide Sequence of rBoNT/B(0)
ATGCCGGTGACGATTAACAACTTCAACTACAACGACCCGATTGACAACAACAACATTATCATGATGGAACCGCCG
TTTGCACGCGGCACGGGCCGTTATTACAAAGCGTTTAAAATCACCGATCGTATTTGGATTATCCCGGAACGCTAC
ACGTTTGGTTATAAACCGGAAGACTTCAACAAAAGCTCTGGCATCTTCAACCGTGATGTTTGCGAATACTACGAT
CCGGACTACCTGAACACCAACGATAAGAAAAACATTTTTCTGCAAACGATGATCAAACTGTTCAATCGCATTAAA
AGCAAACCGCTGGGTGAAAAACTGCTGGAAATGATTATCAATGGCATTCCGTATCTGGGTGATCGTCGCGTGCCG
CTGGAAGAATTTAACACCAATATCGCGAGTGTTACGGTCAACAAACTGATTTCCAATCCGGGTGAAGTCGAACGT
AAAAAAGGCATCTTCGCCAACCTGATCATCTTCGGCCCGGGTCCGGTGCTGAACGAAAATGAAACCATTGATATC
GGTATTCAGAACCATTTTGCCTCACGCGAAGGCTTCGGCGGTATTATGCAAATGAAATTTTGCCCGGAATATGTG
TCGGTTTTCAACAATGTTCAGGAAAACAAAGGTGCAAGCATCTTTAATCGTCGCGGCTATTTCTCTGATCCGGCT
CTGATCCTGATGCACCAACTGATTtATGTGCTGCACGGCCTGTATGGTATCAAAGTGGATGACCTGCCGATCGTT
CCGAACGAGAAAAAATTTTTCATGCAGAGCACCGACGCAATTCAAGCTGAAGAACTGTATACGTTTGGCGGTCAG
GACCCGTCTATTATCACCCCGAGCACCGACAAAAGCATCTACGATAAAGTGCTGCAAAACTTTCGTGGCATTGTT
GACCGCCTGAATAAAGTCCTGGTGTGTATCTCTGATCCGAACATCAACATCAACATCTACAAAAACAAATTCAAA
GACAAATACAAATTCGTTGAAGATTCTGAAGGCAAATATAGTATTGACGTCGAATCCTTTGATAAACTGTACAAA
AGTCTGATGTTCGGTTTCACCGAAACGAACATCGCGGAAAACTACAAAATCAAAACCCGCGCCTCCTATTTCAGC
GACTCTCTGCCGCCGGTTAAAATCAAAAATCTGCTGGATAACGAAATTTATACGATCGAAGAAGGTTTCAACATC
AGCGATAAAGACATGGAAAAAGAATACCGTGGCCAGAATAAAGCAATCAACAAACAGGCGTATGAAGAAATTAGT
AAAGAACATCTGGCGGTCTACAAAATTCAGATGTGCAAATCCGTGAAAGCCCCGGGTATTTGTATCGATGTTGAC
AATGAAGACCTGTTTTTCATCGCCGATAAAAACAGTTTTTCCGATGACCTGTCAAAAAATGAACGCATCGAATAC
AACACCCAATCGAACTACATCGAAAACGATTTCCCGATCAACGAACTGATTCTGGATACGGACCTGATTAGTAAA
ATCGAACTGCCGTCAGAAAACACCGAATCGCTGACGGACTTTAATGTTGATGTCCCGGTGTATGAAAAACAGCCG
GCAATTAAGAAAATTTTTACCGATGAAAACACGATCTTCCAGTACCTGTACAGCCAAACCTTTCCGCTGGACATT
CGCGATATCTCTCTGACGAGTTCCTTTGATGACGCACTGCTGTTCAGCAACAAAGTGTACTCCTTTTTCTCAATG
GATTACATCAAAACCGCTAACAAAGTGGTTGAAGCGGGCCTGTTTGCCGGTTGGGTGAAACAGATCGTTAACGAT
TTCGTCATCGAAGCCAACAAAAGTAACACGATGGATAAAATTGCTGATATCTCCCTGATTGTCCCGTATATTGGC
CTGGCACTGAATGTGGGTAACGAAACGGCGAAAGGCAATTTTGAAAACGCCTTCGAAATTGCAGGCGCTTCAATC
CTGCTGGAATTTATTCCGGAACTGCTGATCCCGGTCGTGGGTGCGTTCCTGCTGGAATCTTACATCGACAACAAA
AACAAAATCATCAAAACCATTGATAACGCGCTGACGAAACGTAACGAAAAATGGTCAGATATGTACGGCCTGATT
GTTGCCCAGTGGCTGAGCACCGTCAACACGCAATTTTACACCATCAAAGAAGGTATGTACAAAGCGCTGAATTAT
CAGGCGCAAGCCCTGGAAGAAATCATCAAATACCGCTACAACATCTACAGCGAAAAAGAAAAATCTAACATCAAC
ATCGACTTTAATGATATCAACAGCAAACTGAACGAAGGTATCAACCAGGCAATCGATAACATCAACAACTTCATC
AACGGCTGCTCAGTGTCGTATCTGATGAAGAAAATGATCCCGCTGGCTGTTGAAAAACTGCTGGATTTTGACAAC
ACCCTGAAGAAAAACCTGCTGAACTACATCGATGAAAACAAACTGTACCTGATCGGCTCAGCCGAATACGAAAAA
TCGAAAGTGAACAAATACCTGAAAACCATCATGCCGTTTGACCTGAGTATTTACACCAACGATACGATCCTGATC
GAAATGTTCAACAAATACAACTCCGAAATTCTGAACAATATTATCCTGAACCTGCGTTACAAAGACAACAATCTG
ATCGATCTGAGCGGCTATGGTGCAAAAGTTGAAGTCTACGACGGTGTCGAACTGAACGATAAAAACCAGTTCAAA
CTGACCTCATCGGCTAACTCAAAAATTCGTGTGACGCAGAACCAAAACATCATCTTCAACTCGGTCTTTCTGGAC
TTCAGCGTGTCTTTCTGGATTCGCATCCCGAAATATAAAAATGATGGCATCCAGAACTACATCCATAACGAATAC
ACCATCATCAACTGTATGAAAAACAACAGTGGTTGGAAAATTTCCATCCGTGGCAACCGCATTATCTGGACCCTG
ATTGATATCAATGGTAAAACGAAAAGCGTGTTTTTCGAATACAACATCCGTGAAGATATCTCTGAATACATCAAT
CGCTGGTTTTTCGTGACCATTACGAACAATCTGAACAATGCGAAAATCTATATCAACGGCAAACTGGAAAGTAAT
ACCGACATCAAAGATATTCGTGAAGTTATCGCCAACGGTGAAATCATCTTCAAACTGGATGGCGACATCGATCGC
ACCCAGTTCATTTGGATGAAATACTTCTCCATCTTCAACACGGAACTGAGTCAGTCCAATATCGAAGAACGCTAC
AAAATCCAATCATACTCGGAATACCTGAAAGATTTCTGGGGTAACCCGCTGATGTACAACAAAGAATACTACATG
TTCAACGCGGGCAACAAAAACTCATACATCAAACTGAAAAAAGATTCGCCGGTGGGTGAAATCCTGACCCGTAGC
AAATACAACCAGAACTCTAAATACATCAACTATCGCGATCTGTACATTGGCGAAAAATTTATTATCCGTCGCAAA
AGCAACTCTCAGAGTATTAATGATGACATCGTGCGTAAAGAAGACTACATCTATCTGGATTTCTTTAATCTGAAC
CAAGAATGGCGCGTTTATACCTACAAATACTTCAAAAAAGAAGAAGAGAAACTGTTCCTGGCCCCGATTAGCGAC
AGCGATGAATTTTACAACACCATCCAGATCAAAGAATACGATGAACAGCCGACGTATAGTTGCCAACTGCTGTTC
AAAAAAGACGAAGAATCCACCGATGAAATTGGCCTGATTGGTATCCACCGTTTCTATGAAAGCGGTATCGTTTTC
GAAGAATACAAAGATTACTTCTGTATCTCTAAATGGTATCTGAAAGAAGTCAAACGCAAACCGTACAACCTGAAA
CTGGGCTGCAACTGGCAATTTATCCCGAAAGACGAAGGCTGGACCGAA
SEQ ID NO: 10-Polypeptide Sequence of rBoNT/B(0)
MPVTINNENYNDPIDNNNIIMMEPPFARGTGRYYKAFKITDRIWIIPERYTFGYKPEDENKSSGIFNRDVCEYYD
PDYLNTNDKKNIFLQTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVENNVQENKGASIFNRRGYFSDPA
LILMHQLIYVLHGLYGIKVDDLPIVPNEKKFFMQSTDAIQAEELYTFGGQDPSIITPSTDKSIYDKVLQNERGIV
DRLNKVLVCISDPNININIYKNKFKDKYKFVEDSEGKYSIDVESFDKLYKSLMFGFTETNIAENYKIKTRASYFS
DSLPPVKIKNLLDNEIYTIEEGFNISDKDMEKEYRGQNKAINKQAYEEISKEHLAVYKIQMCKSVKAPGICIDVD
NEDLFFIADKNSFSDDLSKNERIEYNTQSNYIENDFPINELILDTDLISKIELPSENTESLTDFNVDVPVYEKQP
AIKKIFTDENTIFQYLYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLIPVVGAFLLESYIDNK
NKIIKTIDNALTKRNEKWSDMYGLIVAQWLSTVNTQFYTIKEGMYKALNYQAQALEEIIKYRYNIYSEKEKSNIN
IDENDINSKLNEGINQAIDNINNFINGCSVSYLMKKMIPLAVEKLLDFDNTLKKNLLNYIDENKLYLIGSAEYFK
SKVNKYLKTIMPFDLSIYINDTILIEMFNKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNSGWKISIRGNRIIWTL
IDINGKTKSVFFEYNIREDISEYINRWFFVTITNNLNNAKIYINGKLESNTDIKDIREVIANGEIIFKLDGDIDR
TQFIWMKYFSIFNTELSQSNIEERYKIQSYSEYLKDFWGNPLMYNKEYYMFNAGNKNSYIKLKKDSPVGEILTRS
KYNQNSKYINYRDLYIGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCISKWYLKEVKRKPYNLK
LGCNWQFIPKDEGWTE
SEQ ID NO: 11-Nucleotide Sequence of rBoNT/C(0)
ATGCCGATCACGATTAATAATTTCAACTATAGCGATCCGGTGGACAATAAGAATATTCTGTATCTGGATACTCAT
CTGAATACGCTGGCTAACGAACCGGAGAAAGCGTTCCGCATCACAGGCAACATCTGGGTTATTCCCGATCGCTTT
TCACGCAACAGCAACCCTAATCTGAACAAACCTCCTCGTGTCACCAGTCCTAAATCCGGTTATTACGACCCAAAC
TATCTGAGTACGGATAGCGATAAAGATCCCTTTCTGAAAGAGATCATTAAGCTGTTCAAACGCATTAACTCTCGC
GAAATTGGGGAAGAGCTGATCTATCGGCTTTCGACAGATATCCCGTTCCCAGGTAACAATAATACCCCGATTAAT
ACTTTCGACTTTGATGTTGATTTCAATTCTGTGGATGTGAAAACGCGTCAAGGCAATAATTGGGTGAAAACTGGT
AGCATTAACCCGAGTGTAATTATCACAGGTCCCCGTGAGAACATCATCGACCCGGAAACCTCTACCTTCAAGCTG
ACGAACAACACGTTTGCTGCACAGGAAGGGTTTGGTGCCCTGTCAATCATTTCCATCTCACCGCGTTTCATGTTA
ACCTACTCCAATGCCACAAATGATGTTGGCGAAGGACGTTTTAGCAAATCAGAATTTTGCATGGACCCAATTCTC
ATTCTGATGggCacGCTGAACaATGCGATGCACAACTTGTATGGCATTGCTATTCCAAACGATCAAACCATTAGC
TCCGTTACCAGTAATATCTTCTATAGCCAGTATAATGTCAAATTGGAGTATGCCGAAATTTACGCCTTTGGAGGC
CCGACCATTGACCTGATTCCGAAATCTGCACGCAAATACTTCGAAGAAAAGGCGTTAGATTACTATCGCAGCATC
GCGAAACGCCTGAACTCGATTACCACGGCCAATCCGTCGTCGTTCAACAAATACATTGGTGAATATAAACAGAAA
CTGATTCGCAAATATCGGTTTGTCGTAGAAAGCTCTGGTGAAGTGACTGTAAACCGCAACAAATTTGTCGAACTC
TACAACGAGTTGACCCAAATCTTTACCGAGTTTAACTACGCAAAGATCTATAACGTACAGAACCGCAAGATTTAT
CTTAGCAATGTATACACACCGGTTACTGCGAACATCTTAGACGACAATGTGTATGATATTCAGAATGGCTTTAAC
ATCCCGAAATCAAATCTGAACGTTCTGTTTATGGGCCAGAACCTGAGTCGTAATCCAGCACTGCGTAAAGTGAAC
CCGGAAAATATGCTCTACTTGTTTACCAAATTTTGCCACAAAGCGATTGATGGCCGCTCTCTCTATAACAAAACG
CTGGATTGTCGTGAGTTACTTGTGAAGAACACTGATTTACCGTTCATTGGGGATATCTCCGACGTGAAAACCGAT
ATCTTCCTGCGCAAAGACATTAATGAAGAAACGGAAGTCATCTATTACCCCGACAATGTGAGCGTTGATCAGGTC
ATTTTATCGAAGAACACCTCCGAACATGGTCAGTTGGATTTGCTGTACCCTAGCATTGACTCGGAGAGTGAAATC
CTTCCGGGCGAAAATCAAGTGTTTTACGACAACCGTACCCAAAATGTTGATTATTTGAATTCTTATTACTACCTG
GAATCTCAGAAATTGAGCGACAATGTGGAAGATTTCACGTTCACACGCTCCATTGAGGAAGCGCTGGATAATAGC
GCGAAAGTGTATACGTATTTCCCTACCTTGGCGAATAAAGTAAATGCTGGTGTCCAGGGAGGCTTATTTCTGATG
TGGGCGAATGATGTGGTAGAAGATTTTACGACCAATATTTTGCGTAAGGACACCTTAGATAAAATTAGCGATGTT
AGCGCCATCATCCCCTATATTGGCCCAGCACTGAATATCTCGAACTCTGTGCGTCGCGGAAACTTCACCGAAGCA
TTTGCGGTGACCGGGGTTACTATTCTGTTGGAAGCCTTTCCGGAGTTTACTATTCCGGCGCTGGGTGCGTTTGTG
ATTTATTCGAAAGTACAAGAACGCAATGAAATTATCAAAACCATCGATAATTGCCTGGAACAACGCATTAAACGC
TGGAAGGATTCTTATGAATGGATGATGGGCACCTGGTTATCCCGTATTATCACACAGTTTAACAACATCTCGTAT
CAGATGTACGATTCACTGAACTACCAAGCAGGGGCGATCAAAGCCAAGATCGACTTAGAATACAAGAAATATTCA
GGTAGCGATAAAGAGAATATTAAAAGCCAGGTTGAAAACCTGAAGAACTCTCTGGATGTCAAAATTTCAGAGGCT
ATGAACAACATTAACAAATTTATCCGCGAATGTAGCGTCACGTATCTGTTTAAAAACATGCTCCCGAAAGTGATT
GATGAGCTCAACGAGTTTGATCGCAACACAAAGGCCAAACTGATTAACCTGATTGATAGTCACAATATTATTTTA
GTCGGTGAAGTTGACAAGCTGAAGGCTAAGGTCAATAACAGCTTTCAGAACACTATTCCGTTTAATATTTTCTCC
TATACGAACAATAGTCTGCTGAAAGACATTATCAACGAATACTTCAACAATATTAATGACAGCAAAATTCTGAGC
CTGCAGAATCGTAAGAATACGCTGGTAGATACCAGTGGATATAATGCGGAAGTCTCAGAAGAGGGTGATGTACAG
CTGAACCCGATCTTTCCGTTCGACTTTAAACTGGGGTCTAGTGGTGAAGATCGCGGTAAAGTGATCGTTACCCAA
AACGAGAACATTGTGTATAACAGCATGTACGAGAGTTTCTCAATTTCTTTCTGGATTCGCATCAATAAATGGGTT
TCTAATTTGCCTGGCTATACCATCATTGATAGCGTCAAAAACAACTCGGGCTGGTCGATTGGCATTATTAGCAAC
TTTCTGGTGTTTACCCTGAAACAGAATGAGGATTCGGAACAGAGCATTAACTTCTCCTACGACATCAGCAACAAT
GCACCAGGGTATAACAAATGGTTCTTCGTAACGGTGACGAACAATATGATGGGCAATATGAAAATCTACATTAAC
GGGAAACTTATCGACACCATTAAAGTGAAAGAGCTTACTGGGATCAATTTTAGTAAAACCATTACCTTTGAGATC
AACAAAATTCCGGACACGGGTCTGATTACCTCCGATTCGGATAATATCAATATGTGGATTCGCGACTTTTATATC
TTCGCCAAAGAACTTGATGGCAAAGATATCAACATTTTGTTTAATTCCCTGCAGTATACCAATGTCGTTAAGGAC
TATTGGGGCAATGATCTCCGCTACAATAAAGAATACTACATGGTTAACATCGACTATCTCAATCGCTACATGTAT
GCTAACTCGCGTCAAATTGTGTTTAACACACGTCGTAACAACAACGATTTTAACGAAGGTTATAAAATCATTATC
AAACGGATCCGCGGCAATACGAACGATACTCGTGTTCGTGGCGGTGACATTCTGTATTTCGACATGACGATTAAT
AATAAAGCGTACAATCTGTTCATGAAGAACGAAACCATGTACGCCGATAACCATTCCACTGAAGATATCTACGCA
ATCGGACTTCGCGAACAGACCAAAGACATTAACGACAACATCATCTTTCAGATTCAACCGATGAATAATACCTAC
TACTATGCCTCCCAGATCTTCAAAAGTAATTTCAACGGCGAAAACATTTCAGGCATTTGCTCAATCGGCACTTAT
CGGTTCCGGTTAGGTGGTGATTGGTATCGTCACAACTACCTTGTTCCCACAGTGAAACAAGGCAACTATGCATCG
CTCTTAGAAAGCACATCTACGCATTGGGGTTTTGTGCCAGTCAGTGAA
SEQ ID NO: 12-Polypeptide Sequence of rBoNT/C(0)
MPITINNFNYSDPVDNKNILYLDTHLNTLANEPEKAFRITGNIWVIPDRFSRNSNPNLNKPPRVTSPKSGYYDPN
YLSTDSDKDPFLKEIIKLFKRINSREIGEELIYRLSTDIPFPGNNNTPINTFDFDVDFNSVDVKTRQGNNWVKTG
SINPSVIITGPRENIIDPETSTFKLTNNTFAAQEGFGALSIISISPRFMLTYSNATNDVGEGRFSKSEFCMDPIL
ILMGTLNNAMHNLYGIAIPNDQTISSVTSNIFYSQYNVKLEYAEIYAFGGPTIDLIPKSARKYFEEKALDYYRSI
AKRLNSITTANPSSFNKYIGEYKQKLIRKYRFVVESSGEVTVNRNKEVELYNELTQIFTEFNYAKIYNVQNRKIY
LSNVYTPVTANILDDNVYDIQNGFNIPKSNLNVLFMGQNLSRNPALRKVNPENMLYLFTKFCHKAIDGRSLYNKT
LDCRELLVKNTDLPFIGDISDVKTDIFLRKDINEETEVIYYPDNVSVDQVILSKNTSEHGQLDLLYPSIDSESEI
LPGENQVFYDNRTQNVDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILLEAFPEFTIPALGAFV
IYSKVQERNEIIKTIDNCLEQRIKRWKDSYEWMMGTWLSRIITQFNNISYQMYDSLNYQAGAIKAKIDLEYKKYS
GSDKENIKSQVENLKNSLDVKISEAMNNINKFIRECSVTYLFKNMLPKVIDELNEFDRNTKAKLINLIDSHNIIL
VGEVDKLKAKVNNSFQNTIPFNIFSYTNNSLLKDIINEYENNINDSKILSLQNRKNTLVDTSGYNAEVSEEGDVQ
LNPIFPFDFKLGSSGEDRGKVIVTQNENIVYNSMYESFSISFWIRINKWVSNLPGYTIIDSVKNNSGWSIGIISN
FLVFTLKQNEDSEQSINFSYDISNNAPGYNKWFFVTVTNNMMGNMKIYINGKLIDTIKVKELTGINFSKTITFEI
NKIPDTGLITSDSDNINMWIRDFYIFAKELDGKDINILFNSLQYTNVVKDYWGNDLRYNKEYYMVNIDYLNRYMY
ANSRQIVFNTRRNNNDENEGYKIIIKRIRGNTNDTRVRGGDILYFDMTINNKAYNLFMKNETMYADNHSTEDIYA
IGLREQTKDINDNIIFQIQPMNNTYYYASQIFKSNENGENISGICSIGTYRFRLGGDWYRHNYLVPTVKQGNYAS
LLESTSTHWGFVPVSE
SEQ ID NO: 13-Nucleotide Sequence of rBoNT/E(0)
atgccgaaaatcaactctttcaactacaacgacccggttaacgaccgtaccatcctgtatatcaaaccgggtggt
tgccaggagttctacaaatctttcaacatcatgaaaaacatctggatcatcccggaacgtaacgttatcggtacc
accccgcaggacttccacccgccgacctctctgaaaaacggtgactcttcttactacgacccgaactacctccag
tctgacgaagaaaaagaccgtttcctgaaaatcgttaccaaaatcttcaaccgtatcaacaacaacctgtctggt
ggtatcctgctggaagaactgtctaaagctaacccgtacctgggtaacgacaacaccccggacaaccagttccac
atcggtgacgcttctgctgttgaaatcaaattctctaacggttctcaggacatcctgctgccgaacgttatcatc
atgggtgctgaaccggacctgttcgaaaccaactcttctaacatctctctgcgtaacaactacatgccgtctaac
cacggtttcggttctatcgctatcgttaccttctctccggaatactctttccgtttcaacgacaacagcatgaac
gagttcatccaggacccggctctgaccctgatgcaccaactgatctactctctgcacggtctgtacggtgctaaa
ggtatcaccaccaaatacaccatcacccagaaacagaacccgctgatcaccaacatccgtggtaccaacatcgaa
gagttcctgaccttcggtggtaccgacctgaacatcatcacctctgctcagtctaacgacatctacaccaacctg
ctggctgactacaaaaaaatcgcttctaaactgtctaaagttcaggtttctaacccgctgctgaacccgtacaaa
gacgttttcgaagctaaatacggtctggacaaagacgcttctggtatctactctgttaacatcaacaaattcaac
gacatcttcaaaaaactgtactctttcaccgagttcgacctggcgaccaaattccaggttaaatgccgtcagacc
tacatcggtcagtacaaatacttcaaactgtctaacctgctgaacgactctatctacaacatctctgaaggttac
aacatcaacaacctgaaagttaacttccgtggtcagaacgctaacctgaacccgcgtatcatcaccccgatcacc
ggtcgtggtctggttaaaaaaatcatccgtttctgcAAGAATATTGTAAGCGTTAAAGGAATAAGAAAAAGTATC
tgcatcgaaatcaacaacggtgaactgttcttcgttgcttctgaaaactcttacaacgacgacaacatcaacacc
ccgaaagaaatcgacgacaccgttacctctaacaacaactacgaaaacgacctggaccaggttatcctgaacttc
aactctgaatctgctccgggtctgtctgacgaaaaactgaacctgaccatccagaacgacgcttacatcccgaaa
tacgactctaacggtacctctgacatcgaacagcacgacgttaacgaactgaacgttttcttctacctggacgct
cagaaagttccggaaggtgaaaacaacgttaacctgacctcttctatcgacaccgctctgctggaacagccgaaa
atctacaccttcttctcttctgagttcatcaacaacgttaacaaaccggttcaggctgctctgttcgtttcttgg
attcagcaggttctggttgacttcaccaccgaagctaaccagaaatctaccgttgacaaaatcgctgacatctct
atcgttgttccgtacatcggtctggctctgaacatcggtaacgaagctcagaaaggtaacttcaaagacgctctg
gaactgctgggtgctggtatcctgctggagttcgaaccggaactgctgatcccgaccatcctggttttcaccatc
aaatctttcctgggttcttctgacaacaaaaacaaagttatcaaagctatcaacaacgctctgaaagaacgtgac
gaaaaatggaaagaagtttactctttcatcgtttctaactggatgaccaaaatcaacacccagttcaacaaacgt
aaagaacagatgtaccaggctctccagaaccaggttaacgctatcaaaaccatcatcgaatctaaatacaactct
tacaccctggaagaaaaaaacgaactgaccaacaaatacgacatcaaacagatcgaaaacgaactgaaccagaaa
gtttctatcgctatgaacaacatcgaccgtttcctgaccgaatcttctatctcttacctgatgaaactcatcaac
gaagttaaaatcaacaaactgcgtgaatacgacgaaaacgttaaaacctacctgctgaactacatcatccagcac
ggttctatcctgggtgaatctcagcaggaactgaactctatggttaccgacaccctgaacaactctatcccgttc
aaactgtcttcttacaccgacgacaaaatcctGATCTCTTACTTCAACAAATTCTTTAAAcgcATTAAGAGTTCA
TCGGTTctgaatATGCGGTACAAAAATGATAAAtatGTCGATACTTCTGGATATgatAGCAATATCAACATTAAC
GGCGACGTGTATAAATATccgACAAATAAAAACCAGTTTGGGATATATAACGACAAGctgTCGGAGGTCAATatt
TCTCAAAACGACtatATCattTACGATAATaaaTATAAAAACTTTAGCATTAGTtttTGGGTTcgtATACCTAAT
tatGACAATaaaattGTAAATGTGAATAACGAGTATACCATTATAAACTGTATGcgcGACAATAACAGTGGTTGG
AAGGTATCGctgAACCATAATGAGATTATCTGGACCctgcagGATAATgcaGGTATAAACCAGAAACTGGCTTTT
AACTATGGAAACGCAAATGGGATCTCAGATTACATTaataaaTGGatttttGTTaccATTACGAACGATcgcTTA
GGCGACTCAAAACTTTATATTAATggcAATctgATAGATCAGAAATCAATCTTAAATTTGGGCAATATTCATGTC
TCTgatAACATCTTGTTCAAGATCGTTAATTGCAGTTACACTcgtTATATTGGCATTCGTTACTTTAATATCTTC
gataaaGAActgGACGAGACGGAAATCcagACTCTGTATTCAAACGAGCCCAATACTAATATATTGAAAGATTTT
TGGGGTAACTATCTTTTATATGATAAAGAATACTATCTCCTGaatGTATTGAAGCCAAACAATTTCATAGATAGA
CGCAAGGATAGCACATTAAGTATCAACAATATCAGATCTACTATActgttaGCAAATCGCCTcTACTCCggtATT
AAAGTGAAGATTcagCGGGTTAATAACTCCAGTACCAATGATAATCTGGTCCGTAAGAACGATCAGGTATACATC
aatTTCGTCGCGAGCAAAACTcatCTCTTCCCGCTTTACGCCgatACAGCTACGACAAACAAGGAAAAAACCATA
AAAATTTCCAGCTCCGGAAACAGATTCAATCAAGTAGTTGTAATGAACTCTGTGGGTaatAATTGTACGATGAAC
TTTaagAATAACAATGGGAACAATattGGACTTTTGGGCTTcAAAGCCGACACAGTGGTGGCGTCCACCTGGTAT
TACACGcacATGcggGACCATACGAATTCGAACGGTTGCTTCTGGAACTTTATCTCGGAAgaaCACGGGTGGCAA
GAAAAA
SEQ ID NO: 14-Polypeptide Sequence of rBoNT/E(0)
MPKINSFNYNDPVNDRTILYIKPGGCQEFYKSFNIMKNIWIIPERNVIGTTPQDFHPPTSLKNGDSSYYDPNYLQ
SDEEKDRFLKIVTKIFNRINNNLSGGILLEELSKANPYLGNDNTPDNQFHIGDASAVEIKFSNGSQDILLPNVII
MGAEPDLFETNSSNISLRNNYMPSNHGFGSIAIVTFSPEYSFRENDNSMNEFIQDPALTLMHQLIYSLHGLYGAK
GITTKYTITQKQNPLITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLATKFQVKCRQTYIGQYKYFKLSNLLNDSIYNISEGY
NINNLKVNFRGQNANLNPRIITPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSDIEQHDVNELNVFFYLDA
QKVPEGENNVNLTSSIDTALLEQPKIYTFFSSEFINNVNKPVQAALFVSWIQQVLVDETTEANQKSTVDKIADIS
IVVPYIGLALNIGNEAQKGNEKDALELLGAGILLEFEPELLIPTILVFTIKSFLGSSDNKNKVIKAINNALKERD
EKWKEVYSFIVSNWMTKINTQFNKRKEQMYQALQNQVNAIKTIIESKYNSYTLEEKNELTNKYDIKQIENELNQK
VSIAMNNIDRFLTESSISYLMKLINEVKINKLREYDENVKTYLLNYIIQHGSILGESQQELNSMVTDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDQKSILNLGNIHVSDNILFKIVNCSYTRYIGIRYFNIF
DKELDETEIQTLYSNEPNTNILKDFWGNYLLYDKEYYLLNVLKPNNFIDRRKDSTLSINNIRSTILLANRLYSGI
KVKIQRVNNSSTNDNLVRKNDQVYINFVASKTHLFPLYADTATTNKEKTIKISSSGNRFNQVVVMNSVGNNCTMN
FKNNNGNNIGLLGFKADTVVASTWYYTHMRDHTNSNGCFWNFISEEHGWQEK
SEQ ID NO: 15-Nucleotide Sequence of rBoNT/F(0)
ATGCCGGTGGTCATCAACAGCTTCAACTACAACGACCCAGTAAACGACGACACGATCCTGTATATGCAAATCCCG
TATGAAGAGAAGAGCAAGAAGTACTATAAGGCCTTTGAAATCATGCGCAATGTGTGGATTATTCCGGAGCGTAAT
ACGATTGGTACTGACCCAAGCGACTTCGATCCACCTGCGTCTTTGGAAAACGGCTCGTCCGCATATTACGACCCG
AATTACCTGACCACCGATGCGGAGAAAGATCGTTATTTGAAAACCACCATCAAGCTGTTCAAACGCATTAACAGC
AATCCGGCAGGTGAGGTCCTGCTGCAAGAGATTAGCTACGCAAAGCCTTATCTGGGTAATGAGCATACGCCTATT
AACGAGTTTCACCCGGTTACCCGCACTACCAGCGTTAACATCAAGTCCTCGACCAACGTGAAGTCTAGCATTATC
CTGAACCTGCTGGTTCTGGGTGCCGGTCCGGACATCTTCGAAAACTCTAGCTACCCGGTGCGTAAACTGATGGAT
AGCGGCGGTGTTTATGACCCGAGCAATGACGGTTTTGGCAGCATCAATATCGTGACGTTTAGCCCGGAGTACGAG
TACACCTTCAATGATATCAGCGGTGGTTACAATTCTTCTACCGAGAGCTTCATCGCCGACCCGGCGATCAGCCTG
GCACACCAACTGATCTATGCATTGCATGGCTTGTACGGTGCCCGTGGTGTGACGTATAAAGAGACTATCAAGGTT
AAGCAGGCACCTCTGATGATTGCGGAAAAGCCGATTCGCCTGGAAGAGTTCCTGACCTTCGGCGGTCAAGATTTG
AACATCATTACCTCGGCCATGAAAGAGAAAATCTATAACAATTTGCTGGCCAACTATGAAAAGATTGCAACGCGC
TTGTCTCGTGTTAACTCCGCTCCGCCGGAATACGACATTAATGAGTACAAAGACTACTTTCAATGGAAATATGGC
CTGGACAAAAATGCGGATGGTTCTTATACCGTGAATGAAAACAAATTCAATGAAATCTACAAGAAACTGTACAGC
TTCACCGAAATCGATCTGGCGAACAAGTTCAAAGTCAAATGTCGTAATACCTACTTCATCAAATATGGCTTCCTG
AAAGTCCCGAACCTGCTGGACGATGACATCTATACCGTCAGCGAAGGCTTCAACATCGGCAATCTGGCCGTGAAT
AATCGTGGTCAGAACATCAAACTGAATCCGAAAATCATTGACTCCATCCCAGACAAGGGCCTGGTTGAGAAAATC
GTGAAGTTCTGCAAAAGCGTTATTCCGCGTAAAGGTACGAAAGCACCGCCTCGCCTGTGCATTCGCGTTAACAAC
CGTGAGTTGTTCTTTGTGGCATCTGAAAGCAGCTACAACGAGAACGACATCAACACCCCTAAAGAAATTGATGAT
ACCACGAACCTGAATAACAATTATCGCAACAATCTGGACGAGGTGATCCTGGATTACAATTCGGAAACCATTCCG
CAAATTAGCAATCAGACGCTGAACACCCTGGTTCAGGACGATAGCTACGTTCCGCGTTACGACTCCAATGGTACT
AGCGAGATTGAAGAACACAACGTAGTGGACTTGAACGTTTTCTTTTATCTGCACGCCCAGAAGGTTCCGGAGGGC
GAAACCAATATTAGCCTGACCAGCTCGATCGACACCGCGCTGTCTGAGGAGAGCCAAGTCTACACCTTTTTCAGC
AGCGAGTTTATCAACACTATTAACAAGCCAGTTCATGCTGCATTGTTTATCTCTTGGATTAACCAGGTGATTCGC
GACTTTACGACGGAGGCGACCCAGAAGTCTACCTTCGACAAAATTGCAGACATCTCCCTGGTCGTCCCATACGTC
GGCCTGGCGTTGAATATTGGCAATGAAGTTCAAAAAGAGAACTTCAAAGAAGCGTTCGAGCTGCTGGGTGCAGGC
ATCCTGCTGGAGTTCGTGCCGGAACTGTTGATCCCGACCATCCTGGTGTTCACCATTAAGAGCTTCATTGGATCC
TCCGAGAATAAGAACAAGATCATCAAGGCGATCAATAACAGCCTGATGGAGCGTGAAACGAAGTGGAAAGAAATC
TATAGCTGGATTGTTAGCAATTGGCTGACTCGTATTAACACGCAATTCAACAAGCGTAAAGAGCAAATGTACCAA
GCCCTGCAAAACCAAGTTGACGCCATCAAAACGGTAATTGAATACAAGTACAACAATTACACGAGCGATGAGCGC
AACCGCCTGGAAAGCGAATACAACATCAACAACATTCGCGAAGAATTGAACAAGAAAGTGAGCCTGGCGATGGAG
AACATTGAGCGTTTTATCACCGAAAGCAGCATCTTTTACCTGATGAAATTGATTAATGAGGCGAAAGTCTCGAAA
CTGCGTGAGTACGACGAAGGTGTGAAAGAGTATCTGCTGGATTACATTAGCGAGCACCGTAGCATCTTGGGTAAC
TCGGTTCAGGAGCTGAACGATCTGGTGACCTCTACCCTGAACAATAGCATCCCGTTCGAACTGAGCAGCTATACC
AATGACAAGATTCTGATTCTGTATTTCAATAAACTGTATAAGAAGATCAAGGATAACAGCATTCTGGATATGCGT
TACGAAAACAATAAGTTTATCGACATTTCTGGTTACGGCAGCAACATTTCCATCAATGGCGATGTCTACATCTAC
AGCACCAATCGCAACCAGTTCGGCATCTACTCTAGCAAACCGAGCGAAGTTAACATCGCACAGAACAATGATATT
ATTTATAACGGTCGTTATCAAAACTTCTCTATCAGCTTTTGGGTCCGTATCCCGAAGTACTTCAATAAAGTCAAT
CTGAATAATGAATACACGATCATCGACTGCATTCGCAATAACAACAGCGGTTGGAAAATCAGCCTGAATTACAAC
AAAATTATTTGGACCCTGCAAGATACGGCGGGTAACAATCAGAAACTGGTGTTTAACTACACGCAAATGATCAGC
ATTTCTGACTATATCAACAAGTGGATCTTTGTTACCATCACCAATAATCGTCTGGGCAATAGCCGTATTTACATC
AACGGTAACCTGATTGATGAGAAAAGCATCAGCAACCTGGGCGATATTCACGTCAGCGACAACATTCTGTTCAAA
ATTGTTGGTTGTAACGATACCCGTTACGTCGGCATCCGTTATTTCAAGGTTTTCGATACGGAGCTGGGTAAAACG
GAAATCGAAACGTTGTACTCCGATGAACCAGATCCGAGCATTCTGAAGGACTTTTGGGGTAACTACTTGCTGTAC
AATAAACGTTACTATCTGCTGAATCTGTTGCGCACCGACAAGAGCATTACCCAAAACAGCAATTTCCTGAACATT
AATCAGCAACGCGGCGTATACCAAAAACCGAACATCTTCAGCAATACGCGCCTGTATACTGGTGTTGAAGTGATC
ATTCGTAAGAACGGTAGCACCGACATTAGCAACACGGACAATTTCGTCCGTAAGAATGACCTGGCGTACATTAAC
GTCGTGGACCGTGATGTCGAGTATCGTCTGTACGCAGACATCAGCATTGCGAAACCGGAAAAGATTATCAAGCTG
ATCCGTACCAGCAACAGCAACAACAGCCTGGGTCAGATCATTGTGATGGACAGCATTGGTAATAACTGCACGATG
AACTTCCAGAACAACAATGGTGGTAATATCGGTCTGCTGGGTTTTCACAGCAATAATCTGGTTGCTTCCAGCTGG
TACTACAATAACATTCGTAAAAACACGTCTAGCAATGGTTGTTTTTGGAGCTTTATCAGCAAAGAGCACGGCTGG
CAAGAAAAT
SEQ ID NO: 16-Polypeptide Sequence of rBoNT/F(0)
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTFNDISGGYNSSTESFIADPAISL
AHQLIYALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYENKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDCIRNNNSGWKISLNYN
KIIWTLQDTAGNNQKLVFNYTQMISISDYINKWIFVTITNNRLGNSRIYINGNLIDEKSISNLGDIHVSDNILFK
IVGCNDTRYVGIRYFKVFDTELGKTEIETLYSDEPDPSILKDFWGNYLLYNKRYYLLNLLRTDKSITQNSNFLNI
NQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGW
QEN
SEQ ID NO: 17-Nucleotide Sequence of rBoNT/A(0) (His-tagged)
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAAAGCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACACCAGCTG
ATTTATGCCGGTCATCGTCTGTATGGTATTGCCATTAATCCGAATCGTGTGTTCAAAGTGAATACCAACGCCTAT
TATGAAATGAGCGGTCTGGAAGTGAGTTTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTCGAATTT
TACAAACTGCTGTGTGTTCGTGGCATTATTACCAGCAAAACCAAAAGTCTGGATAAAGGCTACAATAAAGCCCTG
AATGATCTGTGCATTAAGGTGAATAATTGGGACCTGTTTTTTAGCCCGAGCGAGGATAATTTCACCAACGATCTG
AACAAAGGCGAAGAAATTACCAGCGATACCAATATTGAAGCAGCCGAAGAAAACATTAGCCTGGATCTGATTCAG
CAGTATTATCTGACCTTCAACTTCGATAATGAGCCGGAAAATATCAGCATTGAAAACCTGAGCAGCGATATTATT
GGCCAGCTGGAACTGATGCCGAATATTGAACGTTTTCCGAACGGCAAAAAATACGAGCTGGATAAATACACCATG
TTCCATTATCTGCGTGCCCAAGAATTTGAACATGGTAAAAGCCGTATTGCACTGACCAATAGCGTTAATGAAGCA
CTGCTGAACCCGAGCCGTGTTTATACCTTTTTTAGCAGCGATTACGTGAAAAAGGTTAACAAAGCAACCGAAGCA
GCCATGTTTTTAGGTTGGGTTGAACAGCTGGTTTATGATTTCACCGATGAAACCAGCGAAGTTAGCACCACCGAT
AAAATTGCAGATATTACCATCATCATCCCGTATATCGGTCCGGCACTGAATATTGGCAATATGCTGTATAAAGAC
GATTTTGTGGGTGCCCTGATTTTTAGCGGTGCAGTTATTCTGCTGGAATTTATTCCGGAAATTGCCATTCCGGTT
CTGGGCACCTTTGCACTGGTGAGCTATATTGCAAATAAAGTTCTGACCGTGCAGACCATCGATAATGCACTGAGC
AAACGTAACGAAAAATGGGATGAAGTGTACAAGTATATCGTGACCAATTGGCTGGCAAAAGTTAACACCCAGATT
GACCTGATTCGCAAGAAGATGAAAGAAGCACTGGAAAATCAGGCAGAAGCAACCAAAGCCATTATCAACTATCAG
TATAACCAGTACACCGAAGAAGAGAAAAATAACATCAACTTCAACATCGACGATCTGTCCAGCAAACTGAACGAA
AGCATCAACAAAGCCATGATTAACATTAACAAATTTCTGAACCAGTGCAGCGTGAGCTATCTGATGAATAGCATG
ATTCCGTATGGTGTGAAACGTCTGGAAGATTTTGATGCAAGCCTGAAAGATGCCCTGCTGAAATATATCTATGAT
AATCGTGGCACCCTGATTGGTCAGGTTGATCGTCTGAAAGATAAAGTGAACAACACCCTGAGTACCGATATTCCT
TTTCAGCTGAGCAAATATGTGGATAATCAGCGTCTGCTGTCAACCTTTACCGAATACATTAAGAACATCATCAAC
ACCAGCATTCTGAACCTGCGTTATGAAAGCAATCATCTGATTGATCTGAGCCGTTATGCCAGCAAAATCAATATA
GGCAGCAAGGTTAACTTCGACCCGATTGACAAAAATCAGATACAGCTGTTTAATCTGGAAAGCAGCAAAATTGAG
GTGATCCTGAAAAACGCCATTGTGTATAATAGCATGTACGAGAATTTCTCGACCAGCTTTTGGATTCGTATCCCG
AAATACTTTAATAGCATCAGCCTGAACAACGAGTACACCATTATTAACTGCATGGAAAACAATAGCGGCTGGAAA
GTTAGCCTGAATTATGGCGAAATTATCTGGACCCTGCAGGATACCCAAGAAATCAAACAGCGTGTGGTTTTCAAA
TACAGCCAGATGATTAATATCAGCGACTATATCAACCGCTGGATTTTTGTGACCATTACCAATAATCGCCTGAAT
AACAGCAAGATCTATATTAACGGTCGTCTGATTGACCAGAAACCGATTAGTAATCTGGGTAATATTCATGCGAGC
AACAACATCATGTTTAAACTGGATGGTTGTCGTGATACCCATCGTTATATTTGGATCAAGTACTTCAACCTGTTC
GATAAAGAGTTGAACGAAAAAGAAATTAAAGACCTGTATGATAACCAGAGCAACAGCGGTATTCTGAAGGATTTT
TGGGGAGATTATCTGCAGTATGACAAACCGTATTATATGCTGAATCTGTACGACCCGAATAAATACGTGGATGTG
AATAATGTTGGCATCCGTGGTTATATGTACCTGAAAGGTCCGCGTGGTAGCGTTATGACCACAAACATTTATCTG
AATAGCAGCCTGTATCGCGGAACCAAATTCATCATTAAAAAGTATGCCAGCGGCAACAAGGATAATATTGTGCGT
AATAATGATCGCGTGTACATTAACGTTGTGGTGAAGAATAAAGAATATCGCCTGGCAACCAATGCAAGCCAGGCA
GGCGTTGAAAAAATTCTGAGTGCCCTGGAAATTCCGGATGTTGGTAATCTGAGCCAGGTTGTTGTGATGAAAAGC
AAAAATGATCAGGGCATCACCAACAAGTGCAAAATGAATCTGCAGGACAATAACGGCAACGATATTGGTTTTATT
GGCTTCCACCAGTTCAACAATATTGCGAAACTGGTTGCAAGCAATTGGTATAATCGTCAGATTGAACGTAGCAGT
CGTACCCTGGGTTGTAGCTGGGAATTTATCCCTGTGGATGATGGTTGGGGTGAACGTCCGCTGGAAAACCTGTAT
TTTCAAGGTGCAAGTCATCATCACCATCACCACCATCATTAA
SEQ ID NO: 18-Polypeptide Sequence of rBoNT/A(0) (His-tagged)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPLENLYFQGASHHHHHHHH
SEQ ID NO: 19-Nucleotide Sequence of rLH_{N}/A (His-tagged)
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAATCCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACATGAACTG
ATTCATGCCGGTCATCGTCTGTATGGTATTGCAATTAATCCGAACCGTGTGTTCAAAGTGAATACCAACGCATAT
TATGAAATGAGCGGTCTGGAAGTGTCATTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTCGAATTT
TACAAACTGCTGTGTGTTCGTGGCATTATTACCAGCAAAACCAAAAGTCTGGATAAAGGCTACAATAAAGCCCTG
AATGATCTGTGCATTAAGGTGAATAATTGGGACCTGTTTTTTAGCCCGAGCGAGGATAATTTCACCAACGATCTG
AACAAAGGCGAAGAAATTACCAGCGATACCAATATTGAAGCAGCCGAAGAAAACATTAGCCTGGATCTGATTCAG
CAGTATTATCTGACCTTCAACTTCGATAATGAGCCGGAAAATATCAGCATTGAAAACCTGAGCAGCGATATTATT
GGCCAGCTGGAACTGATGCCGAATATTGAACGTTTTCCGAACGGCAAAAAATACGAGCTGGATAAATACACCATG
TTCCATTATCTGCGTGCCCAAGAATTTGAACATGGTAAAAGCCGTATTGCACTGACCAATAGCGTTAATGAAGCA
CTGCTGAACCCGAGCCGTGTTTATACCTTTTTTAGCAGCGATTACGTGAAAAAGGTTAACAAAGCAACCGAAGCA
GCCATGTTTTTAGGTTGGGTTGAACAGCTGGTTTATGATTTCACCGATGAAACCAGCGAAGTTAGCACCACCGAT
AAAATTGCAGATATTACCATCATCATCCCGTATATCGGTCCGGCACTGAATATTGGCAATATGCTGTATAAAGAC
GATTTTGTGGGTGCCCTGATTTTTAGCGGTGCAGTTATTCTGCTGGAATTTATTCCGGAAATTGCCATTCCGGTT
CTGGGCACCTTTGCACTGGTGAGCTATATTGCAAATAAAGTTCTGACCGTGCAGACCATCGATAATGCACTGAGC
AAACGTAACGAAAAATGGGATGAAGTGTACAAGTATATCGTGACCAATTGGCTGGCAAAAGTTAACACCCAGATT
GACCTGATTCGCAAGAAGATGAAAGAAGCACTGGAAAATCAGGCAGAAGCAACCAAAGCCATTATCAACTATCAG
TATAACCAGTACACCGAAGAAGAGAAAAATAACATCAACTTCAACATCGACGATCTGTCCAGCAAACTGAACGAA
AGCATCAACAAAGCCATGATTAACATTAACAAATTTCTGAACCAGTGCAGCGTGAGCTATCTGATGAATAGCATG
ATTCCGTATGGTGTGAAACGTCTGGAAGATTTTGATGCAAGCCTGAAAGATGCCCTGCTGAAATATATCTATGAT
AATCGTGGCACCCTGATTGGTCAGGTTGATCGTCTGAAAGATAAAGTGAACAACACCCTGAGTACCGATATTCCT
TTTCAGCTGAGCAAATATGTGGATAATCAGCGTCTGCTGTCAACCGAAAATCTGTATTTCCAGGGTGCAAGTCAT
CATCACCATCACCACCATCATTAA
SEQ ID NO: 20-Polypeptide Sequence of rLH_{N}/A (His-tagged)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNEDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTENLYFQGASHHHHHHHH
SEQ ID NO: 21-Nucleotide Sequence of rH_{C}/A (His-tagged)
ATGCATCATCACCATCACCACGAAAATCTATACTTCCAAGGAAAAAACATCATCAATACTAGCATTCTGAACCTG
CGTTACGAGAGCAATCATCTGATTGATCTGAGCCGTTATGCAAGCAAGATCAACATCGGTAGCAAGGTCAATTTT
GACCCGATCGATAAGAACCAGATCCAGCTGTTTAATCTGGAATCGAGCAAAATTGAGGTTATCCTGAAAAACGCC
ATTGTCTACAACTCCATGTACGAGAATTTCTCCACCAGCTTCTGGATTCGCATCCCGAAATACTTCAACAGCATT
AGCCTGAACAACGAGTATACTATCATCAACTGTATGGAGAACAACAGCGGTTGGAAGGTGTCTCTGAACTATGGT
GAGATCATTTGGACCTTGCAGGACACCCAAGAGATCAAGCAGCGCGTCGTGTTCAAGTACTCTCAAATGATCAAC
ATTTCCGATTACATTAATCGTTGGATCTTCGTGACCATTACGAATAACCGTCTGAATAACAGCAAGATTTACATC
AATGGTCGCTTGATCGATCAGAAACCGATTAGCAACCTGGGTAATATCCACGCAAGCAACAACATTATGTTCAAA
TTGGACGGTTGCCGCGATACCCATCGTTATATCTGGATCAAGTATTTCAACCTGTTTGATAAAGAACTGAATGAG
AAGGAGATCAAAGATTTGTATGACAACCAATCTAACAGCGGCATTTTGAAGGACTTCTGGGGCGATTATCTGCAA
TACGATAAGCCGTACTATATGCTGAACCTGTATGATCCGAACAAATATGTGGATGTCAATAATGTGGGTATTCGT
GGTTACATGTATTTGAAGGGTCCGCGTGGCAGCGTTATGACGACCAACATTTACCTGAACTCTAGCCTGTACCGT
GGTACGAAATTCATCATTAAGAAATATGCCAGCGGCAACAAAGATAACATTGTGCGTAATAACGATCGTGTCTAC
ATCAACGTGGTCGTGAAGAATAAAGAGTACCGTCTGGCGACCAACGCTTCGCAGGCGGGTGTTGAGAAAATTCTG
AGCGCGTTGGAGATCCCTGATGTCGGTAATCTGAGCCAAGTCGTGGTTATGAAGAGCAAGAACGACCAGGGTATC
ACTAACAAGTGCAAGATGAACCTGCAAGACAACAATGGTAACGACATCGGCTTTATTGGTTTCCACCAGTTCAAC
AATATTGCTAAACTGGTAGCGAGCAATTGGTACAATCGTCAGATTGAGCGCAGCAGCCGTACTTTGGGCTGTAGC
TGGGAGTTTATCCCGGTCGATGATGGTTGGGGCGAACGTCCGCTGTAA
SEQ ID NO: 22-Polypeptide Sequence of rH_{C}/A (His-tagged)
MHHHHHHENLYFQGKNIINTSILNLRYESNHLIDLSRYASKINIGSKVNFDPIDKNQIQLENLESSKIEVILKNA
IVYNSMYENFSTSFWIRIPKYENSISLNNEYTIINCMENNSGWKVSLNYGEIIWTLQDTQEIKQRVVFKYSQMIN
ISDYINRWIFVTITNNRLNNSKIYINGRLIDQKPISNLGNIHASNNIMFKLDGCRDTHRYIWIKYFNLFDKELNE
KEIKDLYDNQSNSGILKDFWGDYLQYDKPYYMLNLYDPNKYVDVNNVGIRGYMYLKGPRGSVMTTNIYLNSSLYR
GTKFIIKKYASGNKDNIVRNNDRVYINVVVKNKEYRLATNASQAGVEKILSALEIPDVGNLSQVVVMKSKNDQGI
TNKCKMNLQDNNGNDIGFIGFHQFNNIAKLVASNWYNRQIERSSRTLGCSWEFIPVDDGWGERPL
SEQ ID NO: 23-Nucleotide Sequence of rLC/A (His-tagged)
ATGCCGTTTGTGAACAAGCAGTTCAACTATAAAGATCCGGTTAATGGTGTGGATATCGCCTATATCAAAATTCCG
AATGCAGGTCAGATGCAGCCGGTTAAAGCCTTTAAAATCCATAACAAAATTTGGGTGATTCCGGAACGTGATACC
TTTACCAATCCGGAAGAAGGTGATCTGAATCCGCCTCCGGAAGCAAAACAGGTTCCGGTTAGCTATTATGATAGC
ACCTATCTGAGCACCGATAACGAGAAAGATAACTATCTGAAAGGTGTGACCAAACTGTTTGAACGCATTTATAGT
ACCGATCTGGGTCGTATGCTGCTGACCAGCATTGTTCGTGGTATTCCGTTTTGGGGTGGTAGCACCATTGATACC
GAACTGAAAGTTATTGACACCAACTGCATTAATGTGATTCAGCCGGATGGTAGCTATCGTAGCGAAGAACTGAAT
CTGGTTATTATTGGTCCGAGCGCAGATATCATTCAGTTTGAATGTAAATCCTTTGGCCACGAAGTTCTGAATCTG
ACCCGTAATGGTTATGGTAGTACCCAGTATATTCGTTTCAGTCCGGATTTTACCTTTGGCTTTGAAGAAAGCCTG
GAAGTTGATACAAATCCGCTGTTAGGTGCAGGTAAATTTGCAACCGATCCGGCAGTTACCCTGGCACATGAACTG
ATTCATGCCGGTCATCGTCTGTATGGTATTGCAATTAATCCGAACCGTGTGTTCAAAGTGAATACCAACGCATAT
TATGAAATGAGCGGTCTGGAAGTGTCATTTGAAGAACTGCGTACCTTTGGTGGTCATGATGCCAAATTTATCGAT
AGCCTGCAAGAAAATGAATTTCGCCTGTACTACTATAACAAATTCAAGGATATTGCGAGCACCCTGAATAAAGCC
AAAAGCATTGTTGGCACCACCGCAAGCCTGCAGTATATGAAAAATGTGTTTAAAGAAAAATATCTGCTGAGCGAA
GATACCAGCGGTAAATTTAGCGTTGACAAACTGAAATTCGATAAACTGTACAAGATGCTGACCGAGATTTATACC
GAAGATAACTTCGTGAAGTTTTTCAAAGTGCTGAACCGCAAAACCTACCTGAACTTTGATAAAGCCGTGTTCAAA
ATCAACATCGTGCCGAAAGTGAACTATACCATCTATGATGGTTTTAACCTGCGCAATACCAATCTGGCAGCAAAC
TTTAATGGTCAGAACACCGAAATCAACAACATGAACTTTACCAAACTGAAGAACTTCACCGGTCTGTTTGAAGAG
AATCTGTATTTCCAGGGTGCAAGTCATCATCACCATCACCACCATCATTAA
SEQ ID NO: 24-Polypeptide Sequence of rLC/A (His-tagged)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEENLYFQGASHHHHHHHH
SEQ ID NO: 25-Nucleotide Sequence of rBoNT/FA(0) (His-tagged)
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CACCAGCTGATTTATGTTCTGCATGGTCTGTATGGCACCTATATCACCGAATACAAAATTACCCCGAATGTGGTT
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCAATACCAAAAACAGCCTGTGCATTACCGTTAATAATCGCGACCTGTTTTTTATCGCA
AGCCAAGAAAGCTATGGCGAGAATACCATTAACACCTATAAAGAGATTGACGATACCACCACACTGGATCCGAGC
TTTGAAGATATTCTGGATAAAGTGATCCTGAACTTCAACGAACAGGTTATTCCGCAGATGCCGAATCGTAATGTT
AGCACCGATATTCAGAAAGACAACTACATCCCGAAATACGATTATAACCGCACCGACATTATCGATAGCTATGAA
GTTGGTCGCAACTACAACACCTTTTTCTATCTGAATGCCCAGAAATTTAGCCCGAACGAAAGCAATATTACCCTG
ACCAGCAGCTTTGATACAGGTCTGTTAGAAGGTAGCAAAGTGTATACCTTTTTCAGCAGCGATTTCATTAACAAC
ATCAACAAACCGGTTCAGGCCCTGCTGTTTATTGAATGGGTTAAACAGGTGATTCGCGATTTTACCACCGAAGCA
ACCAAAACCTCAACCGTTGATAAACTGAAAGATATTAGCCTGGTGGTGCCGTATATTGGTCTGGCACTGAATATT
GGTGATGAGATCTACAAACAGCATTTTGCAGAAGCAGTTGAACTGGTTGGTGCAGGTCTGCTGCTGGAATTTTCA
CCGGAATTTCTTATTCCGACGCTGCTGATTTTTACCATCAAAGGTTATCTGACCGGTAGCATTCGCGATAAAGAC
AAAATCATTAAAACCCTGGATAACGCCCTGAATGTTCGTGATCAGAAATGGAAAGAACTGTATCGTTGGGTTGTT
AGCAAATGGCTGACCACCATTAATACGCAGTTCAACAAACGCAAAGAACAAATGTACAAAGCCCTGAAAAATCAG
GCCACCGCCATTAAAAAGATCATCGAGAACAAATATAACAACTATACCACCGATGAAAAAAGCAAGATCGATAGC
AGCTATAACATCAACGAAATTGAACGCACCCTGAACGAAAAAATCAATCTGGCCATGAAAAACATCGAGCAGTTT
ATTACCGAAAGCAGCATTGCCTATCTGATCAATATCATCAACAACGAAACGATCCAGAAACTGAAAAGCTATGAT
GACCTGGTTCGTCGTTATCTGCTGGGTTATATTCGTAATCATAGCAGCATTCTGGGCAATAGCGTTGAAGAACTG
AATTCCAAAGTGAACAACCATCTGGATAATGGCATTCCGTTTGAACTGAGCAGTTATACCAATGATAGCCTGCTG
ATCCGCTACTTCAATAAAAACTATGGCGAACTGAAGTACAACTGCATTCTGAACATCAAATATGAGATGGATCGT
GACAAACTGGTTGATAGCAGCGGTTATCGTAGCCGTATCAATATTGGTACAGGCGTCAAATTTAGCGAGATCGAT
AAAAATCAAGTGCAGCTGAGCAATCTGGAATCCAGCAAAATTGAAGTCATTCTGAATAACGGCGTCATCTATAAC
AGCATGTATGAAAACTTTTCGACCAGCTTTTGGATTCGCATTCCGAAATACTTTCGCAACATCAATAACGAGTAC
AAGATCATCAGCTGTATGCAGAATAATAGCGGTTGGGAAGTGAGCCTGAATTTTAGCAATATGAACTCGAAAATC
ATCTGGACCCTGCAGGATACCGAAGGTATCAAAAAAACCGTTGTGTTTCAGTACACCCAGAACATTAACATTAGC
GACTATATCAACCGCTGGATCTTTGTGACCATTACAAATAATCGTCTGAGCAACAGCAAAATCTACATTAATGGT
CGCCTGATCAACGAAGAAAGCATTAGCGATCTGGGTAATATCCATGCCAGCAACAACATTATGTTTAAACTGGAT
GGTTGCCGTGATCCGCATCGTTATATCTGGATTAAATACTTTAACCTGTTTGACAAAGAGCTGAACAAGAAAGAA
ATTAAAGATCTGTACGACAACCAGAGCAATAGCGGTATTCTGAAAGATTTCTGGGGTGATTATCTGCAGTATGAC
AAACCGTATTATATGCTGAATCTGTATGACCCGAATAAGTATCTGGATGTGAATAATGTTGGCATCCGTGGCTAT
ATGTATCTGAAAGGTCCGCGTGGTCGTATTGTGACCACCAACATTTATCTGAATAGCACCCTGTATATGGGCACC
AAATTCATCATTAAGAAATATGCCAGCGGCAACAAAGATAACATTGTGCGTAATAATGATCGCGTGTATATTAAC
GTGGTGGTGAAGAATAAAGAATATCGCCTGGCAACCAATGCAAGCCAGGCAGGCGTTGAAAAAATTCTGAGCGCA
GTTGAAATCCCGGATGTTGGTAATCTGAGCCAGGTTGTTGTGATGAAAAGCGAAAATGATCAGGGCATTCGCAAC
AAGTGTAAAATGAATCTGCAAGACAATAACGGCAACGATATTGGCTTTATCGGCTTTCACCAGTTTAATAACATT
GCAAAACTGGTGGCCAGCAACTGGTATAACCGTCAGATTGGTAAAGCAAGCCGTACCTTTGGTTGTAGCTGGGAA
TTTATCCCGGTTGATGATGGTTGGGGTGAAAGCAGCCTGGAAAATCTGTATTTCCAGGGTGCCAGTCATCATCAC
CACCATCACCATCACTGA
SEQ ID NO: 26-Polypeptide Sequence of rBoNT/FA(0) (His-tagged)
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HQLIYVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSNTKNSLCITVNNRDLFFIA
SQESYGENTINTYKEIDDTTTLDPSFEDILDKVILNFNEQVIPQMPNRNVSTDIQKDNYIPKYDYNRTDIIDSYE
VGRNYNTFFYLNAQKFSPNESNITLTSSFDTGLLEGSKVYTFFSSDFINNINKPVQALLFIEWVKQVIRDETTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYFNKNYGELKYNCILNIKYEMDRDKLVDSSGYRSRINIGTGVKFSEID
KNQVQLSNLESSKIEVILNNGVIYNSMYENFSTSFWIRIPKYFRNINNEYKIISCMQNNSGWEVSLNFSNMNSKI
IWTLQDTEGIKKTVVFQYTQNINISDYINRWIFVTITNNRLSNSKIYINGRLINEESISDLGNIHASNNIMFKLD
GCRDPHRYIWIKYFNLFDKELNKKEIKDLYDNQSNSGILKDFWGDYLQYDKPYYMLNLYDPNKYLDVNNVGIRGY
MYLKGPRGRIVTTNIYLNSTLYMGTKFIIKKYASGNKDNIVRNNDRVYINVVVKNKEYRLATNASQAGVEKILSA
VEIPDVGNLSQVVVMKSENDQGIRNKCKMNLQDNNGNDIGFIGFHQFNNIAKLVASNWYNRQIGKASRTFGCSWE
FIPVDDGWGESSLENLYFQGASHHHHHHHH
SEQ ID NO: 27-Nucleotide Sequence of rLHN/FA (His-tagged)
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CATGAACTGATTCATGTTCTGCATGGTCTGTATGGCACCTATATTACCGAATACAAAATTACCCCGAATGTGGTG
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCAATACCAAAAACAGCCTGTGCATTACCGTTAATAATCGCGACCTGTTTTTTATCGCA
AGCCAAGAAAGCTATGGCGAGAATACCATTAACACCTATAAAGAGATTGACGATACCACCACACTGGATCCGAGC
TTTGAAGATATTCTGGATAAAGTGATCCTGAACTTCAACGAACAGGTTATTCCGCAGATGCCGAATCGTAATGTT
AGCACCGATATTCAGAAAGACAACTACATCCCGAAATACGATTATAACCGCACCGACATTATCGATAGCTATGAA
GTTGGTCGCAACTACAACACCTTTTTCTATCTGAATGCCCAGAAATTTAGCCCGAACGAAAGCAATATTACCCTG
ACCAGCAGCTTTGATACAGGTCTGTTAGAAGGTAGCAAAGTGTATACCTTTTTCAGCAGCGATTTCATTAACAAC
ATCAACAAACCGGTTCAGGCCCTGCTGTTTATTGAATGGGTTAAACAGGTGATTCGCGATTTTACCACCGAAGCA
ACCAAAACCTCAACCGTTGATAAACTGAAAGATATTAGCCTGGTGGTGCCGTATATTGGTCTGGCACTGAATATT
GGTGATGAGATCTACAAACAGCATTTTGCAGAAGCAGTTGAACTGGTTGGTGCAGGTCTGCTGCTGGAATTTTCA
CCGGAATTTCTTATTCCGACGCTGCTGATTTTTACCATCAAAGGTTATCTGACCGGTAGCATTCGCGATAAAGAC
AAAATCATTAAAACCCTGGATAACGCCCTGAATGTTCGTGATCAGAAATGGAAAGAACTGTATCGTTGGGTTGTT
AGCAAATGGCTGACCACCATTAATACGCAGTTCAACAAACGCAAAGAACAAATGTACAAAGCCCTGAAAAATCAG
GCCACCGCCATTAAAAAGATCATCGAGAACAAATATAACAACTATACCACCGATGAAAAAAGCAAGATCGATAGC
AGCTATAACATCAACGAAATTGAACGCACCCTGAACGAAAAAATCAATCTGGCCATGAAAAACATCGAGCAGTTT
ATTACAGAAAGCAGCATTGCCTACCTGATCAATATCATCAACAACGAAACCATTCAGAAACTGAAAAGCTATGAT
GACCTGGTTCGTCGTTATCTGCTGGGTTATATTCGTAATCATAGCAGCATTCTGGGCAATAGCGTTGAAGAACTG
AATTCCAAAGTGAACAACCATCTGGATAATGGCATTCCGTTTGAACTGAGCAGTTATACCAATGATAGCCTGCTG
ATCCGCTACTTCAATAAAAACTATGGCGAAGAGAACCTGTATTTCCAGGGTGCCAGTCATCATCACCACCATCAC
CATCACTGA
SEQ ID NO: 28-Polypeptide Sequence of rLH_{N}/FA (His-tagged)
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGENIGALSINYQGQNIGSDINSIKKLQGQGVVSRVVRLCSNSNTKNSLCITVNNRDLFFIA
SQESYGENTINTYKEIDDTTTLDPSFEDILDKVILNFNEQVIPQMPNRNVSTDIQKDNYIPKYDYNRTDIIDSYE
VGRNYNTFFYLNAQKFSPNESNITLTSSFDTGLLEGSKVYTFFSSDFINNINKPVQALLFIEWVKQVIRDETTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYFNKNYGEENLYFQGASHHHHHHHH
SEQ ID NO: 29-Nucleotide Sequence of rH_{C}/FA (His-tagged)
ATGCTGAAGTATAACTGCATCCTGAACATCAAATATGAGATGGATCGTGATAAACTGGTTGATAGCAGCGGTTAT
CGTAGCCGTATCAATATTGGCACCGGTGTGAAATTTAGCGAGATCGATAAAAATCAGGTGCAGCTGAGCAATCTG
GAAAGCAGCAAAATTGAAGTGATTCTGAATAACGGCGTGATCTACAATAGCATGTATGAAAACTTTTCGACCAGC
TTCTGGATTCGCATTCCGAAATACTTTCGCAACATCAACAACGAGTACAAGATTATCAGCTGTATGCAGAATAAT
AGCGGTTGGGAAGTTAGCCTGAATTTCAGCAATATGAACAGCAAAATCATTTGGACCCTGCAGGATACCGAAGGT
ATCAAAAAAACCGTTGTGTTTCAGTACACCCAGAACATTAACATCAGCGATTACATTAACCGCTGGATCTTTGTG
ACCATTACCAATAATCGTCTGAGCAACAGCAAGATCTATATTAACGGTCGCCTGATTAACGAAGAGAGCATTAGC
GATCTGGGTAATATTCATGCCAGCAACAACATCATGTTTAAACTGGATGGTTGTCGTGATCCGCATCGTTATATT
TGGATCAAATACTTCAACCTGTTTGATAAAGAACTGAACAAAAAAGAAATCAAAGACCTGTATGATAACCAGAGC
AATAGCGGCATTCTGAAAGATTTTTGGGGTGATTATCTGCAGTATGACAAACCGTATTACATGCTGAATCTGTAC
GATCCGAACAAATATCTGGATGTGAATAATGTGGGTATCCGTGGCTATATGTATCTGAAAGGTCCGCGTGGTCGT
ATTGTTACCACCAACATTTATCTGAATAGCACCCTGTATATGGGCACCAAATTCATCATTAAAAAGTATGCCAGC
GGCAACAAAGATAACATTGTGCGTAATAATGATCGCGTGTATATCAATGTGGTGGTGAAGAATAAAGAATATCGT
CTGGCCACCAATGCAAGCCAGGCAGGCGTTGAAAAAATTCTGAGCGCAGTTGAAATTCCGGATGTTGGTAATCTG
AGCCAGGTTGTTGTTATGAAAAGCGAAAATGATCAGGGCATTCGCAACAAATGCAAAATGAATCTGCAGGACAAT
AACGGCAACGATATTGGTTTTATTGGCTTCCACCAGTTCAACAACATTGCAAAACTGGTGGCGAGCAATTGGTAT
AATCGTCAGATTGGTAAAGCAAGCCGTACCTTTGGTTGTAGCTGGGAATTTATTCCGGTTGATGATGGTTGGGGT
GAAAGCAGCCTGGAAAATCTGTATTTTCAGGGTGCAAGTCATCATCACCACCATCACCATCATTAA
SEQ ID NO: 30-Polypeptide Sequence of rH_{C}/FA (His-tagged)
MLKYNCILNIKYEMDRDKLVDSSGYRSRINIGTGVKFSEIDKNQVQLSNLESSKIEVILNNGVIYNSMYENFSTS
FWIRIPKYFRNINNEYKIISCMQNNSGWEVSLNFSNMNSKIIWTLQDTEGIKKTVVFQYTQNINISDYINRWIFV
TITNNRLSNSKIYINGRLINEESISDLGNIHASNNIMFKLDGCRDPHRYIWIKYFNLFDKELNKKEIKDLYDNQS
NSGILKDFWGDYLQYDKPYYMLNLYDPNKYLDVNNVGIRGYMYLKGPRGRIVTTNIYLNSTLYMGTKFIIKKYAS
GNKDNIVRNNDRVYINVVVKNKEYRLATNASQAGVEKILSAVEIPDVGNLSQVVVMKSENDQGIRNKCKMNLQDN
NGNDIGFIGFHQFNNIAKLVASNWYNRQIGKASRTFGCSWEFIPVDDGWGESSLENLYFQGASHHHHHHHH
SEQ ID NO: 31-Nucleotide Sequence of rLC/FA (His-tagged)
ATGCCGGTTGTGATTAACAGCTTCAATTATGATGATCCGGTGAACGATAACACCATCATTTATATCCGTCCGCCT
TATTATGAAACCAGCAACACCTATTTCAAAGCCTTCCAGATTATGGATAACGTGTGGATTATTCCGGAACGTTAT
CGTCTGGGTATTGATCCGAGCCTGTTTAATCCGCCTGTTAGCCTGAAAGCAGGTAGTGATGGTTATTTTGATCCG
AATTATCTGAGCACCAACACCGAGAAAAACAAATACCTGCAGATTATGATCAAGCTGTTCAAACGCATTAATAGC
AAACCGGCAGGTCAGATTCTGCTGGAAGAAATCAAAAATGCAATTCCGTATCTGGGCAACAGCTATACCCAAGAA
GAACAGTTTACCACCAATAATCGTACCGTGAGCTTTAATGTTAAACTGGCCAATGGTAATATCGTTCAGCAGATG
GCAAATCTGATTATTTGGGGTCCGGGTCCTGATCTGACCACAAATAAAACCGGTGGTATCATCTATAGCCCGTAT
CAGAGCATGGAAGCAACCCCGTATAAAGATGGTTTTGGTAGCATTATGACCGTGGAATTTAGTCCGGAATATGCA
ACCGCCTTTAACGATATTTCAATTGCAAGCCATAGTCCGTCGCTGTTTATCAAAGATCCGGCACTGATTCTGATG
CATGAACTGATTCATGTTCTGCATGGTCTGTATGGCACCTATATTACCGAATACAAAATTACCCCGAATGTGGTG
CAGAGCTATATGAAAGTTACCAAACCGATTACCAGCGCAGAATTTCTGACCTTTGGTGGTCGTGATCGCAATATT
GTTCCGCAGAGCATTCAGAGCCAGCTGTATAACAAAGTTCTGAGCGATTATAAACGTATTGCCAGCCGTCTGAAT
AAAGTTAATACCGCAACCGCACTGATCAACATCGATGAATTCAAAAACCTGTACGAGTGGAAATACCAGTTTGCC
AAAGATAGCAATGGTGTGTATAGCGTGGATCTGAACAAATTTGAGCAGCTGTACAAAAAAATCTATAGCTTCACC
GAATTCAACCTGGCCTATGAGTTTAAAATCAAAACCCGTCTGGGTTATCTGGCCGAAAATTTTGGTCCGTTTTAT
CTGCCGAATCTGCTGGATGATAGCATTTATACCGAAGTGGATGGTTTTAACATTGGTGCACTGAGCATTAACTAT
CAGGGTCAGAATATTGGCAGCGATATCAACAGCATCAAAAAACTGCAAGGTCAGGGTGTTGTTAGCCGTGTTGTT
CGTCTGTGTAGCAATAGCGAAAATCTGTATTTTCAGGGTGCCAGTCATCATCACCACCATCACCATCACTGA
SEQ ID NO: 32-Polypeptide Sequence of rLC/FA (His-tagged)
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY
LPNLLDDSIYTEVDGFNIGALSINYQGQNIGSDINSIKKLQGQGVVSRVVRLCSNSENLYFQGASHHHHHHHH
SEQ ID NO: 33-Nucleotide Sequence of rBoNT/F(0) (His-tagged)
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACACCAGCTGATTTATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGTGATTCCGCGTAAAGGCACCAAAGCACCGCCTCGTCTGTGTATTCGTGTGAATAAT
CGTGAACTGTTTTTTGTTGCAAGCGAGAGCAGCTATAACGAGAATGATATTAACACCCCGAAAGAGATTGACGAT
ACCACCAATCTGAATAACAACTATCGCAACAATCTGGATGAAGTGATCCTGGATTATAACAGCGAAACCATTCCG
CAGATTAGCAATCAGACCCTGAATACCCTGGTTCAGGATGATAGCTATGTTCCGCGTTATGATAGCAATGGCACC
AGCGAAATTGAAGAACATAATGTGGTTGATCTGAACGTGTTCTTTTATCTGCATGCACAGAAAGTGCCGGAAGGT
GAAACCAATATTAGCCTGACCAGCAGCATTGATACCGCACTGAGCGAAGAAAGCCAGGTTTATACCTTTTTTAGC
AGCGAATTCATCAACACCATTAACAAACCGGTTCATGCAGCACTGTTTATTAGCTGGATTAATCAGGTGATTCGC
GATTTTACCACCGAAGCAACCCAGAAAAGCACCTTTGATAAAATTGCCGATATTAGTCTGGTGGTGCCGTATGTT
GGTCTGGCACTGAATATTGGTAATGAAGTGCAGAAAGAGAACTTTAAAGAAGCCTTCGAACTGTTAGGTGCCGGT
ATTCTGCTGGAATTTGTGCCGGAACTGCTGATTCCGACCATTCTGGTTTTTACCATTAAGAGCTTTATTGGCAGC
AGCGAGAACAAGAACAAAATCATTAAAGCCATCAACAACAGCCTGATGGAACGCGAAACCAAATGGAAAGAAATT
TACAGCTGGATTGTGAGCAATTGGCTGACCCGTATCAATACCCAGTTTAACAAACGCAAAGAACAAATGTATCAG
GCCCTGCAGAATCAGGTTGATGCAATTAAAACCGTGATCGAATACAAATACAACAACTATACCAGCGACGAACGT
AATCGCCTGGAAAGCGAATACAACATTAATAACATTCGCGAAGAACTGAACAAAAAAGTGAGCCTGGCAATGGAA
AACATCGAACGTTTTATTACCGAAAGCAGCATCTTCTACCTGATGAAACTGATTAACGAAGCCAAAGTTAGCAAA
CTGCGCGAATATGATGAAGGCGTTAAAGAATATCTGCTGGACTATATTAGCGAACATCGTAGCATTCTGGGTAAT
AGCGTTCAAGAGCTGAATGATCTGGTTACCAGCACACTGAATAATAGCATTCCGTTTGAACTGAGCAGCTACACC
AACGATAAAATCCTGATCCTGTACTTCAACAAACTGTACAAGAAGATCAAGGACAACAGCATACTGGATATGCGC
TATGAAAACAACAAGTTCATTGATATCAGCGGCTATGGTAGCAACATTAGCATTAATGGTGATGTGTATATCTAC
AGCACCAACCGCAATCAGTTTGGTATTTATAGCAGCAAACCGAGCGAAGTTAATATTGCGCAGAATAACGATATC
ATCTACAACGGTCGCTATCAGAACTTTAGCATTAGCTTTTGGGTTCGCATTCCGAAATACTTTAACAAGGTGAAC
CTGAACAACGAGTACACCATTATTGATTGCATTCGCAATAATAACAGCGGCTGGAAAATCAGCCTGAACTATAAC
AAAATTATCTGGACCCTGCAGGATACCGCAGGTAATAATCAGAAACTGGTGTTTAACTACACCCAGATGATTAGC
ATCAGCGACTATATCAACAAATGGATCTTTGTGACCATTACCAACAATCGTCTGGGTAACAGCCGCATTTATATC
AATGGCAATCTGATCGACGAAAAAAGCATTTCAAATCTGGGCGATATTCACGTGAGCGATAACATTCTGTTCAAA
ATTGTTGGCTGCAACGATACCCGTTATGTTGGTATTCGTTACTTCAAAGTGTTTGATACGGAACTGGGCAAAACG
GAAATTGAAACCCTGTATAGTGATGAACCGGATCCGAGCATTCTGAAAGATTTTTGGGGTAATTATCTGCTGTAC
AACAAACGCTACTATCTGCTGAACCTGCTGCGTACCGATAAAAGCATTACACAGAATAGCAACTTTCTGAACATC
AATCAGCAGCGTGGTGTTTATCAGAAACCGAACATTTTTAGCAACACCCGTCTGTATACCGGTGTGGAAGTTATT
ATTCGTAAAAACGGTAGCACCGATATCAGCAACACCGATAACTTTGTGCGTAAAAATGACCTGGCCTATATTAAC
GTTGTTGATCGTGATGTTGAGTATCGTCTGTATGCGGATATTAGCATTGCCAAACCGGAAAAGATTATCAAACTG
ATCCGTACCAGCAACAGCAATAATTCACTGGGTCAGATTATCGTGATGGACAGCATTGGTAACAATTGCACCATG
AATTTCCAGAACAATAACGGTGGTAATATTGGCCTGCTGGGCTTTCATAGCAATAATCTGGTTGCAAGCAGCTGG
TATTACAACAACATCCGTAAAAATACCAGCAGTAATGGTTGCTTTTGGAGCTTTATCAGTAAAGAACATGGCTGG
CAAGAAAACGAGAACCTGTATTTTCAGGGTGCAAGTCATCATCACCATCACCACCATCATTAA
SEQ ID NO: 34-Polypeptide Sequence of rBoNT/F(0) (His-tagged)
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHQLIYALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTEDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYENKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDCIRNNNSGWKISLNYN
KIIWTLQDTAGNNQKLVFNYTQMISISDYINKWIFVTITNNRLGNSRIYINGNLIDEKSISNLGDIHVSDNILFK
IVGCNDTRYVGIRYFKVFDTELGKTEIETLYSDEPDPSILKDFWGNYLLYNKRYYLLNLLRTDKSITQNSNFLNI
NQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGW
QENENLYFQGASHHHHHHHH
SEQ ID NO: 35-Nucleotide Sequence of rL_{H}N/F (His-tagged)
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACATGAACTGATTCATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGTGATTCCGCGTAAAGGCACCAAAGCACCGCCTCGTCTGTGTATTCGTGTGAATAAT
CGTGAACTGTTTTTTGTTGCAAGCGAGAGCAGCTATAACGAGAATGATATTAACACCCCGAAAGAGATTGACGAT
ACCACCAATCTGAATAACAACTATCGCAACAATCTGGATGAAGTGATCCTGGATTATAACAGCGAAACCATTCCG
CAGATTAGCAATCAGACCCTGAATACCCTGGTTCAGGATGATAGCTATGTTCCGCGTTATGATAGCAATGGCACC
AGCGAAATTGAAGAACATAATGTGGTTGATCTGAACGTGTTCTTTTATCTGCATGCACAGAAAGTGCCGGAAGGT
GAAACCAATATTAGCCTGACCAGCAGCATTGATACCGCACTGAGCGAAGAAAGCCAGGTTTATACCTTTTTTAGC
AGCGAATTCATCAACACCATTAACAAACCGGTTCATGCAGCACTGTTTATTAGCTGGATTAATCAGGTGATTCGC
GATTTTACCACCGAAGCAACCCAGAAAAGCACCTTTGATAAAATTGCCGATATTAGTCTGGTGGTGCCGTATGTT
GGTCTGGCACTGAATATTGGTAATGAAGTGCAGAAAGAGAACTTTAAAGAAGCCTTCGAACTGTTAGGTGCCGGT
ATTCTGCTGGAATTTGTGCCGGAACTGCTGATTCCGACCATTCTGGTTTTTACCATTAAGAGCTTTATTGGCAGC
AGCGAGAACAAGAACAAAATCATTAAAGCCATCAACAACAGCCTGATGGAACGCGAAACCAAATGGAAAGAAATT
TACAGCTGGATTGTGAGCAATTGGCTGACCCGTATCAATACCCAGTTTAACAAACGCAAAGAACAAATGTATCAG
GCCCTGCAGAATCAGGTTGATGCAATTAAAACCGTGATCGAATACAAATACAACAACTATACCAGCGACGAACGT
AATCGCCTGGAAAGCGAATACAACATTAATAACATTCGCGAAGAACTGAACAAAAAAGTGAGCCTGGCAATGGAA
AACATCGAACGTTTTATTACCGAAAGCAGCATCTTCTACCTGATGAAACTGATTAACGAAGCCAAAGTTAGCAAA
CTGCGCGAATATGATGAAGGCGTTAAAGAATATCTGCTGGACTATATTAGCGAACATCGTAGCATTCTGGGTAAT
AGCGTTCAAGAGCTGAATGATCTGGTTACCAGCACACTGAATAATAGCATTCCGTTTGAACTGAGCAGCTACACC
AACGATAAAATCCTGATCCTGTACTTCAACAAACTGTACAAGAAAGAAAACCTGTATTTTCAGGGTGCAAGCCAT
CATCACCACCATCACCATCATTAA
SEQ ID NO: 36-Polypeptide Sequence of rL_{H}N/F (His-tagged)
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGFNIGNLAVNNRGQNIKLNPKIIDSIPDKGLVEKIVKFCKSVIPRKGTKAPPRLCIRVNN
RELFFVASESSYNENDINTPKEIDDTTNLNNNYRNNLDEVILDYNSETIPQISNQTLNTLVQDDSYVPRYDSNGT
SEIEEHNVVDLNVFFYLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELLIPTILVFTIKSFIGS
SENKNKIIKAINNSLMERETKWKEIYSWIVSNWLTRINTQFNKRKEQMYQALQNQVDAIKTVIEYKYNNYTSDER
NRLESEYNINNIREELNKKVSLAMENIERFITESSIFYLMKLINEAKVSKLREYDEGVKEYLLDYISEHRSILGN
SVQELNDLVTSTLNNSIPFELSSYTNDKILILYENKLYKKENLYFQGASHHHHHHHH
SEQ ID NO: 37-Nucleotide Sequence of rH_{C}/F (His-tagged)
ATGATCAAGGATAACAGCATTCTGGATATGCGCTATGAGAACAACAAATTCATTGATATTAGCGGCTATGGCAGC
AACATTAGCATTAATGGTGATGTGTATATCTACAGCACCAACCGTAATCAGTTTGGCATTTATAGCAGCAAACCG
AGCGAAGTTAATATTGCCCAGAACAACGATATCATCTATAACGGTCGCTATCAGAACTTCAGCATTAGCTTTTGG
GTTCGCATTCCGAAATACTTCAATAAGGTGAACCTGAACAACGAGTATACCATCATTGATTGCATTCGCAATAAT
AACAGCGGCTGGAAAATTAGCCTGAACTACAACAAAATTATCTGGACCCTGCAGGATACCGCAGGTAATAATCAG
AAACTGGTGTTTAACTACACCCAGATGATTAGCATCAGCGACTATATCAACAAATGGATCTTTGTGACCATTACC
AATAATCGCCTGGGTAATAGCCGCATTTATATCAATGGTAACCTGATCGATGAGAAAAGCATTAGCAATCTGGGT
GATATTCATGTGAGCGATAACATCCTGTTTAAAATCGTGGGTTGTAACGATACCCGTTATGTTGGTATTCGCTAC
TTCAAAGTGTTTGATACCGAACTGGGTAAAACCGAAATTGAAACCCTGTATAGTGATGAACCGGATCCGAGCATT
CTGAAAGATTTTTGGGGTAATTATCTGCTGTACAACAAACGCTACTATCTGCTGAATCTGCTGCGTACCGATAAA
TCAATTACCCAGAATAGCAACTTCCTGAACATTAATCAGCAGCGTGGTGTTTATCAGAAACCGAACATTTTTAGC
AACACCCGTCTGTATACCGGTGTGGAAGTTATTATTCGTAAAAATGGCAGCACCGATATCAGCAACACCGATAAC
TTTGTTCGCAAAAATGATCTGGCGTATATCAACGTTGTTGATCGTGATGTTGAATATCGTCTGTATGCCGATATT
AGCATTGCCAAACCGGAAAAAATCATCAAACTGATCCGTACCAGCAACAGCAATAATTCACTGGGTCAGATTATT
GTGATGGATAGCATTGGTAATAACTGCACCATGAACTTTCAGAACAATAACGGTGGTAATATTGGTCTGCTGGGC
TTTCATAGTAATAATCTGGTTGCAAGCAGCTGGTATTATAACAACATCCGTAAAAATACCAGCAGCAATGGTTGC
TTTTGGAGCTTTATTAGCAAAGAACATGGCTGGCAAGAAAACGAGAATCTGTATTTTCAGGGTGCAAGTCATCAT
CACCACCATCACCATCATTAA
SEQ ID NO: 38-Polypeptide Sequence of rH_{C}/F (His-tagged)
MIKDNSILDMRYENNKFIDISGYGSNISINGDVYIYSTNRNQFGIYSSKPSEVNIAQNNDIIYNGRYQNFSISFW
VRIPKYFNKVNLNNEYTIIDCIRNNNSGWKISLNYNKIIWTLQDTAGNNQKLVFNYTQMISISDYINKWIFVTIT
NNRLGNSRIYINGNLIDEKSISNLGDIHVSDNILFKIVGCNDTRYVGIRYFKVEDTELGKTEIETLYSDEPDPSI
LKDFWGNYLLYNKRYYLLNLLRTDKSITQNSNFLNINQQRGVYQKPNIFSNTRLYTGVEVIIRKNGSTDISNTDN
FVRKNDLAYINVVDRDVEYRLYADISIAKPEKIIKLIRTSNSNNSLGQIIVMDSIGNNCTMNFQNNNGGNIGLLG
FHSNNLVASSWYYNNIRKNTSSNGCFWSFISKEHGWQENENLYFQGASHHHHHHHH
SEQ ID NO: 39-Nucleotide Sequence of rLC/F (His-tagged)
ATGCCGGTTGTGATTAACAGCTTCAATTATAACGATCCGGTGAACGATGATACCATCCTGTATATGCAGATTCCG
TATGAAGAGAAAAGCAAAAAGTACTACAAAGCCTTTGAGATCATGCGCAACGTTTGGATTATTCCGGAACGTAAT
ACCATTGGCACCGATCCGAGCGATTTTGATCCGCCTGCAAGCCTGGAAAATGGTAGCAGCGCATATTATGATCCG
AATTATCTGACCACCGATGCCGAAAAAGATCGTTATCTGAAAACCACCATCAAACTGTTCAAACGCATTAATAGC
AATCCGGCAGGCGAAGTTCTGCTGCAAGAAATTAGCTATGCAAAACCGTATCTGGGCAATGAACATACCCCGATT
AATGAATTTCATCCGGTTACACGTACCACGAGCGTTAACATTAAAAGCAGCACCAATGTGAAGTCCAGCATTATT
CTGAATCTGCTGGTTTTAGGTGCAGGTCCGGATATTTTTGAAAATTCAAGCTATCCGGTGCGCAAACTGATGGAT
AGCGGTGGTGTGTATGATCCGTCAAATGATGGTTTTGGCAGCATTAACATTGTGACCTTTAGTCCGGAATATGAA
TACACCTTCAACGATATTAGCGGTGGCTATAATAGCAGCACCGAAAGTTTTATTGCAGATCCGGCAATTAGCCTG
GCACATGAACTGATTCATGCACTGCATGGTCTGTATGGTGCACGTGGTGTTACCTATAAAGAAACCATTAAAGTT
AAACAGGCACCGCTGATGATTGCGGAAAAACCGATTCGTCTGGAAGAATTTCTGACCTTTGGTGGTCAGGATCTG
AACATTATTACCAGCGCAATGAAAGAGAAAATCTATAATAACCTGCTGGCCAACTATGAGAAAATTGCAACCCGT
CTGAGCCGTGTTAATAGCGCACCTCCTGAATATGATATCAACGAGTATAAAGACTATTTTCAGTGGAAATACGGC
CTGGATAAAAATGCAGATGGTAGCTATACCGTGAACGAGAACAAATTTAACGAGATCTACAAAAAACTGTATAGC
TTCACCGAAATCGATCTGGCCAACAAATTCAAAGTGAAATGCCGCAACACCTACTTCATCAAATATGGCTTTCTG
AAAGTTCCGAACCTGCTTGATGATGATATCTATACCGTTAGCGAAGGCTTTAACATTGGTAATCTGGCCGTTAAT
AATCGCGGTCAGAACATTAAACTGAACCCGAAAATTATCGATAGCATCCCGGATAAAGGCCTGGTTGAAAAAATT
GTGAAATTCTGCAAAAGCGAGAACCTGTATTTTCAGGGTGCAAGTCATCATCACCATCACCACCATCATTAA
SEQ ID NO: 40-Polypeptide Sequence of rLC/F (His-tagged)
MPVVINSFNYNDPVNDDTILYMQIPYEEKSKKYYKAFEIMRNVWIIPERNTIGTDPSDFDPPASLENGSSAYYDP
NYLTTDAEKDRYLKTTIKLFKRINSNPAGEVLLQEISYAKPYLGNEHTPINEFHPVTRTTSVNIKSSTNVKSSII
LNLLVLGAGPDIFENSSYPVRKLMDSGGVYDPSNDGFGSINIVTFSPEYEYTENDISGGYNSSTESFIADPAISL
AHELIHALHGLYGARGVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKFKVKCRNTYFIKYGFL
KVPNLLDDDIYTVSEGENIGNLAVNNRGQNIKLNPKIIDSIPDKGLVEKIVKFCKSENLYFQGASHHHHHHHH
SEQ ID NO: 41-Nucleotide Sequence of Cationic rH_{C}/A (His-tagged)
ATGATCATCAACACCAGCATTCTGAACCTGCGTTATGAAAGCAAACATCTGATTGATCTGAGCCGTTATGCCAGC
AAAATCAATATAGGCAGCAAGGTTAACTTCGACCCGATTGACAAAAATCAGATACAGCTGTTTAATCTGGAAAGC
AGCAAAATTGAGGTGATCCTGAAAAAAGCGATCGTGTATAATAGCATGTACGAGAATTTTTCGACCAGCTTTTGG
ATTCGCATCCCGAAATACTTTAACAAGATTAGCCTGAACAACGAGTATACCATCATTAACTGCATGGAAAACAAT
AGCGGTTGGAAAGTCAGCCTGAATTATGGCGAAATTATCTGGACCCTGCAGGATACCAAAGAAATCAAACAGCGT
GTGGTGTTCAAATACAGCCAGATGATTAATATCAGCGACTATATCAACCGCTGGATTTTTGTGACCATTACCAAT
AATCGGCTGAACAAGAGCAAGATCTATATTAACGGTCGTCTGATTGACCAGAAACCGATTAGTAATCTGGGTAAT
ATTCATGCGAGCAACAAAATCATGTTTAAACTGGATGGTTGCCGTGATACCCATCGTTATATTTGGATCAAATAC
TTCAACCTGTTCGATAAAGAGTTGAACGAAAAAGAAATTAAAGACCTGTACGATAACCAGAGCAATAGCGGCATA
CTGAAAGATTTTTGGGGAGATTATCTGCAGTATGACAAACCGTATTATATGCTGAATCTGTACGACCCGAATAAA
TACGTGGATGTTAATAATGTGGGCATCCGTGGTTATATGTACCTGAAAGGTCCGCGTGGTAGCGTTATGACCACA
AACATTTATCTGAATAGCAGCCTGTATCGCGGAACCAAATTCATCATTAAAAAGTATGCCAGCGGCAACAAGGAT
AATATTGTGCGTAATAATGATCGCGTGTACATTAACGTTGTGGTGAAGAATAAAGAATATCGCCTGGCAACCAAT
GCAAGCCAGGCAGGCGTTGAAAAAATTCTGAGTGCCCTGGAAATTCCGGATGTTGGTAATCTGAGCCAGGTTGTT
GTGATGAAAAGCAAAAACGATAAAGGCATCACCAACAAATGCAAGATGAATCTGCAGGACAATAACGGCAATGAT
ATTGGCTTCATTGGCTTTCACCAGTTTAACAACATTGCAAAACTGGTTGCGAGCAATTGGTATAATCGTCAGATT
GAACGTAGCAGTCGTACCCTGGGTTGTAGCTGGGAATTTATCCCTGTGGATGATGGTTGGGGTGAACGTCCGCTG
AAGCTTGCGGCCGCACTCGAGCACCACCACCACCACCACTGA
SEQ ID NO: 42-Polypeptide Sequence of Cationic rH_{C}/A (His-tagged)
MIINTSILNLRYESKHLIDLSRYASKINIGSKVNFDPIDKNQIQLFNLESSKIEVILKKAIVYNSMYENFSTSFW
IRIPKYFNKISLNNEYTIINCMENNSGWKVSLNYGEIIWTLQDTKEIKQRVVFKYSQMINISDYINRWIFVTITN
NRLNKSKIYINGRLIDQKPISNLGNIHASNKIMFKLDGCRDTHRYIWIKYENLEDKELNEKEIKDLYDNQSNSGI
LKDFWGDYLQYDKPYYMLNLYDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDKGITNKCKMNLQDNNGND
IGFIGFHQFNNIAKLVASNWYNRQIERSSRTLGCSWEFIPVDDGWGERPLKLAAALEHHHHHH
SEQ ID NO: 43-Nucleotide Sequence of rH_{C}/AB (His-tagged)
ATGATTCTGAACAATATTATCCTGAACCTGCGTTACAAAGACAACAATCTGATCGATCTGAGCGGCTATGGTGCA
AAAGTTGAAGTCTACGACGGTGTCGAACTGAACGATAAAAACCAGTTCAAACTGACCTCATCGGCTAACTCAAAA
ATTCGTGTGACGCAGAACCAAAACATCATCTTCAACTCGGTCTTTCTGGACTTCAGCGTGTCTTTCTGGATTCGC
ATCCCGAAATATAAAAATGATGGCATCCAGAACTACATCCATAACGAATACACCATCATCAACTGTATGAAAAAC
AACAGTGGTTGGAAAATTTCCATCCGTGGCAACCGCATTATCTGGACCCTGATTGATATCAATGGTAAAACGAAA
AGCGTGTTTTTCGAATACAACATCCGTGAAGATATCTCTGAATACATCAATCGCTGGTTTTTCGTGACCATTACG
AACAATCTGAACAATGCGAAAATCTATATCAACGGCAAACTGGAAAGTAATACCGACATCAAAGATATTCGTGAA
GTTATCGCCAACGGTGAAATCATCTTCAAACTGGATGGCGACATCGATCGCACCCAGTTCATTTGGATGAAATAC
TTCTCCATCTTCAACACGGAACTGAGTCAGTCCAATATCGAAGAACGCTACAAAATCCAATCATACTCGGAATAC
CTGAAAGATTTCTGGGGTAACCCGCTGATGTACAACAAAGAATACTACATGTTCAACGCGGGCAACAAAAACTCA
TACATCAAACTGAAAAAAGATTCGCCGGTGGGTGAAATCCTGACCCGTAGCAAATACAACCAGAACTCTAAATAC
ATCAACTATCGCGATCTGTACATTGGCGAAAAATTTATTATCCGTCGCAAAAGCAACTCTCAGAGTATTAATGAT
GACATCGTGCGTAAAGAAGACTACATCTATCTGGATTTCTTTAATCTGAACCAAGAATGGCGCGTTTATACCTAC
AAATACTTCAAAAAAGAAGAAATGAAACTGTTCCTGGCCCCGATTTACGACAGCGATGAATTTTACAACACCATC
CAGATCAAAGAATACGATGAACAGCCGACGTATAGTTGCCAACTGCTGTTCAAAAAAGACGAAGAATCCACCGAT
GAAATTGGCCTGATTGGTATCCACCGTTTCTATGAAAGCGGTATCGTTTTCGAAGAATACAAAGATTACTTCTGT
ATCTCTAAATGGTATCTGAAAGAAGTCAAACGCAAACCGTACAACCTGAAACTGGGCTGCAACTGGCAATTTATC
CCGAAAGACGAAGGCTGGACCGAAAAGCTTGCGGCCGCACTCGAGCACCACCACCACCACCACTGA
SEQ ID NO: 44-Polypeptide Sequence of rH_{C}/AB (His-tagged)
MILNNIILNLRYKDNNLIDLSGYGAKVEVYDGVELNDKNQFKLTSSANSKIRVTQNQNIIFNSVFLDFSVSFWIR
IPKYKNDGIQNYIHNEYTIINCMKNNSGWKISIRGNRIIWTLIDINGKTKSVFFEYNIREDISEYINRWFFVTIT
NNLNNAKIYINGKLESNTDIKDIREVIANGEIIFKLDGDIDRTQFIWMKYFSIFNTELSQSNIEERYKIQSYSEY
LKDFWGNPLMYNKEYYMFNAGNKNSYIKLKKDSPVGEILTRSKYNQNSKYINYRDLYIGEKFIIRRKSNSQSIND
DIVRKEDYIYLDFFNLNQEWRVYTYKYFKKEEMKLFLAPIYDSDEFYNTIQIKEYDEQPTYSCQLLFKKDEESTD
EIGLIGIHRFYESGIVFEEYKDYFCISKWYLKEVKRKPYNLKLGCNWQFIPKDEGWTEKLAAALEHHHHHH
SEQ ID NO: 45-Nucleotide Sequence of rH_{C}/A Variant Y1117V H1253K (His-tagged)
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTTCaAaCAGTTCAACAATATTGCTAAACTGGTAGCGAGCAATTGGTACAATCGTCAGATT
GAGCGCAGCAGCCGTACTTTGGGCTGTAGCTGGGAGTTTATCCCGGTCGATGATGGTTGGGGCGAACGTCCGCTG
CACCATCACCATCACCATCACCATCACCATT
SEQ ID NO: 46-Polypeptide Sequence of rH_{C}/A Variant Y1117V H1253K (His-tagged)
MIINTSILNLRYESNHLIDLSRYASKINIGSKVNFDPIDKNQIQLENLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGFKQFNNIAKLVASNWYNRQIERSSRTLGCSWEFIPVDDGWGERPLHHHHHHHHHH
SEQ ID NO: 47-Nucleotide Sequence of rH_{C}/A Variant Y1117V F1252Y H1253K L1278F
(His-tagged)
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTaCaAaCAGTTCAACAATATTGCTAAACTGGTAGCGAGCAATTGGTACAATCGTCAGATT
GAGCGCAGCAGCCGTACTTTtGGCTGTAGCTGGGAGTTTATCCCGGTCGATGATGGTTGGGGCGAACGTCCGCTG
CACCATCACCATCACCATCACCATCACCATTAA
SEQ ID NO: 48-Polypeptide Sequence of rH_{C}/A Variant Y1117V F1252Y H1253K
L1278F (His-tagged)
MIINTSILNLRYESNHLIDLSRYASKINIGSKVNFDPIDKNQIQLENLESSKIEVILKNAIVYNSMYENESTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGYKQFNNIAKLVASNWYNRQIERSSRTFGCSWEFIPVDDGWGERPLHHHHHHHHHH
SEQ ID NO: 49-Nucleotide Sequence of rH_{C}/A Variant Y1117V F1252Y H1253K L1278H
(His-tagged)
ATGATCATCAATACTAGCATTCTGAACCTGCGTTACGAGAGCAATCATCTGATTGATCTGAGCCGTTATGCAAGC
AAGATCAACATCGGTAGCAAGGTCAATTTTGACCCGATCGATAAGAACCAGATCCAGCTGTTTAATCTGGAATCG
AGCAAAATTGAGGTTATCCTGAAAAACGCCATTGTCTACAACTCCATGTACGAGAATTTCTCCACCAGCTTCTGG
ATTCGCATCCCGAAATACTTCAACAGCATTAGCCTGAACAACGAGTATACTATCATCAACTGTATGGAGAACAAC
AGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAAT
AACCGTCTGAATAACAGCAAGATTTACATCAATGGTCGCTTGATCGATCAGAAACCGATTAGCAACCTGGGTAAT
ATCCACGCAAGCAACAACATTATGTTCAAATTGGACGGTTGCCGCGATACCCATCGTTATATCTGGATCAAGTAT
TTCAACCTGTTTGATAAAGAACTGAATGAGAAGGAGATCAAAGATTTGTATGACAACCAATCTAACAGCGGCATT
TTGAAGGACTTCTGGGGCGATTATCTGCAATACGATAAGCCGTACTATATGCTGAACCTGgtTGATCCGAACAAA
TATGTGGATGTCAATAATGTGGGTATTCGTGGTTACATGTATTTGAAGGGTCCGCGTGGCAGCGTTATGACGACC
AACATTTACCTGAACTCTAGCCTGTACCGTGGTACGAAATTCATCATTAAGAAATATGCCAGCGGCAACAAAGAT
AACATTGTGCGTAATAACGATCGTGTCTACATCAACGTGGTCGTGAAGAATAAAGAGTACCGTCTGGCGACCAAC
GCTTCGCAGGCGGGTGTTGAGAAAATTCTGAGCGCGTTGGAGATCCCTGATGTCGGTAATCTGAGCCAAGTCGTG
GTTATGAAGAGCAAGAACGACCAGGGTATCACTAACAAGTGCAAGATGAACCTGCAAGACAACAATGGTAACGAC
ATCGGCTTTATTGGTTaCaAaCAGTTCAACAATATTGCTAAACTGGTAGCGAGCAATTGGTACAATCGTCAGATT
GAGCGCAGCAGCCGTACTcatGGCTGTAGCTGGGAGTTTATCCCGGTCGATGATGGTTGGGGCGAACGTCCGCTG
CACCATCACCATCACCAT
SEQ ID NO: 50-Polypeptide Sequence of rH_{C}/A Variant Y1117V F1252Y H1253K
L1278H (His-tagged)
MIINTSILNLRYESNHLIDLSRYASKINIGSKVNFDPIDKNQIQLFNLESSKIEVILKNAIVYNSMYENFSTSFW
IRIPKYFNSISLNNEYTIINCMENNSGWKVSLNYGEIIWTLQDTQEIKQRVVFKYSQMINISDYINRWIFVTITN
NRLNNSKIYINGRLIDQKPISNLGNIHASNNIMFKLDGCRDTHRYIWIKYFNLFDKELNEKEIKDLYDNQSNSGI
LKDFWGDYLQYDKPYYMLNLVDPNKYVDVNNVGIRGYMYLKGPRGSVMTTNIYLNSSLYRGTKFIIKKYASGNKD
NIVRNNDRVYINVVVKNKEYRLATNASQAGVEKILSALEIPDVGNLSQVVVMKSKNDQGITNKCKMNLQDNNGND
IGFIGYKQFNNIAKLVASNWYNRQIERSSRTHGCSWEFIPVDDGWGERPLHHHHHH
SEQ ID NO: 51-Polypeptide Sequence of BoNT/A-UniProt P10845
MPFVNKQFNYKDPVNGVDIAYIKIPNVGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTENEDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKELNQCSVSYLMNSMIPYGVKRLEDEDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENFSTSFWIRIPKYENSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYENLEDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 52-Polypeptide Sequence of BoNT/B-UniProt P10844
MPVTINNENYNDPIDNNNIIMMEPPFARGTGRYYKAFKITDRIWIIPERYTFGYKPEDFN
KSSGIFNRDVCEYYDPDYLNTNDKKNIFLQTMIKLENRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVENNVQENKGASIFNRRGYFSDPALILMHELIHVLHGLY
GIKVDDLPIVPNEKKFFMQSTDAIQAEELYTFGGQDPSIITPSTDKSIYDKVLQNERGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGENISDKDMEKEYRGQNKAINKQA
YEEISKEHLAVYKIQMCKSVKAPGICIDVDNEDLFFIADKNSESDDLSKNERIEYNTQSN
YIENDEPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDENDINSKLNEGINQAIDNINNFINGCSV
SYLMKKMIPLAVEKLLDFDNTLKKNLLNYIDENKLYLIGSAEYEKSKVNKYLKTIMPFDL
SIYTNDTILIEMENKYNSEILNNIILNLRYKDNNLIDLSGYGAKVEVYDGVELNDKNQEK
LTSSANSKIRVTQNQNIIFNSVELDESVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIENTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
SEQ ID NO: 53-Polypeptide Sequence of BoNT/C-UniProt P18640
MPITINNFNYSDPVDNKNILYLDTHLNTLANEPEKAFRITGNIWVIPDRFSRNSNPNLNK
PPRVTSPKSGYYDPNYLSTDSDKDPFLKEIIKLFKRINSREIGEELIYRLSTDIPFPGNN
NTPINTFDFDVDENSVDVKTRQGNNWVKTGSINPSVIITGPRENIIDPETSTFKLTNNTF
AAQEGFGALSIISISPREMLTYSNATNDVGEGRESKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSENKYIGEYKQKLIRKYRFVVESSGEVTVNRNKEVELYNELTQIFTE
FNYAKIYNVQNRKIYLSNVYTPVTANILDDNVYDIQNGENIPKSNLNVLEMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEEDRNTKAKLINLIDSHNIILVGEVDKLKAKVNNSF
QNTIPFNIFSYTNNSLLKDIINEYENNINDSKILSLQNRKNTLVDTSGYNAEVSEEGDVQ
LNPIFPFDEKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILFNSLQYTNVVKDYWGNDLRYNKEYYMVNIDYLNRYMYANSRQIVENTRRNNN
DENEGYKIIIKRIRGNTNDTRVRGGDILYFDMTINNKAYNLFMKNETMYADNHSTEDIYA
IGLREQTKDINDNIIFQIQPMNNTYYYASQIFKSNENGENISGICSIGTYRERLGGDWYR
HNYLVPTVKQGNYASLLESTSTHWGFVPVSE
SEQ ID NO: 54-Polypeptide Sequence of BoNT/D-UniProt P19321
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPEMGDS
STPEDTFDFTRHTTNIAVEKFENGSWKVTNIITPSVLIFGPLPNILDYTASLTLQGQQSN
PSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNEDKDNTGNFVVNIDKENSLYSDLTNVMSE
VVYSSQYNVKNRTHYFSRHYLPVFANILDDNIYTIRDGENLINKGENIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANE
VVEDFTTNIMKKDTLDKISDVSVIIPYIGPALNIGNSALRGNENQAFATAGVAFLLEGFP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKEDLRTKTELINLIDSHNIILVGEVDRLKAKVNESFENTM
PFNIFSYTNNSLLKDIINEYENSINDSKILSLQNKKNALVDTSGYNAEVRVGDNVQLNTI
YTNDFKLSSSGDKIIVNLNNNILYSAIYENSSVSFWIKISKDLINSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVEGIDENIDENQMLWIRDENIFSKELSNEDINIVYEGQI
LRNVIKDYWGNPLKEDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLS
TSSFWKFISRDPGWVE
SEQ ID NO: 55-Polypeptide Sequence of BoNT/E-UniProt Q00496
MPKINSFNYNDPVNDRTILYIKPGGCQEFYKSENIMKNIWIIPERNVIGTTPQDFHPPTS
LKNGDSSYYDPNYLQSDEEKDRELKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTESPEYSFRENDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLRTKFQVKCRQTYIGQYKYFKL
SNLLNDSIYNISEGYNINNLKVNFRGQNANLNPRIITPITGRGLVKKIIRFCKNIVSVKG
IRKSICIEINNGELFFVASENSYNDDNINTPKEIDDTVTSNNNYENDLDQVILNENSESA
PGLSDEKLNLTIQNDAYIPKYDSNGTSDIEQHDVNELNVEFYLDAQKVPEGENNVNLTSS
IDTALLEQPKIYTFFSSEFINNVNKPVQAALFVSWIQQVLVDFTTEANQKSTVDKIADIS
IVVPYIGLALNIGNEAQKGNEKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRELTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYENIFDKELDETEIQTLYSNEPNTNILKDEWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNCTMNF
KNNNGNNIGLLGFKADTVVASTWYYTHMRDHTNSNGCFWNFISEEHGWQEK
SEQ ID NO: 56-Polypeptide Sequence of BoNT/F-UniProt A7GBG3
MPVVINSFNYNDPVNDDTILYMQIPYEEKSKKYYKAFEIMRNVWIIPERNTIGTDPSDFD
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTENDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTEDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGN
SRIYINGNLIDEKSISNLGDIHVSDNILFKIVGCNDTRYVGIRYFKVEDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
SEQ ID NO: 57-Polypeptide Sequence of BoNT/G-UniProt Q60393
MPVNIKXFNYNDPINNDDIIMMEPENDPGPGTYYKAFRIIDRIWIVPERFTYGFQPDQFN
ASTGVFSKDVYEYYDPTYLKTDAEKDKFLKTMIKLENRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVENNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKEDKLYKALMFGFTETNL
AGEYGIKTRYSYFSEYLPPIKTEKLLDNTIYTQNEGENIASKNLKTEFNGQNKAVNKEAY
EEISLEHLVIYRIAMCKPVMYKNTGKSEQCIIVNNEDLFFIANKDSFSKDLAKAETIAYN
TQNNTIENNESIDQLILDNDLSSGIDLPNENTEPFTNEDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENEKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDENDIDFKLNQSINLAINNIDDFI
NQCSISYLMNRMIPLAVKKLKDEDDNLKRDLLEYIDTNELYLLDEVNILKSKVNRHLKDS
IPFDLSLYTKDTILIQVENNYISNISSNAILSLSYRGGRLIDSSGYGATMNVGSDVIFND
IGNGQFKLNNSENSNITAHQSKFVVYDSMFDNESINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDENIFGRELNATEVS
SLYWIQSSTNTLKDFWGNPLRYDTQYYLENQGMQNIYIKYFSKASMGETAPRTNENNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
SEQ ID NO: 58-Polypeptide Sequence of TeNT-UniProt P04958
MPITINNFRYSDPVNNDTIIMMEPPYCKGLDIYYKAFKITDRIWIVPERYEFGTKPEDFN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGENIESKDLKSEYKGQNMRVNT
NAFRNVDGSGLVSKLIGLCKKIIPPTNIRENLYNRTASLTDLGGELCIKIKNEDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVESTPIPFSYSKNLDCWVDNEEDIDVILKKSTILNLDINNDIISDIS
GENSSVITYPDAQLVPGINGKAIHLVNNESSEVIVHKAMDIEYNDMENNFTVSFWLRVPK
VSASHLEQYGTNEYSIISSMKKHSLSIGSGWSVSLKGNNLIWTLKDSAGEVRQITERDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWTND
SEQ ID NO: 59-Polypeptide Sequence of BoNT/X
MKLEINKENYNDPIDGINVITMRPPRHSDKINKGKGPFKAFQVIKNIWIVPERYNETNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNEDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGENISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDESDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNEDLETKKTLDK
FIKEKEDILGTNLSSSLRRKVSIRLNKNIAFDINDIPESEFDDLINQYKNEIEDYEVLNL
GAEDGKIKDLSGTTSDINIGSDIELADGRENKAIKIKGSENSTIKIAMNKYLRFSATDNF
SISFWIKHPKPTNLLNNGIEYTLVENENQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYENSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKTNWYFIPKDEGWDED
SEQ ID NO: 60-Nucleotide Sequence of mrBoNT/A
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAAGCATCTGATTGATCTGAGCCGTTATGCTAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAAGGCCATTGTCTACAACTCCATGTACGAGAATTTCTCCACCAGCTTCTGGATTCGCATCCCG
AAATACTTCAACAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AAGAGCAAGATTTACATCAATGGTCGCTTGATCGATCAGAAACCGATTAGCAACCTGGGTAATATCCACGCAAGC
AACAAGATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACAAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 61-Polypeptide Sequence of mrBoNT/A
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYENKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 62-Polypeptide Sequence of Unmodified/native BoNT/A1
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNEDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 63-Polypeptide Sequence of mrBoNT/AB
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
SEQ ID NO: 64-Polypeptide Sequence of mrBoNT/AB(0)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHQL
IYAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNEDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
SEQ ID NO: 65-Polypeptide Sequence of mrBoNT/A(0)
MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHQLIYAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGENLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESKHLIDLSRYASKINI
GSKVNFDPIDKNQIQLENLESSKIEVILKKAIVYNSMYENFSTSFWIRIP
KYFNKISLNNEYTIINCMENNSGWKVSLNYGEIIWTLQDTKEIKQRVVFK
YSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFI
GFHQFNNIAKLVASNWYNRQIERSSRTLGCSWEFIPVDDGWGERPL

### EXAMPLES

### Example 1—Suppression of Visceral Pain Following Administration of Clostridial Neurotoxin to a Dermatome

### Introduction to Animal Model

In order to demonstrate that dermatome administration of clostridial neurotoxin can suppress visceral pain, the inventors chose to study pain caused by interstitial cystitis/bladder pain syndrome (IC/BPS) as an exemplary condition. The data demonstrates that appropriate dermatome administration (e.g. to a T12, L1, S2, S3 or S4 dermatome in the case of targeting pain in the bladder) can provide for visceral pain suppression.

Thus, by administering neurotoxin to an appropriate dermatome, visceral pain in any given viscera/organ may be suppressed. By way of example, administration of clostridial neurotoxin to the T11 dermatome can be used to suppress visceral pain of the ovary or fallopian tube(s), because visceral pain from the ovary/fallopian tubes in integrated at the same area from which the T11 spinal nerve emerges. This demonstrates utility in treating visceral pain cause by conditions such as endometriosis.

We now turn more particularly to the animal model and condition studied presently. Interstitial cystitis/bladder pain syndrome (IC/BPS) is a chronic inflammatory disease characterized by visceral pain and urinary symptoms such as urinary frequency. Cyclophosphamide (CYP) induced inflammatory visceral pain is commonly used as an experimental model for IC/BPS in rodents. In rats, CYP treatment leads to accumulation of its toxic metabolite acrolein in the urinary bladder mediating urinary bladder inflammation and visceral pain.

This model is mostly based on single intraperitoneal (i.p.) injection of CYP to induce acute IC/BPS (useful for investigating the acute condition).

We developed a chronic rat model of CYP-induced IC/BPS consisting of 3 injections (40 mg/kg, i.p.) every 3 days. In our protocol, no severe weight loss occurs and the survival rate is 100%. This model shows long-lasting visceral pain which is characterized by both allodynia (painful response to a normally innocuous stimulus) and hyperalgesia (increased response to a noxious stimulus).

The aim of the present study was to evaluate the effects of a single intradermal (i.d.) administration of Dysport (25 U/kg) on visceral pain in the CYP-induced chronic cystitis model in female Sprague-Dawley rats.

### Study Design

### Protocol Design

At D-10, pharmacological treatment was performed as described below (under “pharmacological treatment”).
At D-1, in order to decrease the level of stress due to the new environment, rats were acclimatized to the individual Plexiglas box (von Frey set up) for a minimum of 30 min and to the application of the von Frey filaments. Acclimatization was performed under the same experimental conditions as for pain evaluation described below under “Assessment of visceral pain using von Frey filaments”.
At D0 (basal), before the first injection if CYP, von Frey testing was performed in order to obtain basal values for nociceptive behaviour (see “Assessment of visceral pain using von Frey filaments”).
At D0 (after basal), D3 and D6, chronic cystitis was induced as described below under “Induction of chronic cystitis”
At D7 and D10, von Frey testing was performed to analyze effects of Dysport on CYP-induced chronic visceral pain.
At D10, just after the last von Frey testing, rats were sacrificed and urinary bladders were collected and weighed. Lumbosacral, thoracic and cervical vertebrae, brain and skin were also collected. Collected tissues were processed for histology as described below under “Tissues collection for histological processing”.

### Experimental Groups

Two (2) experimental groups were included as described in the table below:

| Group | Treatment (i.d.) | Injection (i.p.) | n |
|---|---|---|---|
| 1 | Vehicle (20 μL) | CYP | 10 |
| 2 | Dysport (25 U/kg) | CYP | 10 |

### Substances

The test substance was Dysport, supplied in white lyophilisate from IPSEN Innovation, and was stored as −20° C. and protected from light.

Preparation of formulation: formulation was made fresh on the day of administration. One vial containing 519 U of Dysport was solubilized in 1.038 mL of saline to obtain a final concentration of 500 U/mL. Working formulation was prepared according to the mean of rat's body weights from the Dysport group (i.e. 235.7 g at D-11 which is corresponding to a concentration of 294.625 U/mL). Thus, 295 μL of mother solution was added to 205 μL of saline.

Saline was used as vehicle. Saline was purchased from B-Braun via Centravet (Lapalisse, France).

Isoflurane (Aerrane®) was purchased from Baxter Laboratories (Maurepas, France).

Dolethal® was purchased from Vetoquinol via Centravet (Lapalisse, France).

CYP was purchased from Sigma-Aldrich (Saint-Quentin Fallavier, France; batch n° MKCG5464).

Formaldehyde 4% was purchased from VWR (Fontenay sous bois, France).

Phosphate buffered saline (PBS) was purchased from Sigma-Aldrich.

### Study Protocols

### Pharmacological Treatments

Prior to the experiment, the animals were randomly assigned to treatment groups using the block method, which consists of distributing at least one animal per treatment or control (vehicle) in the same block. Intradermal administration was performed as follows: anaesthesia was induced by 5% isoflurane and then maintained with 3% isoflurane for the duration of the procedure. The abdominal area designed for mechanical stimulation of each animal was shaved. Dysport or vehicle (20 μL/rat) was administered via intradermal route. Injection was performed with an insulin syringe at 1 site in the ventral area which is the site of von Frey test (see FIG. 3).

### Assessment of Visceral Pain Using Von Frey Filaments

Standardized conditions including single-experimenter testing of all animals were applied to minimize variability behavior-based pain testing.

Visceral pain was evaluated in a blinded manner by applying to the lower abdomen, close to the urinary bladder, a set of 8 calibrated von Frey filaments of increasing forces (1, 2, 4, 6, 8, 10, 15 and 26 g) with an interstimulus interval of 5 seconds. Animals were placed on a raised wire mesh floor under individual transparent Plexiglas box and acclimatized for at least 30 minutes before starting the von Frey test.

Filaments were then applied for 1-2 seconds through the mesh floor with enough strength to cause the filament to slightly bend. Each filament was tested 3 times. Care was taken to stimulate different areas within the lower abdominal region in the vicinity of the urinary bladder to avoid desensitization.

Nociceptive behaviours were scored for each animal and each filament as follows:

| Score | Behaviour |
|---|---|
| 0 | No response |
| 1 | Retraction of the abdomen |
| 2 | Trampling or change of position |
| 3 | flinching or abdominal curvature or licking of the site |
| | stimulated with von Frey filaments |

### Induction of Chronic Cystitis

To induce chronic cystitis, at D0, D3 and D6, rats were weighted and an intraperitoneal (i.p.) injection of CYP at a dose of 40 mg/kg in a final volume of 5 mL/kg was performed. CYP was prepared fresh in saline at a final concentration of 8 mg/mL.

### Tissues Collection for Histological Processing

Animals were deep anesthetized by injection of Dolethal® (pentobarbital solution at 182.2 mg/mL, 0.25 mL/rat, i.p.). Sedated rats were then euthanatized by a second lethal injection of Dolethal® (0.25 mL/rat). Both i.p. injections were made outside the area of skin collection.

The skin including the injection site with the underlying abdominal muscles was first collected. The piece of around 2-3 of length was stapled on a plastic sheet and placed into a 500 mL jar containing 4% buffered formaldehyde.

Bladder was then excised and lipoid tissue surrounding the bladder was cut away. Bladder was weighed and placed into a tissue processing embedding cassette. Cassette was immerged into 4% buffered formaldehyde.

Finally the brain, and the lumbosacral, thoracic and cervical vertebrae (with spinal cord+DRGs) were collected. The 3 segments and the brain were placed together in the jar with the skin. To collect the lumbrosacral, thoracic and cervical vertebrae (with spinal cord and DRGs), with scissors, all the organs around the spine were cut in taking care that the ribs could still be seen. The ventral part faced up. The tissue above the thoraco-lumbar area was scratched with a scalpel. The cervical part was separated from the thoracic part by cutting with a scalpel between the third and fourth last thoracic intervertebral discs/ribs. The third/fourth ribs remained attached to the lumbar part.

Bladder and all others collected tissues were fixed at room temperature for around 24 h and 48h, respectively. Then samples were transferred into 4% buffered formaldehyde (¼)/PBS (¾) solution and stored at +4° C.

Immunohistochemistry (IHC) for cleaved SNAP25 (c-SNAP25_{A}) was performed with antibody EF14007, 1/6000 dilution, incubated with sample overnight—same for any related IHC described herein.

### Laboratory Equipment

Mechanical stimulation was performed using von Frey filaments of different forces (reference: Bio-VFM; BioSeb ID Tech, Vitrolles, France). Filaments were purchased pre-calibrated.
Animals were weighed using a LS620C balance (PRECISA, Dietikon, Switzerland).
Urinary bladders were weighed using a Scout® Pro balance (OHAUS, Nanikon, Switzerland).

### Expression and Analysis of Results

All raw data were entered into an Excel® spreadsheet. All data entered have been compared with raw data by two persons before data analysis. Results are expressed as mean values±standard error of the mean (SEM).

### Visceral Pain

| Nociceptive | Expres- | |
|---|---|---|
| parameter | sion | Description |
| Nociceptive | g | von Frey force for which the stimulus |
| threshold | | is perceived as painful (score ≥1 |
| | | is obtained) |
| Nociceptive | % | % of the maximal response for each |
| scores | | filament (on the 3 pooled |
| | | applications*) |
| Area under the | % | calculated from the curve of % nocicep- |
| curve (AUC) 1-6 g - | scores × | tive scores plotted against painless (1 |
| allodynia - | g | to 6 g) von Frey forces |
| Area under the curve | | calculated from the curve of % nocicep- |
| (AUC) 6-26 g - | | tive scores plotted against painful (6 |
| hyperalgesia - | | to 26 g) von Frey forces |

*For example, at a given von Frey force, for an animal with a score of 1 at the first application, 1 at the second and 2 at the third, the summation of its scores is 4. The maximal pooled score being 9 (3 + 3 + 3), a pooled score of 4 equals 44% of the maximal response (100 × 4/9).

### Bladder Weight

Results of bladder weight are expressed as follows: Bladder weight in g; Bladder weight as a ratio of body weight in g/kg.

### Statistical Analysis

Statistical analysis and graphs were performed using GraphPad Prism® (GraphPad Software Inc., La Jolla, CA, USA). A P value <0.05 was accepted for statistical significance. Except when two-way analysis was applied, before carrying out any statistical test, the data were tested for normal distribution (Shapiro-Wilk normality test) and their variance evaluated (F test). The appropriate statistical test was consequently applied.

### Effects of CYP

As no non-pathological experimental group (i.e. saline) was included in the study, effects of CYP were statistically analyzed by grouped comparison between basal value (i.e. before CYP injection) and post-CYP values (i.e. D7 and D10) within the Vehicle group.
Friedman test was used as non-parametric test of analysis of variance (ANOVA) with repeated measures (RM) followed by Dunn's multiple comparisons versus basal value.
One-way RM ANOVA was used as parametric test with Holm-Sidak's multiple comparisons versus basal value.
Two-way RM ANOVA with both factors was used followed by Sidak's multiple comparisons versus basal value was used for nociceptive scores.

### Effects of Dysport

To analyse effects of Dysport, individual pairwise comparisons were made versus vehicle group.
Unpaired t test was used as parametric t test.
Mann-Whitney test was used as non-parametric t test.
A Two-way RM ANOVA was used for nociceptive scores.

It is of note that when individual pairwise comparison was made, statistical significance was indicated by the symbol “*” and non-significance by “ns”. For grouped comparison statistical significance was indicated by the symbol “#”.

### Results

Repeated CYP Injections (40 mg/kg, i.p.) Induced Chronic Visceral Pain

In order to validate our CYP-induced visceral pain model, we compared the nociceptive parameters post-CYP injection (i.e. at D7 and D10) with the corresponding basal value (i.e. at D0) within the Vehicle group.

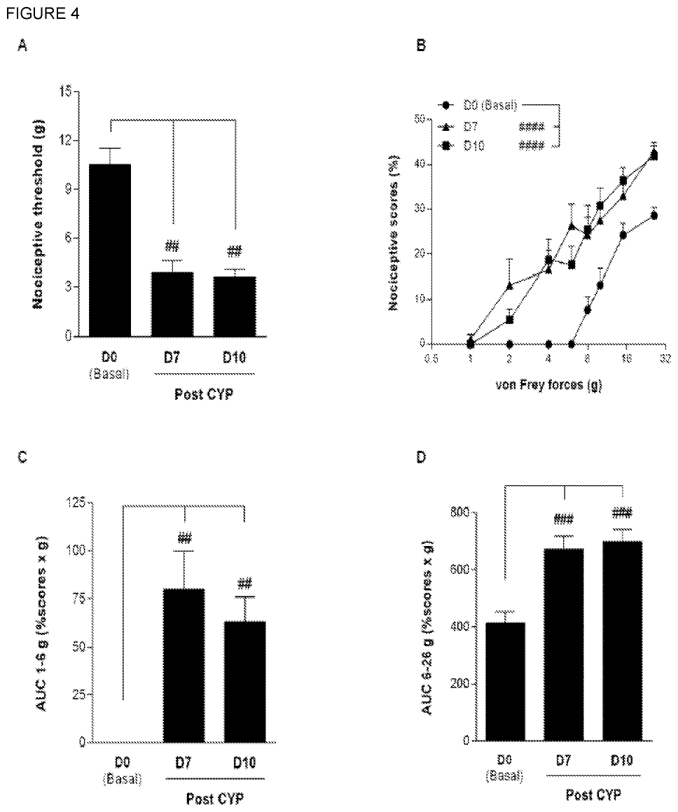

At both evaluated time points, CYP elicited a significant decrease of nociceptive threshold as compared to basal value (P<0.01, FIG. 4A).

Furthermore, CYP-injected rats exhibited a significant increase of nociceptive scores at D7 and D10 (P<0.0001, FIG. 4B).

CYP effects on nociceptive scores were paralleled with a significant increase in corresponding AUCs 1-6 and 6-26 g at D7 and D10 (P<0.01 and P<0.001, FIGS. 4C and D, for AUC 1-6 and 6-26 g, respectively).

For all nociceptive parameters, similar level of significance was obtained for both time points (FIG. 4).

### Dysport (25 U/Rat, i.d.) Diminished CYP-Induced Chronic Visceral Pain

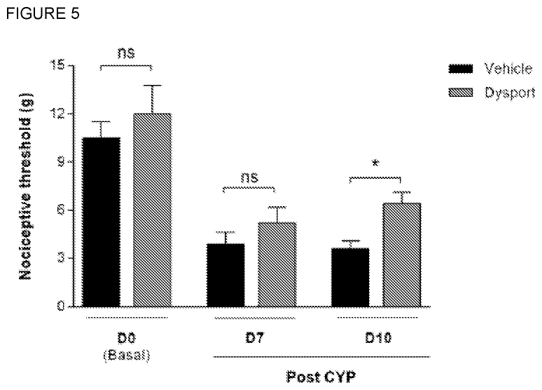

Before CYP injection (i.e. basal values), no significant difference in all nociceptive parameters was observed between both experimental groups (P>0.05, FIGS. 5, 6A and 7; “D0”). It is of note that basal assessment was performed on vehicle- or Dysport-treated rats.

In CYP-injected rats, Dysport treatment at 25 U/kg led to an increase in nociceptive threshold at D7 and D10, in particular reaching statistical significance at D10 (P>0.05 and P<0.05, FIG. 5 for “D7” and “D10”, respectively).

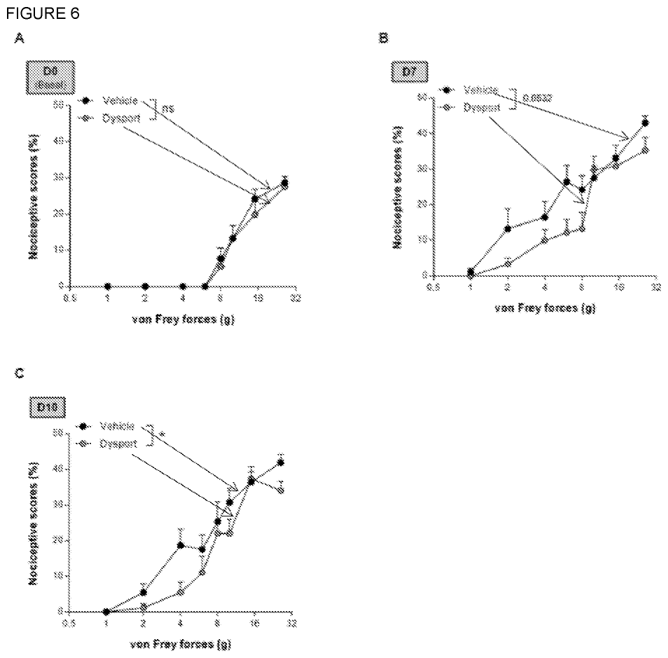

In accordance with these findings, as compared to Vehicle, a decrease in nociceptive scores was observed in Dysport-treated rats at both time points (FIGS. 6B and C). At D7, Dysport effect was above the statistical significance threshold (P=0.0532, FIG. 6B), with particular significance seen at D10 (P<0.05, FIG. 6C).

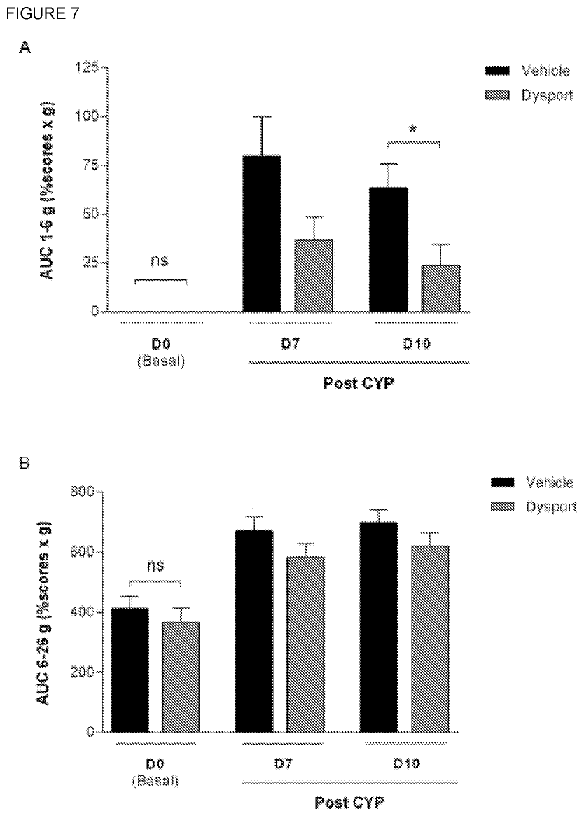

A decrease in nociceptive scores elicited by Dysport treatment was associated with decrease in AUC 1-6 g at both time points (FIG. 7A). Similarly to above results, the effect of Dysport was at close to a statistically significant level at D7 (P=0.0812, FIG. 7A; “D7”), with particular significance at D10 (P<0.05, FIG. 7A; “D10”).

Finally, a tendency towards a decreased AUC 6-26 g was observed at D7 and D10 in the Dysport group as compared to vehicle but, for both time points (P>0.05, FIG. 7B; “D7” and “D10”), although the effect was more pronounced for AUC 1-6 g.

c-SNAP 25 Immunohistochemistry (IHC) Results in Thoracic Spinal Cord

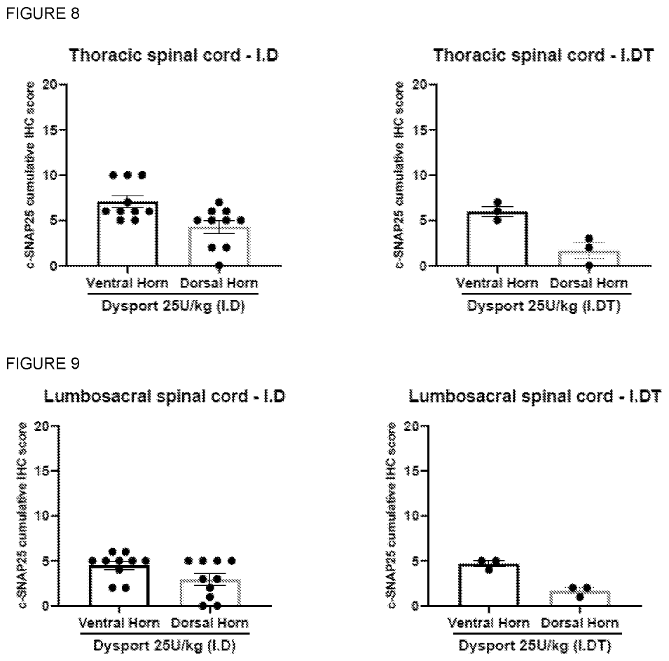

Following intradermal (I.D.) injection of Dysport to a dermatome nerve, c-SNAP25 staining was seen in the thoracic spinal cord, at both ventral and dorsal horns. Interestingly, such c-SNAP25 staining was also seen following or intradetrusor (I.DT) injection (injection into the detrusor muscle of the bladder), for example similar localization, distribution and severity of c-SNAP25 staining following 1.D and I.DT administrations were seen in the thoracic spinal cord. This suggests that I.D Dysport administration at the abdomen level resulted in the same retrograde transport (i.e. to the same area of spinal cord) as I.DT administration, the latter believe to be due to retrograde axonal transport via visceral afferents. See FIG. 8.

c-SNAP25 IHC Results in Lumbosacral Spinal Cord

Following intradermal (I.D.) injection of Dysport, c-SNAP25 staining was seen in the lumbrosacral spinal cord, at both ventral and dorsal horns. Interestingly, such c-SNAP25 staining was also seen following or intradetrusor (I.DT) injection (injection into the detrusor muscle of the bladder), for example similar localization, distribution and severity of c-SNAP25 staining following 1.D and I.DT administrations were seen in the lumbrosacral spinal cord. This suggests that I.D Dysport administration at the abdomen level resulted in the same retrograde transport (i.e. to the same area of spinal cord) as I.DT administration, the latter believe to be due to retrograde axonal transport via visceral afferents. See FIG. 9.

c-SNAP25 IHC Results in Spinal Cord of Pig

To complement this rodent model data, the inventors also analysed SNAP-25 cleavage in the spinal cord of pigs following Dysport injection. Male domestic pigs weighing 11-13 kg were used in the following study. The pig advantageously shares similarities with human skin in terms of structure, thickness, innervation, pigmentation, collagen and lipid composition, wound-healing and immune responses.

Detailed materials and methods with regard to pig handling and experimentation with the pig model is described in PCT/GB2021/051838, which is incorporated herein by reference. Briefly, Dysport was provided in vials containing 500U. For dosing, vials of 500U were reconstituted with saline (0.9% NaCl). Dysport or saline (negative control) were injected intradermally using 30G needles attached to 1 ml syringes and into 10 sites spread around an incision on the left leg of the pig (20 U Dysport injected into each site)—6 animals (8-10 weeks old) were used.

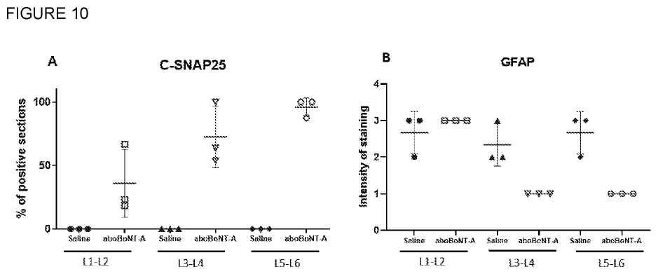

Immunohistochemistry was performed on tissue/skin samples at the site of Dysport injection (left leg of the pig) and at different regions of the spinal cord (see FIG. 10). Cleaved SNAP-25 was not detected in the nerves of skin samples on samples collected 5-7 days after the injection of Dysport. Cleaved SNAP-25 was visualised in the ipsilateral dorsal horn of the spinal cord, e.g. in lumbar regions L5-L6 (see FIG. 10) of Dysport injected pigs (but not saline injected pigs). The spinal nerves (L5-L6) of these lumbar regions map to and innervate dermatomes on the left leg (e.g. where the Dysport was injection), and are indicative of BoNT/A activity in the spinal cord and that BoNT/A is transported to this region of the spinal cord after the intradermal injection to the dermatome. The localisation of cleaved SNAP-25 staining in the ipsilateral dorsal horn was different when compared to cleaved SNAP-25 staining in pigs where Dysport was injected (intradermally) to their left flank, indicating that injecting into different dermatomes allows for BoNT/A activity in different parts of the spinal cord.

Furthermore, expression levels of a marker of astrocyte activation, GFAP, were decreased in the spinal cord, particularly in the dorsal horns, of pigs (those treated with Dysport when compared to untreated) in the same areas that an increase in SNAP-25 cleavage was seen.

### Conclusions

The aim of the present study was to evaluate the effects of intradermal administration of botulinum toxin (Dysport) at 25 U/kg on visceral pain in the chronic CYP-induced cystitis model in female Sprague-Dawley rats.

Consistent with previous and published data, 3 injections of CYP (40 mg/kg, i.p.) induced chronic visceral pain (up to 10 days). Effects of CYP were characterized by allodynia (i.e. decreased nociceptive threshold and increased AUC 1-6 g) and hyperalgesia (i.e. increased AUC 6-26 g).

This study showed that single injection of clostridial neurotoxin (e.g. BoNT/A aka Dysport) to a dermatome nerve, particularly via intradermal injection, provided anti-nociceptive properties on CYP-induced visceral pain. Allodynia and hyperalgesia induced by CYP were lowered by clostridial neurotoxin/Dysport (intradermal) injection, with allodynia being a particular target for suppression. Dysport effect was long lasting as clostridial neurotoxin/Dysport was still efficient 20 days after its administration.

### Example 2—Suppression of Pain Caused by Bladder Pain Syndrome

A patient presents with severe pelvic pain perceived as visceral pain, and abnormal urination frequency. The patient is diagnosed with interstitial cystitis/bladder pain syndrome (IC/BPS), and the bladder is identified as the organ contributing to the visceral pain.

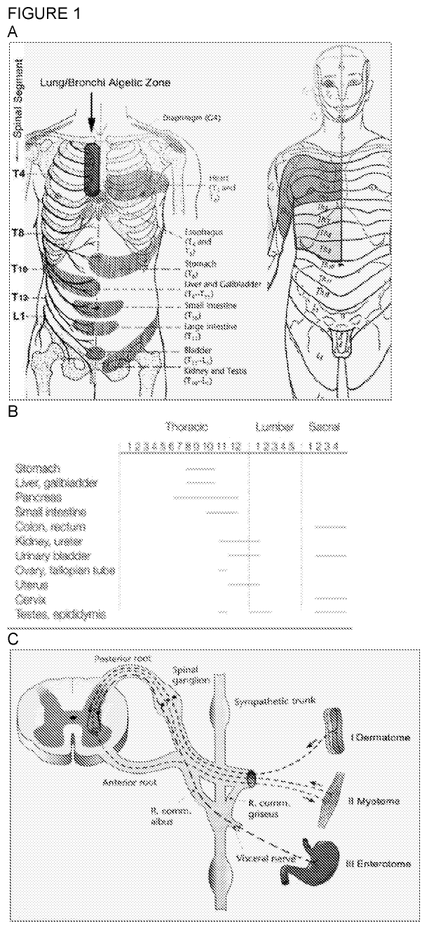

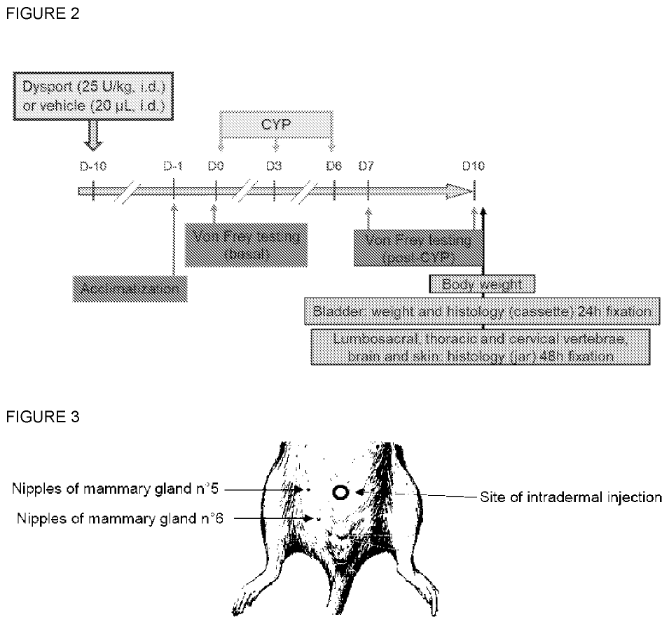

In order to determine which dermatome to administer a clostridial neurotoxin to in order to suppress the visceral (bladder) pain, a map of the dermatomes and their corresponding organs/viscera is consulted, such as that outlined in the American Spinal Injury Association's (ASIA) worksheet produced as the International Standards for Neurological Classification of Spinal Cord Injury (ISNCSCI). At the time of writing, the 2019 edition of said worksheet is obtainable here: https://asia-spinalinjury.org/wp-content/uploads/2019/10/ASIA-ISCOS-Worksheet_10.2019_PRINT-Page-1-2.pdf. Further information is derived from Wall and Melzack's “Textbook of Pain” (edited by McMahon et al.; 6th edition)—see chapter 53, and Figure 53-1 (partially adapted herein as FIG. 1B). This figure illustrates the segmental level of visceral innervation and the corresponding dermatomes that various viscera e.g. the dermatomes that ‘map’ to the viscera. It is noted that T12, L1, S2, S3 and S4 dermatomes map to the urinary bladder.

Thus, clostridial neurotoxin (5,000 pg Dysport) is administered to the T12 dermatome by intradermal administration to the skin in the proximity of the intersection of the midclavicular line and the midpoint of the inguinal ligament. After 10 days, levels of visceral (bladder) pain are assessed, and found to be significantly reduced. Suppression of visceral (bladder) pain is also noted at 30 days post-administration of the clostridial neurotoxin and at 90 days post-administration.

### Example 3—Suppression of Pain Caused by Endometriosis

A female patient presents with severe pelvic pain, perceived as visceral pain, worsening during menstruation. The patient is diagnosed by her GP with endometriosis, and the ovaries are identified as the organ contributing to the visceral pain.

In order to determine which dermatome to administer a clostridial neurotoxin to in order to suppress the visceral (ovary) pain, a map of the dermatomes and their corresponding organs/viscera is consulted, such as that outlined in the American Spinal Injury Association's (ASIA) worksheet produced as the International Standards for Neurological Classification of Spinal Cord Injury (ISNCSCI). At the time of writing, the 2019 edition of said worksheet is obtainable here: https://asia-spinalinjury.org/wp-content/uploads/2019/10/ASIA-ISCOS-Worksheet_10.2019_PRINT-Page-1-2.pdf. Further information is derived from Wall and Melzack's “Textbook of Pain” (edited by McMahon et al.; 6th edition)—see chapter 53, and Figure 53-1 (partially adapted herein as FIG. 1B). This figure illustrates the segmental level of visceral innervation and the corresponding dermatomes that various viscera e.g. the dermatomes that ‘map’ to the viscera. It is noted that the T11 dermatome maps to the ovaries.

Thus, clostridial neurotoxin (5,000 pg Dysport) is administered to the T11 dermatome by intradermal administration to the skin in the proximity of the intersection of the midclavicular line, at the horizontal level midway between the level of the umbilicus and the inguinal ligament. After 10 days, levels of visceral (ovary) pain are assessed, and found to be significantly reduced. Suppression of visceral (ovary) pain is also noted at 30 days post-administration of the clostridial neurotoxin and at 90 days post-administration.

### Example 4—Suppression of Pain Caused by Pancreatitis

A patient presents with severe abdominal pain, perceived as visceral pain. Inflammation of the pancreas is noted, the patient is diagnosed by his GP with pancreatitis, and the pancreas is identified as the organ contributing to the visceral pain.

In order to determine which dermatome to administer a clostridial neurotoxin to in order to suppress the visceral (pancreas) pain, a map of the dermatomes and their corresponding organs/viscera is consulted, such as that outlined in the American Spinal Injury Association's (ASIA) worksheet produced as the International Standards for Neurological Classification of Spinal Cord Injury (ISNCSCI). At the time of writing, the 2019 edition of said worksheet is obtainable here: https://asia-spinalinjury.org/wp-content/uploads/2019/10/ASIA-ISCOS-Worksheet_10.2019_PRINT-Page-1-2.pdf. Further information is derived from Wall and Melzack's “Textbook of Pain” (edited by McMahon et al.; 6th edition)—see chapter 53, and Figure 53-1 (adapted herein as FIG. 1A). This figure illustrates the segmental level of visceral innervation and the corresponding dermatomes that various viscera e.g. the dermatomes that ‘map’ to the viscera. It is noted that the T7-T12 dermatomes map to the pancreas.

Thus, clostridial neurotoxin (5,000 pg Dysport) is administered to the T10 dermatome by intradermal administration to the skin in the proximity of the intersection of the midclavicular line, at the horizontal level of the umbilicus. After 10 days, levels of visceral (pancreas) pain are assessed, and found to be significantly reduced. Suppression of visceral (pancreas) pain is also noted at 30 days post-administration of the clostridial neurotoxin and up to 90 days post-administration.

### Example 5—Suppression of Pain Caused by Gastric Ulcers

A patient presents with severe abdominal pain, perceived as visceral pain. The patient is diagnosed by his GP with gastric ulcers, and the stomach is identified as the organ contributing to the visceral pain.

In order to determine which dermatome to administer a clostridial neurotoxin to in order to suppress the visceral (stomach) pain, a map of the dermatomes and their corresponding organs/viscera is consulted, such as that outlined in the American Spinal Injury Association's (ASIA) worksheet produced as the International Standards for Neurological Classification of Spinal Cord Injury (ISNCSCI). At the time of writing, the 2019 edition of said worksheet is obtainable here: https://asia-spinalinjury.org/wp-content/uploads/2019/10/ASIA-ISCOS-Worksheet_10.2019_PRINT-Page-1-2.pdf. Further information is derived from Wall and Melzack's “Textbook of Pain” (edited by McMahon et al.; 6th edition)—see chapter 53, and Figure 53-1 (partially adapted herein as FIG. 1B). This figure illustrates the segmental level of visceral innervation and the corresponding dermatomes that various viscera e.g. the dermatomes that ‘map’ to the viscera. It is noted that the T8-T10 dermatomes map to the pancreas.

Thus, clostridial neurotoxin (5,000 pg Dysport) is administered to the T8 dermatome by intradermal administration to the skin in the proximity of the intersection of the midclavicular line and the horizontal level at one half the distance between the level of the xiphoid process and the level of the umbilicus. After 10 days, levels of visceral (stomach) pain are assessed, and found to be significantly reduced. Suppression of visceral (stomach) pain is also noted at 30 days post-administration of the clostridial neurotoxin and at 90 days post-administration.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A clostridial neurotoxin for use in a method of suppressing visceral pain in a patient, the method comprising:
a. identifying an organ in the patient that is causing said visceral pain;
b. mapping said organ to a specific dermatome nerve according to the following instructions, thereby matching said organ to one or more specifically designated dermatome nerve(s):
i. urinary bladder maps to the T11, T12, L1, S1, S2, S3 and/or S4 dermatome nerve(s);
ii. fallopian tube(s) maps to the T11 dermatome nerve(s);
iii. uterus maps to the T12 and/or L1 dermatome nerve(s);
iv. cervix maps to the S2, S3 and/or S4 dermatome nerve(s);
v. stomach maps to the T8, T9 and/or T10 dermatome nerve(s);
vi. liver maps to the T8, T9, T10 and/or T11 dermatome nerve(s);
vii. gall bladder maps to the T8, T9, T10 and/or T11 dermatome nerve(s);
viii. pancreas maps to the T7, T8, T9, T10, T11 and/or T12 dermatome nerve(s);
ix. small intestine maps to the T10, T11 and/or T12 dermatome nerve(s);
x. colon maps to the T11, S1, S2, S3 and/or S4 dermatome nerve(s);
xi. kidney maps to the T10, T11, T12 and/or L1 dermatome nerve(s);
xii. ureter maps to the T11, T12 and/or L1 dermatome nerve(s);
xiii. ovary maps to the T11 dermatome nerve(s);
xiv. testes maps to the T10, T11, L1, L2, S1, S2, S3 and/or S4 dermatome nerve(s);
xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain,
wherein the clostridial neurotoxin is administered by intradermal administration.

2. A method of suppressing visceral pain in a patient, the method comprising:
a. identifying an organ in the patient that is causing said visceral pain;
b. mapping said organ to a specific dermatome nerve according to the following instructions, thereby matching said organ to one or more specifically designated dermatome nerve(s):
i. urinary bladder maps to the T11, T12, L1, S1, S2, S3 and/or S4 dermatome nerve(s);
ii. fallopian tube(s) maps to the T11 dermatome nerve(s);
iii. uterus maps to the T12 and/or L1 dermatome nerve(s);
iv. cervix maps to the S2, S3 and/or S4 dermatome nerve(s);
v. stomach maps to the T8, T9 and/or T10 dermatome nerve(s);
vi. liver maps to the T8, T9, T10 and/or T11 dermatome nerve(s);
vii. gall bladder maps to the T8, T9, T10 and/or T11 dermatome nerve(s);
viii. pancreas maps to the T7, T8, T9, T10, T11 and/or T12 dermatome nerve(s);
ix. small intestine maps to the T10, T11 and/or T12 dermatome nerve(s);
x. colon maps to the T11, S1, S2, S3 and/or S4 dermatome nerve(s);
xi. kidney maps to the T10, T11, T12 and/or L1 dermatome nerve(s);
xii. ureter maps to the T11, T12 and/or L1 dermatome nerve(s);
xiii. ovary maps to the T11 dermatome nerve(s);
xiv. testes maps to the T10, T11, L1, L2, S1, S2, S3 and/or S4 dermatome nerve(s);
xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain,
wherein the clostridial neurotoxin is administered by intradermal administration.

3. Use of a clostridial neurotoxin in the manufacture of a medicament for suppressing visceral pain in a patient, wherein suppressing visceral pain comprises:
a. identifying an organ in the patient that is causing said visceral pain;
b. mapping said organ to a specific dermatome nerve according to the following instructions, thereby matching said organ to one or more specifically designated dermatome nerve(s):
i. urinary bladder maps to the T11, T12, L1, S1, S2, S3 and/or S4 dermatome nerve(s);
ii. fallopian tube(s) maps to the T11 dermatome nerve(s);
iii. uterus maps to the T12 and/or L1 dermatome nerve(s);
iv. cervix maps to the S2, S3 and/or S4 dermatome nerve(s);
v. stomach maps to the T8, T9 and/or T10 dermatome nerve(s);
vi. liver maps to the T8, T9, T10 and/or T11 dermatome nerve(s);
vii. gall bladder maps to the T8, T9, T10 and/or T11 dermatome nerve(s);
viii. pancreas maps to the T7, T8, T9, T10, T11 and/or T12 dermatome nerve(s);
ix. small intestine maps to the T10, T11 and/or T12 dermatome nerve(s);
x. colon maps to the T11, S1, S2, S3 and/or S4 dermatome nerve(s);
xi. kidney maps to the T10, T11, T12 and/or L1 dermatome nerve(s);
xii. ureter maps to the T11, T12 and/or L1 dermatome nerve(s);
xiii. ovary maps to the T11 dermatome nerve(s);
xiv. testes maps to the T10, T11, L1, L2, S1, S2, S3 and/or S4 dermatome nerve(s);
xv. epididymis maps to the T10, T11, L1, L2, S1, S2, S3 and/or S4 dermatome nerve(s); and
c. administering a therapeutically effective amount of a clostridial neurotoxin at a site proximal to said one or more specifically designated dermatome nerve(s), wherein following administration of said clostridial neurotoxin to the patient, the clostridial neurotoxin is transported via retrograde axonal transport to the spinal cord where it binds sensory afferent nerve cells, enters said cells by receptor-mediated endocytosis and suppresses neurotransmitter release therefrom, thereby suppressing said visceral pain,
wherein the clostridial neurotoxin is administered by intradermal administration.

4. The clostridial neurotoxin for use according to claim 1, the method according to claim 2, or the use according to claim 3, wherein said visceral pain is caused by a condition selected from bladder pain syndrome (interstitial cystitis), bladder stones, cirrhosis, coeliac disease, Crohn's disease, endometriosis, gallbladder stones, gastric ulcers, irritable bowel syndrome, kidney stones, pancreatitis, peritonitis and ulcerative colitis.

5. The clostridial neurotoxin for use, method or use according to any one of the preceding claims, wherein said visceral pain is caused by bladder pain syndrome (interstitial cystitis).

6. The clostridial neurotoxin for use, method or use according to claim 5, wherein said dermatome nerve(s) is selected from the T12, L1, S2, S3 and S4 dermatome nerve(s).

7. The clostridial neurotoxin for use, method or use according to any one of claims 1-4, wherein said visceral pain is caused by endometriosis.

8. The clostridial neurotoxin for use, method or use according to claim 7, wherein the visceral pain is perceived as dysmenorrhea, chronic pelvic pain (CPP), dyspareunia, and/or chronic back pain.

9. The clostridial neurotoxin for use, method or use according to claim 7 or claim 8, wherein said dermatome nerve(s) is selected from the T11, T12, L1, S2, S3 and S4 dermatome nerve(s).

10. The clostridial neurotoxin for use, method or use according to any one of the preceding claims, wherein said visceral pain comprises or consists of allodynia.

11. The clostridial neurotoxin for use, method or use according to any one of the preceding claims, wherein said visceral pain is suppressed for at least 30 days following administration of the clostridial neurotoxin.

12. The clostridial neurotoxin for use, method or use according to any one of the preceding claims, wherein said visceral pain is suppressed for at least 40 days following administration of the clostridial neurotoxin.

13. The clostridial neurotoxin for use, method or use according to any one of the preceding claims, wherein the clostridial neurotoxin is administered to more than one dermatome nerve(s) that that maps to the organ identified as contributing to the visceral pain.

14. The clostridial neurotoxin for use, method or use according to any one of the preceding claims, wherein the clostridial neurotoxin is administered at 4 or more injection sites per treatment session.

15. The clostridial neurotoxin for use, method or use according to any one of the preceding claims, wherein the clostridial neurotoxin is a botulinum neurotoxin (BoNT).

16. The clostridial neurotoxin for use, method or use according to any one of the preceding claims, wherein the clostridial neurotoxin is botulinum neurotoxin serotype A (BoNT/A).

17. The clostridial neurotoxin for use, method or use according to any one of the preceding claims, wherein the clostridial neurotoxin is a chimeric neurotoxin.
